SEQUENCE LISTING (228 sequences)

SEQ ID NO: 1 - protein, 337 aa, Lobesia botrana
  misc_feature (308)..(308): Xaa is Asp or Ala
  misc_feature (317)..(317): Xaa is Asp or His
MVPRAASEETDLKEATQLEPRKYEIVYTNVIYFTYWHIAGLYGLYLCFTSAKWETIVFAWAWYVLGELGVIAGAHRLWAHRTYKAKMPLQIILMLFNCIGFQNTATDWVRDHRVHHKHSDTDADPHNSQRGFFFSHVGWLLTRKHRLVKEKGEAVDMTDIYSNPVLRFQKKYSLPLIGTLCFALPTLLPAYCWGEAVGTAWNINLLRYCLNLNGTFLTNSAAHKFGSKPYDKTIPPTQNLLVSFMTLGEGFHNYHHVFSWDYRAAELGNTYLNMTTIFIDFFALIGWAYDLKTVPEDVIKKRMARTGDGTNLWGWGDKDMTKEDVVDTEIRFPAKKE

SEQ ID NO: 2 - protein, 358 aa, Lobesia botrana
MAPNKKEIAEPAPDIDNEFRALVAPQAGPRKYTVVYMTVLNFLYWHLGGLYGLYLCFTSAKWATIFFNAILHVLAVWGITAGAHRLWSHRSYKAKLPLQILLMLMHTLAFQNPAIHWARDHRLHHKYSDTDADPHNATRGFFYSHIGWLLVRKHPEAIKRGKTIDVTDIIQNPVLQIQKKYYLYLSGMLSFVLPALIPMFFWGETLNNAHHVSLLRLIIVFNVTFFVNSAAHKYGNKPYDMNMSPTQSVTLSLATLGECFHNYHHVFPFDYRTAELGNNYINFTTKFIDFFAAIGWAYDLKYASDDMIMKRSNRTGDGTDLWGRVKSTNDEQAPKNVWGWGDEDMVEEHKKVLLSQNQ

SEQ ID NO: 3 - protein, 371 aa, Choristoneura parallela
MDFLNEIDNCPERLRKPEKMAPNVTEENGVLFESDAATPDLALARTPVEQADDSPRIYVWRNIILFAYLHLAAIYGGYLFLFSAKWQTDIFAYLLYVASGLGITAGAHRLWAHKSYKAKWPLRLILTIFNTIAFQDSAIDWARDHRMHHKYSETDADPHNATRGFFFSHIGWLLVRKHPELKRKGKGLDLSDLYSDPILRFQKKYYMILMPLACFILPTVIPVYMWNETWSNAFFVAALFRYTFILNVTWLVNSAAHKWGDKPYDKSIKPSENMSVSLFAFGEGFHNYHHTFPWDYKTAELGNHRLNFTTKFINFFAKIGWAYDMKTVSQEIVQQRVKRTGDGSHHLWGWGDKDHAQEEINAAIRINPKDD

SEQ ID NO: 4 - protein, 371 aa, Artificial Sequence
  DST192 G100L
MDFLNEIDNCPERLRKPEKMAPNVTEENGVLFESDAATPDLALARTPVEQADDSPRIYVWRNIILFAYLHLAAIYGGYLFLFSAKWQTDIFAYLLYVASLLGITAGAHRLWAHKSYKAKWPLRLILTIFNTIAFQDSAIDWARDHRMHHKYSETDADPHNATRGFFFSHIGWLLVRKHPELKRKGKGLDLSDLYSDPILRFQKKYYMILMPLACFILPTVIPVYMWNETWSNAFFVAALFRYTFILNVTWLVNSAAHKWGDKPYDKSIKPSENMSVSLFAFGEGFHNYHHTFPWDYKTAELGNHRLNFTTKFINFFAKIGWAYDMKTVSQEIVQQRVKRTGDGSHHLWGWGDKDHAQEEINAAIRINPKDD

SEQ ID NO: 5 - protein, 329 aa, Ostrinia latipennis
MAQYATSAEGLPVKDECFEDKEIKSNSFPKLEILYFNVMTFTLLHLSALYGLYLGFTSVKWATIGIGIVLYFFAEIGITAGAHRLWSHRSYKAKLPLEILLMVFNSMARQNTALSWARDHRLHHKCPDTNGDPHNANRGFFYSHVGWLLTKKSDEVIKQGKLCDVADLYSNPVLRFQKKYAVPFIGMLCFVLPTLIPMYFWGETLNNAWHFNMFRYVVNLNATFCVNSVVHKWGYKPYDKNIRPTQNVLLNFAVLGEAFHNFHHVFPWDYRAAELGNQKMNPTTLFIDFFAWIGWAYDLKTASKEMVKSRSERTGDGTDLWGHDADKLK

SEQ ID NO: 6 - protein, 329 aa, Artificial Sequence
  DST109 V230A
MAQYATSAEGLPVKDECFEDKEIKSNSFPKLEILYFNVMTFTLLHLSALYGLYLGFTSVKWATIGIGIVLYFFAEIGITAGAHRLWSHRSYKAKLPLEILLMVFNSMAFQNTALSWARDHRLHHKCPDTNGDPHNANRGFFYSHVGWLLTKKSDEVIKQGKLCDVADLYSNPVLRFQKKYAVPFIGMLCFVLPTLIPMYFWGETLNNAWHFNMFRYVVNLNATFCVNSVAHKWGYKPYDKNIRPTQNVLLNFAVLGEAFHNFHHVFPWDYRAAELGNQKMNPTTLFIDFFAWIGWAYDLKTASKEMVKSRSERTGDGTDLWGHDADKLK

SEQ ID NO: 7 - protein, 319 aa, Ostrinia latipennis
MAETSSDPNETPADENLPKNKRGSKIIYKYVTSFTILHLAAFYGLYLCLTKAKWLTIGFSLLMYELSILGISAAAHRLWSHRSYKAKFPLQVILMVLNCMSFQNSALNWCRDHRVHHKCSDTDGDPHNASRGFFFSHIGWLMTHKSEEVKRQGNSIDMSDLYNNPVLRFQKKYAVPLIGSICFIFPTLIPVYFWGETINNAWHMNILRYVMALHATLCINSVAHRWGYKTYDKNILPSQNMLLNTAMLGEGFHNYHHVFPWDYQSSELGNRKFNPTTWFIDFFAWVGWAYDLKTTSEEVIRKRAERTGDGTDLWGAKRN

SEQ ID NO: 8 - protein, 361 aa, Drosophila melanogaster
MAPYSRIYHQDKSSRETGVLFEDDAQTVDSDLTTDRFQLKRAEKRRLPLVWRNIILFALVHLAALYGLHSIFTRAKLATTLFAAGLYIIGMLGVTAGAHRLWAHRTYKAKWPLRLLLVIFNTIAFQDAVYHWARDHRVHHKYSETDADPHNATRGFFFSHVGWLLCKKHPDIKEKGRGLDLSDLRADPILMFQRKHYYILMPLACFVLPTVIPMVYWNETLASSWFVATMFRWCFQLNMTWLVNSAAHKFGNRPYDKTMNPTQNAFVSAFTFGEGWHNYHHAFPWDYKTAEWGCYSLNITTAFIDLFAKIGWAYDLKTVAPDVIQRRVLRTGDGSHELWGWGDKDLTAEDARNVLLVDKSR

SEQ ID NO: 9 - protein, 352 aa, Dendrolimus punctatus
MAPKEADVNGVLFESDATTPDMALPTTPVQQADNYPKKLVWRNILLFAYLHLAALYGGYLFLFSAKWQTDIFAYILYIMSGLGITAGAHRLWAHKSYKAKWPLRLILVLFNTLAFQDSAIDWARDHRMHHKYSETDADPHNATRGFFFSHIGWLLVRKHPELKKKGKGLDISDLYADPILRFQKKYYLLLMPLGCFILPTVIPVYLWNETWSNAFLVAALFRYAVILNVTWLVNSAAHKWGDKPYDKSIKPSENLSVALFALGEGFHNYHHTFPWDYKTAELGNNRLNFTTTFINFFAKIGWAYDMKTVSDEIIQNRVKRTGDGSHHLWGWGDKDHSKEEINAAIRINPKDD

SEQ ID NO: 10 - protein, 352 aa, Dendrolimus punctatus
MAPKEADVNGVLFESDATTPDMALPTTPVQQADNYPKKLVWRNILLFAYLHLAALYGGYLFLFSAKWQTDIFAYILYIMSGLGITAGAHRLWAHKSYKAKWPLRLILVLFNTLAFQDSAIDWARDHRMHHKYSETDADPHNATRGFFFSHTGWLLVRKHPELKKKGKGLDISDLYADPILRFQKKYYLLLMPLGCFILPTVIPVYLWNETWSNAFLVAALFRYAVILNVTWLVNSAAHKWGDKPYDKSIKPSENLSVALFALGEGFHNYHHTFPWDYKTAELGNNRLNFTTTFINFFAKIGWAYDMKTVSDEIIQNRVKRTGDGSHHLWGWGDKDHSKEEINAAIRINPKDD

SEQ ID NO: 11 - protein, 352 aa, Dendrolimus punctatus
MAPKEADVNGVLFESDATTPDMALPTTPVQQADNYPKKLVWRNILLFAYLHLAALYGGYLFLFSAKWQTDIFAYILYIMSGLGITAGAHRLWAHKSYKAKWPLRLILVLFNTLAFQDSAIDWARDHRMHHKYSETDADPHNATRGFFFSHIGWLLVRKHPELKKKGKGLDVSDLYADPILRFQQKYYLLLMPLGCFILPTVIPVYLWNETWSNAFLVAALFRYAVILNVTWLVNSAAHKWGDRPYDKSIKPSENLSVALFALGEGFHNYHHTFPWDYKTAELGNNRLNFTTTFINFFAKIGWAYDMKTVSDEIIQNRVKRTGDGSHHLWGWGDKDHSKEEINAAIRINPKDD

SEQ ID NO: 12 - protein, 352 aa, Cydia pomonella
MAPNVTDVNGVLFESDAATPDLALANAPVQQADDSPRIYVWRNIILFAYLHIAALYGGYLFLVSAKWQTDIFAYFLYVASGLGITAGAHRLWAHKSYKAKWPLRLILVIFNTIAFQDSAIDWARDHRMHHKYSETDADPHNATRGFFFSHIGWLLVRKHPELKRKGKGLDLSDLYADPILRFQKKYYLILMPLACFVLPTVIPVYLWNETWTNAFFVAALFRYAFILNVTWLVNSAAHKWGDKPYDKSIKPSENISVSLFAFGEGFHNYHHTFPWDYKTAELSSNRLNFTTKFINFFAKIGWAYDMKTVSDEIIQKRVNRTGDGSHHLWGWGDKDHSKEEVNAAVRINPKDD

SEQ ID NO: 13 - protein, 323 aa, Ostrinia latipennis
MSEITSDPNENPTEKNIPKNKGGTKIIYQYVISFTYLHLAALYGLYLCFTSVKWLTIGFGLLLYHLTLTGITAGAHRLWSHRSYKAKFPLQVILMVLNSMAFQNTIITWARDHRVHHKCSDTDGDPHNANRGFFFSHIGWLMTKKSEEVIKKGKTIDMFDLYNNPVLRFQRKYALPVIGTLCFILPTLIPMYFWGFTLNNAWHMNILRYVTVLHVTLCVNSVVHKWGYKPYDKNILPSQNVLMSTFALGEGFHNYHHVFPWDYQTAELGNKKFNPTTWFIDFFAWVGWAYDLKTASEEVVRKRAERTGDGTDLWGAPRDNNNY

SEQ ID NO: 14 - protein, 331 aa, Artificial Sequence
  Desaturase sequence
  misc_feature (2)..(2): Xaa is Ala or Ser
  misc_feature (3)..(3): Xaa is Glu or Gln
  misc_feature (4)..(5): Xaa can be any naturally occurring amino acid
  misc_feature (6)..(6): Xaa is Ser or Thr
  misc_feature (7)..(6): Xaa is Asp or Ser
  misc_feature (8)..(8): Xaa is Pro or Ala
  misc_feature (9)..(9): Xaa is Asn or Glu
  misc_feature (10)..(10): Xaa is Glu or Gly
  misc_feature (11)..(11): Xaa can be any naturally occurring amino acid
  misc_feature (13)..(15): Xaa can be any naturally occurring amino acid
  misc_feature (16)..(16): Xaa is Asn or Glu
  misc_feature (17)..(17): Xaa can be any naturally occurring amino acid
  misc_feature (18)..(18): Xaa is Pro or Phe
  misc_feature (19)..(19): Xaa is Lys or Glu
  misc_feature (20)..(20): Xaa is Asn or Asp
  misc_feature (22)..(28): Optional amino acids
  misc_feature (29)..(29): Xaa can be any naturally occurring amino acid
  misc_feature (30)..(30): Xaa is Gly or Lys
  misc_feature (31)..(31): Xaa can be any naturally occurring amino acid
  misc_feature (32)..(32): Xaa is Lys or Glu
  misc_feature (33)..(33): Xaa can be any naturally occurring amino acid
  misc_feature (34)..(34): Xaa is Ile or Leu
  misc_feature (35)..(35): Xaa can be any naturally occurring amino acid
  misc_feature (37)..(37): Xaa is Tyr or Asn
  misc_feature (38)..(38): Xaa can be any naturally occurring amino acid
  misc_feature (40)..(40): Xaa is Ser or Thr
  misc_feature (41)..(41): Xaa can be any naturally occurring amino acid
  misc_feature (44)..(44): Xaa can be any naturally occurring amino acid
  misc_feature (47)..(47): Xaa is Ala or Ser
  misc_feature (48)..(48): Xaa can be any naturally occurring amino acid
  misc_feature (49)..(49): Xaa is Leu or Phe
  misc_feature (50)..(50): Xaa can be any naturally occurring amino acid
  misc_feature (55)..(55): Xaa is Cys or Gly
  misc_feature (56)..(56): Xaa is Phe or Leu
  misc_feature (57)..(57): Xaa can be any naturally occurring amino acid
  misc_feature (58)..(58): Xaa is Ser or Lys
  misc_feature (59)..(59): Xaa is Val or Ala
  misc_feature (60)..(60): Xaa can be any naturally occurring amino acid
  misc_feature (62)..(62): Xaa is Leu or Ala
  misc_feature (63)..(63): Xaa can be any naturally occurring amino acid
  misc_feature (66)..(66): Xaa is Phe or Ile
  misc_feature (67)..(67): Xaa is Gly or Ser
  misc_feature (68)..(68): Xaa is Leu or Ile
  misc_feature (69)..(69): Xaa is Leu or Val
  misc_feature (70)..(70): Xaa is Leu or Met
  misc_feature (71)..(71): Xaa is Tyr, Asn, His, Phe, Cys, Ser, or Leu
  misc_feature (72)..(72): Xaa can be any naturally occurring amino acid
  misc_feature (73)..(73): Xaa is Leu or Phe
  misc_feature (74)..(74): Xaa can be any naturally occurring amino acid
  misc_feature (75)..(75): Xaa is Ile, Leu, Glu, Gly, Thr, Ala, or Val
  misc_feature (76)..(77): Xaa can be any naturally occurring amino acid
  misc_feature (78)..(78): Xaa is Ile, Val, or Ala
  misc_feature (79)..(79): Xaa is Thr or Ser
  misc_feature (80)..(80): Xaa can be any naturally occurring amino acid
  misc_feature (81)..(81): Xaa is Gly or Ala
  misc_feature (82)..(82): Xaa can be any naturally occurring amino acid
  misc_feature (95)..(95): Xaa is Phe or Leu
  misc_feature (96)..(96): Xaa can be any naturally occurring amino acid
  misc_feature (98)..(98): Xaa is Asn or Glu
  misc_feature (99)..(99): Xaa is Val or Ile
  misc_feature (100)..(100): Xaa is Ile or Leu
  misc_feature (101)..(101): Xaa can be any naturally occurring amino acid
  misc_feature (104)..(104): Xaa is Leu or Phe
  misc_feature (105)..(105): Xaa can be any naturally occurring amino acid
  misc_feature (106)..(106): Xaa is Ser or Cys
  misc_feature (107)..(107): Xaa can be any naturally occurring amino acid
  misc_feature (108)..(108): Xaa is Ser or Ala
  misc_feature (109)..(109): Xaa can be any naturally occurring amino acid
  misc_feature (111)..(111): Xaa is Asn, His, Lys, Asp, or Ser
  misc_feature (112)..(112): Xaa is Thr or Ser
  misc_feature (113)..(113): Xaa is Ala or Ile
  misc_feature (114)..(114): Xaa is Leu or Ile
  misc_feature (115)..(116): Xaa can be any naturally occurring amino acid
  misc_feature (117)..(117): Xaa is Ala or Cys
  misc_feature (118)..(118): Xaa can be any naturally occurring amino acid
  misc_feature (122)..(122): Xaa is Val or Leu
  misc_feature (123)..(123): Xaa can be any naturally occurring amino acid
  misc_feature (127)..(127): Xaa is Ser or Pro
  misc_feature (128)..(128): Xaa can be any naturally occurring amino acid
  misc_feature (130)..(130): Xaa is Asp or Asn
  misc_feature (131)..(131): Xaa can be any naturally occurring amino acid
  misc_feature (137)..(137): Xaa is Asn or Ser
  misc_feature (142)..(142): Xaa is Phe or Tyr
  misc_feature (143)..(143): Xaa can be any naturally occurring amino acid
  misc_feature (145)..(145): Xaa is Ile or Val
  misc_feature (146)..(146): Xaa can be any naturally occurring amino acid
  misc_feature (149)..(149): Xaa is Met or Leu
  misc_feature (150)..(150): Xaa can be any naturally occurring amino acid
  misc_feature (151)..(151): Xaa is Lys, His, Arg, or Ser
  misc_feature (152)..(152): Xaa can be any naturally occurring amino acid
  misc_feature (154)..(154): Xaa is Glu or Asp
  misc_feature (155)..(155): Xaa can be any naturally occurring amino acid
  misc_feature (157)..(157): Xaa is Ile, Lys, Val, or Leu
  misc_feature (158)..(158): Xaa is Lys or Arg
  misc_feature (159)..(159): Xaa is Gln or Lys
  misc_feature (160)..(160): Xaa can be any naturally occurring amino acid
  misc_feature (161)..(161): Xaa is Lys or Asn
  misc_feature (162)..(162): Xaa can be any naturally occurring amino acid
  misc_feature (163)..(163): Xaa is Ile or Cys
  misc_feature (164)..(164): Xaa can be any naturally occurring amino acid
  misc_feature (165)..(165): Xaa is Met or Val
  misc_feature (166)..(167): Xaa can be any naturally occurring amino acid
  misc_feature (170)..(170): Xaa is Asn or Ser
  misc_feature (171)..(171): Xaa can be any naturally occurring amino acid
  misc_feature (178)..(178): Xaa is Lys or Arg
  misc_feature (179)..(179): Xaa can be any naturally occurring amino acid
  misc_feature (182)..(182): Xaa is Val or Leu
  misc_feature (183)..(183): Xaa can be any naturally occurring amino acid
  misc_feature (185)..(185): Xaa can be any naturally occurring amino acid
  misc_feature (188)..(188): Xaa is Leu or Ile
  misc_feature (189)..(189): Xaa can be any naturally occurring amino acid
  misc_feature (191)..(191): Xaa is Ile or Val
  misc_feature (192)..(192): Xaa is Leu or Phe
  misc_feature (193)..(193): Xaa can be any naturally occurring amino acid
  misc_feature (198)..(198): Xaa is Met or Val
  misc_feature (199)..(199): Xaa can be any naturally occurring amino acid
  misc_feature (211)..(211): Xaa is Met or Phe
  misc_feature (212)..(212): Xaa can be any naturally occurring amino acid
  misc_feature (213)..(213): Xaa is Ile or Met
  misc_feature (214)..(214): Xaa is Leu or Phe
  misc_feature (215)..(215): Xaa can be any naturally occurring amino acid
  misc_feature (219)..(220): Xaa can be any naturally occurring amino acid
  misc_feature (221)..(221): Xaa is His or Asn
  misc_feature (222)..(222): Xaa is Ala or Val
  misc_feature (223)..(223): Xaa can be any naturally occurring amino acid
  misc_feature (224)..(224): Xaa is Leu, Phe, Tyr, or Trp
  misc_feature (225)..(225): Xaa is Cys, Leu, Met, Phe, or Ser
  misc_feature (226)..(226): Xaa is Val, Ile, Leu, Glu, Gly, Thr, or Ala
  misc_feature (227)..(227): Xaa can be any naturally occurring amino acid
  misc_feature (230)..(230): Xaa is Val or Ala
  misc_feature (231)..(231): Xaa can be any naturally occurring amino acid
  misc_feature (232)..(232): Xaa is Lys or Arg
  misc_feature (233)..(233): Xaa can be any naturally occurring amino acid
  misc_feature (237)..(237): Xaa is Pro or Thr
  misc_feature (238)..(238): Xaa can be any naturally occurring amino acid
  misc_feature (243)..(243): Xaa is Leu or Arg
  misc_feature (244)..(244): Xaa can be any naturally occurring amino acid
  misc_feature (245)..(245): Xaa is Ser or Thr
  misc_feature (248)..(248): Xaa is Met or Val
  misc_feature (249)..(249): Xaa can be any naturally occurring amino acid
  misc_feature (250)..(250): Xaa is Leu or Met
  misc_feature (251)..(251): Xaa is Asn or Ser
  misc_feature (252)..(252): Xaa is Thr or Phe
  misc_feature (253)..(252): Xaa is Ala or Phe
  misc_feature (253)..(253): Xaa can be any naturally occurring amino acid
  misc_feature (254)..(254): Xaa is Met, Cys, Thr, Ala, Val, Ser, or Cys
  misc_feature (255)..(255): Xaa can be any naturally occurring amino acid
  misc_feature (258)..(258): Xaa is Gly or Ala
  misc_feature (259)..(259): Xaa can be any naturally occurring amino acid
  misc_feature (262)..(262): Xaa is Tyr or Phe
  misc_feature (263)..(263): Xaa can be any naturally occurring amino acid
  misc_feature (271)..(271): Xaa is Gln or Arg
  misc_feature (272)..(272): Xaa can be any naturally occurring amino acid
  misc_feature (273)..(273): Xaa is Ala or Ser
  misc_feature (274)..(274): Xaa can be any naturally occurring amino acid
  misc_feature (279)..(279): Xaa can be any naturally occurring amino acid
  misc_feature (280)..(280): Xaa is Phe or Met
  misc_feature (281)..(281): Xaa can be any naturally occurring amino acid
  misc_feature (285)..(285): Xaa is Trp or Leu
  misc_feature (286)..(286): Xaa can be any naturally occurring amino acid
  misc_feature (293)..(293): Xaa is Val or Ile
  misc_feature (294)..(294): Xaa can be any naturally occurring amino acid
  misc_feature (302)..(302): Xaa is Ala or Thr
  misc_feature (303)..(303): Xaa can be any naturally occurring amino acid
  misc_feature (304)..(304): Xaa is Glu or Lys
  misc_feature (305)..(305): Xaa can be any naturally occurring amino acid
  misc_feature (306)..(306): Xaa is Val or Met
  misc_feature (307)..(307): Xaa is Val or Ile
  misc_feature (308)..(308): Xaa is Arg or Lys
  misc_feature (309)..(309): Xaa is Lys or Ser
  misc_feature (310)..(310): Xaa can be any naturally occurring amino acid
  misc_feature (311)..(311): Xaa is Ala or Ser
  misc_feature (312)..(312): Xaa can be any naturally occurring amino acid
  misc_feature (322)..(322): Xaa is Ala or His
  misc_feature (324)..(324): Xaa can be any naturally occurring amino acid
  misc_feature (325)..(325): Xaa is Arg or Ala
  misc_feature (326)..(326): Xaa is Asn or Asp
  misc_feature (327)..(330): Optional amino acids
  misc_feature (331)..(331): Xaa can be any naturally occurring amino acid
MXXXXXDPNEXPXXXXXXXXKXXXXXXXXXXXXIXYXXVXXFTXLHLXAXYGLYLXXTXXKWXTIGXXXXXXXXXXXGXXAXAHRLWSHRSYKAKXPLXXXLMVXNXMXFQXXXXXWXRDHRXHHKCXDTXGDPHNASRGFFXSHXGWLXTXKSXEVXXXGXXXDXXDLYXNPVLRFQXKYAXPXIGXXCFXXPTLIPXYFWGETINNAWHXNXXRYVXXLXXTXXXNSVXHXWGYKXYDKNIXPSQNXLXXXXXLGEXFHNXHHVFPWDYXXXELGNXKXNPTTXFIDFFAWXGWAYDLKTXSXEXXXXRXERTGDGTDLWGXXXXXXXX

SEQ ID NO: 15 - protein, 324 aa, Artificial Sequence
  Consensus desaturase sequence
  misc_feature (4)..(5): Xaa can be any naturally occurring amino acid
  misc_feature (11)..(11): Xaa can be any naturally occurring amino acid
  misc_feature (13)..(15): Xaa can be any naturally occurring amino acid
  misc_feature (17)..(17): Xaa can be any naturally occurring amino acid
  misc_feature (22)..(22): Xaa can be any naturally occurring amino acid
  misc_feature (24)..(24): Xaa can be any naturally occurring amino acid
  misc_feature (29)..(29): Xaa can be any naturally occurring amino acid
  misc_feature (32)..(32): Xaa can be any naturally occurring amino acid
  misc_feature (36)..(36): Xaa can be any naturally occurring amino acid
  misc_feature (65)..(65): Xaa can be any naturally occurring amino acid
  misc_feature (67)..(69): Xaa can be any naturally occurring amino acid
  misc_feature (108)..(108): Xaa can be any naturally occurring amino acid
  misc_feature (155)..(155): Xaa can be any naturally occurring amino acid
  misc_feature (159)..(159): Xaa can be any naturally occurring amino acid
  misc_feature (177)..(177): Xaa can be any naturally occurring amino acid
  misc_feature (180)..(180): Xaa can be any naturally occurring amino acid
  misc_feature (211)..(212): Xaa can be any naturally occurring amino acid
  misc_feature (248)..(248): Xaa can be any naturally occurring amino acid
  misc_feature (266)..(266): Xaa can be any naturally occurring amino acid
  misc_feature (272)..(272): Xaa can be any naturally occurring amino acid
  misc_feature (318)..(318): Xaa can be any naturally occurring amino acid
  misc_feature (321)..(323): Xaa can be any naturally occurring amino acid
MAEXXSDPNEXPXXXNXPKNKXGXKIIYXYVXSFTXLHLAALYGLYLCFTSVKWLTIGFGLLLYXLXXXGITAGAHRLWSHRSYKAKFPLQVILMVLNSMAFQNTALXWARDHRVHHKCSDTDGDPHNANRGFFFSHIGWLMTKKSEEVIKQGKXIDMXDLYNNPVLRFQKKYAVPXIGXLCFILPTLIPMYFWGETLNNAWHMNILRYVXXLYHATLCVNSVVHKWGYKPYDKNILPSQNVLLNTAXLGEGFHNYHHVFPWDYQXAELGNXKFNPTTWFTDFFAWVGWAYDLKTASEEVVRKRAERTGDGTDLWGAXRDXXXY

SEQ ID NO: 16 - protein, 319 aa, Artificial Sequence
  DST014 desaturase
  MISC_FEATURE (64)..(64): Xaa is Tyr, Asn, His, Phe, Cys, Ser, or Leu
  MISC_FEATURE (68)..(68): Xaa is Ile, Leu, Glu, Gly, Thr, Ala, or Val
  MISC_FEATURE (71)..(71): Xaa is Ile, Val, or Ala
  MISC_FEATURE (104)..(104): Xaa is Asn, His, Lys, Asp, or Ser
  MISC_FEATURE (144)..(144): Xaa is His, Lys, Arg, or Ser
  MISC_FEATURE (150)..(150): Xaa is Lys, Ile, Val, or Leu
  MISC_FEATURE (217)..(217): Xaa is Leu, Phe, Tyr, or Trp
  MISC_FEATURE (218)..(218): Xaa is Cys, Leu, Met, Phe, or Ser
  MISC_FEATURE (219)..(219): Xaa is Ile, Leu, Glu, Gly, Thr, Ala, or Val
  MISC_FEATURE (247)..(247): Xaa is Met, Cys, Thr, Ala, Val, Ser, or Cys
MAETSSDPNETPADENLPKNKRGSKIIYKYVTSFTILHLAAFYGLYLCLTKAKWLTIGFSLLMXELSXLGXSAAAHRLWSHRSYKAKFPLQVILMVLNCMSFQXSALNWCRDHRVHHKCSDTDGDPHNASRGFFFSHIGWLMTXKSEEVXRQGNSIDMSDLYNNPVLRFQKKYAVPLIGSICFIFPTLIPVYFWGETINNAWHMNILRYVMALHATXXXNSVAHRWGYKTYDKNILPSQNMLLNTAXLGEGFHNYHHVFPWDYQSSELGNRKFNPTTWFIDFFAWVGWAYDLKTTSEEVIRKRAERTGDGTDLWGAKRN

SEQ ID NO: 17 - protein, 329 aa, Artificial Sequence
  DST109 desaturase
  MISC_FEATURE (230)..(230): Xaa is Val or Ala
  MISC_FEATURE (243)..(243): Xaa is Arg or Ala
  MISC_FEATURE (252)..(252): Xaa is Phe or Ala
MAQYATSAEGLPVKDECFEDKEIKSNSFPKLEILYFNVMTFTLLHLSALYGLYLGFTSVKWATIGIGIVLYFFAEIGITAGAHRLWSHRSYKAKLPLEILLMVFNSMARQNTALSWARDHRLHHKCPDTNGDPHNANRGFFYSHVGWLLTKKSDEVIKQGKLCDVADLYSNPVLRFQKKYAVPFIGMLCFVLPTLIPMYFWGETLNNAWHFNMFRYVVNLNATFCVNSVXHKWGYKPYDKNIXPTQNVLLNXAVLGEAFHNFHHVFPWDYRAAELGNQKMNPTTLFIDFFAWIGWAYDLKTASKEMVKSRSERTGDGTDLWGHDADKLK

SEQ ID NO: 18 - protein, 329 aa, Artificial Sequence
  E11-16 high titer DST109 desaturase
  misc_feature (243)..(243): Xaa is Arg or Ala
  misc_feature (252)..(252): Xaa is Phe or Ala
MAQYATSAEGLPVKDECFEDKEIKSNSFPKLEILYFNVMTFTLLHLSALYGLYLGFTSVKWATIGIGIVLYFFAEIGITAGAHRLWSHRSYKAKLPLEILLMVFNSMARQNTALSWARDHRLHHKCPDTNGDPHNANRGFFYSHVGWLLTKKSDEVIKQGKLCDVADLYSNPVLRFQKKYAVPFIGMLCFVLPTLIPMYFWGETLNNAWHFNMFRYVVNLNATFCVNSVAHKWGYKPYDKNIXPTQNVLLNXAVLGEAFHNFHHVFPWDYRAAELGNQKMNPTTLFIDFFAWIGWAYDLKTASKEMVKSRSERTGDGTDLWGHDADKLK

SEQ ID NO: 19 - protein, 329 aa, Artificial Sequence
  E11-16 high titer DST109 desaturase
  misc_feature (230)..(230): Xaa is Val or Ala
  misc_feature (252)..(252): Xaa is Phe or Ala
MAQYATSAEGLPVKDECFEDKEIKSNSFPKLEILYFNVMTFTLLHLSALYGLYLGFTSVKWATIGIGIVLYFFAEIGITAGAHRLWSHRSYKAKLPLEILLMVFNSMARQNTALSWARDHRLHHKCPDTNGDPHNANRGFFYSHVGWLLTKKSDEVIKQGKLCDVADLYSNPVLRFQKKYAVPFIGMLCFVLPTLIPMYFWGETLNNAWHFNMFRYVVNLNATFCVNSVXHKWGYKPYDKNIAPTQNVLLNXAVLGEAFHNFHHVFPWDYRAAELGNQKMNPTTLFIDFFAWIGWAYDLKTASKEMVKSRSERTGDGTDLWGHDADKLK

SEQ ID NO: 20 - protein, 329 aa, Artificial Sequence
  E11-16 high titer DST109 desaturase
  misc_feature (230)..(230): Xaa is Val or Ala
  misc_feature (243)..(243): Xaa is Arg or Ala
MAQYATSAEGLPVKDECFEDKEIKSNSFPKLEILYFNVMTFTLLHLSALYGLYLGFTSVKWATIGIGIVLYFFAEIGITAGAHRLWSHRSYKAKLPLEILLMVFNSMARQNTALSWARDHRLHHKCPDTNGDPHNANRGFFYSHVGWLLTKKSDEVIKQGKLCDVADLYSNPVLRFQKKYAVPFIGMLCFVLPTLIPMYFWGETLNNAWHFNMFRYVVNLNATFCVNSVXHKWGYKPYDKNIXPTQNVLLNAAVLGEAFHNFHHVFPWDYRAAELGNQKMNPTTLFIDFFAWIGWAYDLKTASKEMVKSRSERTGDGTDLWGHDADKLK

SEQ ID NO: 21 - protein, 329 aa, Artificial Sequence
  E11-16 high titer DST109 desaturase
MAQYATSAEGLPVKDECFEDKEIKSNSFPKLEILYFNVMTFTLLHLSALYGLYLGFTSVKWATIGIGIVLYFFAEIGITAGAHRLWSHRSYKAKLPLEILLMVFNSMARQNTALSWARDHRLHHKCPDTNGDPHNANRGFFYSHVGWLLTKKSDEVIKQGKLCDVADLYSNPVLRFQKKYAVPFIGMLCFVLPTLIPMYFWGETLNNAWHFNMFRYVVNLNATFCVNSVAHKWGYKPYDKNIAPTQNVLLNAAVLGEAFHNFHHVFPWDYRAAELGNQKMNPTTLFIDFFAWIGWAYDLKTASKEMVKSRSERTGDGTDLWGHDADKLK

SEQ ID NO: 22 - protein, 319 aa, Artificial Sequence
  E11-specific desaturase
  MISC_FEATURE (64)..(64): Xaa is Asn, His, or Tyr
  MISC_FEATURE (68)..(68): Xaa is Ile, Leu, or Glu
  MISC_FEATURE (144)..(144): Xaa is His or Lys
  MISC_FEATURE (150)..(150): Xaa is Lys or Ile
  MISC_FEATURE (217)..(217): Xaa is Leu or Phe
  MISC_FEATURE (218)..(218): Xaa is Leu or Cys
  MISC_FEATURE (219)..(219): Xaa is Ile, Leu, or Glu
  misc_feature (247)..(247): Xaa can be any naturally occurring amino acid
MAETSSDPNETPADENLPKNKRGSKIIYKYVTSFTILHLAAFYGLYLCLTKAKWLTIGFSLLMXELSXLGISAAAHRLWSHRSYKAKFPLQVILMVLNCMSFQNSALNWCRDHRVHHKCSDTDGDPHNASRGFFFSHIGWLMTXKSEEVXRQGNSIDMSDLYNNPVLRFQKKYAVPLIGSICFIFPTLIPVYFWGETINNAWHMNILRYVMALHATXXXNSVAHRWGYKTYDKNILPSQNMLLNTAXLGEGFHNYHHVFPWDYQSSELGNRKFNPTTWFIDFFAWVGWAYDLKTTSEEVIRKRAERTGDGTDLWGAKRN

SEQ ID NO: 23 - protein, 319 aa, Artificial Sequence
  Z11-specific desaturase
  MISC_FEATURE (64)..(64): Xaa is Tyr, Phe, or Cys
  MISC_FEATURE (68)..(68): Xaa is Glu or Leu
  MISC_FEATURE (71)..(71): Xaa is Val or Ile
  MISC_FEATURE (144)..(144): Xaa is Arg or Lys
  MISC_FEATURE (150)..(150): Xaa is Lys or Ile
  MISC_FEATURE (217)..(217): Xaa is Leu or Phe
  MISC_FEATURE (218)..(218): Xaa is Leu or Cys
  MISC_FEATURE (219)..(219): Xaa is Glu or Leu
  MISC_FEATURE (247)..(247): Xaa is Thr, Val, or Ala
MAETSSDPNETPADENLPKNKRGSKIIYKYVTSFTILHLAAFYGLYLCLTKAKWLTIGFSLLMXELSXLGXSAAAHRLWSHRSYKAKFPLQVILMVLNCMSFQNSALNWCRDHRVHHKCSDTDGDPHNASRGFFFSHIGWLMTXKSEEVXRQGNSIDMSDLYNNPVLRFQKKYAVPLIGSICFIFPTLIPVYFWGETINNAWHMNILRYVMALHATXXXNSVAHRWGYKTYDKNILPSQNMLLNTAXLGEGFHNYHHVFPWDYQSSELGNRKFNPTTWFIDFFAWVGWAYDLKTTSEEVIRKRAERTGDGTDLWGAKRN

SEQ ID NO: 24 - protein, 319 aa, Artificial Sequence
  Z14-specific desaturase
  MISC_FEATURE (64)..(64): Xaa is Ser or Leu
  MISC_FEATURE (71)..(71): Xaa is Ala or Ile
  MISC_FEATURE (104)..(104): Xaa is His or Asn
  MISC_FEATURE (144)..(144): Xaa is Ser or Lys
  MISC_FEATURE (217)..(217): Xaa is Tyr or Trp
  MISC_FEATURE (218)..(218): Xaa is Phe or Leu
MAETSSDPNETPADENLPKNKRGSKIIYKYVTSFTILHLAAFYGLYLCLTKAKWLTIGFSLLMXELSTLGXSAAAHRLWSHRSYKAKFPLQVILMVLNCMSFQXSALNWCRDHRVHHKCSDTDGDPHNASRGFFFSHIGWLMTXKSEEVKRQGNSIDMSDLYNNPVLRFQKKYAVPLIGSICFIFPTLIPVYFWGETINNAWHMNILRYVMALHATXXTNSVAHRWGYKTYDKNILPSQNMLLNTASLGEGFHNYHHVFPWDYQSSELGNRKFNPTTWFIDFFAWVGWAYDLKTTSEEVIRKRAERTGDGTDLWGAKRN

SEQ ID NO: 25 - protein, 319 aa, Artificial Sequence
  Z9-specific desaturase
  MISC_FEATURE (64)..(64): Xaa is Tyr or His
  MISC_FEATURE (68)..(68): Xaa is Gly or Ala
  MISC_FEATURE (104)..(104): Xaa is Lys or Asp
  MISC_FEATURE (144)..(144): Xaa is Lys or Arg
  MISC_FEATURE (150)..(150): Xaa is Lys or Ile
  MISC_FEATURE (218)..(218): Xaa is Leu or Ser
  MISC_FEATURE (219)..(219): Xaa is Gly or Ala
  MISC_FEATURE (247)..(247): Xaa is Thr, Ala, or Cys
MAETSSDPNETPADENLPKNKRGSKIIYKYVTSFTILHLAAFYGLYLCLTKAKWLTIGFSLLMXELSXLGISAAAHRLWSHRSYKAKFPLQVILMVLNCMSFQXSALNWCRDHRVHHKCSDTDGDPHNASRGFFFSHIGWLMTXKSEEVXRQGNSIDMSDLYNNPVLRFQKKYAVPLIGSICFIFPTLIPVYFWGETINNAWHMNILRYVMALHATWXXNSVAHRWGYKTYDKNILPSQNMLLNTAXLGEGFHNYHHVFPWDYQSSELGNRKFNPTTWFIDFFAWVGWAYDLKTTSEEVIRKRAERTGDGTDLWGAKRN

SEQ ID NO: 26 - protein, 319 aa, Artificial Sequence
  Z6-specific desaturase
  MISC_FEATURE (68)..(68): Xaa is Ile or Val
  MISC_FEATURE (219)..(219): Xaa is Ile or Val
MAETSSDPNETPADENLPKNKRGSKIIYKYVTSFTILHLAAFYGLYLCLTKAKWLTIGFSLLMLELSXLGISAAAHRLWSHRSYKAKFPLQVILMVLNCMSFQSSALNWCRDHRVHHKCSDTDGDPHNASRGFFFSHIGWLMTRKSEEVLRQGNSIDMSDLYNNPVLRFQKKYAVPLIGSICFIFPTLIPVYFWGETINNAWHMNILRYVMALHATFSXNSVAHRWGYKTYDKNILPSQNMLLNTASLGEGFHNYHHVFPWDYQSSELGNRKFNPTTWFIDFFAWVGWAYDLKTTSEEVIRKRAERTGDGTDLWGAKRN

SEQ ID NO: 27 - protein, 319 aa, Artificial Sequence
  Z13-specific desaturase
MAETSSDPNETPADENLPKNKRGSKIIYKYVTSFTILHLAAFYGLYLCLTKAKWLTIGFSLLMFELSGLGISAAAHRLWSHRSYKAKFPLQVILMVLNCMSFQHSALNWCRDHRVHHKCSDTDGDPHNASRGFFFSHIGWLMTRKSEEVVRQGNSIDMSDLYNNPVLRFQKKYAVPLIGSICFIFPTLIPVYFWGETINNAWHMNILRYVMALHATFMGNSVAHRWGYKTYDKNILPSQNMLLNTASLGEGFHNYHHVFPWDYQSSELGNRKFNPTTWFIDFFAWVGWAYDLKTTSEEVIRKRAERTGDGTDLWGAKRN

SEQ ID NO: 28 - protein, 343 aa, Ctenopseustis obliquana
MPPNTKEKEMNELAGISGRNEELAPQAAPRKFEIVYRNIVTFSFMHVAGLYGLYLCFTSAKWQTIVLAFVLYTFAEIGVTAGAHRLWAHKTYKAKTPLQIILMVLNSIAFQNSAIDWVRDHRLHHKYSDTDADPHNATRGFFYSHVGWLLVGKHPEVIKRGKAMEMSDIYSNPVLWFQKKYAVPFIGIICFGLPTLVPMYFWGETLNNAWHITMLRYIANLNVTFLVNSAAHMFGNKPYNKNILPAQNISVSILTFGEGFHNYHHVYPWDYRTAELGNNMLNMTTLFIDFFAWIGWAYDLKTVSSAAIESKAKRTGDGTNLWGWGDKDMLREDQKAAEILFAK

SEQ ID NO: 29 - protein, 347 aa, Planototrix excessana
MPPNTKQKEMYDPAGIPGKNEEMVHLVAPQAAPGKFEIVYRNIATFSYMHVAGLYGLYLCFTSGKWQTMVLAFVLYTFAEIGITAGAHRLWAHKTYKAKMPLQILLMVLNSIAFQNSAIDWVRDHRLHHKYSDTDADPHNATRGFFYSHVGWLLVRKHPEVTKRGKTMDMSDIYSNPVLRFQKKYAVPFIGIICLGLPTLIPMYFWGETLNNAWHITMLRYIANLNVTFLVNSAAHMFGNKPYDKNILPAQNISVSIFTFGEGFHNYHHVFPWDYRAAELGNNMLNMTTLFIDFFAWIGWAYDLKTVSSAVIESRAKRTGDGTNLWGWGDKDMLREDQEAAEILFAK

SEQ ID NO: 30 - protein, 352 aa, Lampronia capitella
MPPNVTEANGVLFENDVQTPDMGLEVAPVQKADERKIQLVWRNIIAFACLHLAAVYGAYLFFTSAIWQTDIFAYILYVMSGLGITAGAHRLWAHKSYKAKWPLRLILVAFNTLAFQDSAIDWARDHRMHHKYSETDADPHNATRGFFFSHIGWLLCRKHPELKRKGQGLDLSDLYADPIIRFQKKYYLLLMPLACFVLPTIIPVYLWGESWKNAFFVAAMFRYTFILNVTWLVNSAAHKWGGKPYDKNIQPAQNISVAIFALGEGFHNYHHTFPWDYKTAELGNNRLNFTTSFINFFASFGWAYDLKTVSDEIIQQRVKRTGDGSHHLRGWGDQDIPAEEAQAALRINRKDD

SEQ ID NO: 31 - protein, 352 aa, Pectinophora gossypiella
MAPNATDVNGVLFESDAVTPDMALPNTPVAQADNTPKQYVWRNIILFAYLHLAAVYGGYLFLFSAKWQTDLFAYILYVMSGLGITAGAHRLWAHKSYKAKWPLRLILVIFNTIAFQDSAIDWARDHRMHHKYSETDADPHNATRGFFFSHIGWLLVRKHPELKKKGQGLDLSDLYADPILRFQKKYYIYLMPLACFVMPTVVPVYLWGESWTNAFFVCALFRYAFILNVTWLVNSAAHKWGDKPYDKSIKPAQNLSVSVFALGEGFHNYHHTFPWDYKTAELGNNSLNFTTNFINFFSKIGWAYDLKTVSDEIVKARVERTGDGSHHLWGWGDKDHDQKEINAAIRINPKDD

SEQ ID NO: 32 - protein, 368 aa, Ameloyis transitella
MHIESENCPGRFKEVNMAPNATDANGVLFETDAATPDLALPHAPVQQADNYPKKYVWRNIILFAYLHIAALYGGYLFLFHAKWQTDIFAYILYVMSGLGITAGAHRLWAHKSYKAKWPLRLILVIFNTLAFQDSAIDWSRDHRMHHKYSETDADPHNATRGFFFSHIGWLLVRKHPELKRKGKGLDLSDLYADPILRFQKKYYLILMPLTCFVLPTVIPVYYWGETWTNAFFVAALFRYAFILNVTWLVNSAAHKWGDKPYDRNIKPSENISVSMFALGEGFHNYHHTFPWDYKTAELGNNMLNFTTNFINFFAKIGWAYDLKTVSDEIVRSRAKRTGDGSHHLWGWGDKDHSREEMAAAIRIHPKDD

SEQ ID NO: 33 - protein, 352 aa, Ameloyis transitella
MAPNATDANGVLFETDAATPDLALPHAPVQQADNYPKKYVWRNIILFAYLHIAALYGGYLFLFHAKWQTDIFAYILYVMSGLGITAGAHRLWAHKSYKAKWPLRLILVIFNTLAFQDSAIDWSRDHRMHHKYSETDADPHNATRGFFFSHIGWLLVRKHPELKRKGKGLDLSDLYADPILRFQKKYYLILMPLTCFVLPTVIPVYYWGETWTNAFFVAALFRYAFILNVTWLVNSAAHKWGDKPYDRNIKPSENISVSMFALGEGFHNYHHTFPWDYKTAELGNNMLNFTTNFINFFAKIGWAYDLKTVSDEIVRSRAKRTGDGSHHLWGWGDKDHSREEMAAAIRIHPKDD

SEQ ID NO: 34 - protein, 355 aa, Danaus plexippus
MAPNSSDANGVLFESDAATPDLALPNTPIQQADSYPRKYVWRNIIAFAYLHIAALYGGYLFLFSAKWQTNIFAYILYVMSGLGITAGAHRLWAHKSYKAKWPLRVILVIFNTLAFQDSAIDWARDHRMHHKYSETDADPHNATRGFFFSHIGWLLVRKHPELKRKGKGLDLSDLYADPILRFQKKYYLLLMPISCFILPTIIPVYLWGETWTNGYFVAAMFRYAFILNVTWLVNSAAHKWGDKPYDKNIQPSENMSVSLFALGEGFHNYHHTFPWDYKTAELGNNRLNFTTTFINFFAKIGWAYDLKTVSDEIVKQRVNRTGDGSHHLWGWGDKDHSKEEIRAAIRINPTDDKEE

SEQ ID NO: 35 - protein, 352 aa, Papillo polytes
MAPKSADANGVLFESDAATPDLALGSAPIMQADKQPRKLVWRNIIAFAYLHLAAVYGGYLFLFHAKWQTDIFAYILYVMSGLGITAGAHRLWAHKSYKAKWPLRLILVIFNTLAFQDSAIDWARDHRMHHKYSETDADPHNATRGFFFSHIGWLLVRKHPELKRKGQGLDLSDLYADPILRFQKKYYLLLMPLACFVLPTVIPVYYWGESWITSFFVAAMFRYAFILNVTWLVNSAAHKWGDKPYDRHIQPAENISVALFALGEGFHNYHHTFPWDYKTAELGNNRLNFTTNFINFFAKIGWAYDLKTVSDEIIRSRVNRTGDGSHHLWGWGDKDHAKEEIDAAIRINPKDD

SEQ ID NO: 36 - protein, 352 aa, Papillo xuthus
MAPKSADANGVLFESDAATPDLALGSAPIMQADKQPRRLVWRNIIAFAYLHLAAIYGGYLFLFHAKWQTDIFAYILYVMSGLGITAGAHRLWAHKSYKAKWPLRLILVIFNTLAFQDSAIDWARDHRMHHKYSETDADPHNATRGFFFSHIGWLLVRKHPELKRKGQGLDLSDLYADPILRFQKKYYLLLMPIACFILPTVIPVYYWGESWVTSFFVAALFRYAFILNVTWLVNSAAHKWGDKPYDKHIQPAENISVSIFALGEGFHNYHHTFPWDYKTAELGNNRLNFTTNFINLFAKIGWAYDLKTVSDEIVRNRVKRTGDGSHHLWGWGDKDHAKEEIDAAIRINPKDD

SEQ ID NO: 37 - protein, 333 aa, Antheraea pernyi
MAPNITDANGVLFESDATTPDLGLPIQPIQQADNRPKQFVWRNIILFAYLHIAALYGGYLFLFHAKWQTDIFAYILYVMSGLGITAGAHRLWAHKSYKAKWPLRLILVIFNTLAFQDSAIDWARDHRMHHKYSETDADPHNASRGFFFSHIGWLLVRKHPELKRKGKGLDLNDLYADPILRFQKKYYLLLMPIACFILPTVIPVYFWSETWSNAFFVAALFRYTFILNVTWLVNSAAHKWGDKPYDKSIKPSENISVSVFALGEGFHNYHHTFPWDYKTAELGNNRLNFTTNFINFFAKFGWAYDLKTVSDEIVQNRVKRTGDGSHHLWGWGG

SEQ ID NO: 38 - protein, 352 aa, Manduca sexta
MPPYTSDANGVLFESDAATPDLALSSTPVQQADNRPKQLVWRNIILFAYLHIAALYGGYLFLVHAKWQTDIFAYLLYVMSGLGITAGAHRLWAHKSYKAKWPLRLILVVFNTMAFQDSAIDWARDHRMHHKYSETDADPHNATRGFFFSHIGWLLVRKHPELKRKGKGLDLSDLYADPILRFQKKYYLILMPITCFVMPTVIPVYLWGESWVNAFFVAALFRYAFILNVTWLVNSAAHKWGDKPYDKSIKPSENISVSMFALGEGFHNYHHTFPWDYKTAELGNNRLNFTTNFINFFAKIGWAYDLKTVSDEIIQQRVQRTGDGSHHLWGWGDKDHDKEEVNAAIRINPKDD

SEQ ID NO: 39 - protein, 352 aa, Plutella xylostella
MAPNASDANGVLFESDAATPDLALPHAPVQRADAYPRQYVWRNILLFAYLHLTALYGGFLFLTSAKWQTDVFAYILYVVSGLGITAGAHRLWAHKSYKAKWPLRLILVIFNTIAFQDSAIDWARDHRMHHKYSETDADPHNATRGFFFSHIGWLLVRKHPELKNKGKGLDLSDLYADPILRFQKKYYLLLMPLACFVMPTVVPVYCWGESWSNAYFVAAMFRYAFILNVTWLVNSAAHKWGDKPYDKAIKPSENISVSIFALGEGFHNYHHTFPWDYKTAELGNNRLNFTTNFINFFAKLGWAYDLKTVSEEIIQQRVNRTGDGSHHLWGWGDTDHDRAEADAAIRINPKDE

SEQ ID NO: 40 - protein, 352 aa, Bombyx mori
MAPNVKDANGVLFENDADTPDLGLSSTPVQQADNYPKKLVWRNIILFTYLHIAALYGGYLFLYHAKWQTDLFAYILYVMSGLGITAGAHRLWAHKSYKAKWPLRVILIIFNSLAFQDSALDWARDHRMHHKYSETDADPHNATRGFFFSHIGWLLVRKHPELKRKGKGLDLSDLYADPILRFQKKYYLILMPIACFILPTVIPVYFWNESWINAFFVATLFRYTFILNVTWLVNSAAHKWGGKPYDKNIKPSENISVSVFALGEGFHNYHHTFPWDYKTAELGNNRLNFTTNFINFFAKIGWAYDLKTVSDEIIKNRVNRTGDGSHYLWGWGDKDLDKEEIKQAIRINPKDN

SEQ ID NO: 41 - protein, 353 aa, Sesamaia inferens
MAPNIAEDVNGVLFESDAATPDLALATPPVQKADNLPKQYVWRNILLFAYLHLAALYGGYLFLFSAKWQTDVFAYILYVVSGLGITAGAHRLWAHKSYKAKWPLKVILVIFNTIAFQDAAMDWARDHRMHHKYSETDADPHNATRGFFFSHIGWLLVKKHPDLKEKGKGLDMSDLRSDPILRFQKKYYLLLMPLACFIMPTVVPVYFWGETWTNAFFVAAMFRYAFILNVTWLVNSAAHKWGDKPYDKSIKPSENMSVAMFALGEGFHNYHHTFPWDYKTAELGNNKLNFTTTFINFFAKLGWAYDMKTVSQDIVKSRVKRTGDGSHNLWGWGDKNQSKEEIAAAIRINPKDD

SEQ ID NO: 42 - protein, 353 aa, Agrotis ipsilon
MAPNISDDVNGVLFESDAATPDLALASPPVQKADNRPKQYVWRNILLFAYLHAAALYGGYLFLTSAKWQTDVFAYILYVMSGLGITAGAHRLWAHKSYKAKWPLKVILIIFNTIAFQDAAMDWARDHRMHHKYSETDADPHNATRGFFFSHIGWLLVRKHPDLKEKGKGLDMSDLQADPILRFQKKYYLLLMPLACFVMPTVIPVYFWGETWNNAFFVAAMFRYAFILNVTWLVNSAAHKWGDKPYDKSIKPSENMSVAMFALGEGFHNYHHTFPWDYKTAELGNNKLNFTTTFINFFAKLGWAYDMKTVSDDIVKNRVKRTGDGSHHLWGWGDKNQSKEEIASAIRINPKDD

SEQ ID NO: 43 - protein, 353 aa, Mamestra brassicae
MAPNISEDANGVLFESDAATPDLALASPPVQKADNRPKQYVWRNIILFAYLHLAALYGGYLFLFSAKWQTDVFAYILYVMSGLGITAGAHRLWAHKSYKAKWPLKVILIIFNTIAFQDAAMDWARDHRMHHKYSETDADPHNATRGFFFSHIGWLLVRKHPDLKKKGKGLDMSDLLNDPILKFQKKYYLLLMPLACFVMPTMIPVYLWGETWTNAFFVAAMFRYAFILNVTWLVNSAAHKWGDKPYDKSIKPSENLSVAMFALGEGFHNYHHTFPWDYKTAELGNQKLNFTTTFINFFAKLGWAYDMKTVSDDIVKNRVKRTGDGSHHLWGWGDKNQSKEEIASAIRINPKDD

SEQ ID NO: 44 - protein, 353 aa, Helicoverpa assulta
MAPNISEDVNGVLFESDAATPDLALSTPPVQKADNRPKQLVWRNILLFAYLHLAAQYGGYLFLFSAKWQTDIFAYILYVISGLGITAGAHRLWAHKSYKAKWPLRVILVIFNTVAFQDAAMDWARDHRMHHKYSETDADPHNATRGFFFSHIGWLLVRKHPDLKEKGKGLDMSDLLADPILRFQKKYYLILMPLACFVMPTVIPVYFWGETWTNAFFVAAMFRYAFILNVTWLVNSAAHKWGDKPYDKSIKPSENLSVAMFALGEGFHNYHHTFPWDYKTAELGNNKLNFTTTFINFFAKIGWAYDLKTVSDDIVKNRVKRTGDGSHHLWGWGDENQSKEEIDAAIRINPKDD

SEQ ID NO: 45 - protein, 353 aa, Helicoverpa zea
MAPNISEDVNGVLFESDAATPDLALSTPPVQKADNRPKQLVWRNILLFAYLHLAALYGGYLFLFSAKWQTDIFAYILYVISGLGITAGAHRLWAHKSYKAKWPLRVILVIFNTVAFQDAAMDWARDHRMHHKYSETDADPHNATRGFFFSHIGWLLVRKHPDLKEKGKGLDMSDLLADPILRFQKKYYLILMPLACFVMPTVIPVYFWGETWTNAFFVAAMFRYAFILNVTWLVNSAAHKWGDKPYDKSIKPSENLSVAMFALGEGFHNYHHTFPWDYKTAELGNNKLNFTTTFINFFAKIGWAYDLKTVSDDIVKNRVKRTGDGSHHLWGWGDENQSKEEIDAAIRINPKDD

SEQ ID NO: 46 - protein, 353 aa, Helicoverpa subflexa
MAPNISEDVNGVLFESDAATPDLALATPPVQKADNRPKQLVWRNILLFAYLHLAAVYGGYLFLFSAKWQTDIFAYILYVISGLGITAGAHRLWAHKSYKAKWPLRVILVIFNTVAFQDAAMDWARDHRMHHKYSETDADPHNATRGFFFSHIGWLLVRKHPDLKEKGKGLDMSDLLADPILRFQKKYYLILMPLACFVMPTVIPVYFWGETWTNAFFVAAMFRYAFILNVTWLVNSAAHKWGDKPYDKSIKPSENLSVAMFALGEGFHNYHHTFPWDYKTAELGNNKLNFTTTFINFFAKIGWAYDLKTVSDDIVKNRVKRTGDGSHHLWGWGDENQSKEEIDAAIRINPKDD

SEQ ID NO: 47 - protein, 353 aa, Heliothis virescens
MAPNISEDVNGVLFESDAATPDLALATPPVQKADNRPKQLVWRNILLFAYLHLAALYGGYLFLFSAKWQTDIFAYILYVISGLGITAGAHRLWAHKSYKAKWPLRVILVIFNTVAFQDAAMDWARDHRMHHKYSETDADPHNATRGFFFSHIGWLLVRKHPDLKEKGKGLDMSDLLADPILRFQKKYYLILMPLACFVMPTVIPVYFWGETWTNAFFVAAMFRYAFILNVTWLVNSAAHKWGDKPYDKSIKPSENLSVAMFALGEGFHNYHHTFPWDYKTAELGNNKLNFTTTFINFFAKIGWAYDLKTVSDDIVKNRVKRTGDGSHHLWGWGDENQSKEEIDAAIRINPKDD

SEQ ID NO: 48 - protein, 352 aa, Choristoneura parallela
MAPNVTEENGVLFESDAATPDLALARTPVEQADDSPRIYVWRNIILFAYLHLAAIYGGYLFLFSAKWQTDIFAYLLYVASGLGITAGAHRLWAHKSYKAKWPLRLILTIFNTIAFQDSAIDWARDHRMHHKYSETDADPHNATRGFFFSHIGWLLVRKHPELKRKGKGLDLSDLYSDPILRFQKKYYMILMPLACFILPTVIPVYMWNETWSNAFFVAALFRYTFILNVTWLVNSAAHKWGDKPYDKSIKPSENMSVSLFAFGEGFHNYHHTFPWDYKTAELGNHRLNFTTKFINFFAKIGWAYDMKTVSQEIVQQRVKRTGDGSHHLWGWGDKDHAQEEINAAIRINPKDD

SEQ ID NO: 49 - protein, 411 aa, Bombyx mori
MPPRLNHFDPDIALYTSTTTLKLLIKLEFVPNRPILRYLLEKKATLMGYHFQNMPPQVDPIPSGVVFESDTQTDDLGLDKDVSVLKKATPKKHELVWFNIIWFLFLHISSLYGLYLVFTSAKWQTNVFAFGVHLMCAIGIGAGSHRLWTHRSFKARTPLRIVLMIWQTMGFQDCIFEWARDHRTHHKYADTDADPHNAERGLFFSHMGWLCCKKSPEVIEGGRRIDLSDLYADPVVMFQKKHYMKMMPILCFVLPTVIPVYFWDETWLNAFFIPTILRYTCGINVVWSVNSFAHTFGYRPYDKSLNPRENIGVWMICVEGFHNYHHTFPWDYRATEHPLYNMLTPTIVFIDAMAMIGQAYDLKTVPHDIIKQRTHRTGDGTHQLWGWDDPEFNEKLKEKYSRVSYAERKCG

SEQ ID NO: 50 - protein, 228 aa, Lobesia botrana
YGLYLCFTSAKWATIFFNVMLHVLAVWGVTAGAHRLWAHRSYKAKLPLQILLMLLNSLAFQTPVSHWARDHRLHHKYSDTDADPHNATRGFFYSHIGWLLVRKHPEAIKRGKTIDVTDIMENPVLQFQKKYYVYLSGLLTFFLPALIPIYYWGETLNNAHHVSLLRLIIGYNVTFFVNSAAHKYGNKPYDKHMSPTQSVTLSLATLGECFHNYHHVFPFDYRTAELGN

SEQ ID NO: 51 - protein, 128 aa, Lobesia botrana
MKIPCRSFKRNKYYLYLSGPLTFILPALIPVYCWKETLNNAYHISLLRLITGYNIAFSVNSAAHKFGNKPYDKHMSPTQSVTLSLATLGECFHNYHHVFPFDYRTAEFGNNYINFTAKSIDFFARMGW

SEQ ID NO: 52 - protein, 140 aa, Lobesia botrana
FVLYIMSGLGITAGAHRLWAHKSYKARLPLRILLTFFNTMAFQDSVLDWARDHRMHHKYSETDADPHNATRGFFFSHVGWLLVRKHPQIKAKGHTIDMSDLLADPVLRFQKRYYLTLMPLVCFILPSYIPTLWGESLWNA

SEQ ID NO: 53 - protein, 103 aa, Lobesia botrana
TVQVLLHLVSIYGLYLIITFNVKFLTTLFAFLTIYTSGFGITAGVHRLWSHRAYQARTPLRVLLALLFTVTGQRDIYTWALDHRVHHKYTETAADPHDIRRGF

SEQ ID NO: 54 - protein, 350 aa, Lobesia botrana
MPPQEQPAAWVLDESDAATEDKDVAAHVPPSAEKRKLEIVWRNVTLFVFLHVGAVYGGYLFFTQAMWATKIFTFVLYVMSGLGITAGAHRLWAHKSYKARLPLRILLTFFNTMAFQDSVLDWARDHRMHHKYSETDADPHNATRGFFFSHVGWLLVRKHPQIKAKGHTIDMSDLLADPVLRFQKRYYLTLMPLVCFILPSYIPTLWGESLWNAYFVSAIFRYCYVLNVTWLVNSAAHKWGDKPYDKNINPVETKPVSLVVFGEGFHNYHHTFPWDYKTAELGGYSLNLSKLFIDTMAKIGWAYDLKSVSPDIVEKRVKRTGDGSHHVWGWDDAPTEQKVAATIVNPDKTE

SEQ ID NO: 55 - protein, 371 aa, Lobesia botrana
MEFLSEIENCSERLRKTEKMPPNVTDVNGVLFESDAATPDLALATPPVQKADNTPRVYVWRNIILFGYLHIAALYGGYLFLVSAKWQTDIFAYLLYVASGLGITAGAHRLWAHKSYKAKWPLRVILTIFNTIAFQDSAIDWARDHRMHHKYSETDADPHNATRGFFFSHIGWLLVRKHPELKRKGKGLDLSDLYADPILRFQKKYYLILMPLACFVMPTLIPVYFWGETWTNAFFVAALFRYAFILNITWLVNSAAHKWGDKPYDKTIKPSENLSVSLFAFGEGFHNYHHTFPWDYKTAELGNHRLNFTTKFINFFAKLGWAYDMKTVSDEIVQKRVQRTGDGSHHLWGWGDKDQSKDEVDAAIRINPKDD

SEQ ID NO: 56 - protein, 361 aa, Lobesia botrana
MRSVSTRVNKAMVPRLEVNTGVKTVDEEQLPSRDENKPRISKLVKINIVSFGYLHIAALYGLYLCFTSAKWATIGLAFFLLVVGEIGITAGAHRLWSHRTYKANRALEILLIIMNSVAFQNSAIHWIRDHRLHHKYSDTNADPHNASRGFFYSHIGWLLVRKHSDVINKGKMIDMSDIYNNPVLTFQKKYAIPFIGTICFGLPTLIPMYCWGESLNNAWHINIMRYVLCLNITFLVNSAAHFFGQKPYDKNILPGQNILVSILAVGEGFHNYHHVFPWDYRTAELGNNMLNLTTKFIDFFAWLGWAYELKTVSTNAIDLRAKRTGDGTNLWGWGDEHMSQEEMEASDILFVNKENEKTHTH

SEQ ID NO: 57 - protein, 330 aa, Lobesia botrana
MENSKSEVREEVPFRHTLVKHNVIKFGYMHLAALYGVYLCFTSAKWPTLLWAFLLLEFAKIGITAGAHRLWCHRSYKAKLPLEILLLIFNSIAYMNTATYWVRDHRVHHKFADTDADPHNVNRGFWFSQIGWLFVRKHPDVVEKGATVFMDDIHQNKLLRFQKKYAFFVIGLWAYVIPTVVPMYFWGESLNNSWHICTMLRHVLTINQIFLVNSIGHSWGNKPYDKNIRAVENVAVSLMSTGECFHNYHHVFPFDYKASELGMTKFNAATMFINFFAWVGWAYDLKTIPDELIIARSKRTGDGRNLWGWGDVDQTEEETKGVKYLYTKNE

SEQ ID NO: 58 - protein, 325 aa, Lobesia botrana
MEKESKTKSSWFRTFEKKLGFQNEIKWVNTILIAIYHMLFVYWPYHYAFPVKWQTIVFAAIMYVASGFGVTGGAHRLWTHKAYKATLPLKIFMLMLYSSAGQNSLLNWVRDHRVHHKFSDTDADPHNANRGLFFSHIGWLMLKKNDEVIRKGKQIDMSDIENDPVLRFHEKYFQWFKLLFCYYLPTMINVWCWGEDWKTAVAWQCFIRYITTFHSELTVNSLAHAFGYKPYNKDLKPAENRFVATVSLGEGWHNYHHAFPFDYKAAEHFDFFNFGTTFIRWFEKLGLAYDLRQATPEMINAISKRLGDGTPVHFPLDSDGERAVG

SEQ ID NO: 59 - protein, 313 aa, Lobesia botrana
MEKNEIRNQTQSSSWFRNLEKRLGFRTDIKWGNVIHITLYHALVFYMAINYAFPVKWQTILFGIYTYITAGYGVTAGAHRLWSHRSYKVTLPLKIWTLICYSMAGQNSVLNWVRDHRVHHKHSDTDADPHNINRGLFFAHVGWLMMKKNEEVVRRGKEIDLSDIENDPVMRFHEKYFGLSKILLCYAVPLAINTWCLGEAWPTAAAWHLMRHLGTLHATFSLNSLAHAYGYQPYNKNMRPTQNRFVATVTFGEGWHNYHHVFPFDYKGAEFGDVLNFTTAYIRGYRYLGLAYDLKEATPEMIRSVAARVGDGS

SEQ ID NO: 60 - protein, 357 aa, Lobesia botrana
MGVTVGAHRLWAHRAYKARWPMRLLLALLQTMAFQNHIYEWVRDHRVHHKYTETDADPHNARRGFFFSHIGWLMVRKHKEVIEKGAAIDMSDLEKDPIVMFQKKTYLVVMPFLCFLLPSWVPVHFWGEDPWTSWYVASIWRYTLSLNFTWLVNSAAHIWGNKPFDNVRGATNWSPPVWSTQVPMGGHGAGGSAIICGIFKSATRKKLRMIGKDGVLFRRQRPSLGSKPSIGATDNVTVAICAVGEGWHNYHHVFPWDYKAAELGNYRTNVSTAIIDLAAKYGWAYDLKTVSAQMIQNRVARTGDGTHPSVAKTVEELDHSEDEHDHHHPENPVWGWDDKDMPEEDRQLVEISHQKSE

SEQ ID NO: 61 - protein, 335 aa, Lobesia botrana
MGVTVGAHRLWAHRAYKARWPMRLLLALLQTMAFQNHIYEWVRDHRVHHKYTETDADPHNARRGFFFSHIGWLMVRKHKEVIEKGAAIDMSDLEKDPIVMFQKKTYLVVMPFLCFLLPSWVPVHFWGEDPWTSWYVASIWRYTLSLNFTWLVNSAAHIWGNKPFDNVRGATNWSPPVWSSQVPMGGHGEGGSAIICGIFKSATGKKNIGATDNVTVAICAVGEGWHNYHHVFPWDYKAAELGNYRTNVSTAIIDLAAKYGWAYDLKTVSAQMIQNRVARTGDGTHPSVAKTVEELDHSEDEHDHHHPENPVWGWDDKDMPEEDRQLVEISHQKSE

SEQ ID NO: 62 - protein, 390 aa, Lobesia botrana
MLTAGLCDIFIVRLRKTEKMPPNVTDVNGVLFESDAATPDLALATPPVQKADNTTQSLRVAKHNSVSVIFTSPLCMGATCSLCRRNGKPIFLPTYYMWHRDWASQQARTGSGHTSPIKQNGLSESFSPSSTLLHSSSMVGLSIYIGINHMFCHQDSAIDWARDHRMHHKYSETDADPHNATRGFFFSHIGWLLVRKHPELKRKGKGLDLSDLYADPILRFQKKYYLILMPLACFVMPTLIPVYFWGETWTNAFFVAALFRYAFILNITWLVNSAAHKWGDKPYDKTIKPSENLSVSLFAFGEGFHNYHHTFPWDYKTAELGNHRLNFTTKFINFFAKLGWAYDMKTVSDEIVQKRVQRTGDGSHHLWGWGDKDQSKDEVDAAIRINPKDD

SEQ ID NO: 63 - protein, 362 aa, Lobesia botrana
MPPQVDPTPSGVVFESDTQTADLGLDKDVSVLKKATPKQHEYVWGNIIWFLYLHIASLYGLYLVFTSAKWQTNVFAFAVHLMCAIGIGAGSHRLWTHRSFKARTPLRIVLMLWQTMGFQDCIFEWARDHRTHHKYADTDADPHNAERGLFFSHMGWLCVKKSPEVIEGGKRIDLTDLYEDPVVMFQKKHYLKLMPLLCFVLPTVIPVYCWNETWLNAFLIPTILRYTCGINVVWSVNSFAHVFGYRPYDKSLNPRENLAVWMVCVEGFHNYHHTFPWDYRATELPLYNMLTPTIVFIEAMAKIGQAYDLKSVSPEIVKQRSQRTGDGSHQIWGWDDPEFTEKLKEKLGAVSNTERGEKVKSG

SEQ ID NO: 64 - protein, 400 aa, Lobesia botrana
MTATDNLISPPEADGDILSRIEDLCDSWNKVKNEETEKPQIPLQLPKTIGTDYSYRHEIVWKNAIGFLILHILAVWGLGVLLTGVVAVCLFSTEGVTIGAHRLYTHKSFKATPFLRALLILAQTTAGQNSMFIWVRDHRLHHRYSDTDADPHNSKRGFFFCHIGWLMSKKHPYVIELGRKIDMSDLEADWMVMFQKKYYYWLYFLLAVFIPTWVPVHYFGESWKNSLLVCYIFRYMVQVNGTWLVNSAAHLCGTRPYDRGLQPVESWFVSFISLGEGWHNYHHAFPWDYKAAELSTKFNQSAKIIKFFEDIGLAYDLKTASPEMVAKRIVRTGDGTHIDLGNEEARAAVTAPGIVHPLNPFYTVTYQPPSGTLKHEGLPLFHEKDVFNSNGNFLKRSARA

SEQ ID NO: 65 - protein, 346 aa, Lobesia botrana
MASKSHREVRMQQTFASGNKSPFRDLVGPQADPSWRWRVSWFNVLLFSYSYSSALQGLYLCFTSAQWATIFLNAILLVASLQGALIGAHRLWSHRSFKAKLPLQILLLVLQTLSVQYSVYNWVRDHRLHHRHSDTDADPHNATRGFFFSHIGWLLVQKHPEVVKRGKATDMSDIINNPLLMFQKKYWAILSFLISLVIPTATPMYFWGENFKNALCVNALRYNLCLNFTWSVNSVAHTFGSRPYDGSIQSRQNMAVTVVTLGEGFHNYHHAFPRDYAAGELGEWFNFSKIFIDFFAWIGWAYDLKTVDKKTVLKRVLEKGDGSYRCKTENKTKINGSKIGNNMLLL

SEQ ID NO: 66 - protein, 348 aa, Lobesia botrana
MMMACLPKTGAAMQKARANRDLFEVFVPVITRSRYLQSKTNSQLKAYHFLAWAWYVLGELGVIAGAHRLWAHRTYKAKMPLQIILMLFNCIGFQNTATDWVRDHRVHHKHSDTDADPHNSQRGSVSFDLHHSAVELYTYRAMFFFSHVGWLLTRKHRLVKEKGEAVDMTDIYSNPVLRFQKKYSLPLIGTLCFALPTLLPAYCWGEAVGTAWNINLLRYCLNLNGTFLTNSAAHKFGSKPYDKTIPPTQNLLVSFMTLGEGFHNYHHVFSWDYRAAELGNTYLNMTTIFIDFFALIGWAYDLKTVPEDVIKKRMARTGDGTNLWGWGDKDMTKEDVVDTEIRFPAKKE

SEQ ID NO: 67 - protein, 324 aa, Lobesia botrana
MSSTDIYSAEVYPKLKAPQADNHKFRICYTNVAVVLYHHLAGLYGLYLCFSCQWATIILAGILQEAGMLGLMAGAHRLWAHRSYKAKLPLQIILIIFHSIANQYTAYNWVRDHRLHHRYSDTDGDPHNATRGFFFSHVGWVLVQKHPEVHRRGASTDMSDLEKNPVVMFQKKYKVPFIALITYVIPTLVPIYFWNETFTNAWHINILRMVVCLNCTFLINSAAHIYGSRPYDKDVRATENMAVNVATLGEGFHNYHHVFPWDYRAEEFGRRLNVTRAFIELFARVGWAYGLKTVGRGAVVARCGRRGDGSYVEQKYDDKGLRDE

SEQ ID NO: 68 - protein, 330 aa, Lobesia botrana
MDDSKSEEVPFRHTLVKHNVIKFGYMHLAALYGVYLCFTSAKWQTLLWAFLLLEFAKIGITAGAHRLWCHRSYKAKLPLEILLLIFNSIAYMNTATYWVRDHRVHHKFADTDADPHNVNRGFWFSQIGWLFVRKHPDVVEKGKTVYMDDIHKNPLLRFQKKYAFLVIGLWAYVIPTVVPMYFWGETLSNSWHICTMLRHVLTINQIFLVNSIGHSWGNKPYDKNIRAVENIAVSLMSTGECFHNYHHVFPFDYKASELGMTKFNAATLFINFFAWVGWAYDLKTIPDELITARSKRTGDGRNLWGWGDVDQTEEDTKGVKYLYTTNGKIE

SEQ ID NO: 69 - protein, 334 aa, Lobesia botrana
MINPTCDSTCKANGLTNEAFQNLLEISTDSTLKRKYHDGSQEEFWMQVYVENNAVGQIAIKRLVRFNSSLASSIYTYITAGYGVTAGAHRLWSHRSYKVTLPLKIWTLICYSMAGQNSVLNWVRDHRVHHKHSDTDADPHNINRGLFFAHVGWLMMKKNEEVVRRGKEIDLSDIENDPVMRFHEKYFGLSKILLCYAVPLAINTWCLGEAWPTAAAWHLMRHLGTLHATFSLNSLAHAYGYQPYNKNMRPTQNRFVATVTFGEGWHNYHHVFPFDYKGAEFGDVLNFTTAYIRGYRYLGLAYDLKEATPEMIRSVAARVGDGSRYSVPTKEANQ

SEQ ID NO: 70 - protein, 345 aa, Lobesia botrana
MAPQSEEIDTSSGDIKDASTELLKMPETRVTPRKYTIVYYTVVLLVYYHLAALYALYLCWTSVKWQTVFFNVILHVAAISGVTIGAHRLWSHRAFKAKMPLQILLMLLNSMASQNTACYWARDHRLHHKFNDTDADPYNSTRGLFYSHIGWLVVKKHPDVVKKGKEIDMSDLYGNPVLKFQNKYYIVLTLALGFVLPTLLPIYYWQETLTNAYHVSLLRFVIGLHAIFFVNSVAHKYGNKPYDKYSSATQSIPVSVIALGECYHNFHHAFPFDYRSSEIGNNYINFGTHIIDFFAWIGWAYDLKVASEDMVLKRANKTGDGTDLWGRVKGVEKPNEDWVDKDLMR

SEQ ID NO: 71 - protein, 327 aa, Lobesia botrana
MAPQSEEIDTSSGDIKDASTELLKMPETRVTPRKYTIVYYTVVLLVYYHLAALYALYLCWTSVKWQTVFFNVILHVAAISGVTIGAHRLWSHRAFKAKMPLQILLMLLNSMASQNTACYWARDHRLHHKFNDTDADPYNSTRGLFYSHIGWLVVKKHPDVVKKGKEIDMSDLYGNPVLKFQNKYYIVLTLALGFVLPTLLPIYYWQETLTNAYHVSLLRFVIGLHAIFFVNSVAHKYGNKPYDKYSSATQSIPLSLIALGECYHNFHHAFPFDYRSSEIGNNYINFGTHIIDFFAWIGWAYDLKVASEDMVSKRANKTGDGTDLWGR

SEQ ID NO: 72 - protein, 358 aa, Lobesia botrana; identical to SEQ ID NO: 2

SEQ ID NO: 73 - protein, 370 aa, Lobesia botrana
MYMGDGNCLPSELKMTPNPRNDVTEDKPLQEDYLEKLVAPQASPIKFEASYVNLIMFTYFHTAGAYGLYLAFIEARWATIFEEIEKISSLFPAFALYEAGIVGVTAGAHRLWAHRTYKAKLPLQILLMFLHSFSCQYSAFHWARDHRLHHKYSDTDGDPHNATRGFFFSHIGWLLVKKHPEAKRRLRGIDVKDLLENRVLQFQKKYATPFIGTICFILPTLVPMYFWDEPFRTAWNVNILRVIVLLHATFLVNSAAHAFGNKPYDASITPSQSISISIATLGEGYHNFHHVFPWDYRAAELGNNVLNFTTLFIDFFARIGWAYDLKTVGNDAIAKRAERTGDGTNLWGWGDKDLPKKHEEIARVSFEKAE

SEQ ID NO: 74 - protein, 316 aa, Lobesia botrana
MTPNPRNDVTEDKPLQEDYLEKLVAPQASPIKFEASYVNLIMFTYFHTAGAYGLYLAFIEARWATIFLGSLILMFLHSFSCQYSAFHWARDHRLHHKYSDTDGDPHNATRGFFFSHIGWLLVKKHPEAKRRLRRIDVKDLLENRVLQFQKKYATPFIGTICFILPTLVPMYFWDEPFRTAWNVNILRVIVLLHATFLVNSAAHAFGNKPYDASITPSQSISISIATLGEGYHNFHHVFPWDYRAAELGNNVLNFTTLFIDFCARIGWAYDLKTVGNDAIAKRAERTGDGTNLWGWGDKDLPKKHEEIARVSFEKAE

SEQ ID NO: 75 - protein, 370 aa, Lobesia botrana
MSPSNATNTLVEAPKYIEDITECRVCRTTHKPSKGDICFNNNNLETKDKHEINHNNNKETKYNTRQHSWLKRWEKWMGFETELIWLNLISISVFHIVTVVWFTYNLWIGSYPKWQSTLYAYFIGNVAGFGVTAGVHRYWCHRSYKATLPLQWILVICYSVAGQNPIPEWVRDHRVHHKFSESSADPHDAGRGFFFAHVGWLMMRKHPDVISEGRKIDMSDITNDPVVQFHTKYFKLFKTAFCFVLPTLVPVWGWNETWEYAILSQVCLRYLMSLNCTWAVNSFAHLWGNKPYDPNITPVENWGVSAVALGEGWHNYHHTFPWDYKAAELAYTLNLTTLLLDGFARIGWAHNLKQATPELIQAVVNRKHKS

SEQ ID NO: 76 - protein, 358 aa, Lobesia botrana
MSPSNATNTLVEAPKYIEDITECRVCRTTHPPSKGDICFNNNNLETKDKHEINHNNNKETKCNTRQHSWLKRWEKWMGFETELIWLNLISISVFHIVTVVWFTYNLWIGSYPKWQSTLYAYFIGNVAGFGVTAGVHRYWCHRSYKATLPLQWILVICYSVAGQNPIPEWVRDHRVHHKFSESSADPHDAGRGFFFAHVGWLMMRKHPDVISEGRKIDMSDITNDPVTAFCFVLPTLVPVWGWNETWEYAILSQVCLRYLMSLNCTWAVNSFAHLWGNKPYDPNITPVENWGVSAVALGEGWHNYHHTFPWDYKAAELAYTLNLTTLLLDGFARIGWAHNLKQATPELIQAVVNRKHKS

SEQ ID NO: 77 - protein, 4 aa, Artificial Sequence
  PPTQ desaturase motif
PPTQ

SEQ ID NO: 78 - protein, 4 aa, Artificial Sequence
  SPTQ motif
SPTQ

SEQ ID NO: 79 - protein, 5 aa, Artificial Sequence
  Desaturase selectivity determinant
  MISC_FEATURE (5)..(5): Xaa is Asn, His, Tyr, Phe, Cys, Ser, or Leu
SLLMX

SEQ ID NO: 80 - protein, 5 aa, Artificial Sequence
  Desaturase selectivity determinant
  MISC_FEATURE (5)..(5): Xaa is Ile, Leu, Glu, Gly, Thr, Ala, or Val
YELSX

SEQ ID NO: 81 - protein, 5 aa, Artificial Sequence
  Desaturase selectivity determinant
  MISC_FEATURE (5)..(5): Xaa is Asn, His, Lys, Asp, or Ser
MSFQX

SEQ ID NO: 82 - protein, 5 aa, Artificial Sequence
  Desaturase selectivity determinant
  MISC_FEATURE (5)..(5): Xaa is His, Lys, Arg, or Ser
WLMTX

SEQ ID NO: 83 - protein, 5 aa, Artificial Sequence
  Desaturase selectivity determinant
  MISC_FEATURE (5)..(5): Xaa is Lys, Ile, Val, or Leu
SEEVX

SEQ ID NO: 84 - protein, 5 aa, Artificial Sequence
  Desaturase selectivity determinant
  MISC_FEATURE (5)..(5): Xaa is Leu, Phe, Tyr, or Trp
LHATX

SEQ ID NO: 85 - protein, 5 aa, Artificial Sequence
  Desaturase selectivity determinant
  MISC_FEATURE: Xaa is Leu, Cys, Met, Phe, or Ser
  misc_feature (5)..(5): Xaa can be any naturally occurring amino acid
HATLX

SEQ ID NO: 86 - protein, 5 aa, Artificial Sequence
  Desaturase selectivity determinant
  MISC_FEATURE (5)..(5): Xaa is Met, Cys, Ala, Val, Thr, or Ser
LNTAX

SEQ ID NO: 87 - protein, 5 aa, Artificial Sequence
  Desaturase selectivity determinant
  MISC_FEATURE: Xaa is Ile, Val, or Ala
  misc_feature (5)..(5): Xaa can be any naturally occurring amino acid
SILGX

SEQ ID NO: 88 - protein, 5 aa, Artificial Sequence
  Desaturase selectivity determinant
  MISC_FEATURE (5)..(5): Xaa is Ile, Leu, Glu, Gly, Thr, Ala, or Val
YELSX

SEQ ID NO: 89 - protein, 9 aa, Artificial Sequence
  Desaturase binding pocket region 1
  misc_feature (1)..(1): Xaa is Ala, Ser, or Thr
  misc_feature (2)..(2): Xaa is Ile, Leu, or Glu
  misc_feature (3)..(3): Xaa is Leu, Thr, or Ile
  misc_feature (6)..(6): Xaa is Thr or Ser
  misc_feature (8)..(8): Xaa is Ala or Gly
XXXGIXAXA

SEQ ID NO: 90 - protein, 10 aa, Artificial Sequence
  Desaturase binding pocket region 2
  misc_feature (1)..(1): Xaa is Leu or Phe
  misc_feature (3)..(3): Xaa is Val or Ile
  misc_feature (7)..(7): Xaa is Val or Ala
  misc_feature (9)..(9): Xaa is Lys or Arg
XCXNSVXHXW

SEQ ID NO: 91 - protein, 10 aa, Artificial Sequence
  Desaturase binding pocket region 3
  misc_feature (1)..(1): Xaa is Leu or Met
  misc_feature (2)..(2): Xaa is Asn or Ser
  misc_feature (3)..(3): Xaa is Thr or Phe
  misc_feature (4)..(4): Xaa is Ala or Phe
  misc_feature (5)..(5): Xaa is Met, Ala, or Val
  misc_feature (9)..(9): Xaa is Gly or Ala
XXXXXLGEXF

SEQ ID NO: 92 - protein, 10 aa, Artificial Sequence
  Desaturase binding pocket region 4
  misc_feature (7)..(7): Xaa is Gln or Arg
  misc_feature (8)..(8): Xaa is Ser, Thr, or Ala
  misc_feature (9)..(9): Xaa is Ser or Ala
VFPWDYXXXE

SEQ ID NO: 93 - protein, 9 aa, Ostrinia latipennis
SILGISAAA

SEQ ID NO: 94 - protein, 10 aa, Ostrinia latipennis
LCINSVAHRW

SEQ ID NO: 95 - protein, 10 aa, Ostrinia latipennis
LNTAMLGEGF

SEQ ID NO: 96 - protein, 10 aa, Ostrinia latipennis
LNFAVLGEAF

SEQ ID NO: 97 - protein, 9 aa, Ostrinia latipennis
AEIGITAGA

SEQ ID NO: 98 - protein, 10 aa, Ostrinia latipennis
FCVNSVVHKW

SEQ ID NO: 99 - protein, 10 aa, Ostrinia latipennis
LNFAVLGEAF

SEQ ID NO: 100 - protein, 10 aa, Ostrinia latipennis
VFPWDYRAAE

SEQ ID NO: 101 - protein, 111 aa, Lobesia botrana
HKYATKPSDTYISATQSIPVSVIALGECYHNFHHAFPFDYRSSEIGNNYINFGTHIIDFFAWIGWAYDLKVASEDMVLKRANKTGDGTDLWGRVKGVEKPNEDWVDKDLMR

SEQ ID NO: 102 - protein, 259 aa, Lobesia botrana
YHLAALYALYLCWTSVKWQTVFFNVILHVAAISGVTIGAHRLWSHRAFKAKMPLQILLMLLNSMASQNTACYWARDHRLHHKFNDTDADPYNSTRGLFYSHIGWLVVKKHPDVVKKGKEIDMSDLYGNPVLKFQNKYYIVLTLALGFVLPTLLPIYYWQETLTNAYHMSLLRFVIGLHAIFFVNSVAHKYGNKPYDKYSSATQSIPVSVIALGECYHNFHHAFPFDYRSSEIGNNYINFGTHIIDFFAWIGWAYDLKVA

SEQ ID NO: 103 - protein, 114 aa, Unknown
  Sequence isolated via Lobesia botrana RNA-seq assay
EHAVVPDSSDPLRNTRTTHANVLERLFIAPYYVNYHLEHHLLFYVPCYNLPQVHRLLGESRHAGRMEVQQGYAAVLRLATARPDRDDRPGQLVSNARRARAGAAVDANQTNGGF

SEQ ID NO: 104 - protein, 131 aa, Unknown
  Sequence isolated via Lobesia botrana RNA-seq assay
IHIADVVKALAISRTDAAKRLARWREQGWMSRVGAGAYVPASIDTLGSERVLDDAWVLVPALFAPAYIAGRSAAEHWDLTEQIFKDIVVVTGQNIRERHQKRQGFEFTLKHLKPEKIFGTKPVWRHHTKVP

SEQ ID NO: 105 - protein, 138 aa, Unknown
  Sequence isolated via Lobesia botrana RNA-seq assay
TAAPIPLPTSPTSGLSPATSPLAIRIGSWLAPEARIKAFLKNSATALCAAIVVLSSFSLGAEAARRHHRSHHHHLPAQPAAPPRALPYPQLPLPFEISGAQYLPLPWTEVKGWNDDDHLAAYKTFRASCKSINAQTGT

SEQ ID NO: 106 - protein, 100 aa, Unknown
  Sequence isolated via Lobesia botrana RNA-seq assay
SLVEMSLGVAIGALTFTGSVIAFLKLSARMSGAPIILPFRHVINIGLALALVFFIVGLVLSGSAFDFWMIVILALALGVLMIIPIGGADMPVVISMLNSY

SEQ ID NO: 107 - protein, 335 aa, Unknown
  Sequence isolated via Lobesia botrana RNA-seq assay
PADDPHDDVMYPSAIPIVLVHLGCLAAIWSGVTWQAVAICVSLYVVRMFAIGAGYHRYFSHKAFATSRVFQFILACLAQSTAQKSVLWWAAKHRHHHLHSDTELDVHSPRQRGFVYSHLGWIFYREHDATDLVKVEDLARYPELMWLHRLELLPAVVLAVLCFLVAGWPGLVVGFLWSTVLLYHATFCINSLAHVHGRKRYVTGDNSRNNWLLAFFTLGEGWHNNHHAYQSSVRQGFRWWEVDVTYYVLTILSWIGIVWNMKAPPEQVLRNEQPLGARVINRAARELAARFDAQTVAHAISSARRRADLAQMQLPTLQDILNRAHDGVDALTHLH

SEQ ID NO: 108 - protein, 176 aa, Unknown
  Sequence isolated via Lobesia botrana RNA-seq assay
AFRSRGLNLAVGYLSAFIIGLPYEYYCLFHWDHHRYTQDPDRDPELIVGAKPATDTQLAIAYSGLLQVAGRLWLMLGHAVTGKVVVPWIPENKRATIVTEARTYVALYALLLALSLWFASALLLWLWIVPLFVGQFFLRPYLYAEHTGCDTTRSAFQNTRTTYTGAIVKWFAWNMP

SEQ ID NO: 109 - protein, 165 aa, Unknown
  Sequence isolated via Lobesia botrana RNA-seq assay
MSNRTRLPDTFSRLAWSNLAAQSAEQIALAAAPIVAVLTLGVAEGQTGLLQTALTLPFVLFAIPAGLLADRISRRALMAGAEALRAVALGAIVLLLALGALNLPLLALLGFAAVCGTVVYSVAAPALVPSLVSADLLPAANARIELARTIAFASGPALGGALVGW

SEQ ID NO: 110 - protein, 113 aa, Unknown
  Sequence isolated via Lobesia botrana RNA-seq assay
LAFIEARWATIFLAFALYEAGIVGVTAGAHRLWAHRTYKAKLPLQILLMFLHSFSCQYSAFHWARDHRLHHKYSDTDGDPHNATRGFFFSHIGWLLVKKHPEAKRRLRGIDVK

SEQ ID NO: 111 - protein, 360 aa, Lobesia botrana
MVDIAPNGQKIAEPVADIDHEFRPLVAPQAGPRKYTVVYMTVLNFLYWHLGGFYGLYLCFTSAKWATIFFNVMLHVLAVWGVTAGAHRLWAHRSYKAKLPLQILLMLLNSLAFQTPVSHWARDHRLHHKYSDTDADPHNATRGFFYSHIGWLLVRKHPEAIKRGKTIDVTDIMENPVLQFQKKYYVYLSGLLTFFLPALIPIYYWGETLNNAHHVSLLRLIIGYNVTFFVNSAAHKYGNKPYDKHMSPTQSVTLSLATLGECFHNYHHVFPFDYRTAELGNNYINLTTKFIDFFAVIGWAYDLKYASDDMIRKRSKRTGDGTDLWGRVSGTEGKLTPGNIWGWGDKDMPEEHKEILLSQQ

SEQ ID NO: 112 - protein, 734 aa, Lobesia botrana
MTEVTKVNPDLQRERDNCTFNVTELTNLIDGGVQKTEERRKREEMVLKEGIHLDEVPSEYLSHKEKYELAVKKACYLFKMIRRLQEEENTGMENYQYVTYLHWSSQYESESTVGIEQSPSELNNASAALNYLKKLRVARKKRAAAIAKVHFKHCLGIYHREVLGGSLGSAILRDGSPLTLHYVMFIPTIMGQATVEQQVLWIGRAFNCDIIGTYAQTELGHGTFIRGLETTATYDPSTKEFVLHSPTLTSYKWWPGGLAHTANYCIVVAQLYTQGKCHGIHPFIVQLRDEETHMPLPGIKVGEIGAKLGMNGTNNGFLGFDKVRIPREHMLMKNAKVLEDGTYVRAPSSKLTYGTMMFVRVVLVNDVCSYMAKAVTIATRYSAVRHQSQPKPDEPEPQILEYVTQQHKLAIGIATVHAFRLSASWLWNMYNNVTAELDAGDLERLPELHALSCCLKAVSTADASECVERCRLACGGHGYMLSSNFPLMYGMVTAACTYEGENTVMHLQTARYLVKAWQQAQGGNPLTPTVAYIGTIGAGRRSPSWDNTVEGIILGFQRVAAGKIAQCVANIEKRQRTGMSYEDAWNMTSVQLVSASEAHCRAFILRTYFEETENQIGSVSPALRAVLLQLVDLYVVFWALQRVGDLLRFTSISERDIEQLQSWYEDLLIKLRVNAVGLVDAFDIRDEILNSALGAYDGRAYERLMAEALKSPLNAEPVNQSFHKYLKPFMQGKL

SEQ ID NO: 113 - protein, 687 aa, Bicyclus anynana
MEDLSFVPELPSGPLDVYRNNASFNWKRLKLALEGDFDLLKLKYKIWRTLEKDPLFAHSPITLPVEEQKRITQLQLKKINEYKFIPKEMFNASYSKRTRTIMTINEAVQSLNPSVSVKMAIGIYLFSNALLSLGTERHLKFYEATIKHREILASFSLTEIAHGSDARLMRTTATYDPSTREFVVHTPDFEAAKCWVGNLGRTCTHTLLFAQLITPDGTNHGLHGFVVPIRKPDTLETYPGLIVGDMGEKIGVNGIDNGFIMFNQYRIPRENLLNRTADVTEDGTYESSFSEPSRILGAALENLSAGRIGIMQESCHSLSSAVTIAVRYAATRTQFGERDRETPLIEYELHQWRLFGYVSAAIVFRLYIDLFAHTYLNIVEKSNAGHRVDNLSEAVSEIHAMVSSSKPLLTWTTLRAAQQCREACGGHGYLKCANLGDIRSNHEPTVTYEGDNNVLSQQAGNWLLRQYEAAIAGKPVDSPLGTVAFLKDLRTIKDRTFTCKTTKELKDPQFIISTYKWLLCWMLKYTHEKNEANLSQGLTKVQARTKSQVYRWKTLTKVYAEYITLVFCLDNIDKKERELQPMLLKLFCLYGLWSLDENLVELYQGGFAKGEECARLVRDSVLELCGEVKNEIVSVADALAPTDFVLNSVLAKSDGKLYQNLQKAFFSHPGVFERTPWWRDVVPASKL

SEQ ID NO: 114 - protein, 668 aa, Plutella xylostella
MSAKVNPDLQRERDKCTFNVTELTNLIDGGVQNTEERKRQEDLLLKEGIHIEEVPSEYLSHKEKYELAVKKACLLFKVIRRMQEEENTGMENYRSVLGGNLGSAILADGSPLTLHYVMFIPTILGQATVEQQAYWIGRAFNLDIIGTYAQTELGHGTFIRGLETTATYDPSTKEFVLHSPTLTSYKWWPGGLAHTANYCIVMAQLYTKGQCHGLHPFIVQLRDEETHMPLKGIKIGEIGVKLGMNGTNNGFLGFEHVRIPRENMLMKNSKVLEDGTYVHSLSSKLTYGTMMFVRVVLVTDMCNYMAKAVTIATRYSAVRRQSQPKPDEPEPQILDYVTQQHKLMIGISTVHAFRLSANWLWHMYNNVIAELDTGDLERLPELHALSCCLKAVTTADAAECVERCRLSCGGHGYMLSSSLPTTYGLVTAACTYEGENTVLLLQTARYLVKAWQQAMGGNALTPTVSYISEVSTGRRSPPWDNSVQGVIHGFHRVAAGKIGMCVANIEKRQKSGLCYEDAWNMTSVQLASASESHCRAILLSTYYSETEKLASKVTPALKTVLLQLVDLYVVYWALQRVGDLLRFTSISERDIEQLQNWYEDLLTRLRPNAVGLVDAFDFRDEILHSALGSYDGRVYERLMEEALKSPLNAQPVNDSFHKYLKPFMQGKL

SEQ ID NO: 115 - protein, 687 aa, Plutella xylostella
MADLSFIPDLPSGPLDCYRSTASFDWKQLKLSLEGDLDLLKLKYKIWKTLEKDLLFAPPSVTPSVEEQKRITQLQLKKINNYKFMTYEMFKSSYSKRTRAIMTINEAVQSVNPSVSVKMAIGIYLFSNALLSLGTERHYKFFEATIWNREILASFALTEVAHGSDARLMCTTASYDPKSREFVVHTPDFKAAKCWVGNLGRTCTHTLLFAQLITPDGTNHGLHGFVVPVRDPATLETYPGLVVGDMGEKIGVNGIDNGFIMFNQYRIPRENLLNRTADVTEDGVYESSFSEPSKMLGAALENLSAGRIGIMQESCHTISSAVAIAIRYAATRRQFGAKDDEIPLLEYELHQWRLFGYVSAAVVFRVYIEKFARIYLNIVEKSNAGLKVDNLSAAVSEIHAMVSCSKPLLTWTTLDAVQQCREACGGHGYLKCANLGDLRCNHEPTVTYEGDNNVLSQQAGNWLLRQYEQAAGGVDSPLGTVTFLTRCQDILEDKFSCERTDQLKDVKFITSTYQWLLCWLLKETHQQYESEVSKGQSKFEAKSKCQVYRWKTLSRVYAEYLAIIFCSESVSQEETGLRPVLNKLLVMYGLWSLDQHLVELYQGGFASGDSLARLLRAAILEMCAQLKPEVVSVVDALAPTDFVLNSVLGKSDGNLYQNLQKALFGGPGAMERASWWREVVNSAPSKL

SEQ ID NO: 116 - protein, 661 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKKLYLANFVEPVGLNYSMFIPTLLNQGTTAQQEKWMRPSQELQIIGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKL

SEQ ID NO: 117 - protein, 661 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKNSVHRGHPEPLDLHLGMFLPTLLHQATAEQQERFFMPAWNLEITGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKL

SEQ ID NO: 118 - protein, 664 aa, Arabidopsis thaliana
MEGIDHLADERNKAEFDVEDMKIVWAGSRHAFEVSDRIARLVASDPVFEKSNRARLSRKELFKSTLRKCAHAFKRIIELRLNEEEAGRLRHFIDQPAYVDLHWGMFVPAIKGQGTEEQQKKWLSLANKMQIIGCYAQTELGHGSNVQGLETTATFDPKTDEFVIHTPTQTASKWWPGGLGKVSTHAVVYARLITNGKDYGIHGFIVQLRSLEDHSPLPNITVGDIGTKMGNGAYNSMDNGFLMFDHVRIPRDQMLMRLSKVTREGEYVPSDVPKQLVYGTMVYVRQTIVADASNALSRAVCIATRYSAVRRQFGAHNGGIETQVIDYKTQQNRLFPLLASAYAFRFVGEWLKWLYTDVTERLAASDFATLPEAHACTAGLKSLTTTATADGIEECRKLCGGHGYLWCSGLPELFAVYVPACTYEGDNVVLQLQVARFLMKTVAQLGSGKVPVGTTAYMGRAAHLLQCRSGVQKAEDWLNPDVVLEAFEARALRMAVTCAKNLSKFENQEQGFQELLADLVEAAIAHCQLIVVSKFIAKLEQDIGGKGVKKQLNNLCYIYALYLLHKHLGDFLSTNCITPKQASLANDQLRSLYTQVRPNAVALVDAFNYTDHYLNSVLGRYDGNVYPKLFEEALKDPLNDSVVPDGYQEYLRPVLQQQLRTARL

SEQ ID NO: 119 - protein, 692 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKAIHKLTEYLSFQVRNVAKELVDAFELPDHVTRAPIAMQSDAYSQYTQVVGF

SEQ ID NO: 120 - protein, 661 aa, Lobesia botrana
MAAKVTINEDLIKERNKCNFDIEELTYIIDGGKEKTLDRRNIETIALKEEYLRKDKSEECLSLKERYELAIEKSVLYATLFRNHVQTGDLDIEAIGSSFLRRTADAYFNEVSPFAIHLGMFVTTILGQGTPDQQAEWLHKALDLQTELGHGSFIRGLETTATYDPDTEQFVLHTPTLSAHKWWAGGLGKTANHAIVIAQTYSKGECYGTHPFIVQIRDLDTHVPLPGIRVGEIGPKMGYVTADNGFLGFDHFRIPRENMLMKNAQILKDGTYVKKSHKKMTYGTMVFIRVILVNDAAFNLAKAVTIAVRYSAVRRQCRPNPDAPEPQVLDYVTQQHKLFIGIATAHALQLTSDWLWRTFNAVQADMRNGKDDNLPELHALSSCLKAVCTSDSLVLIERCRFACGGHGYILSSRLPGIYAYGTAIRTHEGDYTILMLQCARFLVKAFELVEAGNAVSPTVSYLSESISKAGSWDSSLEGIIRSFQRLAAGKISAAVKSIRRRVESGMSYDDAWQLSTVQLVSAAEAHCRAVISHTYWSETLRLTATSTPNVKTVMHQLTSLYVTYWALERTGDLLLYSKISEADIISLQQRYEELLALIRPNAVGLVDAFDIRDEILQSTLGAYDGLVYKRLMDEALKSPLNAEPVNRSFHKYLKPFMQGKL

SEQ ID NO: 121 - protein, 666 aa, Lobesia botrana
MGEVNKDLQRERDKCTFDVKALTTFIDGTPQRTLARKYREKIMLSEKELSEGIPEEYLSHKERYENGIKKSVILFKLVRRLQEEGRSNITNYRDVISGLLGSSSTRDGSSFGVHYIMFMPAILNQGTEEQQAQWLPRAWNCSIIGSYAQTELGHGTFIRGLETTATYDPSTQEFVLHSPTLTSYKWWPGGLGHTANYCIVVAQLYTKGQCHGIHSFIIQVRDEDTHVPLPGIKVGDIGAKLGLNSVDNGFLGFDHFRIPRDQMLMKHAQVLEDGTYVRNARNSKLNYGAMVFVRVVIVYDMVNYLSKAVTIATRFSAVRRQSQLKEGEPEPQILDYVTQQHKIFIGIAACHALRLTANMLWETFHDVTEQLVGGNLERLPELHALACCLKAVSTADTAMYVERCRLACGGHGYMQSSNLPMTYGLVTAACTYEGENTVLLLQTARYLVKTWQSGMESLTPMVAYLKTASAPDFCARWENSVEGIVRGLQKVAMGKVARCVKNIKKRMDSGMGPEDAWNATSVQLVSAAEAHCRVVITATFFDEIKKLDASMPPEVKLVLGQLMELYALYWALEKLNDLLLYTSISTQDVDELQFRYEELLAKLRPNAVGLVDGFDIIDGILQSTLGAYDGRVYERLMDEALKSPLNAEPVNQSFHKYLKPFMQGKL

SEQ ID NO: 122 - protein, 878 aa, Lobesia botrana
MTEVTKVNPDLQRERDNCTFNVTELTNLIDGGVQKTEERRKREEMVLKEGIHLDEVPSEYLSHKEKYELAVKKACYLFKMIRRLQEEENTGMENYQYVTYLHWSSQYESESTVGIEQSPSELNNASAALNYLKKLRAARKKRAAAIAKVHFKHCLGIYHREVLGGSLGSAILRDGSPLTLHYVMFIPTIMGQATVEQQVLWIGRAFNCDIIGTYAQTELGHGTFIRGLETTATYDPSTKEFVLHSPTLTSYKWWPGGLPNPSNFVPTTKNLFPLTIHTPGQVEPLQPPPLVRAFLEALSKFNVSADGFPVCRIALPIYRVRNAVAHTANYCIVVAQLYTQGKCHGIHPFIVQLRDEETHMPLPGIKVGEIGAKLGMNGTNNGFLGFDKVRIPREHMLMKNAKVLEDGTYVRAPSSKLTYGTMMFVRVVLVNDVCSYMAKAVTIATRYSAVRHQSQPKPDEPEPQILEYVTQQHKLAIGIATVHAFRLSASWLWNMYNNVTAELDAGDLERLPEIAVGIATVHAFRLSASWLWNMYNNVTAELDAGDLERLPEIAAGIATVHAFRLSASWLWNMYNNVTAELDAGDLERLPELHALSCCLKAVSTADASECVERCRLACGGHGYMLSSNFPLMYGMVTAACTYEGENTVMHLQTARYLVKAWQQAQGGNPLTPTVAYIGSIGAGRRSPPWDNTVEGIILGFQRVAAGKIAQCVANIEKRQRTGMSYEDAWNMTSVQLVSASEAHCRAFILRTYFEETENQIGSVSPALRAVLLQLVDLYVVFWALQRVGDLLRFTSISERDIEQLQSWYEDLLIKLRVNAVGLVDAFDIRDEILNSALGAYDGRAYERLMAEALKSPLNAEPVNQSFHKYLKPFMQGKL

SEQ ID NO: 123 - protein, 629 aa, Lobesia botrana
MVVSKKVNEDLQKERDKCTFNVQELTHYLDGGEKITRQRKATEDKVLSVKGLRDEIPEEYLSHKERYENAIRKAVIMHQTLGDLDQNNLSEDEKVRLGIRTTISSAIFKDNSPLMLHYSMFTNVLTGQGDEEQQKYWLPKVKNMEIIGTYAQTELGHGTFIRGLETTAVYDPKNEEFVLNSPKLTAYKWWPGGLAHTANHCVVMAHTYSQGKSCGIQPFIVQIRDLETHMPMNGVKLGEIGAKLGFNTVNNGFLGFENHRIPRNRMLMRYSQILKDGTFKSGPNSKLTYGTMVYVRVTILCHMANALGKAVTIATRYAAVRRQSQPKPNEPEPQILDYVTQQHKLFISIATSHAFYLNCHRLWALYSKVNKELAAGNLDNLAELHALACCLKAVSTTDGSVQVERCRLACGGHGYMLSSNMPQTYANVTAAYTYEGENTVLHLQTARALVKAGTQVAMGQKVSPSMEYLADRSKMTKWSNSVQGIIKGFQKVAAGKVEACLESLQRHTGNGQSPEDAWNLTSVQLVAASEAHARLVLLSVYKSELDATTFTLSGETTTVLQQLVELYATYWALERLGDLLLRVCRRLSKVYECPRVGYAVSNMTRDRNFGADDRRMTSSYIQSVLAFLV

SEQ ID NO: 124 - protein, 714 aa, Lobesia botrana
MIGLEMQANSPASRLGRTYTPITPLIAAANRACKKNLYTNQILLKFQDKVWKFMLAHPLFKHSVATPTLDEQRQIATKRMYLLHNADLVPLEEIAMHPRLFQSFTEAIFMFDSSVAVKLSLTFRMFTNTIRGSGRQHHYHLIEDCDNGKIGGCFALTEIAHGSNAKGMRTTATYDVEGRCFVMHTPDFEAAKCWVGSLGKCATHAIVYAMLISKGKNHGLHSFVVPIRDPKTLRPFAGVTVGDIGEKIGLNGVDNGFLMFNKYRLPKEAALDKLGGVDENGDYKTPFRDPSKRFGASLGILSGGRVHITSISTNYLQKAVVIAVRYSAVRRQFGPENAVEETPVLEYQQQQIRLLPYLAATFAMRVFCNWFGEVHVQMTIDNLVGTDEQAAARGIEMHALSSAVKPVCGWTARDGLQNCRECCGGHGYLRVVMLACLAHYNARKKAFKRLKCMDRYRCSVLMPTSAIGDLRNDNDANCTYEGENSVLLQQTSNWLLGVWARRRQPGFADTPFGSIQFLDGAQALLDESCQWNSAEEIVQTVACGAHVQMADHLYAEADGGQGLPAENPGQEQLRGPERLAVLQRRHTVRGLWRALQFQDAACRAVLLKLVALYGAFLLEKHMATLYIGGFFTKQQGLLLREGVLSLCSALSPQAVSLADTLAPPDWCLHSVLGYADGEAYKHIQDSIMTYPGSMTRPEWWRDVAHWETYVPAKL

SEQ ID NO: 125 - protein, 700 aa, Yarrowia lipolytica
MNPNNTGTIEINGKEYNTFTEPPVAMAQERAKTSFPVREMTYFLDGGEKNTLKNEQIMEEIERDPLFNNDNYYDLNKEQIRELTMERVAKLSLFVRDQPEDDIKKRFALIGIADMGTYTRLGVHYGLFFGAVRGTGTAEQFGHWISKGAGDLRKFYGCFSMTELGHGSNLAGLETTAIYDEETDEFIINTPHIAATKWWIGGAAHTATHTVVFARLIVKGKDYGVKTFVVQLRNINDHSLKVGISIGDIGKKMGRDGIDNGWIQFTNVRIPRQNLLMKYTKVDREGNVTQPPLAQLTYGSLITGRVSMASDSHQVGKRFITIALRYACIRRQFSTTPGQPETKIIDYPYHQRRLLPLLAYVYALKMTADEVGALFSRTMLKMDDLKPDDKAGLNEVVSDVKELFSVSAGLKAFSTWACADVIDKTRQACGGHGYSGYNGFGQAYADWVVQCTWEGDNNILTLSAGRALIQSAVALRKGEPVGNAVSYLKRYKDLANAKLNGRSLTDPKVLVEAWEVAAGNIINRATDQYEKLIGEGLNADQAFEVLSQQRFQAAKVHTRRHLIAAFFSRIDTEAGEAIKQPLLNLALLFALWSIEEDSGLFLREGFLEPKDIDTVTELVNKYCTTVREEVIGYTDAFNLSDYFINAPIGCYDGDAYRHYFQKVNEQNPARDPRPPYYASTLKPFLFREEEDDDICELDEE

SEQ ID NO: 126 - protein, 325 aa, Yarrowia lipolytica
MSAVPIEFNVPSVDRPFGIYLWAIFDQAWEKLFGWPASSFIFVRNDPNIPFSSTPPVIIAIIVYYIVIFGGREVMRNLSPIRLNWLFQIHNIFLTLLSGMLLLLLVEQLFPIIVRQGILYAICDYGSWTQPIVFCYYLNYLTKYFELIDTVFLVLRKKKLTFLHTYHHGATALLCYTQLIGKTSVSWVPITLNLFVHVVMYFYYFLAARGIRVWWKEWVTRLQIIQFVIDLGFVYFASYTYFTSTYWPWMPNMGSCAGEEFAAIYGCGLLTSYLFLFIAFYINSYRKPSSKGPSKPVVAVDGPVGGVNAQTGASRGQTTTRSRRA

SEQ ID NO: 127 - protein, 304 aa, Yarrowia lipolytica
MLSSISPDLYSSFSFKNSLAEAMPSVPHELINSKTLSWMYNASLDIRVPLTIGTIYAVSVHLTNSSERIKKRQPIAFAKTALFKWLCVLHNAGLCLYSAWTFVGILNAVKHAYQITGDSSAPFSFNTLWGSFCSRDSLWVTGLNYYGYWFYLSKFYEVVDTMIILAKGKPSSMLQTYHHTGAMFSMWAGIRFASPPIWIFVVFNSLIHTIMYFYYTLTTLKIKVPKILKASLTTAQITQIVGGGILAASHAFIYYKDHQTETVCSCLTTQGQFFALAVNVIYLSPLAYLFIAFWIRSYLKAKSN

SEQ ID NO: 128 - protein, 509 aa, Lobesia botrana
MANSVAELMLMLSGTAGTEVLVERLLVTCPDISRLYLLLRVKKGISPVQRLKELKQSQVFDVVRQRCPQQLEKLVVLEGDVATPGLGLTADAVSQLQDVSIVFHSAATLKFDEELGKAVDQNVRSVMRLLDICDTLPNMQAFIHVSTAYSNAELSTVAERVYPPPAPLPAVLALTEHVPSETLAQYTQQYIAPKPNTYTFTKALAETALEQHGNKGYPIAIFRPTIVISAVKNPFPGWVENLNGPSGVVVATGKGLLHVFRTKSGAVADFLPVDIAVDTLIAVGWETATDMPKQVRVYNCSTGDNPTSFDHFERRLRQDITHHPLDGAFWYPSGRSVESRWARNVYELVLQTIPLHIAEYTARAIGVKTPVNLITAGKRLTAMLDALEFFSTREYRFENGNVKALMRRLSPEDKKIYNLDVMSINWDDHYVNFVKGTRKYLLKEKDQDIEAARKHINKMWLLHNTFKFVLLLLFLRVIMQNRFARNIVYGTLRLLLSLFSTVYHRLLHA

SEQ ID NO: 129 - protein, 516 aa, Lobesia botrana
MSMGDGNCLPSCDPSARLPAPVQEIPRNHISTMAPSMSVAEYYADKTLFITGATGFMGKVLVEKLLRCCPDIKRIYMLMRAKKGQSSSERLEGFLKCRVFDRLHEDNPKCFSKLQLIPGDILAVDMGLTAEDRDRLRKEVQIIFHCAACVRFDMPLRDAADLNTRGTQRVLDLTDGMSQLEVFVHVSTAYCRCELPVLEERVYATAHKPQDVLKCVEWMDDELLTHLQPKMIDPQPNTYAYTKSLTESLVAQYEGKFPIVIARPSIVTAAFKEPMPGWVDNLNGPTGLLVGAGKGVIRTMHCDDRLQADVIPVDLVVNGCVLVGYKTGLDKQPKEIQVVNLTESGRNPLTWGAALDMGRVHVQEFPFSVCLWYPGGSPKTSLLLHQIALLFTHIIPAYLVDLLLLVLGKKTFMVRTQKRISYGLEVLQYYTTKEWHFKNENFVALRNVISKDDDAIFYTDIKYLKNVISSPHGKGPYGGWRLHPSIFVSPSNGAESDASADRHKGPFDLDYHKYYL

SEQ ID NO: 130 - protein, 427 aa, Lobesia botrana
MSAVTFQSRLHAFMFISQMAEGSQVQAFYSGKNFFITGGTGFVGLCLIEKILRSLPDAGKAYLLMRPKKGKKIEERLEEIAKNPIFETLLQTKTPEIFKKLVPVAGDVGEPNLGLSPEDRQTLVDNVNVVIHSAATLDFEDNLKPTVRINVLGTRRVVDLAKEIKNLKHLKALVHVSSAYVNSYLHEAREKLYDAPEDVEHVIALVDTLSDEALAVVEPKLLKTHPNTYTFTKHLAEHEVANYSDRLPCTIVRPSMIVAAWKEPVPGWTCSKVGPQGFLMGASKGVVRRLPLAKENIADYIPVDVVVNELLEKFIFTEWKFHNTKSLELSDKLNDTDRKLFYIDIGDIDWKAFFVALHLGVRRYLNREKESTLEAARGKDTILLVAHVIWQLLVIYLAWYLIACLIGLTMTQSAWCVPALYILYCYL

SEQ ID NO: 131 - protein, 536 aa, Lobesia botrana
MELESCVRIADIENAAMNPTAPILDYSEDHEFVSANQLDDYYAEEDRVDAKEEEKRRLTKGTAPRTEIQTFYRGQCVLLTGGTGFLGKAHFTTGYTSGRLILESIRKWVHWMRLPYAVVTIEKLLRSCGDIESIYVLIRSKKGKDARTRLNEMLDDFLFHRAHKENPKGIYKVIPVVGDMELPGLGLSEEDRKTLVSKVTIILNAAATVKFDEKLSVATNINVRGTKEVIALAKECKKLKALTHVSTAFSNTHVKCVEEKFYEPPMSVEALEAIAAIDENLLEDITPKLLGKYPNTYCFTKAIAEEAVRKYGEGMPICIIRPSIVVSTYEEPVRGWTDSVYGPTGLMVGIGCGVLRTMYMDVEKRADMVPVDLVVNALLASAWHTAKNFRENQTSEIPIYNFVSGAQNPLKWGTFIEMNQKYGIDKPTTKAVWYYGLTPTSSYPLFLFLNFFLHFLPAALIDTYSALTGKKRVMLKFYNKVMRLANILFYFSTQDWSFSDRLVRGMWGALDAADKVVFPFSVAEMSWDYHAETFIV

SEQ ID NO: 132 - protein, 519 aa, Lobesia botrana
MASQMSIKEFYKGRNILVTGGTGFMGKVLIEKLLYSIPEIGNIYVVMRSKKGKSIKQRLDEMHRSPVFDRLKNEQPNAYKKVIPIQGDVLYEDLGISSKDLEILSEEVSIIFHFAATLRLESPLKDSVSMNTAGTKRTLSIAKKFRNLTLFVHLSTAFCYPDYKVLEEKFHYPAADPHDVMRLCEWLDEKQLALLTPSLLVNHPNTYTYTKRLAESLVNDAFPELPCVIARPSIVCPAHADPVPGWVDNLNGPVGVMLGAGKGVIRTMLCDGTLQAQVIPVDVAINGLIALGMIEGSKTTKPEVIPIYNLNIGHQQPTSWGEVLNIAKDYGRKFPLSWPLWYPNGDITTNYALHETKRILFHLVPAYFIDFLLLITGQKRFMLRVQDRISQGLELLQYFTMRPWSFPCPNFDAICQVLSKEDDLVYPTNAREFDKVEYLIKSVEGGRVFCLKEDPTKFKINRTYHNFLFVLDLLVKIVFWCFILSFFAAWFEPVRDLLSLGEPVVKHLPFLGKAVFETN

SEQ ID NO: 133 - DNA, 1011 bp, Lobesia botrana
atggttcctcgagctgcgtcagaagaaaccgacctaaaagaggcaacccagttggaaccgagaaagtatgaaatagtgtatacaaacgttatatacttcacgtactggcatatagccgggctctatgggctgtatctctgcttcacttcggctaaatgggagactattgtttttgcctgggcgtggtacgtgctaggcgagctgggcgtgatagccggcgcccatcgtctctgggcccacaggacctataaagccaagatgcccctgcagatcatcctcatgctgttcaactgtataggcttccaaaacacggcaacagactgggtgcgggaccaccgtgtgcaccacaaacacagcgacactgacgccgacccgcacaactctcaacgagggttcttcttctcccacgtggggtggctgctcaccaggaagcaccgcctcgtgaaggagaaaggcgaggccgtggacatgacagacatatacagcaaccccgtactgaggttccagaaaaaatactcattaccgctaattggcactctctgcttcgccctccccactcttctacccgcgtactgctggggcgaagctgtcggcacagcctggaacatcaacctgcttcgctactgcctcaacctgaatggcactttcttgaccaacagcgcggctcacaagttcggcagtaagccctacgacaagaccatcccgccgacgcagaaccttctggtctcctttatgaccctgggcgaagggttccataactaccatcacgtgttctcctgggactaccgcgccgcagagctcggcaacacctacctaaacatgacaactatcttcattgactttttcgccctcatcggctgggcttacgacctgaagacggtcccagaggacgtcatcaagaagaggatggccaggactggcgaygcaccaatctttgggggtgggggaaaagatatgacgaaggaagatgtggtggatacagagattaggttccctgcgaaaaaagaatga

SEQ ID NO: 134 - DNA, 1077 bp, Lobesia botrana
atggccccaaacaagaaggagatcgccgagcccgcccctgacatcgataatgagttccgggccctggtggctccccaggccggccccagaaagtacaccgtggtgtatatgacagtgctgaacttcctgtactggcacctgggcgggctgtacggcctgtatctgtgctttacctccgccaagtgggccacaatcttctttaatgcaatcctgcacgtgctggccgtgtggggaatcaccgccggcgcccaccggctgtggagccacagatcctataaggccaagctgccactccagatcctgctgatgctgatgcacacactggcctttcagaacccagcaatccactgggcacgggatcacagactgcaccacaagtacagcgacaccgatgccgaccctcacaatgccacacggggcttcttttattcccacatcggctggctgctggtgaggaagcaccctgaggccatcaagcgcggcaagaccatcgatgtgacagacatcatccagaaccccgtgctccagatccagaagaagtactatctgtacctgtctggcatgctgagcttcgtgctgcctgccctgatcccaatgttcttttggggcgagacactgaacaatgcccaccacgtgtctctgctgagactgatcatcgtgtttaacgtgacattctttgtgaatagcgccgcccacaagtacggcaacaagccctatgatatgaatatgtcccctacccagtctgtgacactgagcctggccaccctgggcgagtgtttccacaactaccaccacgtgttcccctttgactataggacagccgagctgggcaacaactacatcaatttcaccacaaagtttatcgatttctttgccgccatcggctgggcctacgacctgaagtatgcctccgacgatatgatcatgaagaggtctaaccgcaccggcgatggaacagacctgtggggaagggtgaagtccaccaacgacgagcaggcccctaagaacgtgtggggctggggcgatgaggacatggtggaggagcacaagaaggtgctgctgtctcagaatcagtga

SEQ ID NO: 135 - DNA, 1116 bp, Choristoneura parallela
atggattttctgaacgagatcgacaattgccccgagcggctgagaaagccagagaagatggcccccaacgtgaccgaggagaatggcgtgctgttcgagtccgatgcagcaaccccagacctggccctggcaaggacacctgtggagcaggccgacgattctccaaggatctacgtgtggcgcaacatcatcctgtttgcctatctgcacctggccgccatctacggcggctatctgtttctgttctccgccaagtggcagaccgatatcttcgcctacctgctgtatgtggcatctggactgggaatcacagcaggagcacacaggctgtgggcacacaagagctacaaggccaagtggcctctgcgcctgatcctgaccatctttaacacaatcgcctttcaggacagcgccatcgattgggccagggaccaccgcatgcaccacaagtattccgagaccgacgccgatccacacaatgccacacggggcttctttttctctcacatcggatggctgctggtgcggaagcacccagagctgaagagaaagggcaagggcctggacctgtctgatctgtacagcgatcccatcctgagatttcagaagaagtactatatgatcctgatgcctctggcctgtttcatcctgcccaccgtgatccccgtgtatatgtggaacgagacatggagcaatgcctttttcgtggccgccctgtttaggtataccttcatcctgaacgtgacatggctggtgaattccgccgcccacaagtggggcgataagccttacgacaagtccatcaagccatctgagaacatgagcgtgtccctgtttgccttcggcgagggctttcacaattaccaccacaccttcccttgggactataagacagccgagctgggcaaccaccggctgaacttcaccacaaagttcatcaacttcttcgccaagatcggctgggcctatgatatgaagaccgtgtctcaggagatcgtgcagcagcgggtgaagagaacaggcgacggaagccaccacctgtggggatggggcgacaaggatcacgcacaggaggagatcaacgccgccatccgcatcaatccaaaggacgattga

SEQ ID NO: 136 - DNA, 1116 bp, Artificial Sequence
  DST192 G100L
atggattttctgaacgagatcgacaattgccccgagcggctgagaaagccagagaagatggcccccaacgtgaccgaggagaatggcgtgctgttcgagtccgatgcagcaaccccagacctggccctggcaaggacacctgtggagcaggccgacgattctccaaggatctacgtgtggcgcaacatcatcctgtttgcctatctgcacctggccgccatctacggcggctatctgtttctgttctccgccaagtggcagaccgatatcttcgcctacctgctgtatgtggcatctcttctgggaatcacagcaggagcacacaggctgtgggcacacaagagctacaaggccaagtggcctctgcgcctgatcctgaccatctttaacacaatcgcctttcaggacagcgccatcgattgggccagggaccaccgcatgcaccacaagtattccgagaccgacgccgatccacacaatgccacacggggcttctttttctctcacatcggatggctgctggtgcggaagcacccagagctgaagagaaagggcaagggcctggacctgtctgatctgtacagcgatcccatcctgagatttcagaagaagtactatatgatcctgatgcctctggcctgtttcatcctgcccaccgtgatccccgtgtatatgtggaacgagacatggagcaatgcctttttcgtggccgccctgtttaggtataccttcatcctgaacgtgacatggctggtgaattccgccgcccacaagtggggcgataagccttacgacaagtccatcaagccatctgagaacatgagcgtgtccctgtttgccttcggcgagggctttcacaattaccaccacaccttcccttgggactataagacagccgagctgggcaaccaccggctgaacttcaccacaaagttcatcaacttcttcgccaagatcggctgggcctatgatatgaagaccgtgtctcaggagatcgtgcagcagcgggtgaagagaacaggcgacggaagccaccacctgtggggatggggcgacaaggatcacgcacaggaggagatcaacgccgccatccgcatcaatccaaaggacgattga

SEQ ID NO: 137 - DNA, 990 bp, Ostrinia latipennis
atggcacagtacgccacctctgccgagggactgcctgtgaaggacgagtgcttcgaggataaggagatcaagagcaactcctttccaaagctggagatcctgtatttcaatgtgatgacctttacactgctgcacctgtccgccctgtacggcctgtatctgggctttacctctgtgaagtgggccacaatcggcatcggcatcgtgctgtacttctttgccgagatcggaatcaccgcaggagcacacaggctgtggtcccacagatcttataaggccaagctgcccctggagatcctgctgatggtgttcaacagcatggcctttcagaataccgccctgtcctgggcaagggaccacagactgcaccacaagtgcccagacacaaacggcgatccccacaacgccaatcggggcttcttttactctcacgtgggctggctgctgacaaagaagagcgatgaagtgatcaagcagggcaagctgtgcgacgtggccgatctgtacagcaatcccgtgctgagattccagaagaagtatgccgtgcctttcatcggcatgctgtgctttgtgctgcccaccctgatccctatgtacttttggggcgagacactgaacaatgcctggcacttcaatatgttcagatatgtggtgaacctgaatgccaccttctgcgtgaactccgtggtgcacaagtggggctacaagccttatgacaagaatatccgcccaacacagaacgtgctgctgaatttcgccgtgctgggcgaggccttccacaattttcaccacgtgtttccatgggattacagggcagcagagctgggaaaccagaagatgaatcccaccacactgttcatcgacttctttgcctggatcggctgggcctatgatctgaagaccgcctccaaggagatggtgaagtctaggagcgagaggaccggcgacggaacagatctgtggggacacgacgccgataagctgaagtga

SEQ ID NO: 138 - DNA, 990 bp, Artificial Sequence
  DST109 V230A
atggcacagtacgccacctctgccgagggactgcctgtgaaggacgagtgcttcgaggataaggagatcaagagcaactcctttccaaagctggagatcctgtatttcaatgtgatgacctttacactgctgcacctgtccgccctgtacggcctgtatctgggctttacctctgtgaagtgggccacaatcggcatcggcatcgtgctgtacttctttgccgagatcggaatcaccgcaggagcacacaggctgtggtcccacagatcttataaggccaagctgcccctggagatcctgctgatggtgttcaacagcatggcctttcagaataccgccctgtcctgggcaagggaccacagactgcaccacaagtgcccagacacaaacggcgatccccacaacgccaatcggggcttcttttactctcacgtgggctggctgctgacaaagaagagcgatgaagtgatcaagcagggcaagctgtgcgacgtggccgatctgtacagcaatcccgtgctgagattccagaagaagtatgccgtgcctttcatcggcatgctgtgctttgtgctgcccaccctgatccctatgtacttttggggcgagacactgaacaatgcctggcacttcaatatgttcagatatgtggtgaacctgaatgccaccttctgcgtgaactccgtggcccacaagtggggctacaagccttatgacaagaatatccgcccaacacagaacgtgctgctgaatttcgccgtgctgggcgaggccttccacaattttcaccacgtgtttccatgggattacagggcagcagagctgggaaaccagaagatgaatcccaccacactgttcatcgacttctttgcctggatcggctgggcctatgatctgaagaccgcctccaaggagatggtgaagtctaggagcgagaggaccggcgacggaacagatctgtggggacacgacgccgataagctgaagtga

SEQ ID NO: 139 - DNA, 960 bp, Ostrinia latipennis
atggccgagacaagctccgatcccaacgagacacctgccgacgagaacctgcctaagaataagcggggctctaagatcatctacaagtatgtgaccagctttacaatcctgcacctggccgccttctacggcctgtatctgtgcctgaccaaggccaagtggctgacaatcggctttagcctgctgatgtacgagctgtccatcctgggaatcagcgccgccgcccacaggctgtggtcccacagatcttataaggccaagtttccactgcaggtcatcctgatggtgctgaattgtatgagcttccagaactccgccctgaattggtgcagggatcaccgcgtgcaccacaagtgtagcgacaccgatggcgacccccacaacgccagcagaggcttctttttctcccacatcggctggctgatgacacacaagtccgaggaggtgaagcggcagggcaattctatcgatatgagcgacctgtacaacaatcctgtgctgagatttcagaagaagtatgccgtgccactgatcggctctatctgctttatcttcccaaccctgatccccgtgtacttctggggcgagacaatcaacaatgcctggcacatgaacatcctgagatatgtgatggccctgcacgccaccctgtgcatcaactccgtggcccaccgctggggctacaagacatatgataagaatatcctgccctctcagaacatgctgctgaataccgccatgctgggcgagggctttcacaattaccaccacgtgttcccctgggactatcagtctagcgagctgggcaaccggaagtttaatcctaccacatggttcatcgacttcttcgcctgggtgggctgggcctacgacctgaagaccacatccgaggaagtgatccggaagagagccgagaggaccggcgatggaacagacctgtggggagccaagcgcaactga

SEQ ID NO: 140 - DNA, 1086 bp, Drosophila melanogaster
atggccccttactctcggatctatcaccaggacaagagctccagagagacaggcgtgctgtttgaggacgatgcccagacagtggacagcgatctgaccacagataggttccagctgaagcgcgccgagaagcggagactgccactggtgtggaggaacatcatcctgttcgccctggtgcacctggccgccctgtatggcctgcacagcatctttacccgcgccaagctggccaccacactgttcgcagcaggcctgtatatcatcggcatgctgggagtgaccgcaggagcacacaggctgtgggcccacagaacatacaaggcaaagtggccactgaggctgctgctggtcatcttcaacaccatcgccttccaggacgccgtgtatcactgggcccgggatcacagagtgcaccacaagtactccgagacagacgccgatccccacaatgccacaagaggcttctttttctctcacgtgggctggctgctgtgcaagaagcaccctgacatcaaggagaagggcaggggcctggacctgtccgatctgagggcagatcctatcctgatgtttcagaggaagcactactatatcctgatgccactggcctgcttcgtgctgcccaccgtgatccctatggtgtattggaacgagacactggcctctagctggtttgtggccacaatgtttcggtggtgtttccagctgaacatgacatggctggtgaatagcgccgcccacaagtttggcaatagaccatacgacaagaccatgaaccccacacagaatgccttcgtgtccgcctttaccttcggcgagggctggcacaactaccaccacgccttcccatgggactataagacagccgagtggggctgttactctctgaatatcaccacagcctttatcgatctgttcgccaagatcggctgggcatacgacctgaagaccgtggcaccagatgtgatccagaggcgcgtgctgaggacaggcgatggctcccacgagctgtggggatggggcgacaaggatctgaccgcagaggacgccaggaatgtgctgctggtggataagtctcgctga

SEQ ID NO: 141 - DNA, 1059 bp, Dendrolimus punctatus
atggccccaaaggaggccgatgtgaacggcgtgctgtttgagtctgatgccaccacaccagacatggcactgccaaccacacctgtgcagcaggccgacaactaccccaagaagctggtgtggcggaatatcctgctgtttgcctatctgcacctggccgccctgtacggcggctatctgtttctgttctccgccaagtggcagacagatatcttcgcctacatcctgtatatcatgtctggactgggaatcaccgcaggagcacacaggctgtgggcccacaagagctacaaggccaagtggcctctgcgcctgatcctggtgctgtttaacacactggcctttcaggactctgccatcgattgggcccgggaccacagaatgcaccacaagtatagcgagacagacgccgatcctcacaatgccacccggggcttctttttctcccacatcggctggctgctggtgagaaagcacccagagctgaagaagaagggcaagggcctggacatctctgatctgtacgccgaccccatcctgcggtttcagaagaagtactatctgctgctgatgcctctgggctgcttcatcctgccaacagtgatccccgtgtacctgtggaacgagacctggagcaatgcctttctggtggccgccctgttcagatatgccgtgatcctgaacgtgacctggctggtgaattccgccgcccacaagtggggcgataagccttacgacaagagcatcaagccatccgagaacctgtctgtggccctgtttgccctgggcgagggcttccacaattaccaccacacattcccctgggattataagaccgccgagctgggcaacaataggctgaactttaccacaaccttcatcaacttcttcgccaagatcggctgggcctatgatatgaagacagtgtccgacgagatcatccagaatagggtgaagaggaccggcgacggaagccaccacctgtggggatggggcgacaaggatcactccaaggaggagatcaacgccgccatccgcatcaatcctaaggacgattga

SEQ ID NO: 142 - DNA, 1059 bp, Dendrolimus punctatus
atggccccaaaggaggccgatgtgaacggcgtgctgtttgagtctgatgccaccacaccagacatggcactgccaaccacacctgtgcagcaggccgacaactaccccaagaagctggtgtggcggaatatcctgctgtttgcctatctgcacctggccgccctgtacggcggctatctgtttctgttctccgccaagtggcagaccgatatcttcgcctacatcctgtatatcatgtctggactgggaatcacagcaggagcacacaggctgtgggcacacaagagctacaaggccaagtggcctctgcgcctgatcctggtgctgtttaacaccctggcctttcaggactctgccatcgattgggcccgggaccacagaatgcaccacaagtatagcgagacagacgccgatcctcacaatgccacccggggcttctttttctcccacacaggctggctgctggtgagaaagcacccagagctgaagaagaagggcaagggcctggacatctctgatctgtacgccgaccccatcctgcggtttcagaagaagtactatctgctgctgatgcctctgggctgcttcatcctgccaaccgtgatccccgtgtacctgtggaacgagacatggagcaatgcctttctggtggccgccctgttcagatatgccgtgatcctgaacgtgacctggctggtgaattccgccgcccacaagtggggcgataagccttacgacaagagcatcaagccatccgagaacctgtctgtggccctgtttgccctgggcgagggcttccacaattaccaccacacattcccctgggattataagaccgccgagctgggcaacaataggctgaactttaccacaaccttcatcaacttcttcgccaagatcggctgggcctatgatatgaagaccgtgtccgacgagatcatccagaatagggtgaagaggacaggcgacggaagccaccacctgtggggatggggcgacaaggatcactccaaggaggagatcaacgccgccatccgcatcaatcctaaggacgattga

SEQ ID NO: 143 - DNA, 1059 bp, Dendrolimus punctatus
atggccccaaaggaggccgatgtgaacggcgtgctgtttgagtctgatgccaccacaccagacatggcactgccaaccacacctgtgcagcaggccgacaactaccccaagaagctggtgtggcggaatatcctgctgtttgcctatctgcacctggccgccctgtacggcggctatctgtttctgttctccgccaagtggcagacagatatcttcgcctacatcctgtatatcatgtctggactgggaatcaccgcaggagcacacaggctgtgggcccacaagagctacaaggccaagtggcctctgcgcctgatcctggtgctgtttaacacactggcctttcaggactctgccatcgattgggcccgggaccacagaatgcaccacaagtatagcgagacagacgccgatcctcacaatgccacccggggcttctttttctcccacatcggctggctgctggtgagaaagcacccagagctgaagaagaagggcaagggcctggacgtgtctgatctgtacgccgaccccatcctgagatttcagcagaagtactatctgctgctgatgcctctgggctgcttcatcctgccaacagtgatccccgtgtacctgtggaacgagacctggagcaatgcctttctggtggccgccctgttccggtatgccgtgatcctgaacgtgacctggctggtgaattccgccgcccacaagtggggcgatagaccttacgacaagagcatcaagccatccgagaacctgtctgtggccctgtttgccctgggcgagggcttccacaattaccaccacacattcccctgggattataagaccgccgagctgggcaacaataggctgaactttaccacaaccttcatcaacttcttcgccaagatcggctgggcctatgatatgaagacagtgtccgacgagatcatccagaatagggtgaagaggaccggcgacggaagccaccacctgtggggatggggcgacaaggatcactccaaggaggagatcaacgccgccatccgcatcaatcctaaggacgattga

SEQ ID NO: 144 - DNA, 1059 bp, Cydia pomonella
atggcccctaacgtgaccgatgtgaatggcgtgctgttcgagtccgatgcagcaacacctgacctggccctggcaaacgcaccagtgcagcaggccgacgattctccccggatctacgtgtggagaaatatcatcctgtttgcctatctgcacatcgccgccctgtacggcggctatctgttcctggtgtccgccaagtggcagaccgacatctttgcctacttcctgtatgtggcctctggcctgggaatcacagcaggagcacacaggctgtgggcccacaagagctacaaggccaagtggcccctgagactgatcctggtcatcttcaacaccatcgccttccaggactctgccatcgattgggcccgggaccacagaatgcaccacaagtatagcgagacagacgccgatccccacaatgccacaaggggcttctttttcagccacatcggctggctgctggtgaggaagcaccctgagctgaagcgcaagggcaagggcctggacctgtccgatctgtacgccgatcctatcctgcgctttcagaagaagtactatctgatcctgatgccactggcctgcttcgtgctgcccaccgtgatccccgtgtacctgtggaacgagacatggacaaatgcctttttcgtggccgccctgtttcggtatgccttcatcctgaacgtgacatggctggtgaattctgccgcccacaagtggggcgataagccatacgacaagtccatcaagccctctgagaacatctctgtgagcctgtttgccttcggcgagggctttcacaattaccaccacaccttcccatgggattataagacagccgagctgagctccaaccggctgaacttcaccacaaagttcatcaactttttcgccaagatcggctgggcctatgatatgaagaccgtgagcgacgagatcatccagaagagggtgaatcgcacaggcgacggcagccaccacctgtggggatggggcgacaaggatcactccaaggaggaggtgaacgccgccgtgagaatcaatcccaaggacgattga

SEQ ID NO: 145 - DNA, 972 bp, Ostrinia latipennis
atgtctgagatcaccagcgatcccaatgagaaccctacagagaagaatatccccaagaacaagggcggcaccaagatcatctaccagtatgtgatctcttttacatacctgcacctggccgccctgtacggcctgtatctgtgctttaccagcgtgaagtggctgacaatcggcttcggcctgctgctgtaccacctgaccctgacaggaatcaccgcaggagcacacaggctgtggagccacaagtcctataaggccaagtttcctctgcaggtcatcctgatggtgctgaattccatggccttccagaacaccatcatcacatgggccagggaccaccgcgtgcaccacaagtgttctgacacagatggcgacccacacaatgccaaccggggcttctttttcagccacatcggctggctgatgaccaagaagtccgaggaagtgatcaagaagggcaagacaatcgatatgtttgacctgtacaacaatcccgtgctgcggttccagagaaagtatgccctgcctgtgatcggcaccctgtgctttatcctgccaacactgatccccatgtacttctggggcgagaccctgaacaatgcctggcacatgaatatcctgagatatgtgaccgtgctgcacgtgaccctgtgcgtgaacagcgtggtgcacaagtggggctacaagccttatgataagaatatcctgccatcccagaacgtgctgatgtctacctttgccctgggcgagggcttccacaattaccaccacgtgttcccatgggactatcagacagccgagctgggcaataagaagtttaaccccaccacatggttcatcgacttcttcgcctgggtgggctgggcctacgacctgaagaccgcctccgaggaggtggtgcggaagagagccgagaggaccggcgatggaacagacctgtggggagcacctcgcgataacaataactattga

SEQ ID NO: 146 - DNA, 960 bp, Artificial Sequence
  DST014 desaturase coding sequence
  misc_feature (191)..(192): n is a, c, g, or t
  misc_feature (202)..(204): n is a, c, g, or t
  misc_feature (213)..(213): n is a, c, g, or t
  misc_feature (310)..(310): n is a, c, g, or t
  misc_feature (312)..(312): n is a, c, g, or t
  misc_feature (432)..(432): n is a, c, g, or t
  misc_feature (448)..(448): n is a, c, g, or t
  misc_feature (450)..(450): n is a, c, g, or t
  misc_feature (651)..(651): n is a, c, g, or t
  misc_feature (654)..(657): n is a, c, g, or t
  misc_feature (741)..(741): n is a, c, g, or t
atggccgagacaagctccgatcccaacgagacacctgccgacgagaacctgcctaagaataagcggggctctaagatcatctacaagtatgtgaccagctttacaatcctgcacctggccgccttctacggcctgtatctgtgcctgaccaaggccaagtggctgacaatcggctttagcctgctgatghnngagctgtccnnnctgggarynagcgccgccgcccacaggctgtggtcccacagatcttataaggccaagtttccactgcaggtcatcctgatggtgctgaattgtatgagcttccagnvntccgccctgaattggtgcagggatcaccgcgtgcaccacaagtgtagcgacaccgatggcgacccccacaacgccagcagaggcttctttttctcccacatcggctggctgatgacahvnaagtccgaggaggtgnwncggcagggcaattctatcgatatgagcgacctgtacaacaatcctgtgctgagatttcagaagaagtatgccgtgccactgatcggctctatctgctttatcttcccaaccctgatccccgtgtacttctggggcgagacaatcaacaatgcctggcacatgaacatcctgagatatgtgatggccctgcacgccaccydnhbnnnnaactccgtggcccaccgctggggctacaagacatatgataagaatatcctgccctctcagaacatgctgctgaataccgccdbnctgggcgagggctttcacaattaccaccacgtgttcccctgggactatcagtctagcgagctgggcaaccggaagtttaatcctaccacatggttcatcgacttcttcgcctgggtgggctgggcctacgacctgaagaccacatccgaggaagtgatccggaagagagccgagaggaccggcgatggaacagacctgtggggagccaagcgcaactga

SEQ ID NO: 147 - DNA, 1032 bp, Ctenopseustis obliquana
atgccccctaacaccaaggagaaggagatgaatgagctggccggcatctctggcaggaacgaggagctggctccccaggccgcaccaaggaagttcgagatcgtgtaccgcaatatcgtgacattctcttttatgcacgtggccgggctgtacggcctgtatctgtgctttaccagcgccaagtggcagacaatcgtgctggccttcgtgctgtatacctttgccgagatcggagtgacagcaggagcacacaggctgtgggcccacaagacctacaaggccaagacacccctgcagatcatcctgatggtgctgaacagcatcgccttccagaattccgccatcgattgggtgcgggaccacagactgcaccacaagtattccgacaccgatgccgaccctcacaacgccacaaggggcttcttttactctcacgtgggatggctgctggtgggcaagcacccagaagtgatcaagcgcggcaaggccatggagatgtccgatatctattctaatccagtgctgtggttccagaagaagtacgccgtgcccttcatcggcatcatctgttttggcctgcctaccctggtgccaatgtatttttggggcgagaccctgaacaatgcctggcacatcacaatgctgcggtacatcgccaacctgaatgtgacattcctggtgaacagcgccgcccatatgttcggcaataagccctataacaagaatatcctgcctgcccagaacatctctgtgagcatcctgaccttcggcgagggctttcacaattaccaccacgtgtatccttgggactacagaacagccgagctgggcaacaatatgctgaacatgaccacactgttcatcgatttctttgcctggatcggctgggcctacgacctgaagaccgtgagctccgccgccatcgagtccaaggccaagcggaccggcgatggcacaaatctgtggggctggggcgataaggacatgctgagagaggaccagaaggccgccgagatcctgtttgccaagtga

SEQ ID NO: 148 - DNA, 1059 bp, Planototrix excessana; identical to SEQ ID NO: 143

SEQ ID NO: 149 - DNA, 1059 bp, Lampronia capitella
atgccccctaacgtgaccgaggccaatggcgtgctgttcgagaacgatgtgcagacaccagacatgggactggaggtggcacctgtgcagaaggccgatgagaggaagatccagctggtgtggcgcaatatcatcgcctttgcctgcctgcacctggcagccgtgtacggagcctatctgttctttaccagcgccatctggcagacagacatcttcgcctacatcctgtatgtgatgtccggactgggaatcaccgcaggagcacacaggctgtgggcccacaagtcttacaaggccaagtggcccctgcgcctgatcctggtggcctttaacacactggcctttcaggactctgccatcgattgggcccgggaccacagaatgcaccacaagtatagcgagaccgacgccgatcctcacaatgccacacggggcttctttttctctcacatcggctggctgctgtgccggaagcacccagagctgaagagaaagggccagggcctggacctgagcgatctgtacgccgatccaatcatcagatttcagaagaagtactatctgctgctgatgcccctggcctgtttcgtgctgccaaccatcatccccgtgtacctgtggggcgagagctggaagaacgcctttttcgtggccgccatgtttcggtataccttcatcctgaacgtgacatggctggtgaatagcgccgcacacaagtggggaggcaagccttacgataagaacatccagccagcccagaatatctccgtggccatctttgccctgggcgagggcttccacaattaccaccacacctttccctgggactataagacagccgagctgggcaacaataggctgaactttaccacaagcttcatcaacttcttcgcctccttcggctgggcctatgatctgaagaccgtgtccgacgagatcatccagcagagggtgaagcgcacaggcgacggatctcaccacctgaggggatggggcgaccaggatatccctgccgaggaggcccaggccgccctgcggatcaacagaaaggacgattga

SEQ ID NO: 150 - DNA, 1059 bp, Pectinophora gossypiella
atggccccaaacgccacagacgtgaatggcgtgctgtttgagagcgacgccgtgaccccagatatggcactgccaaacacacctgtggcacaggccgataatacccccaagcagtacgtgtggaggaacatcatcctgtttgcctatctgcacctggccgccgtgtacggcggctatctgtttctgttcagcgccaagtggcagacagacctgttcgcctacatcctgtatgtgatgtccggactgggaatcaccgcaggagcacacaggctgtgggcccacaagtcttacaaggccaagtggcctctgcgcctgatcctggtcatcttcaacaccatcgcctttcaggactccgccatcgactgggcccgggatcacagaatgcaccacaagtattctgagacagacgccgatcctcacaatgccacccggggcttctttttcagccacatcggctggctgctggtgagaaagcacccagagctgaagaagaagggccagggcctggatctgtccgacctgtacgccgatcccatcctgcggtttcagaagaagtactatatctatctgatgcctctggcctgcttcgtgatgccaacagtggtgcccgtgtacctgtggggcgagtcttggaccaacgccttcttcgtgtgcgccctgtttagatatgccttcatcctgaacgtgacctggctggtgaattctgccgcccacaagtggggcgacaagccttacgataagagcatcaagccagcccagaacctgagcgtgtccgtgtttgccctgggcgagggcttccacaattaccaccacaccttcccctgggactataagacagccgagctgggcaacaatagcctgaacttcaccacaaacttcatcaacttcttcagcaagatcggctgggcctacgacctgaagacagtgtccgatgagatcgtgaaggcaagggtggagaggaccggcgatggatcccaccacctgtggggatggggcgacaaggatcacgaccagaaggagatcaacgccgccatccggatcaatcctaaggacgattga

SEQ ID NO: 151 - DNA, 1107 bp, Ameloyis transitella
atgcacatcgagtctgagaactgccccggcaggtttaaggaggtgaacatggcccctaatgccaccgatgccaatggcgtgctgttcgagaccgatgccgccacacctgacctggccctgccacacgcacctgtgcagcaggccgacaactacccaaagaagtacgtgtggcgcaatatcatcctgtttgcctacctgcacatcgccgccctgtacggcggctatctgtttctgttccacgccaagtggcagaccgatatcttcgcctacatcctgtatgtgatgtctggactgggaatcacagcaggagcacacaggctgtgggcccacaagagctacaaggccaagtggcctctgagactgatcctggtcatcttcaacacactggcctttcaggactctgccatcgattggagcagggaccaccgcatgcaccacaagtattccgagaccgacgccgatccccacaatgccacacggggcttctttttctctcacatcggctggctgctggtgcggaagcaccctgagctgaagagaaagggcaagggcctggacctgtccgatctgtatgccgacccaatcctgagatttcagaagaagtactatctgatcctgatgcccctgacctgtttcgtgctgccaacagtgatccccgtgtactattggggcgagacctggacaaacgcctttttcgtggccgccctgtttaggtacgccttcatcctgaacgtgacctggctggtgaatagcgccgcccacaagtggggcgataagccttatgaccgcaacatcaagccatccgagaatatcagcgtgtccatgtttgccctgggcgagggcttccacaactaccaccacaccttcccatgggattataagacagccgagctgggcaacaatatgctgaacttcaccacaaacttcatcaacttcttcgccaagatcggctgggcctacgatctgaagaccgtgtccgacgagatcgtgcggtctagagcaaagaggacaggcgacggaagccaccacctgtggggatggggcgacaaggatcactccagggaggagatggctgccgccatccgcatccaccccaaggacgattga

SEQ ID NO: 152 - DNA, 1059 bp, Ameloyis transitella
atggccccaaacgccaccgatgccaatggcgtgctgtttgagaccgatgccgccacaccagacctggccctgccacacgcacctgtgcagcaggccgacaactaccccaagaagtacgtgtggaggaatatcatcctgtttgcctacctgcacatcgccgccctgtacggcggctatctgtttctgttccacgccaagtggcagaccgatatcttcgcctacatcctgtatgtgatgtctggactgggaatcacagcaggagcacacaggctgtgggcccacaagagctacaaggccaagtggcctctgagactgatcctggtcatcttcaacacactggcctttcaggactctgccatcgattggagcagggaccaccgcatgcaccacaagtattccgagaccgacgccgatcctcacaatgccacacggggcttctttttctctcacatcggctggctgctggtgcggaagcacccagagctgaagagaaagggcaagggcctggacctgtctgatctgtatgccgaccccatcctgagatttcagaagaagtactatctgatcctgatgcctctgacctgcttcgtgctgccaacagtgatccccgtgtactattggggcgagacctggacaaacgcctttttcgtggccgccctgtttaggtacgccttcatcctgaacgtgacctggctggtgaatagcgccgcccacaagtggggcgataagccttatgaccgcaacatcaagccatccgagaatatcagcgtgtccatgtttgccctgggcgagggcttccacaactaccaccacaccttcccctgggattataagacagccgagctgggcaacaatatgctgaacttcaccacaaacttcatcaacttcttcgccaagatcggctgggcctacgatctgaagaccgtgtccgacgagatcgtgcggtctagagcaaagaggacaggcgacggaagccaccacctgtggggatggggcgacaaggatcactccagggaggagatggctgccgccatccgcatccaccctaaggacgattga

SEQ ID NO: 153 - DNA, 1068 bp, Danaus plexippus
atggccccaaacagctccgatgccaatggcgtgctgttcgagtccgatgcagcaacaccagacctggccctgccaaacacccctatccagcaggccgactcttaccccaggaagtacgtgtggcgcaatatcatcgcctttgcctacctgcacatcgccgccctgtacggcggctatctgtttctgttctctgccaagtggcagacaaacatcttcgcctacatcctgtatgtgatgagcggactgggaatcaccgcaggagcacacaggctgtgggcccacaagtcctacaaggccaagtggcctctgagagtgatcctggtcatcttcaacacactggcctttcaggacagcgccatcgattgggcccgggaccacagaatgcaccacaagtattccgagacagacgccgatcctcacaatgccacccggggcttctttttctctcacatcggctggctgctggtgcggaagcacccagagctgaagagaaagggcaagggcctggacctgagcgatctgtacgccgatcccatcctgagatttcagaagaagtactatctgctgctgatgcctatctcctgcttcatcctgccaacaatcatccccgtgtatctgtggggcgagacatggaccaacggctactttgtggccgccatgtttagatatgccttcatcctgaacgtgacctggctggtgaattctgccgcccacaagtggggcgataagccttacgacaagaacatccagccaagcgagaatatgtctgtgagcctgtttgccctgggcgagggcttccacaattaccaccacacattcccctgggattataagaccgccgagctgggcaacaatcggctgaactttaccacaaccttcatcaacttcttcgccaagatcggctgggcctatgatctgaagacagtgagcgacgagatcgtgaagcagagggtgaacaggaccggcgacggatcccaccacctgtggggatggggcgacaaggatcactctaaggaggagatccgggccgccatcagaatcaatcctaccgacgataaggaggagtga

SEQ ID NO: 154 - DNA, 1059 bp, Papillo polytes; identical to SEQ ID NO: 149

SEQ ID NO: 155 - DNA, 1059 bp, Papillo xuthus
atggcccctaagtccgccgatgccaacggcgtgctgttcgagtctgatgcagcaacccctgacctggccctgggatctgccccaatcatgcaggccgacaagcagccccggagactggtgtggcggaatatcatcgccttcgcctacctgcacctggccgccatctacggcggctatctgtttctgttccacgccaagtggcagaccgatatctttgcctacatcctgtatgtgatgtccggactgggaatcacagcaggagcacaccggctgtgggcccacaagtcttacaaggccaagtggcccctgagactgatcctggtcatcttcaacacactggcctttcaggacagcgccatcgattgggcccgggaccacagaatgcaccacaagtattccgagaccgacgccgatccccacaatgccacacggggcttctttttcagccacatcggctggctgctggtgaggaagcaccctgagctgaagaggaagggacagggactggacctgtccgatctgtatgccgatcctatcctgagattccagaagaagtactatctgctgctgatgccaatcgcctgctttatcctgcccaccgtgatccccgtgtactattggggcgagtcttgggtgacaagctttttcgtggccgccctgtttaggtacgccttcatcctgaacgtgacctggctggtgaattccgccgcccacaagtggggcgataagccatatgacaagcacatccagcccgccgagaacatcagcgtgtccatcttcgccctgggcgagggctttcacaattaccaccacaccttcccatgggactataagacagccgagctgggcaacaatcgcctgaactttaccacaaacttcatcaatctgtttgccaagatcggctgggcctacgatctgaagaccgtgtctgacgagatcgtgaggaatcgcgtgaagaggacaggcgatggaagccaccacctgtggggatggggcgacaaggatcacgccaaggaggagatcgacgccgccatccgcatcaaccccaaggacgattga

SEQ ID NO: 156 - DNA, 1002 bp, Antheraea pernyi
atggcccccaacatcaccgatgccaatggcgtgctgtttgagtctgatgccaccacaccagacctgggcctgcccatccagcctatccagcaggccgacaaccggcccaagcagttcgtgtggagaaatatcatcctgttcgcctacctgcacatcgccgccctgtacggcggctatctgtttctgttccacgccaagtggcagaccgatatcttcgcctacatcctgtatgtgatgagcggactgggaatcacagcaggagcacaccggctgtgggcccacaagtcctacaaggccaagtggcctctgagactgatcctggtcatcttcaacaccctggcctttcaggactctgccatcgattgggcccgggaccacagaatgcaccacaagtatagcgagacagacgccgatcctcacaatgccagcaggggcttctttttctcccacatcggctggctgctggtgaggaagcacccagagctgaagcgcaagggcaagggcctggacctgaacgatctgtacgccgaccccatcctgcgcttccagaagaagtactatctgctgctgatgcctatcgcctgctttatcctgccaaccgtgatccccgtgtacttctggtctgagacatggagcaatgcctttttcgtggccgccctgtttcggtataccttcatcctgaacgtgacatggctggtgaattccgccgcccacaagtggggcgataagccttacgacaagtccatcaagccatctgagaacatcagcgtgtccgtgtttgccctgggcgagggcttccacaattaccaccacacctttccctgggattataagacagccgagctgggcaacaatagactgaactttaccacaaacttcatcaacttcttcgccaagttcggctgggcctatgatctgaagaccgtgtccgacgagatcgtgcagaatagggtgaagcgcacaggcgacggctctcaccacctgtggggctggggcggctga

SEQ ID NO: 157 - DNA, 1059 bp, Manduca sexta
atgcccccttacaccagcgacgccaacggcgtgctgtttgagtccgacgcagcaaccccagatctggccctgagctccacacctgtgcagcaggccgataaccggccaaagcagctggtgtggagaaatatcatcctgttcgcctatctgcacatcgccgccctgtacggcggctatctgtttctggtgcacgccaagtggcagaccgacatcttcgcctacctgctgtatgtgatgagcggactgggaatcacagcaggagcacaccggctgtgggcccacaagtcctacaaggccaagtggcctctgagactgatcctggtggtgtttaacacaatggcctttcaggactctgccatcgactgggcccgggatcacagaatgcaccacaagtatagcgagaccgacgccgatccacacaatgccacaaggggcttctttttctctcacatcggctggctgctggtgaggaagcaccccgagctgaagcgcaagggcaagggcctggatctgagcgacctgtacgccgatcccatcctgcgctttcagaagaagtactatctgatcctgatgcctatcacctgcttcgtgatgccaacagtgatccccgtgtacctgtggggcgagtcctgggtgaacgcctttttcgtggccgccctgtttcggtatgccttcatcctgaacgtgacctggctggtgaattctgccgcccacaagtggggcgacaagccttacgataagtccatcaagccatctgagaacatctctgtgagcatgtttgccctgggcgagggcttccacaattaccaccacaccttcccttgggactataagacagccgagctgggcaacaatcggctgaacttcaccacaaacttcatcaacttcttcgccaagatcggctgggcctatgacctgaagaccgtgtccgatgagatcatccagcagagggtgcagcgcacaggcgatggatctcaccacctgtggggatggggcgacaaggatcacgacaaggaggaggtgaacgccgccatcagaatcaatccaaaggacgattga

SEQ ID NO: 158 - DNA, 1059 bp, Plutella xylostella
atggccccaaacgcctccgacgccaatggcgtgctgttcgagtctgacgcagcaacaccagatctggccctgccacacgcacctgtgcagagggcagatgcatacccaaggcagtacgtgtggagaaacatcctgctgtttgcctacctgcacctgaccgccctgtatggcggctttctgttcctgacctccgccaagtggcagacagacgtgttcgcctacatcctgtatgtggtgtctggactgggaatcacagcaggagcacacaggctgtgggcacacaagagctacaaggccaagtggcctctgcgcctgatcctggtcatcttcaacaccatcgcctttcaggacagcgccatcgactgggcccgggatcacagaatgcaccacaagtattccgagaccgacgccgatcctcacaacgccacacggggcttctttttctcccacatcggctggctgctggtgagaaagcacccagagctgaagaataagggcaagggcctggatctgagcgacctgtacgccgaccccatcctgcggtttcagaagaagtactatctgctgctgatgcctctggcctgcttcgtgatgccaacagtggtgcccgtgtattgttggggcgagagctggtccaacgcctactttgtggccgccatgtttagatatgccttcatcctgaacgtgacctggctggtgaattccgccgcccacaagtggggcgacaagccttacgataaggccatcaagccatctgagaacatctctgtgagcatctttgccctgggcgagggcttccacaattaccaccacaccttcccctgggattataagacagccgagctgggcaacaatcggctgaacttcaccacaaacttcatcaacttcttcgccaagctgggctgggcctacgacctgaagaccgtgtctgaggagatcatccagcagagggtgaaccgcacaggcgatggaagccaccacctgtggggatggggcgacaccgatcacgacagggcagaggccgacgccgccatccgcatcaatcctaaggatgagtga

SEQ ID NO: 159 - DNA, 1059 bp, Bombyx mori
atggcccctaacgtgaaggacgccaatggcgtgctgtttgagaacgacgccgatacccctgacctgggcctgagctccacaccagtgcagcaggccgataactaccccaagaagctggtgtggcggaatatcatcctgttcacctatctgcacatcgccgccctgtacggcggctatctgtttctgtaccacgccaagtggcagaccgatctgttcgcctacatcctgtatgtgatgtctggactgggaatcacagcaggagcacacaggctgtgggcacacaagagctataaggccaagtggcccctgcgcgtgatcctgatcatctttaactccctggcctttcaggactctgccctggactgggcccgggatcacagaatgcaccacaagtactccgagaccgacgccgatccccacaatgccacacggggcttctttttctctcacatcggctggctgctggtgcggaagcaccctgagctgaagagaaagggcaagggcctggatctgtctgacctgtatgccgaccctatcctgagatttcagaagaagtactatctgatcctgatgccaatcgcctgctttatcctgcccaccgtgatccccgtgtacttctggaacgagagctggatcaatgcctttttcgtggccaccctgtttagatacacattcatcctgaacgtgacatggctggtgaatagcgccgcacacaagtggggaggcaagccatacgacaagaacatcaagccctccgagaatatctctgtgagcgtgtttgccctgggcgagggcttccacaactaccaccacaccttcccatgggattataagacagccgagctgggcaacaatcggctgaacttcaccacaaacttcatcaacttcttcgccaagatcggctgggcctatgacctgaagaccgtgagcgatgagatcatcaagaacagggtgaatcgcacaggcgacggatcccactacctgtggggatggggcgacaaggatctggacaaggaggagatcaagcaggccatccgcatcaaccccaaggataattga

SEQ ID NO: 160 - DNA, 1062 bp, Sesamaia inferens
atggccccaaacatcgccgaggatgtgaatggcgtgctgttcgagtctgatgcagcaacaccagacctggccctggcaaccccccctgtgcagaaggccgacaacctgcccaagcagtacgtgtggaggaatatcctgctgtttgcctatctgcacctggccgccctgtacggcggctatctgtttctgttcagcgccaagtggcagacagacgtgttcgcctacatcctgtatgtggtgtccggactgggaatcaccgcaggagcacaccgcctgtgggcccacaagtcttacaaggccaagtggcccctgaaagtgatcctggtcatcttcaacaccatcgcctttcaggacgcagcaatggattgggcaagggaccacagaatgcaccacaagtatagcgagacagacgccgatcctcacaatgccacccggggcttctttttctcccacatcggctggctgctggtgaagaagcacccagatctgaaggagaagggcaagggcctggacatgagcgatctgcggtccgaccccatcctgagatttcagaagaagtactatctgctgctgatgcctctggcctgctttatcatgccaacagtggtgcccgtgtacttctggggcgagacatggaccaacgcctttttcgtggccgccatgtttcggtatgccttcatcctgaacgtgacctggctggtgaattccgccgcccacaagtggggcgataagccttacgacaagtctatcaagccaagcgagaacatgagcgtggccatgtttgccctgggcgagggcttccacaattaccaccacacattcccctgggactataagaccgccgagctgggcaacaataagctgaactttaccacaaccttcatcaacttcttcgccaagctgggctgggcctacgatatgaagacagtgtctcaggacatcgtgaagagcagggtgaagaggaccggcgatggatcccacaacctgtggggatggggcgacaagaatcagtctaaggaggagatcgctgccgccatcagaatcaatcctaaggacgattga

SEQ ID NO: 161 - DNA, 1062 bp, Agrotis ipsilon
atggccccaaacatctctgacgatgtgaatggcgtgctgtttgagagcgatgcagcaacaccagacctggccctggcaagcccccctgtgcagaaggccgataaccggcccaagcagtacgtgtggagaaatatcctgctgttcgcatatctgcacgccgccgccctgtacggcggctatctgtttctgacaagcgccaagtggcagaccgacgtgttcgcctacatcctgtatgtgatgtccggactgggaatcacagcaggagcacacaggctgtgggcacacaagtcttacaaggccaagtggcccctgaaagtgatcctgatcatctttaacaccatcgcctttcaggacgcagcaatggattgggcaagggaccacagaatgcaccacaagtatagcgagacagacgccgatcctcacaatgccaccaggggcttctttttctcccacatcggctggctgctggtgcgcaagcacccagatctgaaggagaagggcaagggcctggacatgagcgatctgcaggccgaccccatcctgcggtttcagaagaagtactatctgctgctgatgcctctggcctgctttgtgatgccaacagtgatccccgtgtacttctggggcgagacctggaacaatgcctttttcgtggccgccatgtttagatatgccttcatcctgaacgtgacctggctggtgaattccgccgcccacaagtggggcgataagccttacgacaagagcatcaagccatccgagaacatgagcgtggccatgtttgccctgggcgagggcttccacaattaccaccacacattcccctgggattataagaccgccgagctgggcaacaataagctgaactttaccacaaccttcatcaacttcttcgccaagctgggctgggcctacgacatgaagacagtgtccgacgatatcgtgaagaacagggtgaagcgcaccggcgatggatctcaccacctgtggggatggggcgacaagaaccagagcaaggaggagatcgcctccgccatccggatcaatcctaaggacgattga

SEQ ID NO: 162 - DNA, 1062 bp, Mamestra brassicae
atggccccaaacatctctgaggatgccaatggcgtgctgtttgagagcgatgcagcaacaccagacctggccctggcaagcccacctgtgcagaaggcagacaacaggcccaagcagtacgtgtggagaaatatcatcctgtttgcctatctgcacctggccgccctgtacggcggctatctgtttctgttcagcgccaagtggcagacagacgtgttcgcctacatcctgtatgtgatgtccggactgggaatcaccgcaggagcacacagactgtgggcacacaagtcttacaaggccaagtggcccctgaaagtgatcctgatcatctttaacaccatcgcctttcaggacgcagcaatggattgggcaagggaccacagaatgcaccacaagtatagcgagacagacgccgatcctcacaatgccaccaggggcttctttttctcccacatcggctggctgctggtgcgcaagcacccagacctgaagaagaagggcaagggcctggacatgagcgatctgctgaacgaccccatcctgaagtttcagaagaagtactatctgctgctgatgcctctggcctgcttcgtgatgccaacaatgatccccgtgtacctgtggggcgagacatggaccaatgcctttttcgtggccgccatgtttcggtatgccttcatcctgaacgtgacctggctggtgaattccgccgcccacaagtggggcgataagccttacgacaagagcatcaagccatccgagaacctgtctgtggccatgtttgccctgggcgagggcttccacaattaccaccacacattcccctgggattataagaccgccgagctgggcaaccagaagctgaacttcaccacaaccttcatcaactttttcgccaagctgggctgggcctacgacatgaagacagtgtccgacgatatcgtgaagaatagggtgaagcgcaccggcgatggatctcaccacctgtggggatggggcgacaagaaccagagcaaggaggagatcgcctccgccatccggatcaatcctaaggacgattga

SEQ ID NO: 163 - DNA, 1062 bp, Helicoverpa assulta
atggccccaaacatcagcgaggatgtgaatggcgtgctgttcgagtccgatgccgccacaccagacctggccctgtctaccccacctgtgcagaaggcagacaacaggcccaagcagctggtgtggagaaatatcctgctgtttgcatacctgcacctggcagcacagtacggaggctatctgtttctgttctctgccaagtggcagacagatatcttcgcctacatcctgtatgtgatcagcggactgggaatcaccgcaggagcacaccggctgtgggcccacaagtcctacaaggccaagtggcctctgagagtgatcctggtcatcttcaacaccgtggcctttcaggacgcagcaatggattgggcaagggaccacagaatgcaccacaagtattctgagacagacgccgatcctcacaatgccaccaggggcttctttttcagccacatcggctggctgctggtgcgcaagcacccagatctgaaggagaagggcaagggcctggacatgagcgatctgctggccgaccccatcctgaggtttcagaagaagtactatctgatcctgatgcctctggcctgctttgtgatgccaacagtgatccccgtgtacttctggggcgagacatggaccaacgcctttttcgtggccgccatgtttcgctatgccttcatcctgaacgtgacctggctggtgaattctgccgcccacaagtggggcgataagccttacgacaagagcatcaagccatccgagaacctgtctgtggccatgtttgccctgggcgagggcttccacaattaccaccacacattcccctgggactataagaccgccgagctgggcaacaataagctgaactttaccacaaccttcatcaacttcttcgccaagatcggctgggcctatgatctgaagacagtgtccgacgatatcgtgaagaatagggtgaagaggaccggcgacggaagccaccacctgtggggctggggcgatgagaaccagtccaaggaggagatcgacgccgccatccggatcaatcctaaggacgattga

SEQ ID NO: 164 - DNA, 1062 bp, Helicoverpa zea
atggccccaaacatcagcgaggatgtgaatggcgtgctgttcgagtccgatgccgccacaccagacctggccctgtctaccccacctgtgcagaaggcagacaacaggcccaagcagctggtgtggagaaatatcctgctgtttgcatacctgcacctggccgccctgtacggaggctatctgtttctgttctctgccaagtggcagacagatatcttcgcctacatcctgtatgtgatcagcggactgggaatcaccgcaggagcacaccggctgtgggcccacaagtcctacaaggccaagtggcctctgagagtgatcctggtcatcttcaacaccgtggcctttcaggacgcagcaatggattgggcaagggaccacagaatgcaccacaagtattctgagacagacgccgatcctcacaatgccaccaggggcttctttttcagccacatcggctggctgctggtgcgcaagcacccagatctgaaggagaagggcaagggcctggacatgagcgatctgctggccgaccccatcctgaggtttcagaagaagtactatctgatcctgatgcctctggcctgctttgtgatgccaacagtgatccccgtgtacttctggggcgagacatggaccaacgcctttttcgtggccgccatgtttcgctatgccttcatcctgaacgtgacctggctggtgaattctgccgcccacaagtggggcgataagccttacgacaagagcatcaagccatccgagaacctgtctgtggccatgtttgccctgggcgagggcttccacaattaccaccacacattcccctgggactataagaccgccgagctgggcaacaataagctgaactttaccacaaccttcatcaacttcttcgccaagatcggctgggcctatgatctgaagacagtgtccgacgatatcgtgaagaatagggtgaagaggaccggcgacggaagccaccacctgtggggctggggcgatgagaaccagtccaaggaggagatcgacgccgccatccggatcaatcctaaggacgattga

SEQ ID NO: 165 - DNA, 1062 bp, Helicoverpa subflexa
atggccccaaacatcagcgaggatgtgaatggcgtgctgttcgagtccgatgcagcaacaccagacctggccctggcaaccccacctgtgcagaaggcagacaacaggcccaagcagctggtgtggagaaatatcctgctgtttgcatacctgcacctggcagccgtgtacggaggctatctgtttctgttctccgccaagtggcagacagatatcttcgcctacatcctgtatgtgatctctggactgggaatcaccgcaggagcacacaggctgtgggcccacaagagctacaaggccaagtggcctctgagagtgatcctggtcatcttcaacaccgtggcctttcaggacgcagcaatggattgggcaagggaccacagaatgcaccacaagtattccgagacagacgccgatcctcacaatgccaccaggggcttctttttctctcacatcggctggctgctggtgcgcaagcacccagatctgaaggagaagggcaagggcctggacatgagcgatctgctggccgaccccatcctgaggtttcagaagaagtactatctgatcctgatgcctctggcctgctttgtgatgccaacagtgatccccgtgtacttctggggcgagacatggaccaacgcctttttcgtggccgccatgtttcgctatgccttcatcctgaacgtgacctggctggtgaattccgccgcccacaagtggggcgataagccttacgacaagagcatcaagccatccgagaacctgtctgtggccatgtttgccctgggcgagggcttccacaattaccaccacacattcccctgggactataagaccgccgagctgggcaacaataagctgaactttaccacaaccttcatcaacttcttcgccaagatcggctgggcctatgatctgaagacagtgtctgacgatatcgtgaagaatagggtgaagaggaccggcgacggatctcaccacctgtggggctggggcgatgagaaccagagcaaggaggagatcgacgccgccatccggatcaatcctaaggacgattga

SEQ ID NO: 166 - DNA, 1062 bp, Heliothis virescens
atggccccaaacatcagcgaggatgtgaatggcgtgctgttcgagtccgatgcagcaacaccagacctggccctggcaaccccacctgtgcagaaggcagacaacaggcccaagcagctggtgtggagaaatatcctgctgtttgcatacctgcacctggccgccctgtacggaggctatctgtttctgttctccgccaagtggcagacagatatcttcgcctacatcctgtatgtgatctctggactgggaatcaccgcaggagcacacaggctgtgggcccacaagagctacaaggccaagtggcctctgagagtgatcctggtcatcttcaacaccgtggcctttcaggacgcagcaatggattgggcaagggaccacagaatgcaccacaagtattccgagacagacgccgatcctcacaatgccaccaggggcttctttttctctcacatcggctggctgctggtgcgcaagcacccagatctgaaggagaagggcaagggcctggacatgagcgatctgctggccgaccccatcctgaggtttcagaagaagtactatctgatcctgatgcctctggcctgctttgtgatgccaacagtgatccccgtgtacttctggggcgagacatggaccaacgcctttttcgtggccgccatgtttcgctatgccttcatcctgaacgtgacctggctggtgaattccgccgcccacaagtggggcgataagccttacgacaagagcatcaagccatccgagaacctgtctgtggccatgtttgccctgggcgagggcttccacaattaccaccacacattcccctgggactataagaccgccgagctgggcaacaataagctgaactttaccacaaccttcatcaacttcttcgccaagatcggctgggcctatgatctgaagacagtgtctgacgatatcgtgaagaatagggtgaagaggaccggcgacggatctcaccacctgtggggctggggcgatgagaaccagagcaaggaggagatcgacgccgccatccggatcaatcctaaggacgattga

SEQ ID NO: 167 - DNA, 1059 bp, Choristoneura parallela
atggcccctaacgtgaccgaggagaatggcgtgctgttcgagtccgatgcagcaacccctgacctggccctggcaaggacaccagtggagcaggccgacgattctccccggatctacgtgtggagaaacatcatcctgtttgcctatctgcacctggccgccatctacggcggctatctgtttctgttctccgccaagtggcagaccgatatcttcgcctacctgctgtatgtggcctctggactgggaatcacagcaggagcacacaggctgtgggcccacaagagctacaaggccaagtggcccctgagactgatcctgaccatctttaacacaatcgcctttcaggacagcgccatcgattgggcccgggaccacagaatgcaccacaagtattccgagaccgacgccgatccccacaatgccacaaggggcttctttttctctcacatcggctggctgctggtgaggaagcaccctgagctgaagcgcaagggcaagggcctggacctgtctgatctgtacagcgatcctatcctgcgctttcagaagaagtactatatgatcctgatgccactggcctgcttcatcctgcccaccgtgatccccgtgtatatgtggaacgagacatggagcaatgcctttttcgtggccgccctgtttcggtataccttcatcctgaacgtgacatggctggtgaattccgccgcccacaagtggggcgataagccatacgacaagtccatcaagccctctgagaacatgagcgtgtccctgtttgccttcggcgagggctttcacaattaccaccacaccttcccatgggactataagacagccgagctgggcaaccaccggctgaacttcaccacaaagttcatcaacttcttcgccaagatcggctgggcctatgatatgaagaccgtgtctcaggagatcgtgcagcagagggtgaagaggacaggcgacggaagccaccacctgtggggatggggcgacaaggatcacgcacaggaggagatcaacgccgccatcagaatcaatcccaaggacgattga

SEQ ID NO: 168 - DNA, 1236 bp, Bombyx mori
atgccccctaggctgaaccacttcgaccccgatatcgccctgtacacaagcaccacaaccctgaagctgctgatcaagctggagtttgtgcccaacaggcctatcctgcgctatctgctggagaagaaggccaccctgatgggctaccacttccagaatatgccaccccaggtggatcctatcccaagcggcgtggtgtttgagtccgacacacagaccgacgatctgggcctggacaaggatgtgagcgtgctgaagaaggccacacctaagaagcacgagctggtgtggttcaacatcatctggttcctgtttctgcacatcagctccctgtacggcctgtatctggtgtttacatccgccaagtggcagaccaacgtgttcgcctttggcgtgcacctgatgtgcgcaatcggaatcggagcaggatctcacaggctgtggacacaccgcagcttcaaggcccggaccccactgagaatcgtgctgatgatctggcagaccatgggctttcaggactgtatctttgagtgggccagggaccaccgcacacaccacaagtatgccgacaccgatgccgaccctcacaacgccgagagaggcctgttcttttcccacatgggctggctgtgctgtaagaagtctccagaagtgatcgagggcggccggagaatcgatctgtctgacctgtatgccgatccagtggtcatgttccagaagaagcactacatgaagatgatgcccatcctgtgcttcgtgctgccaacagtgatccccgtgtatttttgggacgagacctggctgaatgccttctttatcccaacaatcctgcggtacacctgtggcatcaacgtggtgtggtctgtgaatagcttcgcccacacctttggctacaggccttatgataagtccctgaacccacgcgagaatatcggcgtgtggatgatctgcgtggagggcttccacaactaccaccacacatttccctgggattatagagccaccgagcaccccctgtacaatatgctgacacctaccatcgtgttcatcgacgccatggccatgatcggccaggcctacgatctgaagacagtgccccacgacatcatcaagcagcggacacacagaaccggcgacggaacccaccagctgtggggatgggacgatcctgagtttaatgagaagctgaaggagaagtactcccgggtgtcttatgccgagagaaagtgtggctga

SEQ ID NO: 169 - DNA, 684 bp, Lobesia botrana
tatggcttgtatctatgcttcacttccgccaaatgggctacgatattctttaacgttatgctccacgttttggcggtgtggggagtaaccgccggggcccaccgtctctgggcgcatcgctcctacaaagcgaagctgcctcttcagatcctcctgatgttgttgaactctctggccttccaaactccagtcagtcattgggctcgggatcacaggctgcaccataaatacagcgacacagacgccgacccacacaacgctaccagggggttcttctactcccacatcggttggcttctcgtgaggaaacacccagaggccatcaagagaggaaagaccatagacgtaactgatattatggaaaatccagtgctgcagtttcaaaagaaatactacgtgtacctctctgggctgctaaccttctttctgccggcgttaatcccaatctattattggggagagactctaaacaacgctcatcacgtctctctgcttcgtcttattataggatacaacgtaacctttttcgtgaacagcgcagctcacaaatatgggaacaaaccatacgacaaacacatgtcgccgacacagagtgtgacattatccctggcaacactaggtgaatgcttccacaactaccatcacgtcttcccattcgattaccgaacagctgagctcggtaac

SEQ ID NO: 170 - DNA, 384 bp, Lobesia botrana
atgaaaatcccgtgccgaagtttcaagagaaataagtactatctataccttagcggtccactgactttcatcctaccagctctaataccggtctactgttggaaggaaaccttaaacaacgcttaccacatctccctccttcgtctcatcactggttacaacatcgcattttccgtgaacagcgctgctcataaatttggcaacaaaccgtacgacaaacacatgtcgccgacacagagtgtgacattgtcgctggcaacactaggagaatgcttccacaactaccatcacgtcttcccattcgattaccgaacggctgagtttggtaacaattacatcaatttcacggcgaaatcgatcgatttctttgctcggatgggatgg

SEQ ID NO: 171 - DNA, 420 bp, Lobesia botrana
ttcgttttgtacataatgtccggccttggtatcacggcgggcgcgcacaggttatgggcccacaagtcttataaagctcgcctgcccctccgcatactgctgacgttcttcaacaccatggctttccaggactctgtccttgactgggcccgcgaccaccgcatgcaccacaagtactccgaaactgacgccgaccctcacaatgcgacccgcggtttcttcttctcccacgttggctggctgctagtgcgcaagcatccgcagatcaaggctaagggacacaccatcgacatgagcgacttgcttgctgatcccgtgctgcgcttccagaagagatactacctcactctgatgcctctcgtctgcttcatcctgcctagctacatacccacgctgtggggagaatctctctggaacgcc

SEQ ID NO: 172 - DNA, 309 bp, Lobesia botrana
acagtgcaggttttgttgcatttagtttcaatatatggactgtatttgataattacgtttaatgtcaagttcctgaccacactttttgcattcctaactatatacacatcggggttcggcatcacggcgggcgtacaccgactgtggtcgcaccgcgcgtaccaggcgcgtacgccgttgcgggttctactggcattgctgttcaccgtcacgggacagcgtgatatttatacctgggccctggaccatcgtgtgcaccacaagtatacggagacggctgcggatccccacgacatacgccgagggttc

SEQ ID NO: 173 - DNA, 1053 bp, Lobesia botrana
atgccacctcaggagcagcctgccgcctgggtgctggacgagtctgatgccgccacagaggacaaggatgtggcagcacacgtgccaccaagcgccgagaagcggaagctggagatcgtgtggagaaacgtgacactgttcgtgttcctgcacgtgggagccgtgtacggaggctatctgttctttacccaggctatgtgggccaccaagatctttacattcgtgctgtatgtgatgtccggcctgggcatcaccgccggcgcacaccggctgtgggcccacaagtcttacaaggcccggctgccactgagaatcctgctgaccttctttaacacaatggcctttcaggacagcgtgctggattgggcccgggaccacagaatgcaccacaagtactccgagacagacgccgatccccacaatgccaccaggggcttctttttcagccacgtgggatggctgctggtgcgcaagcaccctcagatcaaggccaagggccacaccatcgacatgtccgatctgctggccgatcctgtgctgaggtttcagaagcggtactatctgacactgatgccactggtgtgcttcatcctgccttcctatatcccaaccctgtggggcgagtccctgtggaacgcctacttcgtgagcgccatcttcagatactgttatgtgctgaacgtgacatggctggtgaattctgccgcccacaagtggggcgataagccatatgacaagaacatcaatcccgtggagaccaagcctgtgagcctggtggtgtttggcgagggcttccacaactaccaccacacctttccctgggactacaagacagccgagctgggcggctactctctgaatctgagcaagctgttcatcgacacaatggccaagatcggctgggcctacgatctgaagagcgtgtcccccgacatcgtggagaagagggtgaagcgcaccggcgatggctctcaccacgtgtggggctgggacgatgccccaaccgagcagaaggtggccgccacaatcgtgaatcccgacaagaccgagtga

SEQ ID NO: 174 - DNA, 1116 bp, Lobesia botrana
atggagtttctgtctgagatcgagaactgctccgagcggctgagaaagaccgagaagatgccccctaacgtgacagatgtgaatggcgtgctgttcgagtccgatgcagcaacccctgacctggccctggcaacaccaccagtgcagaaggccgacaacaccccaagggtgtacgtgtggcgcaatatcatcctgtttggctatctgcacatcgccgccctgtacggcggctatctgtttctggtgtctgccaagtggcagaccgatatcttcgcctatctgctgtatgtggcaagcggcctgggcatcacagccggcgcacacaggctgtgggcacacaagtcctacaaggccaagtggcctctgcgcgtgatcctgaccatctttaacacaatcgcctttcaggacagcgccatcgattgggccagggaccaccgcatgcaccacaagtattccgagaccgacgccgatccccacaatgccacacggggcttctttttctcccacatcggctggctgctggtgcggaagcaccctgagctgaagagaaagggcaagggcctggacctgtctgatctgtacgccgacccaatcctgagattccagaagaagtactatctgatcctgatgcccctggcctgttttgtgatgcctaccctgatcccagtgtacttctggggcgagacctggacaaatgcctttttcgtggccgccctgtttcggtatgccttcatcctgaacatcacctggctggtgaattctgccgcccacaagtggggcgataagccctacgacaagacaatcaagcctagcgagaacctgagcgtgtccctgtttgccttcggcgagggctttcacaattaccaccacaccttcccatgggactacaagacagccgagctgggcaaccaccggctgaacttcaccacaaagttcatcaactttttcgccaagctgggctgggcctatgatatgaagaccgtgagcgacgagatcgtgcagaagcgggtgcagagaacaggcgacggatcccaccacctgtggggatggggcgacaaggatcagtctaaggacgaggtggatgccgccatccgcatcaatcccaaggacgattga

SEQ ID NO: 175 - DNA, 1086 bp, Lobesia botrana
atgcggagcgtgtccaccagagtgaacaaggccatggtgccaaggctggaagtgaataccggcgtgaagacagtggacgaggagcagctgccatctcgggatgagaacaagcccagaatcagcaagctggtgaagatcaatatcgtgtccttcggctatctgcacatcgccgccctgtacggcctgtatctgtgctttaccagcgccaagtgggccacaatcggcctggccttctttctgctggtggtgggcgagatcggcatcaccgccggcgcacacaggctgtggtcccaccgcacctacaaggccaacagagccctggagatcctgctgatcatcatgaactctgtggccttccagaatagcgccatccactggatcagagaccacagactgcaccacaagtactctgacaccaacgccgatccccacaatgcctccaggggcttcttttattctcacatcggctggctgctggtgcgcaagcactctgatgtgatcaacaagggcaagatgatcgacatgagcgatatctacaacaatcccgtgctgacattccagaagaagtatgccatccctttcatcggcaccatctgctttggcctgcccacactgatccctatgtactgttggggcgagagcctgaacaatgcctggcacatcaatatcatgagatacgtgctgtgcctgaacatcacctttctggtgaattccgccgcccacttctttggccagaagccttacgacaagaacatcctgccaggccagaatatcctggtgtctatcctggccgtgggcgagggcttccacaactaccaccacgtgtttccttgggactatagaacagccgagctgggcaacaatatgctgaatctgaccacaaagttcatcgatttctttgcctggctgggctgggcctatgagctgaagaccgtgtccacaaacgcaatcgacctgagggcaaagaggaccggcgatggaacaaatctgtggggatggggcgacgagcacatgagccaggaggagatggaggcctccgatatcctgtttgtgaacaaggagaatgagaagacccacacacactga

SEQ ID NO: 176 - DNA, 993 bp, Lobesia botrana
atggagaatagcaagtccgaggtgagggaggaggtgcccttccgccacaccctggtgaagcacaacgtgatcaagtttggctacatgcacctggccgccctgtacggcgtgtatctgtgcttcaccagcgccaagtggcctacactgctgtgggccttcctgctgctggagtttgccaagatcggaatcacagccggcgcacacaggctgtggtgtcaccggagctacaaggccaagctgccactggagatcctgctgctgatcttcaacagcatcgcctacatgaataccgccacatattgggtgcgggatcacagagtgcaccacaagtttgccgacaccgatgccgacccacacaacgtgaatcggggcttctggttttcccagatcggctggctgtttgtgagaaagcacccagacgtggtggagaagggagcaaccgtgttcatggacgatatccaccagaataagctgctgcggtttcagaagaagtacgccttctttgtgatcggcctgtgggcctacgtgatccccacagtggtgcctatgtatttctggggcgagtccctgaacaattcttggcacatctgcaccatgctgaggcacgtgctgacaatcaaccagatctttctggtgaatagcatcggccactcctggggcaacaagccttatgataagaatatccgcgccgtggagaacgtggccgtgtctctgatgagcaccggcgagtgtttccacaactaccaccacgtgttcccatttgactataaggcctccgagctgggcatgaccaagttcaatgccgccacaatgtttatcaacttctttgcctgggtgggctgggcctacgatctgaagaccatccccgacgagctgatcatcgccaggtctaagcgcacaggcgatggcagaaatctgtggggatggggcgatgtggaccagaccgaggaggagacaaagggcgtgaagtacctgtatacaaagaacgagtga

SEQ ID NO: 177 - DNA, 978 bp, Lobesia botrana
atggagaaggagagcaagaccaagagctcctggttccggacatttgagaagaagctgggcttccagaacgagatcaagtgggtgaataccatcctgatcgccatctaccacatgctgttcgtgtactggccataccactatgccttccccgtgaagtggcagaccatcgtgtttgccgccatcatgtacgtggcatccggattcggagtgacaggaggagcacacaggctgtggacccacaaggcctacaaggccacactgcctctgaagatctttatgctgatgctgtattctagcgccggccagaacagcctgctgaattgggtgcgggatcacagagtgcaccacaagttctccgacaccgatgcagacccacacaacgcaaataggggactgttcttttcccacatcggctggctgatgctgaagaagaacgatgaagtgatccggaagggcaagcagatcgatatgtctgacatcgagaatgaccccgtgctgagattccacgagaagtacttccagtggtttaagctgctgttttgctactatctgcctaccatgatcaacgtgtggtgttggggcgaggattggaagacagccgtggcctggcagtgctttatccggtatatcaccacattccactccgagctgaccgtgaattctctggcccacgccttcggctacaagccctacaacaaggacctgaagccagccgagaatagatttgtggccacagtgtctctgggcgagggctggcacaactaccaccacgccttccccttcgactacaaggccgccgagcacttcgacttctttaatttcggcaccaccttcatccggtggttcgagaagctgggactggcatacgatctgcgccaggcaacccccgagatgatcaacgccatctctaagaggctgggcgacggcacacccgtgcactttcctctggatagcgacggcgagcgcgccgtgggctga

SEQ ID NO: 178 - DNA, 942 bp, Lobesia botrana
atggagaagaacgagatcaggaatcagacccagagctcctcttggttccgcaacctggagaagaggctgggctttcgcaccgatatcaagtggggcaatgtgatccacatcacactgtaccacgccctggtgttctacatggccatcaactatgcctttccagtgaagtggcagaccatcctgttcggcatctacacctatatcacagccggctatggagtgacagccggcgcacacaggctgtggagccacagatcctacaaggtgaccctgcccctgaagatctggacactgatctgctattccatggccggacagaactctgtgctgaattgggtgagggatcaccgcgtgcaccacaagcactccgacaccgatgccgaccctcacaacatcaatcggggcctgttctttgcccacgtgggctggctgatgatgaagaagaacgaggaggtggtgcggagaggcaaggagatcgatctgtctgacatcgagaatgaccccgtgatgaggttccacgagaagtactttggcctgagcaagatcctgctgtgctatgccgtgccactggccatcaacacctggtgtctgggcgaggcatggccaaccgccgccgcctggcacctgatgaggcacctgggcaccctgcacgccacattttctctgaatagcctggcccacgcctacggctatcagccctacaacaagaatatgaggcctacccagaaccgcttcgtggccaccgtgacatttggcgagggctggcacaattaccaccacgtgttccccttcgactacaagggcgccgagttcggcgacgtgctgaacttcaccacagcctacatccggggctacagatatctgggcctggcctatgatctgaaggaggccacaccagagatgatccggagcgtggccgccagagtgggcgacggctcctga

SEQ ID NO: 179 - DNA, 1074 bp, Lobesia botrana
atgggagtgaccgtgggagcacacaggctgtgggcccacagagcctataaggcccggtggccaatgagactgctgctggccctgctccagacaatggcctttcagaaccacatctatgagtgggtgcgggaccacagagtgcaccacaagtacaccgagacagacgccgatccccacaatgcccggagaggcttctttttctctcacatcggctggctgatggtgaggaagcacaaggaagtgatcgagaagggcgccgccatcgacatgagcgatctggagaaggacccaatcgtgatgtttcagaagaagacctacctggtggtcatgccctttctgtgcttcctgctgccttcctgggtgccagtgcacttctggggcgaggatccttggacatcttggtacgtggccagcatctggaggtataccctgagcctgaactttacatggctggtgaattccgccgcccacatctggggcaacaagcctttcgacaatgtgcgcggagcaaccaactggtccccacccgtgtggtctacacaggtgccaatgggaggacacggagccggaggatctgccatcatctgcggcatctttaagagcgccaccaggaagaagctgcgcatgatcggcaaggacggcgtgctgttcaggcgccagaggccaagcctgggctccaagccttctatcggcgccaccgataacgtgacagtggcaatctgtgcagtgggagagggatggcacaattaccaccacgtgttcccatgggactacaaggccgccgagctgggcaactacagaaccaatgtgagcacagccatcatcgacctggccgccaagtacggctgggcctatgatctgaagaccgtgtccgcccagatgatccagaacagggtggcaaggaccggcgacggaacacacccctccgtggccaagacagtggaggagctggatcactctgaggacgagcacgatcaccaccacccagagaatcccgtgtggggctgggacgataaggacatgcctgaggaggatagacagctggtggagatcagccaccagaagtccgagtga

SEQ ID NO: 180 - DNA, 1008 bp, Lobesia botrana
atgggagtgaccgtgggagcacacaggctgtgggcccacagagcctataaggcaaggtggccaatgaggctgctgctggccctgctccagacaatggcctttcagaaccacatctatgagtgggtgagggaccaccgcgtgcaccacaagtacaccgagacagacgccgatcctcacaatgcccggagaggcttctttttctctcacatcggctggctgatggtgcggaagcacaaggaagtgatcgagaagggcgccgccatcgacatgagcgatctggagaaggaccccatcgtgatgtttcagaagaagacctacctggtggtcatgccttttctgtgcttcctgctgccttcctgggtgccagtgcacttctggggcgaggatccatggacatcctggtacgtggcctctatctggaggtataccctgagcctgaactttacatggctggtgaattccgccgcccacatctggggcaacaagccattcgataatgtgcgcggagcaaccaactggtctccacccgtgtggagctcccaggtgccaatgggaggacacggagagggaggatctgccatcatctgcggcatctttaagagcgccaccggcaagaagaacatcggcgccaccgacaatgtgacagtggcaatctgtgcagtgggagagggatggcacaattaccaccacgtgttcccttgggactacaaggccgccgagctgggcaactaccggaccaatgtgagcacagccatcatcgacctggccgccaagtacggctgggcctatgatctgaagaccgtgtccgcccagatgatccagaacagggtggcaagaaccggcgacggaacacacccttccgtggcaaagacagtggaggagctggatcactctgaggacgagcacgatcaccaccacccagagaatcccgtgtggggctgggacgataaggacatgccagaggaggatagacagctggtggagatctctcaccagaagagcgagtga

SEQ ID NO: 181 - DNA, 1173 bp, Lobesia botrana
atgctgaccgccggcctgtgcgatatctttatcgtgaggctgcgcaagaccgagaagatgccccctaacgtgacagacgtgaatggcgtgctgttcgagagcgatgcagcaacccctgacctggccctggcaacaccaccagtgcagaaggccgataacaccacacagtccctgcgggtggccaagcacaattctgtgagcgtgatctttacctctccactgtgcatgggcgccacatgcagcctgtgccggagaaacggcaagcctatcttcctgccaacctactatatgtggcacagggactgggcatctcagcaggcaaggaccggaagcggccacacatcccctatcaagcagaacggcctgtccgagtcttttagcccaagctccacactgctgcactctagctccatggtgggcctgagcatctacatcggcatcaatcacatgttctgccaccaggactccgccatcgattgggcccgggaccacagaatgcaccacaagtattctgagaccgacgccgatccacacaacgccacacggggcttctttttcagccacatcggatggctgctggtgcggaagcacccagagctgaagagaaagggcaagggcctggacctgtccgatctgtacgccgatcccatcctgagattccagaagaagtactatctgatcctgatgcctctggcctgttttgtgatgcccaccctgatccccgtgtacttctggggcgagacctggacaaatgcctttttcgtggccgccctgtttaggtatgccttcatcctgaacatcacctggctggtgaattccgccgcccacaagtggggcgataagccatacgacaagacaatcaagccctctgagaacctgtccgtgtctctgtttgccttcggcgagggctttcacaattaccaccacaccttcccctgggactacaagacagccgagctgggcaaccaccggctgaacttcaccacaaagttcatcaactttttcgccaagctgggctgggcctatgatatgaagaccgtgagcgacgagatcgtgcagaagagggtgcagaggacaggcgacggatcccaccacctgtggggatggggcgacaaggatcagtctaaggacgaggtggatgccgccatccgcatcaatcctaaggacgattga

SEQ ID NO: 182 - DNA, 1089 bp, Lobesia botrana
atgccacctcaggtggatccaaccccatccggcgtggtgttcgagtctgatacccagacagccgacctgggcctggacaaggacgtgagcgtgctgaagaaggccacacccaagcagcacgagtacgtgtggggcaacatcatctggttcctgtatctgcacatcgcctccctgtacggcctgtatctggtgtttacctctgccaagtggcagacaaacgtgttcgcctttgccgtgcacctgatgtgcgcaatcggaatcggagccggatctcacaggctgtggacccaccgcagctttaaggcccggacacctctgagaatcgtgctgatgctgtggcagaccatgggctttcaggactgcatctttgagtgggcccgggaccacagaacccaccacaagtatgccgacacagatgcagacccacacaacgcagagaggggactgttctttagccacatgggctggctgtgcgtgaagaagagccctgaagtgatcgagggcggcaagcgcatcgatctgacagacctgtatgaggaccctgtggtcatgttccagaagaagcactacctgaagctgatgccactgctgtgctttgtgctgcctaccgtgatcccagtgtattgttggaacgagacatggctgaatgccttcctgatccctaccatcctgcggtacacatgcggcatcaacgtggtgtggtctgtgaatagcttcgcccacgtgtttggctaccggccctatgataagtctctgaaccctagagagaatctggccgtgtggatggtgtgcgtggagggcttccacaactaccaccacacctttccatgggactatagagccacagagctgccactgtacaatatgctgacccccacaatcgtgttcatcgaggccatggccaagatcggccaggcctacgatctgaagagcgtgtccccagagatcgtgaagcagaggagccagaggaccggcgacggatcccaccagatctggggctgggacgatcccgagtttaccgagaagctgaaggagaagctgggcgccgtgtctaatacagagcggggcgagaaggtgaagagcggctga

SEQ ID NO: 183 - DNA, 1203 bp, Lobesia botrana
atgaccgccacagataatctgatcagcccccctgaggccgacggcgatatcctgagccggatcgaggatctgtgcgacagctggaacaaggtgaagaacgaggagaccgagaagcctcagatcccactccagctgcccaagaccatcggcacagactattcctacagacacgagatcgtgtggaagaacgccatcggcttcctgatcctgcacatcctggccgtgtggggcctgggcgtgctgctgacaggcgtggtggccgtgtgcctgtttagcaccgagggagtgacaatcggagcacacaggctgtacacccacaagtcctttaaggccacacctttcctgcgcgccctgctgatcctggcacagaccacagccggccagaatagcatgttcatctgggtgcgggatcacagactgcaccaccggtattccgacaccgatgccgacccccacaactctaagagaggcttctttttctgccacatcggctggctgatgtctaagaagcacccttacgtgatcgagctgggcaggaagatcgatatgagcgacctggaggccgactggatggtcatgtttcagaagaagtactattactggctgtattttctgctggccgtgttcatcccaacctgggtgcccgtgcactacttcggcgagtcttggaagaatagcctgctggtgtgctatatctttcgctacatggtccaggtgaacggaacctggctggtgaactccgccgcccacctgtgcggaacaaggccttatgatcgcggcctccagccagtggagtcttggtttgtgagcttcatctccctgggcgagggctggcacaattatcaccacgccttcccttgggactacaaggccgccgagctgtccaccaagtttaaccagtctgccaagatcatcaagtttttcgaggatatcggcctggcctacgacctgaagacagcctctccagagatggtggccaagaggatcgtgcgcaccggcgatggcacacacatcgacctgggcaatgaggaggcccgggccgccgtgaccgccccaggcatcgtgcaccccctgaaccctttctataccgtgacataccagccaccctccggcacactgaagcacgagggactgccactgtttcacgagaaggacgtgttcaactctaatggcaactttctgaagcggagcgccagagcctga

SEQ ID NO: 184 - DNA, 1041 bp, Lobesia botrana
atggcctctaagagccacagggaggtgcgcatgcagcagacctttgccagcggcaacaagtcccccttcagagacctggtgggaccacaggcagaccctagctggcggtggagagtgtcctggtttaatgtgctgctgttctcctactcttatagcagcgccctccagggcctgtacctgtgctttacctccgcccagtgggccacaatcttcctgaacgccatcctgctggtggcaagcctccagggcgccctgatcggagcacacaggctgtggagccaccgctcctttaaggccaagctgccactccagatcctgctgctggtgctccagaccctgtctgtgcagtacagcgtgtataactgggtgcgggatcacagactgcaccacaggcacagcgacaccgatgcagacccccacaatgccacaagaggcttctttttctcccacatcggctggctgctggtgcagaagcaccctgaggtggtgaagaggggcaaggccacagatatgagcgacatcatcaacaatccactgctgatgtttcagaagaagtactgggccatcctgagcttcctgatcagcctggtcatcccaaccgccacacccatgtacttttggggcgagaacttcaagaatgccctgtgcgtgaacgccctgcggtacaacctgtgcctgaacttcacctggtctgtgaatagcgtggcccacacattcggctctcggccctatgatggctccatccagtctagacagaacatggcagtgaccgtggtgacactgggcgagggctttcacaattaccaccacgccttccctcgcgattatgccgccggagagctgggcgagtggtttaatttctccaagatcttcatcgactttttcgcctggatcggctgggcctacgatctgaagaccgtggacaagaagacagtgctgaagagggtgctggagaagggcgacggctcttatcgctgtaagaccgagaacaagacaaagatcaatggcagcaagatcggcaacaatatgctgctgctgtga

SEQ ID NO: 185 - DNA, 1047 bp, Lobesia botrana
atgatgatggcatgcctgccaaagaccggagcagcaatgcagaaggcaagggccaaccgcgatctgtttgaggtgttcgtgcctgtgatcacccggagccggtacctccagtctaagacaaatagccagctgaaggcctaccacttcctggcctgggcatggtatgtgctgggcgagctgggcgtgatcgccggagcacacaggctgtgggcacaccgcacctataaggccaagatgccactccagatcatcctgatgctgtttaactgtatcggcttccagaataccgccacagattgggtgcgggaccacagagtgcaccacaagcacagcgacacagatgcagacccacacaactcccagaggggaagcgtgagcttcgatctgcaccactccgccgtggagctgtacacctatagagccatgttctttttctctcacgtgggctggctgctgacaaggaagcaccgcctggtgaaggagaagggcgaggccgtggatatgaccgacatctactctaatcctgtgctgaggttccagaagaagtatagcctgccactgatcggcaccctgtgctttgccctgccaacactgctgcccgcctactgttggggagaggcagtgggaacagcatggaacatcaatctgctgcgctattgcctgaacctgaatggcaccttcctgacaaactccgccgcccacaagtttggctctaagccctacgataagaccatcccccctacacagaatctgctggtgagcttcatgaccctgggcgagggcttccacaattaccaccacgtgttttcctgggattatagggccgccgagctgggaaacacctacctgaatatgaccacaatcttcatcgactttttcgccctgatcggctgggcctatgatctgaagacagtgcctgaggacgtgatcaagaagcggatggccagaaccggcgacggcacaaatctgtggggctggggcgataaggacatgaccaaggaggatgtggtggacacagagatcaggtttccagccaagaaggagtga

SEQ ID NO: 186 - DNA, 975 bp, Lobesia botrana
atgagctccaccgatatctactccgccgaggtgtatccaaagctgaaagccccccaggccgacaatcacaagtttcggatctgctacacaaacgtggccgtggtgctgtatcaccacctggcagggctgtacgggctgtatctgtgcttctcttgtcagtgggccaccatcatcctggccggaatcctgcaagaggccggaatgctgggactgatggccggagcacacaggctgtgggcacacagaagctacaaggccaagctgcctctccagatcatcctgatcatctttcactccatcgccaatcagtacacagcctataactgggtgagggatcaccgcctgcaccaccggtattctgacaccgatggcgacccccacaatgccacaagaggcttctttttcagccacgtgggctgggtgctggtgcagaagcaccctgaggtgcacaggagaggagcatctaccgatatgagcgacctggagaagaaccctgtggtcatgtttcagaagaagtacaaggtgccattcatcgccctgatcacctacgtgatccccacactggtgcctatctatttttggaacgagaccttcacaaatgcctggcacatcaacatcctgaggatggtggtgtgcctgaattgtacctttctgatcaactccgccgcccacatctacggatctaggccatatgataaggacgtgagagccaccgagaacatggccgtgaatgtggccacactgggcgagggctttcacaattaccaccacgtgttcccctgggactatagggcagaggagttcggcaggcgcctgaacgtgaccagggcctttatcgagctgttcgcaagggtgggatgggcatacggcctgaagacagtgggaaggggcgccgtggtggccagatgtggccggagaggcgatggcagctacgtggagcagaagtatgacgataagggcctgcgggacgagtga

SEQ ID NO: 187 - DNA, 993 bp, Lobesia botrana
atggacgatagcaagtccgaggaggtgccattccggcacaccctggtgaagcacaatgtgatcaagtttggctacatgcacctggccgccctgtacggcgtgtatctgtgcttcaccagcgccaagtggcagacactgctgtgggccttcctgctgctggagtttgccaagatcggaatcaccgccggcgcacacaggctgtggtgtcaccggagctacaaggccaagctgcccctggagatcctgctgctgatcttcaacagcatcgcctacatgaataccgccacatattgggtgcgggatcacagagtgcaccacaagtttgccgacaccgatgccgaccctcacaacgtgaataggggcttctggttttcccagatcggctggctgtttgtgcgcaagcacccagacgtggtggagaagggcaagacagtgtacatggacgatatccacaagaatcccctgctgaggttccagaagaagtatgccttcctggtcatcggcctgtgggcctacgtgatccccaccgtggtgcctatgtatttctggggcgagacactgtctaacagctggcacatctgcaccatgctgcggcacgtgctgacaatcaaccagatctttctggtgaatagcatcggccactcctggggcaacaagccttacgataagaatatcagagccgtggagaacatcgccgtgtccctgatgtctaccggcgagtgtttccacaactaccaccacgtgttcccatttgactataaggcctccgagctgggcatgaccaagttcaatgccgccacactgtttatcaacttctttgcctgggtgggctgggcctacgatctgaagacaatccccgacgagctgatcaccgccaggtctaagcgcacaggcgatggaaggaatctgtggggatggggcgatgtggaccagaccgaggaggacacaaagggcgtgaagtacctgtataccacaaacggcaagatcgagtga

SEQ ID NO: 188 - DNA, 1005 bp, Lobesia botrana
atgatcaaccctacctgtgattccacatgtaaagccaacggcctgaccaatgaggccttccagaatctgctggagatctccaccgattctacactgaagcggaagtaccacgacggctctcaggaggagttctggatgcaagtgtatgtggagaacaatgccgtgggccagatcgccatcaagaggctggtgcgctttaacagctccctggcctctagcatctacacctatatcacagccggctacggagtgaccgccggcgcacaccggctgtggagccacagatcctacaaggtgaccctgcccctgaagatctggacactgatctgctatagcatggccggacagaactccgtgctgaattgggtgcgggatcacagagtgcaccacaagcacagcgacacagatgccgaccctcacaacatcaataggggcctgttctttgcccacgtgggctggctgatgatgaagaagaacgaggaggtggtgcggagaggcaaggagatcgatctgtctgacatcgagaatgacccagtgatgcgcttccacgagaagtactttggcctgagcaagatcctgctgtgctatgccgtgccactggccatcaacacctggtgtctgggcgaggcatggccaaccgccgccgcctggcacctgatgaggcacctgggcaccctgcacgccacattttctctgaatagcctggcccacgcctacggctatcagccctacaacaagaatatgaggcctacccagaaccgcttcgtggccaccgtgacatttggcgagggctggcacaattaccaccacgtgttccccttcgactacaagggcgccgagttcggcgacgtgctgaactttaccacagcctacatcaggggctaccgctatctgggcctggcctatgatctgaaggaggccacccctgagatgatccggtccgtggcagcaagagtgggcgacggctccaggtattctgtgccaacaaaggaggccaatcagtga

SEQ ID NO: 189 - DNA, 1038 bp, Lobesia botrana
atggccccccagtctgaggagatcgataccagctccggcgatatcaaggacgccagcacagagctgctgaagatgccagagacaagggtgacacccagaaagtacaccatcgtgtactatacagtggtgctgctggtgtactatcacctggccgccctgtacgccctgtatctgtgctggaccagcgtgaagtggcagacagtgttctttaacgtgatcctgcacgtggcagcaatcagcggagtgaccatcggagcacacaggctgtggtcccacagagccttcaaggccaagatgccactccagatcctgctgatgctgctgaacagcatggcctcccagaatacagcctgttactgggcccgggatcacagactgcaccacaagttcaacgacaccgatgccgacccctacaattccacaaggggcctgttttattctcacatcggctggctggtggtgaagaagcaccccgacgtggtgaagaagggcaaggagatcgatatgtccgacctgtacggcaaccctgtgctgaagttccagaataagtactatatcgtgctgaccctggccctgggctttgtgctgcccacactgctgcctatctactattggcaggagacactgacaaatgcctaccacgtgtccctgctgcggtttgtgatcggcctgcacgccatcttctttgtgaactctgtggcccacaagtatggcaataagccttacgataagtattctagcgccacccagtctatcccagtgagcgtgatcgccctgggcgagtgctaccacaacttccaccacgccttcccttttgattacagatcctctgagatcggcaacaattatatcaattttggcacccacatcatcgacttctttgcctggatcggctgggcctatgatctgaaggtggccagcgaggacatggtgctgaagagggccaacaagaccggcgatggaacagacctgtggggaagggtgaagggagtggagaagccaaatgaggactgggtggataaggacctgatgcggtga

SEQ ID NO: 190 - DNA, 984 bp, Lobesia botrana
atggcccctcagtccgaggagatcgataccagctccggcgatatcaaggacgcctctacagagctgctgaagatgccagagacaagggtgacacccagaaagtacaccatcgtgtactatacagtggtgctgctggtgtactatcacctggccgccctgtacgccctgtatctgtgctggacctctgtgaagtggcagacagtgttctttaacgtgatcctgcacgtggcagcaatctctggagtgaccatcggagcacacaggctgtggagccacagagccttcaaggccaagatgccactccagatcctgctgatgctgctgaacagcatggcctcccagaatacagcctgttactgggcccgggatcacagactgcaccacaagttcaacgacaccgatgccgacccctacaatagcacaagaggcctgttttattcccacatcggctggctggtggtgaagaagcaccccgacgtggtgaagaagggcaaggagatcgatatgtccgacctgtacggcaaccctgtgctgaagttccagaataagtactatatcgtgctgaccctggccctgggctttgtgctgcccacactgctgcctatctactattggcaggagacactgacaaacgcctaccacgtgagcctgctgcggtttgtgatcggcctgcacgccatcttctttgtgaactccgtggcccacaagtatggcaataagccttacgacaagtattctagcgccacccagtctatcccactgagcctgatcgccctgggcgagtgctaccacaacttccaccacgccttcccctttgattaccggtcctctgagatcggcaacaattatatcaattttggcacccacatcatcgacttctttgcctggatcggctgggcctatgatctgaaggtggcctccgaggacatggtgtctaagagggccaataagaccggcgatggcacagacctgtggggccgctga

SEQ ID NO: 191 - DNA, 1137 bp, Lobesia botrana
atggccccaaatggcaaggagatcgcacagccagtggcagacaccgataacaagttccgggccctggtggctccccaggccggccctagaaagtacaccgtggtgtatatgacagtgctgaatttcctgtactggcacctgggcgggctgtacggcctgtatctgtgctttacctccgccaagtgggccacactgttctttaacgcaatcctgcacgtgctggccgtgtggggcaacaattgttggggcagccctaccctggtgacaccactgctccagtccgaggccagctccaccgatcctagcaatgccaacgcccactccggcctgccaaagtctagccacagcctgggcagccggagccaggtgagcctggagctgccaacaggcatctactctaacaataacaagtgcttctgtcacaggctgcaccacaagtatagcgacaccgatgccgacccccacaatgccacacgcggcttcttttactctcacatcggctggctgctggtgaggaagcaccccgaggccatcaagcgcggcaagaccatcgatgtgacagacatcatccagaaccccgtgctccagttccagaagaagtactatctgtatctgagcggcatgctgacctttgtgctgccagccctgatccccatgttcttttggggcgagacactgaataacgcccaccacgtgtccctgctgcggctgatcatcgtgttcaatgtgaccttctttgtgaactctgccgcccacaagtacggcaataagccctatgacaagaacatgagccctacccagtccgtgacactgtctctggccaccctgggcgagtgctttcacaattaccaccacgtgttcccttttgattatcggacagccgagctgggcaataactacatcaacttcaccacaaagtttctggacttctttgccgccatcggctgggcctacgatctgaagtatgcctccgacgagatgatcatgaagcggtctaagagaaccggcgatggcacagacctgtggggcagagtgaagagcaccaatgacgataaggcaccaaagaacgtgtggggatggggcgatgaggacatggtggaggagcacaagaatatcctgctgtcccagaaccagtga

SEQ ID NO: 192 - DNA, 1113 bp, Lobesia botrana
atgtacatgggcgatggcaactgcctgccatctgagctgaagatgacccccaaccccagaaatgatgtgacagaggacaagcccctgcaagaggactatctggagaagctggtggctccccaggcctctcccatcaagtttgaggccagctacgtgaatctgatcatgtttacctatttccacacagccggcgcctacggcctgtatctggcctttatcgaagcccggtgggccaccatcttcgaggagatcgagaagatcagctccctgtttccagccttcgccctgtacgaggccggaatcgtgggcgtgaccgccggcgcacacaggctgtgggcccaccgcacatataaggccaagctgcccctccagatcctgctgatgtttctgcactctttcagctgccagtacagcgcctttcactgggccagggatcaccgcctgcaccacaagtattccgacaccgatggcgaccctcacaacgccacacggggcttctttttctcccacatcggctggctgctggtgaagaagcacccagaggccaagcggagactgaggggcatcgatgtgaaggatctgctggagaatcgggtgctccagttccagaagaagtacgccaccccctttatcggcacaatctgtttcatcctgcctaccctggtgccaatgtatttttgggatgagcctttccggacagcctggaacgtgaatatcctgagagtgatcgtgctgctgcacgccacctttctggtgaactccgccgcccacgccttcggcaataagccctacgacgcctctatcaccccttcccagtctatcagcatctccatcgccacactgggcgagggctaccacaactttcaccacgtgttcccttgggattatagggccgccgagctgggaaacaacgtgctgaacttcaccaccctgttcatcgactttttcgccagaatcggctgggcctacgatctgaagacagtgggaaacgacgcaatcgcaaagagggcagagagaaccggcgacggcacaaatctgtggggctggggcgataaggatctgcccaagaagcacgaggagatcgccagggtgagcttcgagaaggccgagtga

SEQ ID NO: 193 - DNA, 951 bp, Lobesia botrana
atgacccccaaccccagaaatgatgtgacagaggacaagcccctgcaagaggattacctggagaagctggtggctccccaggccagccccatcaagtttgaggcctcctacgtgaacctgatcatgtttacctatttccacacagccggcgcctacggcctgtatctggcctttatcgaagcccggtgggccaccatcttcctgggctccctgatcctgatgtttctgcacagcttctcctgccagtactctgccttccactgggcccgggaccacagactgcaccacaagtatagcgacaccgatggcgacccacacaacgccacacggggcttctttttctcccacatcggctggctgctggtgaagaagcacccagaggcaaagcggagactgaggcgcatcgatgtgaaggatctgctggagaatcgggtgctccagttccagaagaagtacgccaccccttttatcggcacaatctgtttcatcctgcctaccctggtgccaatgtatttttgggatgagccattccggacagcctggaacgtgaatatcctgagagtgatcgtgctgctgcacgccacctttctggtgaactctgccgcccacgccttcggcaataagccctacgacgccagcatcaccccttctcagagcatctccatctctatcgccacactgggcgagggctaccacaactttcaccacgtgttcccctgggattatagggccgccgagctgggcaacaacgtgctgaacttcaccaccctgttcatcgacttctgcgcccgcatcggctgggcctatgatctgaagacagtgggcaacgacgcaatcgcaaagagggcagagaggaccggcgatggcacaaatctgtggggctggggcgataaggatctgcccaagaagcacgaggagatcgcccgggtgtctttcgagaaggccgagtga

SEQ ID NO: 194 - DNA, 1113 bp, Lobesia botrana
atgtctcccagcaatgccaccaacacactggtggaggcccctaagtacatcgaggacatcacagagtgccgggtgtgccggaccacacacaagcccagcaagggcgacatctgctttaacaataacaatctggagacaaaggataagcacgagatcaaccacaacaacaacaaggagacaaagtacaacacaaggcagcactcctggctgaagcggtgggagaagtggatgggcttcgagacagagctgatctggctgaacctgatctccatcagcgtgttccacatcgtgaccgtggtgtggttcacatacaatctgtggatcggcagctatcccaagtggcagtccacactgtacgcctacttcatcggcaacgtggccggattcggagtgaccgccggcgtgcacaggtactggtgccacagatcctataaggccacactgcctctccagtggatcctggtcatctgttactctgtggccggacagaaccccatccctgagtgggtgagggaccaccgcgtgcaccacaagttttccgagagctccgccgacccacacgatgccggaaggggcttctttttcgcccacgtgggctggctgatgatgcggaagcacccagacgtgatctctgagggcagaaagatcgacatgagcgatatcacaaacgatcccgtggtgcagttccacaccaagtactttaagctgttcaagacagccttttgtttcgtgctgcctaccctggtgccagtgtggggctggaatgagacatgggagtacgccatcctgtcccaggtgtgcctgagatatctgatgtctctgaattgtacatgggccgtgaacagctttgcccacctgtggggcaataagccatacgatcccaacatcacccctgtggagaattggggcgtgtctgccgtggccctgggcgagggctggcacaactatcaccacacatttccatgggactacaaggccgccgagctggcctataccctgaatctgaccacactgctgctggatggcttcgccaggatcggctgggcccacaacctgaagcaggccacccctgagctgatccaggccgtggtgaatcgcaagcacaagagctga

SEQ ID NO: 195 - DNA, 1077 bp, Lobesia botrana
atgtctccaagcaatgccaccaacacactggtggaggcccccaagtacatcgaggacatcacagagtgccgggtgtgccggaccacacacccacctagcaagggcgacatctgctttaacaataacaatctggagacaaaggataagcacgagatcaaccacaacaataacaaggagacaaagtgtaatacaaggcagcactcctggctgaagcggtgggagaagtggatgggcttcgagacagagctgatctggctgaacctgatctccatcagcgtgttccacatcgtgaccgtggtgtggttcacatacaatctgtggatcggcagctatccaaagtggcagtccaccctgtacgcctacttcatcggcaacgtggccggattcggagtgacagccggcgtgcacaggtactggtgccacagatcctataaggccaccctgcccctccagtggatcctggtcatctgttattctgtggccggacagaacccaatccccgagtgggtgagggaccacagggtgcaccacaagttttccgagagctccgccgaccctcacgatgccggaaggggcttctttttcgcccacgtgggctggctgatgatgcggaagcaccctgacgtgatctctgagggcagaaagatcgacatgagcgatatcaccaacgatccagtgacagccttttgcttcgtgctgcccaccctggtgcccgtgtggggctggaatgagacatgggagtacgccatcctgtcccaggtgtgcctgagatatctgatgtctctgaattgtacatgggccgtgaacagcttcgcccacctgtggggcaataagccttacgatccaaacatcacccccgtggagaattggggcgtgtctgccgtggccctgggcgagggctggcacaactatcaccacacctttccttgggactacaaggccgccgagctggcctatacactgaatctgaccacactgctgctggatggcttcgccaggatcggctgggcacacaacctgaagcaggcaaccccagagctgatccaggcagtggtgaatcgcaagcacaagagctga

SEQ ID NO: 196 - DNA, 518 bp, Lobesia botrana
acacaaatatgcgaccaaaccatccgacacatacatttcagccacccaaagtatccctgtatctgtgatagcgttaggagagtgctaccataatttccaccacgcctttccattcgactatagatcctcagaaataggcaacaattatataaatttcgggacgcatataatagactttttcgcatggattggttgggcttatgatttgaaggttgcgtctgaagatatggttttaaagagagctaacaaaaccggtgatggaacagatttatggggaagggtaaagggggttgagaaaccaaacgaagattgggttgataaagatcttatgcgatgattagtcgacgctgaaactgaccgtgattgaaataactgtgaagtaaataaaacaataaaacggaaaaaaaaaaaaaaaaaaaaaaaaaaaaaaaataaaaaggggttaaaacaaaaaaataaatcgaaaaacaaaacaaaagaaaaaaaaaaaaccattggtaggaggtgtggggggtagg

SEQ ID NO: 197 - DNA, 779 bp, Lobesia botrana
cgcaaccttcaaatcataagcccaaccaatccatgcgaaaaagtctattatatgcgtcccgaaatttatataattgttgcctatttctgaggatctatagtcgaatggaaaggcgtggtggaaattatggtagcactctcctaacgctatcacagatacagggatactttgggtcgctgaactgtatttgtcgtatggtttgttcccatatttgtgagcaacgctgttcacgaagaagattgcgtgtagaccgatgacgaaacggaggagagacatgtggtaagcgttggtaagggtttcttgccaataataaataggaagtaatgttggtaagacgaaaccgagcgcgagggttaagacgatgtagtatttattctgaaacttgagaactgggttgccgtataaatccgacatgtctatttccttccctttcttcaccacatcagggtgctttttcaccaccaaccagccgatgtgggagtagaacaggcctcgagtagaattgtaggggtcggcgtccgtgtcgttgaacttgtggtgtaatctgtggtctctagcccagtagcaagccgtgttctgtgaggccatactgttgagtagcatgagcaggatctgaaggggcatcttggctttgaatgcgcgatgggaccagaggcgatgggcgccgatagtcacgccggaaattgcggccacgtgaagaataacattaaagaaaacagtctgccatttaacagaagtccaacacaaatataatgcatacaatgctgccaagtgatagt

SEQ ID NO: 198 - DNA, 524 bp, Unknown
  Sequence isolated via Lobesia botrana RNA-seq assay
cgccctgccagatccggcaagcgatgacgcacggccataggccgctgtgccttgggccgctgtttcttgggcaatgtctccggatcatgctccaaagggggccgtagacactgtccggggccggaatgtcaagccaagataggaacttcggccatcattcaactggggatgcggcctctcaaaaaccgccgttggtctggttggcatcgaccgccgcccccgcccgcgcccggcgtgcgttgctcaccaattgccccggacggtcatcccggtcgggcctcgcggtcgccagccggagcacggcggcatagccctgctgcacctccatgcggccggcgtggcggctctcgccgagcaggcgatggacctgcggcagattgtagcagggcacgtagaacagcagatgatgctcgaggtgatagttcacgtaatacggggcgatgaacaggcgctcgaggacattggcgtgcgtggtgcgggtattgcgcaagggatcgctgctgtcaggaacgacagcatgctcg

SEQ ID NO: 199 - DNA, 395 bp, Unknown
  Sequence isolated via Lobesia botrana RNA-seq assay
atccacattgcggatgtagtgaaagcgctcgccatctcgcgcaccgacgctgccaagcggctcgcgcgctggcgcgaacaaggctggatgagccgggtcggcgcaggcgcctatgtcccggcatcgattgatactctcggctccgaacgcgtcctcgatgatgcatgggttcttgtacccgcgctttttgcgccagcttatattgccggtcgtagcgccgcagagcattgggatcttaccgagcagatcttcaaggatatcgtggtcgtcaccggacaaaacatccgcgagaggcaccagaaacgacagggcttcgaatttacgctgaagcatctgaagccggagaagattttcggaacaaagccggtctggcgtcaccacaccaaggtgccggt

SEQ ID NO: 200 - DNA, 416 bp, Unknown
  Sequence isolated via Lobesia botrana RNA-seq assay
agtgccggtctgcgcattgatcgacttgcagctcgcgcgaaacgtcttgtaggcggcgagatgatcgtcgtcgttccagcccttcacctcggtccagggcagcggcagatattgcgcgcctgatatttcgaagggcaggggaagctgcggatagggcaaggcccgcggcggagccgcgggctgggccggaaggtggtgatgatgactgcgatggtgccgccgcgccgcctcggcgccgagcgaaaacgacgacagcacgacgatggcagcgcaaagcgccgtcgcgctgttcttcaggaacgccttaattcgcgcttccggtgccaaccagcttccaattcggatcgcgagaggtgatgtcgcgggcgaaagtccagatgtcggtgatgtcggcaacggtatcggcgctgccgtcg

SEQ ID NO: 201 - DNA, 302 bp, Unknown
  Sequence isolated via Lobesia botrana RNA-seq assay
agcctcgtcgaaatgtcgctcggcgtcgccatcggcgcgctgaccttcaccggatcggtaatcgcgttcctgaaactgtcggcgcgaatgagcggcgcgcccatcatcctgccgttccgccacgtcatcaacatcggtctcgctctcgcgctggtgtttttcatcgtcggcctcgtgctgtcgggcagtgcgttcgacttctggatgatcgtgatcctggcgctggcgctcggcgtgctcatgatcatcccgatcggcggcgccgacatgccggtcgtgatctcgatgctgaactcctattc

SEQ ID NO: 202 - DNA, 1008 bp, Unknown
  Sequence isolated via Lobesia botrana RNA-seq assay
agatgcagatgtgtcagcgcatcgacgccgtcatgagcgcgattgaggatgtcctgcagcgtcggcaactgcatctgggcgagatcggcccggcgccgcgccgacgaaatcgcgtgcgcgaccgtctgcgcatcgaatcgcgcggcaagttcgcgcgccgcccggttgatgacccgcgcgccgagcggctgctcgttgcgcagcacctgctcgggtggtgccttcatattccagacgattccgatccaggacaggatcgtcaggacataataggtcacgtccacttcccaccagcgaaagccctgtcgcacactgctctggtaggcgtggtgattgttgtgccagccctccccgagggtgaagaaggccagcagccagttgttgcgggaattgtcgccggtcacgtagcgcttgcgtccatggacatgggcgagggaattgatgcagaaggtcgcgtgatagagcagcaccgtgctccacaggaaaccgacgaccaggcccggccatcccgcaaccaggaagcagagcacggccagcacgacggccggcagcagctcgagccggtgcagccacatcagctccggatagcgcgcgagatcttccacttttacgaggtcggtcgcgtcgtgctcccggtagaagatccagccgagatggctatagacgaagccgcgctgacgcggtgaatgcacgtccagttcggtatcggaatgcagatgatggtgccgatgcttggcggcccaccacaacacgctcttctgcgcggtgctttgcgcaagacaggccaggatgaactgaaacacccggctggtggcaaaggccttgtgcgagaaatagcggtggtagcctgccccgatcgcgaacatgcgcacgacatacagcgacacgcagatcgcgacggcctgccaggtgacgcccgaccagatcgccgcaaggcagccgagatggaccagcacgatcggtattgcggaggggtacatgacgtcgtcgtgcggatcgtcggcgggg

SEQ ID NO: 203 - DNA, 530 bp, Unknown
  Sequence isolated via Lobesia botrana RNA-seq assay
agccttcaggagccgtggcctcaaccttgcggtcggttacctctcggccttcatcatcggattgccctacgaatattactgcctgttccattgggaccaccaccgctacacccaggatcccgacagggacccggaactgatcgtcggcgcgaagccggcaaccgatacgcagctagcgatcgcctatagcggcctgctccaggtcgccggccgcctctggctgatgctcggccacgccgtcaccggcaaggtcgtcgtgccctggatccccgaaaacaagcgcgccaccatcgtgaccgaagcgcggacctatgtcgcgctctatgcactgctgctcgcgctgtcgttgtggtttgcatcggcgctgctgctgtggctctggatcgttccactcttcgtcggacaattcttcctgcggccctatctctatgcggagcacaccggctgcgacacaacacgcagcgcattccagaacacccgcacgacctataccggcgcgatcgtcaaatggttcgcgtggaacatgccct

SEQ ID NO: 204 - DNA, 611 bp, Unknown
  Sequence isolated via Lobesia botrana RNA-seq assay
catctagccgccgaggctcgcgcccgatgcggtgacgcggccttgtcgggaaccgggtgggttggggcccgaagacatggatagtggcgccaatccaaccgcgcaggacatgagacatgtcaaatcgcacccgcctccccgacacattcagccgcctcgcctggtccaatctggcagcacagtcggccgagcagatcgcgctggctgcggctcccattgtcgcagtgctgacgctcggggtcgccgaaggccagaccggcctgctccagaccgcgctcaccctgcccttcgtactcttcgcgattcccgccggcctgctcgccgaccgcatctcgcgccgcgcgctgatggccggcgccgaggcactccgcgcggtcgcgctcggggcaatcgtattgctgctcgcgctcggcgcgctcaatctgccactgctggcgctgctcggctttgcagcggtgtgcggcaccgtcgtctacagcgtggccgcgccggcgctggtgccctcgctggtgagcgcggacctcttgcccgccgcgaatgcgcgcatcgagcttgcacgcaccatcgccttcgccagcggccctgcactcggcggcgcgctggtgggctgg

SEQ ID NO: 205 - DNA, 342 bp, Unknown
  Sequence isolated via Lobesia botrana RNA-seq assay
acctggcttttatagaggcaaggtgggcgactatattcttagcattcgccctctacgaagccgggatagtcggcgtgacggccggcgcccaccgcctctgggcccaccgcacatacaaagccaaactacccctccaaatcctgctcatgttcctccactccttctcctgccaatactccgccttccactgggcccgggaccaccgtctccaccacaagtactccgacacagacggggacccccataacgcgaccagggggttctttttctcgcatattgggtggctgctggtgaagaaacaccctgaagcgaagaggaggttgagggggattgatgttaagg

SEQ ID NO: 206 - DNA, 1083 bp, Lobesia botrana
atggtggatatcgcccctaacggccagaagatcgccgagccagtggccgacatcgatcacgagttccggcccctggtggctccccaggccggcccaagaaagtacaccgtggtgtatatgacagtgctgaactttctgtactggcacctgggcggcttctacggcctgtatctgtgctttacctccgccaagtgggccacaatcttctttaatgtgatgctgcacgtgctggccgtgtggggcgtgaccgccggcgcacacaggctgtgggcacaccggagctacaaggccaagctgccactccagatcctgctgatgctgctgaacagcctggccttccagacacccgtgtcccactgggcaagggaccacaggctgcaccacaagtactccgacaccgatgccgacccccacaatgccacacggggcttcttttattctcacatcggctggctgctggtgaggaagcaccctgaggccatcaagcgcggcaagaccatcgatgtgacagacatcatggagaaccccgtgctccagtttcagaagaagtactacgtgtacctgtccggcctgctgaccttctttctgcccgccctgatccctatctactattggggcgagacactgaacaatgcccaccacgtgagcctgctgcggctgatcatcggctataacgtgaccttctttgtgaattccgccgcccacaagtacggcaataagccatatgataagcacatgagccccacccagtccgtgacactgtctctggccaccctgggcgagtgtttccacaactaccaccacgtgttcccttttgactatagaacagccgagctgggcaacaattacatcaatctgaccacaaagtttatcgatttctttgccgtgatcggctgggcctacgacctgaagtatgcctctgacgatatgatccggaagagaagcaagaggaccggcgatggaacagacctgtggggacgcgtgtctggcaccgagggcaagctgacacccggcaatatctggggctggggcgataaggacatgcctgaggagcacaaggagatcctgctgagccagcagtga

SEQ ID NO: 207 - DNA, 2205 bp, Lobesia botrana
atgaccgaggtgacaaaggtgaacccagacctccagagggagcgcgacaactgtaccttcaatgtgaccgagctgacaaatctgatcgatggcggcgtgcagaagacagaggagcggagaaagcgcgaggagatggtgctgaaggagggcatccacctggacgaggtgccctccgagtacctgtctcacaaggagaagtatgagctggccgtgaagaaggcctgctacctgtttaagatgatccggcggctgcaagaggaggagaacaccggcatggagaattaccagtatgtgacatatctgcactggagctcccagtatgagagcgagtccaccgtgggcatcgagcagtctcccagcgagctgaacaatgccagcgccgccctgaactatctgaagaagctgcgggtggccagaaagaagagggctgccgccatcgccaaggtgcacttcaagcactgtctgggcatctaccacagagaggtgctgggcggatctctgggcagcgccatcctgagggatggatcccccctgaccctgcactatgtgatgttcatccctaccatcatgggccaggccacagtggagcagcaagtgctgtggatcggccgcgcctttaactgcgatatcatcggcacctacgcccagacagagctgggacacggcaccttcatccggggactggagaccacagccacctacgaccccagcacaaaggagtttgtgctgcacagccctaccctgacatcctataagtggtggccaggaggactggcccacaccgccaattactgtatcgtggtggcccagctgtatacacagggcaagtgccacggcatccacccttttatcgtgcagctgagagacgaggagacccacatgcccctgcctggcatcaaggtcggcgagatcggcgccaagctgggcatgaacggcacaaacaatggcttcctgggctttgataaggtgcgcatcccacgggagcacatgctgatgaagaatgccaaggtgctggaggacggcacctacgtgagagccccctctagcaagctgacctatggcacaatgatgttcgtgagggtggtgctggtgaacgacgtgtgcagctacatggccaaggccgtgaccatcgccacacgctattccgccgtgcggcaccagtctcagccaaagcccgacgagcctgagccacagattctggagtacgtgacccagcagcacaagctggccatcggcatcgccacagtgcacgcctttagactgtccgcctcttggctgtggaatatgtataacaatgtgaccgccgagctggacgccggcgatctggagaggctgcctgagctgcacgccctgtcctgctgtctgaaggccgtgtctacagccgatgccagcgagtgcgtggagcggtgtagactggcatgcggaggacacggatacatgctgtcctctaacttcccactgatgtacggcatggtgaccgccgcctgcacatatgagggcgagaataccgtgatgcacctccagacagccagatacctggtgaaggcatggcagcaggcacagggaggaaacccactgacccctacagtggcatatatcggaaccatcggagccggccggagaagcccttcctgggataatacagtggagggcatcatcctgggctttcagagggtggccgccggcaagatcgcacagtgcgtggccaacatcgagaagaggcagcgcaccggcatgagctacgaggacgcctggaatatgacatccgtgcagctggtgtctgccagcgaggcccactgccgggccttcatcctgaggacctactttgaggagacagagaaccagatcggatccgtgtctccagccctgcgcgccgtgctgctccagctggtggatctgtatgtggtgttctgggcactccagagggtgggcgatctgctgcggtttaccagcatctccgagcgggatatcgagcagctccagtcctggtacgaggatctgctgatcaagctgagggtgaacgcagtgggcctggtggacgccttcgatatcagagacgagatcctgaattctgccctgggcgcatacgacggaagagcatatgagaggctgatggccgaggccctgaagtctccactgaacgccgagcccgtgaatcagagcttccacaagtatctgaagccttttatgcagggcaagctgtga

SEQ ID NO: 208 - DNA, 2064 bp, Bicyclus anynana
atggaggacctgtcttttgtgcccgagctgcctagcggaccactggacgtgtaccggaacaatgcctccttcaactggaagagactgaagctggccctggagggcgactttgatctgctgaagctgaagtacaagatctggaggaccctggagaaggaccccctgttcgcccacagccccatcaccctgcctgtggaggagcagaagcgcatcacacagctgcagctgaagaagatcaacgagtacaagttcatccccaaggagatgtttaatgcctcctattctaagcggaccagaacaatcatgaccatcaacgaggccgtgcagtccctgaatcctagcgtgtccgtgaagatggccatcggcatctacctgttttccaatgccctgctgtctctgggcaccgagaggcacctgaagttctatgaggccacaatcaagcaccgcgagatcctggcctcttttagcctgaccgagatcgcacacggaagcgacgcaaggctgatgagaaccacagccacctatgatccctccacacgggagttcgtggtgcacacacctgattttgaggcagcaaagtgctgggtgggaaacctgggcagaacctgtacacacaccctgctgttcgcccagctgatcacaccagacggcaccaatcacggcctgcacggctttgtggtgccaatcaggaagcccgatacactggagacctaccccggcctgatcgtgggcgacatgggagagaagatcggcgtgaacggcatcgataatggcttcatcatgtttaaccagtataggatccctcgcgagaacctgctgaatcgcacagccgacgtgaccgaggatggcacatacgagagctccttctccgagccatctaggatcctgggcgccgccctggagaatctgagcgccggccgcatcggcatcatgcaggagagctgccactccctgtctagcgccgtgaccatcgccgtgaggtacgcagcaacaaggacccagttcggagagagggacagggagacacccctgatcgagtacgagctgcaccagtggcggctgtttggctacgtgagcgccgccatcgtgttcagactgtacatcgacctgtttgcccacacctatctgaacatcgtggagaagagcaatgccggccaccgggtggataacctgtctgaggccgtgagcgagatccacgccatggtgtcctctagcaagccactgctgacctggaccacactgagggcagcacagcagtgcagagaggcatgtggaggacacggatacctgaagtgtgccaacctgggcgacatccggtccaatcacgagcccacagtgacctatgagggcgataacaatgtgctgtctcagcaggccggcaattggctgctgagacagtacgaggcagcaatcgcaggcaagcctgtggactccccactgggcaccgtggccttcctgaaggacctgcggacaatcaaggatcggaccttcacctgcaagaccacaaaggagctgaaggatcctcagttcatcatctctacctacaagtggctgctgtgctggatgctgaagtatacacacgagaagaacgaggccaatctgtcccagggcctgaccaaggtgcaggccaggacaaagtctcaggtgtatcgctggaagacactgaccaaggtgtacgccgagtatatcaccctggtgttctgcctggacaacatcgataagaaggagagagagctgcagcctatgctgctgaagctgttttgtctgtacggcctgtggagcctggacgagaatctggtggagctgtatcagggaggattcgcaaagggagaggagtgcgcacggctggtgagagattctgtgctggagctgtgcggcgaggtgaagaacgagatcgtgagcgtggcagacgcactggcaccaaccgattttgtgctgaacagcgtgctggccaagtccgacggcaagctgtaccagaatctgcagaaggccttctttagccaccccggcgtgttcgagaggacaccttggtggcgcgatgtggtgccagcctccaagctgtga

SEQ ID NO: 209 - DNA, 2007 bp, Plutella xylostella
atgtccgccaaggtgaaccctgatctgcagagggagcgcgacaagtgtaccttcaacgtgaccgagctgacaaatctgatcgatggcggcgtgcagaatacagaggagcggaagagacaggaggacctgctgctgaaggagggcatccacatcgaggaggtgccatccgagtacctgtctcacaaggagaagtatgagctggccgtgaagaaggcctgcctgctgttcaaagtgatccggagaatgcaggaggaggagaacaccggcatggagaattaccggtccgtgctgggcggaaacctgggctccgccatcctggcagatggatctcctctgaccctgcactatgtgatgttcatcccaaccatcctgggccaggcaacagtggagcagcaggcatactggatcggaagggcctttaatctggatatcatcggcacctatgcccagacagagctgggccacggcaccttcatcagaggcctggagaccacagccacctacgacccaagcacaaaggagtttgtgctgcactcccccaccctgaccagctacaagtggtggccaggaggactggcccacaccgccaactactgtatcgtgatggcccagctgtatacaaagggccagtgccacggcctgcaccccttcatcgtgcagctgcgggacgaggagacccacatgcctctgaagggcatcaagatcggcgagatcggcgtgaagctgggcatgaatggcacaaacaatggcttcctgggctttgagcacgtgaggatccccagagagaacatgctgatgaagaattccaaggtgctggaggatggcacctacgtgcactctctgagctccaagctgacctatggcacaatgatgttcgtgagggtggtgctggtgacagatatgtgcaactacatggccaaggccgtgaccatcgccacacgctatagcgccgtgaggcgccagtcccagccaaagcccgatgagcctgagccacagattctggactacgtgacccagcagcacaagctgatgatcggcatcagcacagtgcacgcctttaggctgtccgccaattggctgtggcatatgtacaacaatgtgatcgcagagctggacaccggcgatctggagcgcctgccagagctgcacgcactgtcttgctgtctgaaggcagtgaccacagccgatgccgccgagtgcgtggagaggtgtcgcctgagctgcggaggacacggatacatgctgtctagctccctgccaaccacatacggcctggtgaccgcagcctgtacatatgagggcgagaacaccgtgctgctgctgcagacagccaggtacctggtgaaggcatggcagcaggcaatgggaggaaatgcactgacccctacagtgtcctatatctctgaggtgagcaccggccggagatctcccccttgggataacagcgtgcagggcgtgatccacggattccacagggtggcagcaggcaagatcggaatgtgcgtggccaacatcgagaagagacagaagagcggcctgtgctacgaggacgcctggaatatgacctccgtgcagctggcatctgccagcgagtcccactgcagggcaatcctgctgagcacctactattccgagacagagaagctggccagcaaggtgacccccgccctgaagacagtgctgctgcagctggtggatctgtacgtggtgtattgggcactgcagagggtgggcgacctgctgcgctttacctctatcagcgagcgggatatcgagcagctgcagaactggtacgaggacctgctgacacggctgagacctaatgccgtgggcctggtggacgccttcgacttcagagacgagatcctgcactctgccctgggcagctacgatggccgggtgtatgagagactgatggaggaggccctgaagtctcccctgaacgcccagcctgtgaatgacagcttccacaagtatctgaagccctttatgcagggcaagctgtga

SEQ ID NO: 210 - DNA, 2064 bp, Plutella xylostella
atggccgacctgtctttcatcccagatctgcccagcggccctctggactgctataggtccacagcctcttttgattggaagcagctgaagctgtccctggagggcgacctggatctgctgaagctgaagtacaagatctggaagaccctggagaaggacctgctgttcgcccccccttctgtgacacctagcgtggaggagcagaagcgcatcacccagctgcagctgaagaagatcaacaattacaagttcatgacctatgagatgtttaagagctcctactctaagaggacacgcgccatcatgaccatcaacgaggccgtgcagagcgtgaatccaagcgtgtccgtgaagatggccatcggcatctacctgttcagcaacgccctgctgtccctgggcacagagaggcactataagttctttgaggccaccatctggaatcgcgagatcctggcctcctttgccctgacagaggtggcacacggatctgacgcaaggctgatgtgcaccacagcctcttatgatcctaagagcagagagttcgtggtgcacaccccagacttcaaggcagcaaagtgctgggtgggaaacctgggcaggacctgtacacacaccctgctgttcgcccagctgatcacacctgacggcaccaatcacggcctgcacggctttgtggtgccagtgagagatcccgccacactggagacctacccaggcctggtggtgggcgacatgggagagaagatcggcgtgaacggcatcgataatggcttcatcatgtttaaccagtatcggatccccagagagaacctgctgaatcggacagccgacgtgaccgaggatggcgtgtacgagtctagcttcagcgagccttccaagatgctgggagccgccctggagaatctgagcgccggcaggatcggcatcatgcaggagtcctgccacacaatctcctctgccgtggcaatcgcaatcagatacgcagcaacccggagacagttcggcgccaaggacgatgagatccccctgctggagtacgagctgcaccagtggagactgtttggatacgtgagcgccgcagtggtgttccgggtgtacatcgagaagtttgccagaatctatctgaacatcgtggagaagagcaatgccggcctgaaggtggacaacctgtccgccgccgtgtctgagatccacgccatggtgtcttgtagcaagcctctgctgacctggaccacactggatgcagtgcagcagtgcagggaggcatgtggaggacacggctacctgaagtgcgccaacctgggcgacctgagatgtaatcacgagccaacagtgacctatgagggcgataacaatgtgctgagccagcaggccggcaattggctgctgaggcagtacgagcaggcagcaggaggagtggactcccccctgggcacagtgaccttcctgacaaggtgccaggacattctggaggataagttttcctgtgagcgcaccgaccagctgaaggatgtgaagttcatcacatctacctaccagtggctgctgtgctggctgctgaaggagacccaccagcagtatgagtctgaggtgagcaagggccagagcaagttcgaggccaagtccaagtgccaggtgtataggtggaagacactgtcccgcgtgtacgccgagtatctggccatcatcttttgttccgagtctgtgagccaggaggagaccggactgagacccgtgctgaacaagctgctggtcatgtacggcctgtggagcctggaccagcacctggtggagctgtatcagggcggctttgcctccggcgattctctggcaaggctgctgagggcagcaattctggagatgtgcgcacagctgaagccagaggtggtgtccgtggtggacgcactggcacctaccgatttcgtgctgaactccgtgctgggcaagtctgacggcaacctgtaccagaatctgcagaaggccctgtttggaggcccaggcgcaatggagagggcaagctggtggagagaggtggtgaatagcgccccctccaagctgtga

SEQ ID NO: 211 - DNA, 1986 bp, Rattus norvegicus
atgaatccagatctgaggaaggagagggcaagcgccacattcaacccagagctgatcacccacattctggacggctcccccgagaatacccggagaaggcgcgagatcgagaacctgatcctgaatgaccctgatttccagcacgaggattacaactttctgacacggtctcagagatatgaggtggccgtgaagaagagcgccaccatggtgaagaagatgagggagtacggcatctccgacccagaggagatcatgtggttcaagaagctgtacctggccaattttgtggagcccgtgggcctgaactattctatgttcatccctaccctgctgaaccagggcaccacagcccagcaggagaagtggatgcggcccagccaggagctgcagatcatcggcacctacgcccagacagagatgggacacggaacccacctgagaggactggagaccacagccacctatgatcctaagacacaggagtttatcctgaatagccctaccgtgacatccatcaagtggtggccaggcggcctgggcaagacaagcaaccacgccatcgtgctggcccagctgatcacccagggagagtgctacggactgcacgccttcgtggtgcccatcagagagatcggcacacacaagcccctgcctggcatcaccgtgggcgatatcggccctaagtttggctatgaggagatggataacggctacctgaagatggacaattataggatcccacgcgagaacatgctgatgaagtacgcccaggtgaagcccgacggcacatatgtgaagcctctgtccaataagctgacctacggcacaatggtgttcgtgaggtcttttctggtgggcaacgccgcccagtctctgagcaaggcctgtaccatcgccatccgctattctgccgtgcggagacagtctgagatcaagcagagcgagccagagccccagattctggacttccagacacagcagtacaagctgtttccactgctggccaccgcctatgccttccactttgtgggcaggtacatgaaggagacctatctgcgcatcaatgagagcatcggacagggcgacctgtccgagctgcccgagctgcacgcactgacagcaggcctgaaggccttcaccacatggaccgcaaacgcaggaatcgaggagtgcaggatggcatgtggaggacacggatacagccacagctccggcatcccaaatatctatgtgaccttcacacccgcctgcacctttgagggcgagaacacagtgatgatgctgcagaccgccaggtttctgatgaagatctacgatcaggtgcgctccggcaagctggtgggaggaatggtgtcttatctgaatgacctgcctagccagagaatccagccacagcaggtggccgtgtggccaacaatggtggatatcaactctctggagggactgaccgaggcctacaagctgagggcagcaagactggtggagatcgcagccaagaatctgcagacacacgtgtcccaccggaagtctaaggaggtggcctggaacctgacctccgtggacctggtgagagcctctgaggcccactgccactatgtggtggtgaaggtgttcagcgataagctgcccaagattcaggacaaagccgtgcaggccgtgctgaggaacctgtgcctgctgtactccctgtatggcatctctcagaagggcggcgattttctggagggcagcatcatcacaggagcacagctgtcccaagtgaatgcaaggattctggagctgctgaccctgatcagacctaacgccgtggccctggtggacgccttcgacttcaaggacatgacactgggctccgtgctgggccgctacgacggaaacgtgtatgagaatctgttcgagtgggccaagaagtcccctctgaacaagaccgaggtgcacgagtcttaccacaagcacctgaagccactgcagagcaagctgtga

SEQ ID NO: 212 - DNA, 1986 bp, Rattus norvegicus
atgaatccagatctgaggaaggagcgcgcctctgccacattcaacccagagctgatcacccacattctggacggcagccccgagaatacccggagaaggcgcgagatcgagaacctgatcctgaatgaccctgatttccagcacgaggattacaactttctgacacggtctcagagatatgaggtggccgtgaagaagagcgccaccatggtgaagaagatgagggagtacggcatctccgatccagaggagatcatgtggttcaagaattctgtgcacaggggacacccagagcctctggacctgcacctgggcatgtttctgcccaccctgctgcaccaggcaacagcagagcagcaggagaggttctttatgcctgcctggaacctggagatcaccggcacatacgcccagaccgagatgggacacggaacacacctgaggggactggagaccacagcaacctatgaccccaagacacaggagtttatcctgaatagccccaccgtgacatccatcaagtggtggcctggcggcctgggcaagacaagcaaccacgccatcgtgctggcccagctgatcacccagggagagtgctacggactgcacgccttcgtggtgccaatcagggagatcggcacacacaagccactgcccggcatcaccgtgggcgatatcggacccaagtttggctatgaggagatggataacggctacctgaagatggacaattataggatccctcgcgagaacatgctgatgaagtacgcccaggtgaagccagacggcacctatgtgaagcccctgagcaataagctgacctacggcacaatggtgttcgtgcggtcctttctggtgggcaacgccgcccagtctctgagcaaggcctgtaccatcgccatcagatattccgccgtgcggagacagtctgagatcaagcagagcgagcctgagccacagattctggacttccagacacagcagtacaagctgtttccactgctggccaccgcctatgccttccactttgtgggcaggtacatgaaggagacctatctgcgcatcaatgagagcatcggacagggcgacctgtccgagctgcccgagctgcacgcactgacagcaggcctgaaggccttcaccacatggaccgccaacgccggcatcgaggagtgcaggatggcatgtggaggacacggatacagccacagctccggcatccctaatatctatgtgaccttcacaccagcctgcacctttgagggcgagaacacagtgatgatgctgcagaccgccaggtttctgatgaagatctacgatcaggtgcgctccggcaagctggtgggaggaatggtgtcttatctgaatgacctgcccagccagaggatccagcctcagcaggtggccgtgtggcctacaatggtggatatcaactctctggagggactgaccgaggcctacaagctgagggcagcaagactggtggagatcgcagccaagaatctgcagacacacgtgtcccaccggaagtctaaggaggtggcctggaacctgacctccgtggacctggtgagagcctctgaggcccactgccactatgtggtggtgaaggtgttctccgataagctgcccaagattcaggacaaagccgtgcaggccgtgctgaggaacctgtgcctgctgtactccctgtatggcatctctcagaagggcggcgattttctggagggcagcatcatcacaggagcacagctgtcccaagtgaatgcaaggattctggagctgctgaccctgatcagacctaacgccgtggccctggtggacgccttcgacttcaaggacatgacactgggcagcgtgctgggcagatacgacggcaacgtgtatgagaatctgttcgagtgggccaagaagtcccctctgaacaagaccgaggtgcacgagtcttaccacaagcacctgaagccactgcagagcaagctgtga

SEQ ID NO: 213 - DNA, 1995 bp, Arabidopsis thaliana
atggagggcatcgaccacctggccgatgagcggaataaggccgagttcgacgtggaggatatgaagatcgtgtgggccggcagcagacacgcctttgaggtgtccgataggatcgcacgcctggtggcatccgaccccgtgttcgagaagtccaatagggcccgcctgtctcggaaggagctgttcaagtctaccctgagaaagtgcgcccacgcctttaagaggatcatcgagctgcgcctgaacgaggaggaggcaggccggctgagacacttcatcgaccagcctgcctacgtggatctgcactggggcatgtttgtgccagccatcaagggccagggcaccgaggagcagcagaagaagtggctgagcctggccaataagatgcagatcatcggctgttatgcccagacagagctgggacacggatccaacgtgcagggactggagaccacagccaccttcgacccaaagacagatgagtttgtgatccacacaccaacccagacagcatccaagtggtggccaggaggcctgggcaaggtgtctacccacgccgtggtgtacgccaggctgatcacaaatggcaaggattatggcatccacggcttcatcgtgcagctgcgctctctggaggatcacagccccctgcctaacatcaccgtgggcgacatcggcacaaagatgggcaacggcgcctacaatagcatggacaacggcttcctgatgtttgatcacgtgcggatccccagagaccagatgctgatgcggctgagcaaggtgaccagagagggcgagtatgtgccatccgatgtgcccaagcagctggtgtacggcaccatggtgtatgtgcggcagacaatcgtggcagacgcaagcaacgcactgtccagggccgtgtgcatcgcaacacgctactctgccgtgcggagacagtttggcgcccacaatggcggcatcgagacccaggtcatcgactataagacacagcagaacaggctgttcccactgctggcatccgcctacgccttccgctttgtgggcgagtggctgaagtggctgtataccgatgtgacagagaggctggccgcctctgactttgccaccctgcctgaggcacacgcatgtacagcaggcctgaagagcctgaccacaaccgcaaccgcagatggaatcgaggagtgcaggaagctgtgcggaggacacggatacctgtggtgcagcggactgcccgagctgtttgccgtgtacgtgcctgcctgtacctatgagggcgacaatgtggtgctgcagctgcaggtggccagattcctgatgaagacagtggcccagctgggctctggcaaggtgcctgtgggaacaaccgcatacatgggaagggcagcacacctgctgcagtgccgcagcggagtgcagaaggccgaggactggctgaacccagatgtggtgctggaggccttcgaggcaagggccctgcgcatggccgtgacctgcgccaagaatctgagcaagttcgagaaccaggagcagggctttcaggagctgctggcagatctggtggaggcagcaatcgcccactgtcagctgatcgtggtgtccaagtttatcgccaagctggagcaggacatcggcggcaagggcgtgaagaagcagctgaacaatctgtgctacatctatgccctgtacctgctgcacaagcacctgggcgatttcctgtccaccaattgtatcacacccaagcaggctagcctggccaacgaccagctgcggagcctgtatacccaggtgagacctaacgccgtggccctggtggacgcctttaattacacagatcactatctgaactccgtgctgggccggtacgatggcaacgtgtacccaaagctgttcgaggaggccctgaaggaccctctgaacgattctgtggtgccagacggctaccaggagtatctgagacccgtgctgcagcagcagctgaggaccgcaagactgtga

SEQ ID NO: 214 - DNA, 2079 bp, Arabidopsis thaliana
atggaatcaagacgcgagaagaaccctatgaccgaggaggaatctgacggactgatcgccgcacgccgcatccagcgcctgagcctgcacctgtctcccagcctgacaccatccccctctctgcctctggtgcagaccgagacatgcagcgcccggtccaagaagctggacgtgaacggcgaggccctgagcctgtacatgagaggcaagcacatcgacattcaggagaagatcttcgatttctttaactccaggccagacctgcagacccccatcgagatctctaaggacgatcaccgggagctgtgcatgaatcagctgatcggcctggtgagagaggcaggcgtgcggcctttcagatacgtggccgacgatccagagaagtattttgccatcatggaggccgtgggctctgtggatatgagcctgggcatcaagatgggcgtgcagtactctctgtggggcggcagcgtgatcaacctgggcaccaagaagcaccgcgacaagtacttcgacggcatcgataatctggactatacaggctgctttgccatgaccgagctgcaccacggctccaacgtgcagggcctgcagaccacagccacattcgatccactgaaggacgagtttgtgatcgatacccccaacgacggcgccatcaagtggtggatcggcaatgccgccgtgcacggcaagttcgccacagtgtttgcaaggctgatcctgccaacccacgactccaagggcgtgtctgatatgggcgtgcacgccttcatcgtgcctatcagagatatgaagacacaccagaccctgccaggcgtggagattcaggactgtggccacaaagtgggcctgaacggcgtggacaatggcgccctgcggttcagatctgtgaggatcccacgcgacaacctgctgaatcggtttggcgatgtgagcagagacggcacctacacaagctccctgcccacaatcaataagaggtttggagccaccctgggagagctggtgggcggcagagtgggcctggcatacgcatctgtgggcgtgctgaagatcagcgccaccatcgccatccggtatagcctgctgagacagcagttcggcccccctaagcagcccgaggtgagcattctggactatcagtcccagcagcacaagctgatgcctatgctggcctccacatacgcctatcactttgccaccgtgtacctggtggagaagtattctgagatgaagaagacccacgacgagcagctggtggcagatgtgcacgcactgagcgccggcctgaagagctacgtgacatcctataccgcaaaggccctgtccgtgtgcagggaggcatgtggaggacacggctacgcagcagtgaacaggttcggctccctgcgcaatgaccacgatatcttccagacatttgagggcgataacaccgtgctgctgcagcaggtggcagcagacctgctgaagaggtataaggagaagtttcagggcggcaccctgacagtgacctggtcctacctgcgcgagtctatgaacacatatctgtctcagcctaatccagtgaccgcaagatgggagggagaggatcacctgcgcgaccctaagttccagctggacgcctttaggtaccgcacaagcaggctgctgcagaacgtggcagcaagactgcagaagcactccaagaccctgggaggattcggagcatggaaccggtgcctgaatcacctgctgacactggccgagtctcacatcgagacagtgatcctggccaagtttatcgaggccgtgaagaattgcccagatcctagcgccaaggccgccctgaagctggcatgtgatctgtacgccctggacaggatctggaaggatatcggcacataccgcaacgtggactatgtggcccctaataaggccaaggccatccacaagctgaccgagtatctgtccttccaggtgagaaatgtggccaaggagctggtggacgcctttgagctgccagatcacgtgaccagagcccctattgctatgcagtctgatgcttattcacagtacacccaggtcgtcggattctga

SEQ ID NO: 215 - DNA, 1986 bp, Lobesia botrana
atggccgccaaggtgaccatcaacgaggatctgatcaaggagagaaacaagtgcaattttgacatcgaggagctgacatacatcatcgatggcggcaaggagaagaccctggaccggagaaatatcgagacaatcgccctgaaggaggagtatctgagaaaggataagtccgaggagtgtctgtctctgaaggagaggtacgagctggccatcgagaagtccgtgctgtatgccaccctgtttaggaaccacgtgcagacaggcgacctggatatcgaggccatcggaagctccttcctgaggaggaccgccgacgcctactttaatgaggtgagccctttcgccatccacctgggcatgtttgtgaccacaatcctgggccagggaaccccagatcagcaggccgagtggctgcacaaggccctggacctccagacagagctgggccacggctcctttatccgcggcctggagacaacagccacatatgacccagataccgagcagttcgtgctgcacacccccacactgagcgcccacaagtggtgggccggaggcctgggcaagaccgccaaccacgccatcgtgatcgcccagacctactccaagggcgagtgctatggcacacaccccttcatcgtgcagatcagggacctggatacccacgtgccactgcctggcatcagggtgggagagatcggacccaagatgggctacgtgacagccgataatggcttcctgggctttgaccacttccgcatccctcgggagaacatgctgatgaagaatgcccagatcctgaaggatggcacatacgtgaagaagtcccacaagaagatgacatatggcaccatggtgttcatcagagtgatcctggtgaacgacgccgccttcaatctggcaaaggcagtgaccatcgccgtgcgctactctgccgtgcggagacagtgtcggccaaaccccgatgcccctgagccacaggtgctggactatgtgacacagcagcacaagctgtttatcggaatcgcaacagcacacgcactccagctgaccagcgattggctgtggcgcacattcaatgccgtgcaggccgacatgcggaacggcaaggacgataatctgccagagctgcacgccctgtctagctgcctgaaggccgtgtgcacctctgatagcctggtgctgatcgagcggtgccggttcgcatgtggaggacacggctacatcctgtcctctcgcctgcccggcatctacgcctatggaacagcaatcaggacccacgagggcgactataccatcctgatgctccagtgcgccagatttctggtgaaggccttcgagctggtggaggccggaaacgccgtgtctcccacagtgagctacctgtccgagtctatcagcaaggccggatcttgggacagctccctggagggcatcatcagaagcttccagaggctggccgccggcaagatcagcgccgccgtgaagtccatcaggcgccgggtggagtccggcatgtcttacgacgatgcctggcagctgtccaccgtgcagctggtgtctgccgccgaggcacactgtcgcgccgtgatctctcacacatattggagcgagacactgcggctgaccgccacatccacccctaacgtgaagaccgtgatgcaccagctgacatctctgtacgtgacctattgggcactggagaggacaggcgatctgctgctgtacagcaagatctccgaggccgacatcatctctctccagcagagatatgaggagctgctggccctgatcaggccaaatgccgtgggcctggtggacgccttcgatatcagagacgagatcctccagagcaccctgggcgcctacgatggcctggtgtataagagactgatggacgaggccctgaagagccccctgaacgcagagcctgtgaataggtcctttcacaagtacctgaagcctttcatgcagggcaagctgtga

SEQ ID NO: 216 - DNA, 2001 bp, Lobesia botrana
atgggcgaggtcaataaggatctgcagcgcgagcgggataaatgtactttcgacgtgaaggcactgactactttcatcgacggcaccccacagaggaccctggccagaaagtaccgggagaagatcatgctgtccgagaaggagctgtctgagggcatccctgaggagtacctgagccacaaggagcggtatgagaacggcatcaagaagagcgtgatcctgttcaagctggtgcggaggctgcaagaggagggccgctctaacatcacaaattaccgggatgtgatctccggcctgctgggctcctcttccaccagagacggctcttccttcggcgtgcactatatcatgtttatgcctgccatcctgaatcagggcacagaggagcagcaggcccagtggctgccaagagcctggaactgctctatcatcggctcctatgcccagacagagctgggccacggcacattcatcagaggcctggagacaacagccacctatgacccttccacccaggagtttgtgctgcacagcccaaccctgacaagctacaagtggtggccaggcggcctgggccacaccgccaactactgcatcgtggtggcccagctgtacaccaagggccagtgccacggcatccactcctttatcatccaggtgcgcgatgaggacacccacgtgccactgcctggcatcaaggtgggcgatatcggcgccaagctgggcctgaattccgtggataacggcttcctgggcttcgatcacttcagaatccccagagaccagatgctgatgaagcacgcccaggtgctggaggatggcacctacgtgcggaatgccaggaacagcaagctgaattatggcgccatggtgtttgtgagagtggtaatcgtgtacgacatggtgaactacctgtccaaggccgtgacaatcgccaccaggttttccgccgtgagacggcagtcccagctgaaggagggcgagccagagcctcagatcctagactacgtgacccagcagcacaagatcttcatcggcatcgccgcctgccacgccctgagactgaccgccaacatgctgtgggagacattccacgacgtgacagagcagctggtgggcggcaatctggagagactgccagagctgcacgccctggcctgttgcctgaaggccgtgtccacagccgacaccgccatgtacgtggagagatgcagactggcctgtggcggccacggctacatgcagtccagcaatctgcccatgacctacggcctggtgacagccgcctgtacatatgagggcgagaacaccgtgctgctgctgcaaaccgccagatatctggtgaagacatggcagtccggcatggagagcctgacccctatggtggcctacctgaagaccgcctccgcccctgatttttgcgccagatgggagaactctgtggagggcatcgtgagaggcctgcaaaaggtggccatgggcaaggtggccagatgcgtgaagaatatcaagaagaggatggattctggcatgggcccagaggatgcctggaatgccacctctgtgcagctggtgtctgccgccgaggcccactgccgcgtggtaatcaccgccacattcttcgatgagatcaagaagctggacgcctccatgcccccagaggtgaagctggtgctgggccagctgatggagctgtacgccctgtattgggccctggagaagctgaacgatctgctgctgtatacctctatctccacccaggatgtggacgagctgcaatttcgctacgaggagctgctggccaagctgagacccaatgccgtgggcctggtggatggcttcgacatcatcgatggcatcctgcaatccaccctgggcgcctacgatggcagagtgtacgagagactgatggatgaggccctgaagtctcccctgaatgctgaaccagtgaaccagtccttccacaagtatctgaaaccttttatgcagggcaagctgtga

SEQ ID NO: 217 - DNA, 2637 bp, Lobesia botrana
atgaccgaggtgacaaaggtgaacccagacctccagagggagcgcgacaactgtaccttcaatgtgaccgagctgacaaatctgatcgatggcggcgtgcagaagacagaggagcggagaaagcgcgaggagatggtgctgaaggagggcatccacctggacgaggtgcccagcgagtacctgtcccacaaggagaagtatgagctggccgtgaagaaggcctgctacctgtttaagatgatccggcggctgcaagaggaggagaacaccggcatggagaattaccagtatgtgacatatctgcactggagctcccagtatgagagcgagtccaccgtgggcatcgagcagtctcccagcgagctgaacaatgccagcgccgccctgaattatctgaagaagctgagggcagcacgcaagaagcgggctgccgccatcgccaaggtgcacttcaagcactgtctgggcatctaccacagggaggtgctgggcggctccctgggctctgccatcctgcgggacggctcccctctgaccctgcactatgtgatgtttatcccaaccatcatgggccaggccacagtggagcagcaagtgctgtggatcggccgcgccttcaactgcgatatcatcggcacctacgcccagacagagctgggacacggcaccttcatccggggcctggagacaacagccacctacgacccctccacaaaggagttcgtgctgcactctccaaccctgacaagctataagtggtggccaggaggcctgccaaacccatctaattttgtgcctaccacaaagaacctgttcccactgaccatccacacacccggccaggtggagcctctccagccacctccactggtgagagcctttctggaggccctgtccaagttcaacgtgagcgccgacggattccccgtgtgccggatcgccctgcctatctaccgggtgagaaacgcagtggcccacaccgccaattactgtatcgtggtggcccagctgtatacacagggcaagtgccacggcatccaccctttcatcgtgcagctgcgggacgaggagacacacatgcctctgccaggcatcaaggtcggcgagatcggcgccaagctgggcatgaacggcacaaacaatggcttcctgggctttgataaggtgagaatccccagggagcacatgctgatgaagaatgccaaggtgctggaggacggaacctacgtgcgcgcaccttctagcaagctgacctatggcacaatgatgtttgtgcgggtggtgctggtgaacgacgtgtgcagctacatggccaaggccgtgaccatcgccacaagatattctgccgtgaggcaccagagccagccaaagcctgacgagccagagccacagattctggagtacgtgacccagcagcacaagctggccatcggcatcgccacagtgcacgcctttagactgtccgcctcttggctgtggaacatgtataacaatgtgaccgccgagctggacgccggcgatctggagagactgccagagatcgcagtgggaatcgcaaccgtgcacgccttcaggctgagcgcctcctggctgtggaatatgtacaacaatgtgacagccgagctggatgccggcgatctggagcgcctgcccgagatcgccgccggaattgccaccgtgcacgcctttcggctgtctgccagctggctgtggaacatgtacaataatgtgacagccgagttagacgccggcgatctggagaggctgcctgagctgcacgccctgtcctgctgtctgaaggccgtgagcaccgccgatgcatccgagtgcgtggagaggtgtcgcctggcatgcggaggacacggatacatgctgtcctctaacttccccctgatgtacggcatggtgaccgccgcctgtacatatgagggcgagaataccgtgatgcacctccagacagcccgctacctggtgaaggcatggcagcaggcacagggaggaaaccctctgaccccaacagtggcatatatcggatccatcggagccggccggagatctcccccttgggataataccgtggagggcatcatcctgggctttcagagggtggccgccggcaagatcgcacagtgcgtggccaacatcgagaagcggcagagaaccggcatgagctacgaggacgcctggaatatgacatccgtgcagctggtgtccgcctctgaggcccactgccgcgccttcatcctgaggacctactttgaggagacagagaaccagatcggaagcgtgtcccctgccctgagagccgtgctgctccagctggtggatctgtatgtggtgttttgggccctccagagagtgggcgatctgctgaggttcacctctatcagcgagagggatatcgagcagctccagagctggtacgaggatctgctgatcaagctgagagtgaacgccgtgggcctggtggacgccttcgatatcagggacgagatcctgaattccgccctgggcgcatacgacggaagggcatatgagcggctgatggccgaggccctgaagtctccactgaacgccgagcccgtgaatcagagctttcacaagtatctgaagccattcatgcagggcaagctgtga

SEQ ID NO: 218 - DNA, 1890 bp, Lobesia botrana
atggtggtgtccaagaaggtgaacgaggacctccagaaggagcgggataagtgcacattcaatgtgcaggagctgacccactacctggacggcggcgagaagatcacaaggcagcgcaaggccaccgaggacaaggtgctgtccgtgaagggcctgagagatgagatccctgaggagtacctgtctcacaaggagcggtatgagaacgccatcagaaaggccgtgatcatgcaccagaccctgggcgacctggatcagaacaatctgtctgaggacgagaaggtgaggctgggcatccgcaccacaatcagctccgccatcttcaaggataactctcctctgatgctgcactacagcatgtttacaaatgtgctgaccggacagggcgacgaggagcagcagaagtactggctgccaaaggtgaagaacatggagatcatcggcacatatgcacagaccgagctgggacacggcaccttcatcaggggcctggagaccacagccgtgtacgatcccaagaacgaggagtttgtgctgaatagccctaagctgaccgcctataagtggtggccaggaggactggcccacacagccaatcactgcgtggtcatggcccacacctactcccagggcaagtcttgtggcatccagcccttcatcgtgcagatccgcgacctggagacacacatgcctatgaacggcgtgaagctgggcgagatcggcgccaagctgggctttaataccgtgaacaatggcttcctgggctttgagaaccaccggatcccacggaatagaatgctgatgagatattcccagatcctgaaggatggcaccttcaagagcggccccaactccaagctgacatacggcaccatggtgtatgtgagggtgaccatcctgtgccacatggccaatgccctgggcaaggcagtgacaatcgcaaccagatacgccgccgtgcggagacagagccagcccaagcctaacgagccagagccccagattctggactatgtgacacagcagcacaagctgttcatctctatcgccaccagccacgccttttacctgaactgtcaccggctgtgggccctgtattccaaagtgaataaggagctggccgccggaaacctggacaatctggcagagctgcacgcactggcatgctgtctgaaggccgtgagcaccacagatggatccgtccaggtggagaggtgccgcctggcatgtggaggacacggctacatgctgtctagcaacatgccccagacctatgccaatgtgacagccgcctacacctatgagggcgagaacacagtgctgcacctccagaccgcacgggccctggtgaaggccggaacacaggtggccatgggccagaaggtgagcccttccatggagtacctggccgatagaagcaagatgaccaagtggtctaatagcgtgcagggcatcatcaagggcttccagaaggtggccgccggcaaggtggaggcatgtctggagagcctccagaggcacacaggaaacggacagtccccagaggacgcatggaatctgacctccgtgcagctggtggccgcctctgaggcccacgcacgcctggtgctgctgtccgtgtataagtctgagctggatgccaccaccttcaccctgtctggcgagaccacaaccgtgctccagcagctggtggagctgtacgcaacctattgggcactggagcggctgggcgatctgctgctgcgcgtgtgcaggcgcctgtctaaggtgtacgagtgtcccagagtgggctatgccgtgagcaacatgacacgggatagaaatttcggcgccgacgatcggagaatgacctcctcttacatccagagcgtgctggcctttctggtgtga

SEQ ID NO: 219 - DNA, 2145 bp, Lobesia botrana
atgatcggcctggagatgcaggccaacagccctgcctccagactgggcaggacctacacaccaatcaccccactgatcgccgccgccaaccgggcctgcaagaagaacctgtatacaaatcagatcctgctgaagtttcaggacaaagtgtggaagttcatgctggcccaccccctgtttaagcactccgtggccacccctacactggacgagcagagacagatcgccaccaagaggatgtatctgctgcacaacgccgatctggtgccactggaggagatcgccatgcaccccagactgttccagagctttaccgaggccatcttcatgtttgatagctccgtggccgtgaagctgtctctgacattccggatgtttaccaatacaatcaggggaagcggccggcagcaccactaccacctgatcgaggactgcgataacggcaagatcggcggctgtttcgccctgaccgagatcgcccacggcagcaatgccaagggcatgagaaccacagccacctacgacgtggagggccggtgcttcgtgatgcacacacccgattttgaggcagcaaagtgctgggtgggatccctgggcaagtgtgccacacacgccatcgtgtatgccatgctgatcagcaagggcaagaaccacggcctgcactccttcgtggtgcctatccgcgacccaaagaccctgcggccatttgccggcgtgacagtgggcgacatcggagagaagatcggcctgaacggcgtggataatggcttcctgatgtttaacaagtacagactgcccaaggaggccgccctggacaagctgggaggagtggacgagaatggcgactacaagacccccttcagagatccttctaagaggtttggagcatctctgggcatcctgagcggcggcagagtgcacatcacctccatctctacaaactacctccagaaggccgtggtcatcgccgtgaggtatagcgccgtgcggagacagttcggccctgagaatgccgtggaggagaccccagtgctggagtaccagcagcagcagatccgcctgctgccttatctggccgccacatttgccatgcgggtgttctgcaactggtttggcgaggtgcacgtgcagatgaccatcgacaatctggtgggcacagatgagcaggctgccgccagaggcatcgagatgcacgccctgtctagcgccgtgaagccagtgtgcggatggaccgcaagggatggactccagaactgtcgggagtgctgtggaggacacggatacctgagggtggtcatgctggcttgcctggcccactataatgccagaaagaaggccttcaagaggctgaagtgcatggaccgctaccggtgtagcgtgctgatgccaacctccgccatcggcgatctgaggaacgacaatgatgccaactgtacctatgagggcgagaattctgtgctgctccagcagacaagcaactggctgctgggcgtgtgggcaaggcgccggcagcctggcttcgccgacaccccatttggctccatccagtttctggacggagcacaggccctgctggatgagtcctgccagtggaactctgccgaggagatcgtgcagacagtggcctgtggcgcccacgtgcagatggcagaccacctgtacgcagaggcagatggaggacagggactgccagccgagaatcccggccaggagcagctgagaggaccagagaggctggccgtgctccagagaaggcacaccgtgcgcggactgtggcgggccctccagtttcaggacgccgcctgccgcgccgtgctgctgaagctggtggccctgtacggcgcctttctgctggagaagcacatggccaccctgtatatcggcggcttctttacaaagcagcagggactgctgctgcgggagggcgtgctgtctctgtgcagcgccctgtccccacaggccgtgtccctggcagacaccctggcaccacctgattggtgtctgcactctgtgctgggctacgcagacggagaggcctataagcacattcaggactccatcatgacctaccccggctctatgacaaggcctgagtggtggagggacgtggcacactgggagacatatgtgcctgccaagctgtga

SEQ ID NO: 220 - DNA, 2103 bp, Yarrowia lipolytica
atgaatcctaacaataccggcacaatcgagatcaacggcaaggagtataataccttcacagagccacctgtggcaatggcacaggagagggccaagacaagcttccctgtgcgcgagatgacctactttctggatggcggcgagaagaacacactgaagaatgagcagatcatggaggagatcgagcgggacccactgtttaacaacgataactactacgacctgaataaggagcagatcagggagctgaccatggagcgcgtggccaagctgagcctgttcgtgcgggatcagccagaggacgatatcaagaagagatttgccctgatcggcatcgccgacatgggcacctacacaaggctgggcgtgcactatggactgttctttggagccgtgcgcggaaccggaacagcagagcagttcggacactggatctccaagggagcaggcgatctgagaaagttctatggctgcttttccatgacagagctgggccacggctctaatctggccggcctggagaccacagccatctacgatgaggagaccgacgagtttatcatcaacacaccacacatcgccgccaccaagtggtggatcggaggagccgcccacaccgccacacacaccgtggtgttcgcaaggctgatcgtgaagggcaaggactatggcgtgaagacctttgtggtgcagctgagaaacatcaatgatcacagcctgaaggtcggcatctccatcggcgacatcggcaagaagatgggcagggatggcatcgacaacggctggatccagttcaccaatgtgaggatccccagacagaacctgctgatgaagtatacaaaggtggatagggagggaaacgtgacccagccaccactggcacagctgacatacggctctctgatcaccggcagagtgagcatggcaagcgactcccaccaagtgggcaagcggttcatcacaatcgccctgagatacgcctgcatccggagacagttttccaccacacccggccagcctgagaccaagatcatcgattacccttatcaccagaggcgcctgctgccactgctggcatacgtgtatgccctgaagatgacagccgacgaagtgggcgccctgttctctaggaccatgctgaagatggacgatctgaagcccgacgataaggccggcctgaatgaggtggtgtctgacgtgaaggagctgttctctgtgagcgccggcctgaaggcctttagcacatgggcctgcgccgatgtgatcgacaagaccaggcaggcatgtggaggacacggctactccggctataacggctttggccaggcctatgccgattgggtggtgcagtgtacatgggagggcgacaacaatatcctgaccctgagcgccggcagggccctgatccagtccgccgtggccctgaggaagggagagccagtgggcaatgccgtgtcctacctgaagcggtataaggatctggccaacgccaagctgaatggcagatctctgacagaccctaaggtgctggtggaggcatgggaggtggcagcaggcaacatcatcaatcgggccaccgatcagtacgagaagctgatcggagagggcctgaacgcagaccaggccttcgaggtgctgagccagcagaggtttcaggccgccaaggtgcacacacggagacacctgatcgccgccttcttttcccgcatcgataccgaggccggcgaagccatcaagcagccactgctgaatctggccctgctgttcgccctgtggtccatcgaggaggactctggcctgttcctgagggagggctttctggagcccaaggatatcgacacagtgaccgagctggtgaacaagtactgcaccacagtgcgcgaggaagtgatcggctacaccgatgccttcaatctgtctgactacttcatcaacgcccccatcggctgttacgatggcgacgcctaccggcactatttccagaaggtgaacgagcagaatccagcccgggatcccagacctccatactatgccagcaccctgaagcctttcctgtttagagaggaggaggacgatgacatctgtgagctggacgaggagtga

SEQ ID NO: 221 - DNA, 978 bp, Yarrowia lipolytica
atgagcgccgtgcccatcgagttcaacgtgcccagcgtggacaggcccttcggcatctacctgtgggccatcttcgaccaggcctgggagaagctgttcggctggcccgccagcagcttcatcttcgtgaggaacgaccccaacatccccttcagcagcaccccccccgtgatcatcgccatcatcgtgtactacatcgtgatcttcggcggcagggaggtgatgaggaacctgagccccatcaggctgaactggctgttccagatccacaacatcttcctgaccctgctgagcggcatgctgctgctgctgctggtggagcagctgttccccatcatcgtgaggcagggcatcctgtacgccatctgcgactacggcagctggacccagcccatcgtgttctgctactacctgaactacctgaccaagtacttcgagctgatcgacaccgtgttcctggtgctgaggaagaagaagctgaccttcctgcacacctaccaccacggcgccaccgccctgctgtgctacacccagctgatcggcaagaccagcgtgagctgggtgcccatcaccctgaacctgttcgtgcacgtggtgatgtacttctactacttcctggccgccaggggcatcagggtgtggtggaaggagtgggtgaccaggctgcagatcatccagttcgtgatcgacctgggcttcgtgtacttcgccagctacacctacttcaccagcacctactggccctggatgcccaacatgggcagctgcgccggcgaggagttcgccgccatctacggctgcggcctgctgaccagctacctgttcctgttcatcgccttctacatcaacagctacaggaagcccagcagcaagggccccagcaagcccgtggtggccgtggacggccccgtgggcggcgtgaacgcccagaccggcgccagcaggggccagaccaccaccaggagcaggagggcctga

SEQ ID NO: 222 - DNA, 915 bp, Yarrowia lipolytica
atgctgagcagcatcagccccgacctgtacagcagcttcagcttcaagaacagcctggccgaggccatgcccagcgtgccccacgagctgatcaacagcaagaccctgagctggatgtacaacgccagcctggacatcagggtgcccctgaccatcggcaccatctacgccgtgagcgtgcacctgaccaacagcagcgagaggatcaagaagaggcagcccatcgccttcgccaagaccgccctgttcaagtggctgtgcgtgctgcacaacgccggcctgtgcctgtacagcgcctggaccttcgtgggcatcctgaacgccgtgaagcacgcctaccagatcaccggcgacagcagcgcccccttcagcttcaacaccctgtggggcagcttctgcagcagggacagcctgtgggtgaccggcctgaactactacggctactggttctacctgagcaagttctacgaggtggtggacaccatgatcatcctggccaagggcaagcccagcagcatgctgcagacctaccaccacaccggcgccatgttcagcatgtgggccggcatcaggttcgccagcccccccatctggatcttcgtggtgttcaacagcctgatccacaccatcatgtacttctactacaccctgaccaccctgaagatcaaggtgcccaagatcctgaaggccagcctgaccaccgcccagatcacccagatcgtgggcggcggcatcctggccgccagccacgccttcatctactacaaggaccaccagaccgagaccgtgtgcagctgcctgaccacccagggccagttcttcgccctggccgtgaacgtgatctacctgagccccctggcctacctgttcatcgccttctggatcaggagctacctgaaggccaagagcaactga

SEQ ID NO: 223 - DNA, 1530 bp, Lobesia botrana
atggccaattctgtggccgagctgatgctgatgctgagcggcacagccggcaccgaggtgctggtggagcggctgctggtgacctgtcctgacatctccaggctgtatctgctgctgcgcgtgaagaagggcatctccccagtgcagagactgaaggagctgaagcagtctcaggtgttcgacgtggtgcggcagagatgtccccagcagctggagaagctggtggtgctggagggcgatgtggcaacacctggcctgggcctgaccgccgacgccgtgagccagctgcaagacgtgagcatcgtgttccactctgccgccaccctgaagtttgacgaggagctgggcaaggccgtggatcagaatgtgcggtccgtgatgagactgctggacatctgcgatacactgccaaatatgcaggccttcatccacgtgtctaccgcctacagcaacgcagagctgtccacagtggcagagcgggtgtatccacctccagccccactgcctgccgtgctggccctgaccgagcacgtgccctctgagacactggcccagtacacccagcagtatatcgccccaaagcccaatacctacaccttcaccaaggccctggccgagaccgccctggagcagcacggcaacaagggctatcccatcgccatcttcagacctacaatcgtgatctctgccgtgaagaacccttttccaggctgggtggagaacctgaatggaccaagcggagtggtggtggcaaccggcaagggcctgctgcacgtgttcaggacaaagtctggcgccgtggccgactttctgcctgtggacatcgccgtggataccctgatcgccgtgggctgggagacagccaccgatatgcctaaacaggtgcgggtgtacaactgctccacaggcgacaatccaacctccttcgatcactttgagcggagactgaggcaggacatcacacaccacccactggatggagcattctggtatccaagcggccggagcgtggagtctaggtgggcaagaaacgtgtacgagctggtgctccagaccatcccactgcacatcgccgagtatacagcaagggcaatcggagtgaagacaccagtgaacctgatcaccgccggcaagagactgacagccatgctggatgccctggagttctttagcacccgggagtacagattcgagaacggcaatgtgaaggccctgatgaggcgcctgagccctgaggacaagaagatctataacctggatgtgatgtccatcaattgggacgatcactacgtgaactttgtgaagggcaccaggaagtatctgctgaaggagaaggaccaggatatcgaggccgcccgcaagcacatcaataagatgtggctgctgcacaacacattcaagtttgtgctgctgctgctgttcctgagagtgatcatgcagaacaggtttgcccgcaatatcgtgtacggcaccctgcggctgctgctgagcctgttttccacagtgtatcacagactgctgcacgcctga

SEQ ID NO: 224 - DNA, 1551 bp, Lobesia botrana
atgagcatgggcgatggcaactgcctgccctcttgtgaccctagcgccagactgcccgcccctgtgcaggagatccccagaaatcacatctccacaatggccccatctatgagcgtggccgagtactatgccgataagaccctgttcatcaccggcgccacaggctttatgggcaaggtgctggtggagaagctgctgcgctgctgtcccgacatcaagagaatctacatgctgatgagggccaagaagggacagagctcctctgagcggctggagggcttcctgaagtgccgcgtgtttgatcggctgcacgaggacaaccccaagtgtttctccaagctccagctgatccctggcgatatcctggcagtggacatgggactgaccgccgaggatcgggacagactgaggaaggaggtgcagatcatcttccactgcgccgcctgcgtgcggtttgatatgcctctgcgggatgccgccgacctgaataccagaggcacacagagggtgctggatctgacagacggcatgtctcagctggaggtgtttgtgcacgtgagcaccgcctactgccgctgtgagctgccagtgctggaggagcgggtgtatgccacagcccacaagccccaggatgtgctgaagtgcgtggagtggatggacgatgagctgctgacccacctccagccaaagatgatcgacccacagcccaacacctacgcctatacaaagtccctgaccgagtctctggtggcccagtacgagggcaagttccccatcgtgatcgccagacctagcatcgtgacagccgcctttaaggagcctatgccaggctgggtggacaacctgaatgggcccaccggcctgctggtgggagccggcaagggcgtgatcagaaccatgcactgcgacgatcggctccaggccgatgtgatccctgtggacctggtggtgaatggctgcgtgctggtgggctacaagaccggcctggataagcagcctaaggagatccaggtggtgaacctgacagagtccggccgcaatccactgacctggggagccgccctggacatgggaagggtgcacgtgcaggagttccccttcagcgtgtgcctgtggtatccaggcggcagccccaagacatccctgctgctgcaccagatcgccctgctgttcacccacatcatccccgcctacctggtggatctgctgctgctggtgctgggcaagaagaccttcatggtgcggacccagaagcggatctcttatggcctggaggtgctccagtactataccacaaaggagtggcacttcaagaacgagaattttgtggccctgaggaacgtgatcagcaaggacgatgacgccatcttctacaccgacatcaagtatctgaagaatgtgatcagctcccctcacggcaagggaccatacggaggatggagactgcacccatccatcttcgtgtccccctctaacggagcagagagcgatgcatccgccgacaggcacaagggaccttttgatctggactatcacaagtactatctgtga

SEQ ID NO: 225 - DNA, 1284 bp, Lobesia botrana
atgtccgccgtgacattccagtctaggctgcacgccttcatgtttatcagccagatggcagagggatcccaggtgcaggccttctactctggcaagaacttctttatcacaggaggaaccggatttgtgggactgtgcctgatcgagaagatcctgcgcagcctgcctgacgccggcaaggcctatctgctgatgcggccaaagaagggcaagaagatcgaggagagactggaggagatcgccaagaaccccatcttcgagaccctgctccagaccaagacaccagagatctttaagaagctggtgccagtggccggcgatgtgggagagccaaatctgggcctgagcccagaggacaggcagacactggtggataacgtgaacgtggtcatccactccgccgccaccctggactttgaggataacctgaagccaacagtgagaatcaatgtgctgggcaccaggagagtggtggacctggccaaggagatcaagaacctgaagcacctgaaggccctggtgcacgtgagctccgcctacgtgaattcctatctgcacgaggcaagggagaagctgtacgacgcacccgaggatgtggagcacgtgatcgccctggtggacacactgtctgatgaggccctggcagtggtggagccaaagctgctgaagacccaccctaacacctacacattcaccaagcacctggccgagcacgaggtggccaattattctgataggctgccttgcacaatcgtgcgcccaagcatgatcgtggcagcatggaaggagccagtgcctggctggacctgttccaaagtgggccctcagggctttctgatgggagcatctaagggagtggtgaggcgcctgccactggcaaaggagaacatcgccgactatatccctgtggatgtggtggtgaatgagctgctggagaagttcatctttacagagtggaagttccacaacaccaagtctctggagctgagcgacaagctgaatgacaccgataggaagctgttttacatcgatatcggcgacatcgattggaaggccttctttgtggccctgcacctgggcgtgcggagatatctgaatagggagaaggagagcacactggaggcagcaaggggcaaggacaccatcctgctggtggcccacgtgatctggcagctgctggtcatctacctggcctggtatctgatcgcctgtctgatcggcctgacaatgacccagtccgcctggtgcgtgccagccctgtacatcctgtactgttatctgtga

SEQ ID NO: 226 - DNA, 1611 bp, Lobesia botrana
atggagctggagagctgcgtgagaatcgccgacatcgagaacgccgccatgaatccaaccgcccccattctggactatagcgaggatcacgagttcgtgagcgccaaccagctggacgattactatgccgaggaggacagggtggatgccaaggaggaggagaagcggagactgaccaagggcacagcccctagaaccgagatccagacattctataggggacagtgcgtgctgctgaccggaggaacaggcttcctgggcaaggcacactttaccacaggctacacctctggcaggctgattctggagagcatccgcaagtgggtgcactggatgcggctgccatacgccgtggtgacaatcgagaagctgctgagatcctgtggcgacatcgagtctatctatgtgctgatcaggtccaagaagggcaaggatgccaggacccgcctgaacgagatgctggacgatttcctgtttcaccgcgcccacaaggagaatcctaagggcatctacaaagtgatcccagtggtgggcgacatggagctgcccggcctgggcctgtccgaggaggatcggaagaccctggtgtctaaggtgacaatcatcctgaacgctgccgccaccgtgaagtttgatgagaagctgagcgtggccaccaacatcaatgtgagaggcacaaaggaagtgatcgccctggccaaggagtgcaagaagctgaaggccctgacccacgtgtccacagccttctctaatacccacgtgaagtgcgtggaggagaagttttatgagccccctatgtccgtggaggccctggaagccatcgccgccatcgacgagaatctgctggaggatatcacaccaaagctgctgggcaagtaccccaatacctattgcttcacaaaggccatcgcagaggaggccgtgcgcaagtacggagagggaatgcccatctgtatcatccggcctagcatcgtggtgtccacctacgaggagcctgtgcgcggatggaccgactccgtgtatgggcccaccggcctgatggtgggaatcggatgtggcgtgctgaggacaatgtatatggatgtggagaagcgggccgacatggtgcccgtggatctggtggtgaatgcactgctggcatctgcctggcacaccgcaaagaacttcagagagaatcagacaagcgagatccccatctacaacttcgtgagcggcgcccagaatcctctgaagtggggcaccttcatcgagatgaaccagaagtacggcatcgacaagccaaccacaaaggccgtgtggtactatggcctgacccctacaagctcctatccactgttcctgtttctgaatttctttctgcactttctgcccgccgccctgatcgatacctactctgccctgacaggcaagaagagagtgatgctgaagttctacaacaaagtgatgaggctggccaatatcctgttctacttcagcacccaggactggtctttcagcgatcggctggtgagaggaatgtggggcgccctggacgccgccgataaggtggtgttccctttttctgtggccgagatgagctgggactaccacgccgagacattcatcgtgtga

SEQ ID NO: 227 - DNA, 1560 bp, Lobesia botrana
atggcctcccagatgtctatcaaggagttctacaagggccggaacatcctggtgacaggcggcaccggctttatgggcaaggtgctgatcgagaagctgctgtactctatccctgagatcggcaatatctatgtggtcatgagatctaagaagggcaagagcatcaagcagcgcctggacgagatgcaccggagcccagtgttcgatagactgaagaacgagcagcctaatgcctacaagaaagtgatcccaatccagggcgacgtgctgtatgaggatctgggcatcagctccaaggacctggagatcctgagcgaggaggtgtccatcatcttccactttgcagccaccctgaggctggagagccccctgaaggattctgtgagcatgaacacagccggcaccaagaggacactgtctatcgccaagaagttccgcaatctgaccctgtttgtgcacctgagcacagccttctgctaccctgactataaggtgctggaggagaagtttcactatcccgccgccgaccctcacgatgtgatgaggctgtgcgagtggctggatgagaagcagctggccctgctgacaccatccctgctggtgaaccaccccaatacctacacatataccaagaggctggccgagtccctggtgaacgacgccttccctgagctgccatgcgtgatcgcaaggccttctatcgtgtgcccagcacacgcagacccagtgcctggctgggtggataacctgaatggaccagtgggcgtgatgctgggagccggcaagggcgtgatccggaccatgctgtgcgacggcaccctccaggcccaggtcatccccgtggatgtggccatcaacggcctgatcgccctgggcatgatcgagggcagcaagaccacaaagccagaagtgatccccatctacaacctgaatatcggacaccagcagcctaccagctggggagaggtgctgaatatcgccaaggactatggccggaagttccctctgtcctggccactgtggtaccccaacggcgatatcaccacaaattatgccctgcacgagaccaagagaatcctgtttcacctggtgcccgcctacttcatcgactttctgctgctgatcacaggccagaagcggttcatgctgagagtgcaggataggatctcccagggcctggagctgctccagtacttcaccatgaggccctggtccttcccatgccccaactttgacgccatctgtcaggtgctgtctaaggaggacgatctggtgtaccctacaaatgcccgcgagttcgataaggtggagtatctgatcaagtctgtggagggcggccgggtgttctgcctgaaggaggacccaaccaagtttaagatcaacagaacataccacaatttcctgtttgtgctggatctgctggtgaagatcgtgttctggtgttttatcctgagcttctttgccgcctggtttgagcccgtgcgggatctgctgtccctgggcgagcccgtggtgaagcatctgcccttcctgggcaaggccgtgtttgagaccaattga

SEQ ID NO: 228 - DNA, 644 bp, Lobesia botrana
ccccacctcccctcatttttttttttttattctcgccctcactttcttttataatcttttatatattattaaattaaccttcattttttttttttttttttgttttttttttttttttttttttttttttttttttttttttttttttttttttttccttagaatacgactttatttagaccattctcctgagcagctcgttgatcttgtcctcccggttgccgaagtcaccgccgtcgacgtaatggatggtcttcttgcgccagccgccggtggggttgttcagcttgaagggccagaggaagttgctggcgtacttgaacttgtctccgacggtgaagatctcgtggatgaggtcttcgacgcagatgatgtcgttgttcttgccgagcttgtcctcgatgatcttgttgctggtgatgggcacgcgctgcccgttgatcttggcgaacccgcgcttgtagatcagctctcgcacgctcttcaggttggggtatccccaggtgatgtagggctccgcgatgcgcatcatgttgacggtggccttgttgagccgcacgaacacgccgttgttgatctggcgcagtctgaacagctgcagcactttacggaccttgggagagacctggttgataccactgctt

(19) United States
(12) Patent Application Publication
(10) Pub. No.: US 2023/0031596 A1
(43) Pub. Date: Feb. 2, 2023

(54) BIOSYNTHESIS OF INSECT PHEROMONES AND PRECURSORS THEREOF

(71) Applicant: Provivi, Inc., Santa Monica, CA (US)

(72) Inventors: Weslee Glenn, Santa Monica, CA (US); Micah Sheppard, Santa Monica, CA (US); Kim Nguyen, Santa Monica, CA (US); Kati Wu, Santa Monica, CA (US); Toni Lee, Santa Monica, CA (US); Thomas Heel, Santa Monica, CA (US); David Rozzell, Santa Monica, CA (US); Effendi Leonard, Santa Monica, CA (US)

(21) Appl. No.: 17/785,026

(22) PCT Filed: Dec. 11, 2020
(86) PCT No.: PCT/US2020/064702

§ 371 Date: Jun. 13, 2022

Related U.S. Application Data
(60) Provisional application No. 62/946,967, filed on Dec. 11, 2019.

(51) Int. Cl. C12N 1/16; C12N 15/81; C12N 9/02

(52) U.S. Cl. CPC C12N 1/16; C12N 15/815; C12N 9/0071; C12Y 103/03006

(57) ABSTRACT

This disclosure concerns the metabolic engineering of microorganisms to provide biosynthetic methods for the production of insect pheromones and precursors thereof in a scalable and eco-friendly fermentation reaction; for example, by converting saturated or unsaturated substrate feedstocks utilizing exogenous metabolic machinery.

### CROSS-REFERENCE TO RELATED APPLICATIONS

This application is a national phase entry under 35 U.S.C. § 371 of International Patent Application PCT/US2020/064702, filed Dec. 11, 2020, designating the United States of America and published in English as International Patent Publication WO 2021/119548 A1 on Jun. 17, 2021, which claims the benefit of U.S. Patent Application Ser. No. 62/946,967, filed Dec. 11, 2019, the disclosure of which is hereby incorporated herein in its entirety by this reference.

### STATEMENT ACCORDING TO 37 C.F.R § 1.821(c) OR (e)—SEQUENCE LISTING SUBMITTED AS A TXT FILE

Pursuant to 37 C.F.R. § 1.821(c) or (e), files containing a TXT version of the Sequence Listing has been submitted, titled 22-06-13_SeqList_ST25.txt, created December 11, 202 and 525 kb in size, the contents of which are hereby incorporated by reference.

### FIELD OF THE DISCLOSURE

The present disclosure relates to the production of insect pheromones and precursors thereof, which pheromones may be useful, for example, as effective crop protective agents. More specifically, the disclosure relates to metabolic engineering of microbes to synthesize insect pheromones; for example, from saturated or unsaturated substrate. In particular embodiments, engineered microorganisms produce (E,Z)-7,9-dodecadienyl-CoA (E7Z9-12CoA) and/or (E,Z)-7,9-dodecadienyl acetate (E7Z9-12Ac).

### BACKGROUND

Insect sex pheromones are a diverse group of chemical compounds that are central to mate-finding behavior in insects, and they are very promising for the eco-friendly protection of a wide range of crops. Contrary to classical pesticides, pheromones are specific to one species of pest; other insects, and especially pollinator insects, are unaffected. Furthermore, pheromones are biodegradable and have no known effect upon human health. Those properties make pheromones ideal candidates for modern eco-friendly crop protection.

Lobesia botrana (the European grapevine moth) is an agricultural pest whose larvae feed on the fruits and flowers of Vitis vinifera (wine grape), Rubus fruticosus L (European blackberry), and other economically important crops. Damage renders the fruit unmarketable and increases the likelihood of fungal infection on the plant and its neighbors. While L. botrana is native to Europe, the pest has spread to other locales including the Napa Valley Region in California (first reported in 2009), a state whose wine sales hit $35.2 billion in 2017. To date, several registered insecticides target tortrix larvae including growth regulators, spinosyns (which inhibit nicotinic acetylcholine receptors) and Bacillus thuringiensis (Bt).

The L. botrana sex pheromone (i.e., E7Z9-12Ac) is one of the four geometric isomers of 7,9-dodecadienyl acetate: (E,Z)-7,9-dodecadienyl acetate, (E,E)-7,9-dodecadienyl acetate, (Z,Z)-7,9-dodecadienyl acetate, and (Z,E)-7,9-dodecadienyl acetate. While its usefulness in insect control is known, existing strategies for its production are hampered by lengthy pathways and multiple downstream unit operations with moderate yields. Cahiez et al. (2017) Org. Process Res. Dev. 21:1542-8; European Patent Publication EP 0241335. A one-pot synthetic strategy for E7Z9-12Ac synthesis including iron-catalyzed cross-coupling between a Grignard reagent and a dienol phosphate, followed by acylation, has been developed (Cahiez et al. (2017), supra), but this process is limited by its scalability, and it requires the use and consumption of organic solvents. The foregoing problems in E7Z9-12Ac production limit the deployment of this compound in insect management strategies.

### BRIEF SUMMARY OF THE DISCLOSURE

Described herein is the metabolic engineering of microorganisms (e.g., yeast) that synthesize precursors of the L. botrana pheromone, E7Z9-12Ac (e.g, E7Z9-12CoA and E9Z11-14CoA) from saturated or unsaturated substrates in a fermentation reaction in a regioselective manner, through the introduction of exogenous components of a E7Z9-12CoA biosynthetic pathway. In embodiments, engineered microorganisms herein contain one or more exogenous desaturases, acyl-CoA oxidases, fatty acyl-CoA reductases, enoyl-CoA hydratases, 3-hydroxyacyl-CoA dehydrogenases, conjugases, elongases, thiolases, and/or beta-oxidation enzymes (e.g., of heterologous origin), which may be modified in some examples to provide desired stereoselectivity, regioselectivity, or chain length selectivity. In particular embodiments, an engineered microorganism comprises one or more of a Z11-14 desaturase (e.g., DST299), a Z11-16 desaturase (e.g., DST499), an E9-14 desaturase (e.g., a DST014, a DST024, a DST176, a DST177, a DST178, a DST192, and a DST043), and an E11-16 desaturase (e.g., DST109 V230A). In particular embodiments, an engineered microorganism comprises one or more fatty acyl-CoA oxidase (POX) enzyme (e.g., a RnACOX2, an AtACX1, an AtACX2, a LbPOX1-5, an BnACX3, a PxACX1, and a PxACX3). In particular embodiments, an engineered microorganism comprises one or more conjugase enzyme (for example, an SPTQ (SEQ ID NO:78) motif-containing enzyme (e.g., DST499 and DST500)). Insect pheromones produced from precursors according to the present disclosure may be used to disrupt mating of key agricultural pests, thereby providing significant crop protection.

Embodiments herein include at least one component of a E7Z9-12CoA biosynthetic pathway selected from the group of desaturases consisting of DST499, DST500, DST299, DST109, DST014, DST109 V230A, KPAE, RPTQ2, KPSE1, NPVE, LPGQ, and RAVE; conjugases (e.g., DST500); and acyl-CoA oxidase (POX) enzymes consisting of RnACOX1, RnACOX2, AtACX1, AtACX2, LbPOX1, LbPOX2, LbPOX3, LbPOX4, LbPOX5, LbPOX6, BaACX3, PxACX1, and PxACX3.

As will be understood from the present disclosure (for example, with reference to FIGS. 1-4), these components can be comprised in different combinations in a microorganism to obtain useful pheromones or their precursors. Particular embodiments include at least one desaturase or conjugase and at least one POX. Specific embodiments include DST299 and at least one POX; for example, a combination including DST299, DST500, and at least one of LbPOX5, BaACX3, PxACX1, and PxACX3, a combination including DST500 and at least one of LbPOX5, BaACX3, PxACX1, and PxACX3, a combination including DST014, DST299, and at least one of LbPOX5, BaACX3, PxACX1, and PxACX3, a combination including DST299, DST500, and at least one of LbPOX5, BaACX3, PxACX1, and PxACX3, a combination including DST299, DST109 V230A, and one of LbPOX5, BaACX3, PxACX1, and PxACX3, a combination including DST500 and at least one of LbPOX5, BaACX3, PxACX1, and PxACX3, and a combination including DST499 and at least one of LbPOX5, BaACX3, PxACX1, and PxACX3. In a cell-free recombinant production system (for example and without limitation, a bioreactor or reaction volume), the foregoing components may be added in an ordered fashion, for example, to increase reaction specificity and product yield. In one example, a E7Z9-12CoA may be produced from a 14C substrate utilizing DST299 and DST500, where DST299 may be introduced into the reaction before DST500, or alternatively DST500 may be introduced into the reaction before DST299.

Some embodiments herein include methods for engineering a desaturase with modified substrate selectivity. For example, such methods may include engineering the desaturase sequence of SEQ ID NO:14 to provide a functional desaturase sequence comprising one or more amino acid variants at an amino acid position selected from the group consisting of amino acids 71, 75, 78, 111, 151, 157, 224, 225, 226, 254, 74-82, 224-233, 250-259, and 265-274, such that the functional desaturase sequence is not SEQ ID NO:5 or SEQ ID NO:7. In particular embodiments, the functional desaturase sequence is SEQ ID NO:16. In further embodiments, the functional desaturase sequence is selected from the group consisting of SEQ ID NOs:18-20.

Some embodiments herein include a genetically modified microorganism. In particular embodiments, such a genetically modified microorganism may comprise at least one component of a E7Z9-12CoA biosynthetic pathway selected from the group consisting of DST299, DST109, DST014, DST499, DST500, LbPOX5, BaACX3, PxACX1, and PxACX3. A genetically modified microorganism according to embodiments herein may be a yeast or bacterium, for example, which is suitable for scalable culture. In particular embodiments, the microorganism is yeast (for example, Yarrowia lipolytica (e.g., Y. lipolytica strain H222 (Clib80))). Some embodiments include a culture of the genetically modified microorganisms herein.

Some embodiments herein include biosynthetic methods for producing an insect pheromone or precursor thereof. Such methods may comprise, for example, culturing a genetically modified microorganism as herein described, and feeding the culture with a saturated or unsaturated substrate. In particular embodiments, the method may further comprise isolating an insect pheromone or precursor thereof produced from the substrate. For example, the method may further comprise isolating E7Z9-12CoA from the culture. In other embodiments, a method for producing an insect pheromone or precursor thereof does not utilize significant amounts of organic solvents, proceeds in one step, and results in high yield of a particular product isomer, providing a significant improvement upon conventional production methods.

Also described herein are means for producing an insect pheromone or precursor thereof in a microorganism, as well as genetically modified microorganisms (e.g., yeast such as Y. lipolytica) comprising means for producing an insect pheromone or precursor thereof in a microorganism; cultures of genetically modified microorganisms comprising means for producing an insect pheromone or precursor thereof in a microorganism; and methods for producing an insect pheromone or precursor thereof comprising culturing a genetically modified microorganism and comprising means for producing an insect pheromone or precursor thereof in a microorganism, and feeding the culture with a saturated or unsaturated substrate. Means for producing an insect pheromone or precursor thereof in a microorganism include, inter alia, the polypeptides of SEQ ID NO:1, SEQ ID NO:2, SEQ ID NO:111, SEQ ID NO:6, SEQ ID NO:113, SEQ ID NO:114, SEQ ID NO:115, and SEQ ID NO:116.

The embodiments of the present invention also can include genetically modified microorganisms that comprise at least one heterologous component of a E7Z9-12CoA biosynthetic pathway selected from the group consisting of:

A) a heterologous Z11-14 desaturase that converts a C14 substrate to Z11-14CoA or E9Z11-14CoA, preferably wherein the genetically modified microorganism comprises C14 and/or C16 fatty-acyl CoA oxidase activity;

B) a heterologous Z11-16 desaturase that converts a C16 substrate to Z11-16CoA, r comprises a heterologous E11-16 desaturase that converts a C16 substrate to E11-16CoA, preferably wherein the genetically modified microorganism comprises C14 and/or C16 fatty-acyl CoA oxidase activity;

C) a heterologous conjugase that converts a C14 substrate to E8E10-14CoA or E9Z11-14CoA and converts a C16 substrate to E11Z13-16CoA, preferably wherein the genetically modified microorganism comprises C14 and/or C16 fatty-acyl CoA oxidase activity; and/or

D) a heterologous fatty acyl-CoA oxidase that converts E9Z11-14CoA to E7Z9-12CoA and converts E11Z13-16CoA to E9Z11-14CoA,

optionally wherein the genetically modified microorganism further comprises a heterologous E9-14 desaturase that converts a C14 substrate to E9-14CoA or E9Z11-14CoA, more preferably wherein the C14 substrate is 14CoA or Z11-14CoA,

optionally wherein the genetically modified microorganism comprises at least one further heterologous polypeptide with Z11-14 desaturase activity, Z13-18 desaturase activity, Z11-16 desaturase activity, Z11-18 desaturase activity, Z9-18 desaturase activity, Z13-16 desaturase activity, Z9-16 desaturase activity, Z9-14 desaturase activity, E11-14 desaturase activity, or E11-16 desaturase activity.

The foregoing and other features will become more apparent from the following detailed description of several embodiments, which proceeds with reference to the accompanying figures.

### BRIEF DESCRIPTION OF THE FIGURES

FIG. 1 includes diagrams of alternative biochemical pathways and identifies tools useful to generate E7Z9-12CoA from unsaturated and saturated substrates, according to particular embodiments of the disclosure. In FIG. 1A, pathway entry points are indicated with either a triangle (saturated substrate) or circle (unsaturated substrate). Substrates can be fed as either acids or alkyl esters to engineered microbes at these points along the pathway to E7Z9-12CoA. The diagrams illustrated in FIG. 1B show examples of pathways for the production of E7Z9-12CoA from a 14C substrate (2 upper diagrams) or 16C substrate (lower 2 diagrams) utilizing a class of enzymes described herein with a characteristic SPTQ (SEQ ID NO:78) motif, DST499 and DST500, that exhibit a broad spectrum of desaturase and conjugase activities. In the 2 upper diagrams, E8E10-14CoA can be produced by conjugation of 14CoA by DST500, which can them be isomerized to E9Z11-14CoA for POX oxidation to E7Z9-12CoA (top diagram), or Z11-14CoA can be directly desaturated by DST500 to form E9Z11-14CoA, which can then be converted to E7Z9-12CoA by POX (second diagram from top). Z11-14CoA can be directly added to a reaction or culture, or can be produced from a Z11-14 desaturase, such as DST299. In the two lower diagrams, Z11-16CoA can be produced by desaturation of 16CoA by DST499, followed by oxidation of Z11-16CoA by POX; the resulting Z9-14CoA can then be desaturated to produce E9Z11-14CoA for conversion by POX to E7Z9-12CoA (second diagram from bottom). Alternatively, Z13-16CoA can be conjugated by DST500 to E11Z13-16CoA, which can be converted in two steps by POX to form E7Z9-12CoA (bottom diagram). Z13-16CoA can be directly added to a reaction or culture, or can be produced by elongase (e.g., ELO1 or ELO2) activity from Z11-14CoA. FIG. 1C includes a table showing particular polypeptides utilized in certain embodiments herein with their classification and particular enzymatic activities. FIG. 1D includes a table showing representative enzymes utilized to catalyze particular C14 substrate reaction steps of certain embodiments herein. Similarly, FIG. 1E includes a table showing representative enzymes utilized to catalyze particular C16 substrate reaction steps of certain embodiments herein.

FIG. 2 includes a diagram of the conversion of E7Z9-12CoA to E7Z9-12Ac, according to some embodiments of the disclosure.

FIG. 3 includes diagrams of the conversions of E9-14CoA or Z11-14CoA to E7Z9-12CoA, using a Z11-14 DST or an E9-14 DST, and a POX (FIG. 3A), and pathways from a 14CoA substrate to E7Z9-12CoA (FIG. 3(B-E)) utilizing these conversions according to embodiments herein. FIG. 3B shows alternative pathways to E7Z9-12CoA from 14CoA using an E9-14 DST, a Z11-14 DST, or a conjugase. The initial reaction steps proceed through different intermediates; E9-14CoA for the E9-14 DST, Z11-14CoA for the Z11-14 DST, and E8E10-14CoA for the conjugase. The E9-14CoA intermediate is then desaturated to E9Z11-14CoA by Z11-14 DST (top diagram). The Z11-14CoA intermediate may either be desaturated by the E9-14DST or converted to Z13-16CoA by an elongase (middle diagram). The Z13-16CoA produced by the elongase is then converted to E11Z13-16CoA by an E11-16 DST or conjugase, and subsequently oxidized by POX to form E9Z11-14CoA. The E8E10-14CoA intermediate formed in the conjugase pathway is isomerized to E9Z11-14CoA (bottom diagram). In each of the pathways, the final step is POX oxidation of E9Z11-14CoA to the E7Z9-12CoA product. In FIG. 3C, a representative example is shown of the pathway utilizing an initial E9-14 DST (e.g., DST014), where the Z11-14 DST (e.g., DST299) produces E9Z11-14CoA from the E9-14CoA intermediate. In FIG. 3D, a representative example is shown of the pathway utilizing an initial Z11-14 DST (e.g., DST299), where the conjugase (e.g., DST500) produces E9Z11-14CoA from the E9-14CoA intermediate. FIG. 3E shows a representative example of a pathway through the E8E10-14CoA intermediate, utilizing DST500 to produce E8E10-14CoA from 14CoA, and subsequently producing E9Z11-14CoA (utilizing DST299 in the example shown). In each of the pathways illustrated in FIGS. 3(C-E), E9Z11-14CoA is converted to E7Z9-12CoA by a POX (e.g., LbPOX5, BaACX3, PxACX1, and PxACX3) catalyzes oxidation of E9Z11-14CoA to E7Z9-12CoA.

FIG. 4 includes diagrams of pathways from a 16C substrate to E7Z9-12CoA. FIG. 4A shows alternative pathways from 16CoA to E7Z9-12CoA through an E9Z11-14CoA intermediate. As shown in the top diagram, a two-step process including desaturation of 16CoA by a Z11-16 DST produces a E11Z13-16CoA intermediate, which is then oxidized to E9Z11-14CoA by POX, and again by POX to E7Z9-12CoA. As shown in the middle diagram, desaturation of 16CoA by an E11-16 DST produces an E11-16CoA intermediate, which is then oxidized by POX to yield E9-14CoA. E9-14CoA is in turn desaturated by a Z11-14 DST to produce E9Z11-14CoA, which is oxidized by POX to E7Z9-12CoA. As shown in the bottom diagram, desaturation of 16CoA by a Z13-16 DST produces a Z13-16CoA intermediate, which is then converted by a conjugase to an E11Z13-16CoA intermediate. Alternatively, Z13-16CoA can be obtained from jojoba oil. E11Z13-16CoA is converted by POX to produce E9Z11-14CoA, which is oxidized by POX to E7Z9-12CoA. FIG. 4B includes a diagram of a pathway from Z13-16CoA, which can be obtained from jojoba oil, to E7Z9-12CoA.

FIG. 5 includes the results of a gas chromatography (“GC”) assay detecting E9Z11-14CoA production in a microbial strain. E9-14Acid is fed to a strain harboring a Z11-14 DST (DST299). The strain's endogenous machinery converts E9-14Acid to E9-14CoA, which can be desaturated by heterologous Z11-14 DST activity to form E9Z11-14CoA. The red chromatogram corresponds to an authentic standard of E9Z11-14ME. The blue chromatogram is a no substrate control (i.e., no E9-14Acid was fed to the microbe). Finally, the black chromatogram corresponds to E9-14Acid being fed to a strain with heterologous Z11-14 DST activity. The black chromatogram shows the production of E9Z11-14CoA (red box). Notably, downstream sample processing reduces all fatty CoA analogs within the cell to the corresponding methyl esters (“MEs”); e.g., E9Z11-14ME.

FIG. 6 includes the results of a spiked GC assay confirming the presence of E9Z11-14CoA. E9-14Acid was fed to a strain harboring Z11-14 DST activity, thereby producing E9Z11-14CoA. Downstream sample work-up (i.e., reduction) renders the E9Z11-14ME form. An authentic standard was spiked into the sample to confirm the presence of E9Z11-14ME. A sample overlay was prepared to show that the peak corresponding to E9Z11-14ME (red chromatogram) grows in intensity upon sample spiking (black chromatogram). Arrows point to the E9Z11-14ME peak (red arrow) and its putative isomer (green arrow).

FIG. 7 includes the mass fragmentation of 4-methyl-1,2,4-triazoline-3,5-dione (“MTAD”)-derivatized E9Z11-14ME (from biological sample also used in the GC-flame ionization detection analysis in FIG. 5), confirming the conjugated diene located at carbons 9 and 11.

FIG. 8 includes mass fragmentation patterns from several POX variants (SPV745 (POX2; blue), ACX1 (red) and AtACX2 (black)) that can chain shorten E9Z11-14CoA to E7Z9-12CoA. Sample processing reduces CoA analogs to the ME form. A negative control (SPV459; purple) lacking POX activity is shown for comparison; here no E9Z11-14CoA is chain shortened to E7Z9-12CoA (red rectangle).

FIG. 9 includes the mass fragmentation of E7Z9-12ME (from biological sample shown in FIG. 8). The molecular ion [M]⁺ and the [M-31]⁺ (corresponding to loss of a methoxy group) peaks are labeled.

FIG. 10 includes the mass fragmentation of MTAD-derivatized E7Z9-12ME (from biological sample shown in FIG. 8). The mass fragmentation pattern of the MTAD adducts confirms the conjugated diene located at carbons 7 and 9.

FIG. 11(A-B) includes a representation of DST014 desaturase structure. Conserved amino acid patterns across all groups were identified and highlighted in yellow, and residues likely essential for catalytic activity were highlighted in orange, while the substrate was highlighted in cyan. FIG. 11(A). FIG. 11(B) includes identification of residues conserved among distinct DST groups that putatively guide stereo-, regio-, or chain-length selectivity, plotted on the homology model and highlighted in purple.

FIG. 12 includes a sequence alignment of three DSTs; DST014 is a specific E11-14 DST. DST101 is a Z11-16/14 DST, and DST109 is a Z11-16 DST. Red asterisks mark distinct residues that may govern product profile.

FIG. 13 includes a representation of DST109 homology model, with an alanine scanning heatmap of the enzyme's binding pocket. Alanine scanning mutagenesis was performed on regions 1-4. Light Grey=neutral mutation (Z11-16 levels=WT levels) or already alanine in WT; Blue=Mutation moderately disrupts enzyme (Z11-16 levels >50% of WT levels); Orange=mutation severely disrupts activity (Z11-16 levels 50-200 mg/L); Red=Z11-16 levels (0-50 mg/L)—completely abolish activity. The substrate (18CoA) is shown in light blue and rendered in space filling mode. The two grey spheres are the putative diiron binding site (notably, the crystal structure model finds Zn²⁺ ions bound here).

FIG. 14 includes the results of a GC-FID assay detecting E7Z9-12FAME production in a microbial strain. The chromatograms show increased levels of E7Z9-12FAME (yellow highlight) in the Test Strain (blue trace) (SPV2554, SPV2555 SPV2557, SPV483 and SPV2484; DST299+RnACOX2; [H222 ΔP ΔΔ ΔF, Δxpr2::pTEF-(SEQ ID NO:133)-tXPR2, Δfaol::pTEF-(SEQ ID NO:133)-tXPR2, Δtg13::pTEF-(SEQ ID NO:133)-tXPR2, Δfat1::pTEF-(SEQ ID NO:133)-tXPR2, pox5::pTEF-(SEQ ID NO:133)-tXPR2-URA3]) relative to the negative control (black trace) (SPV1904; DST299; [H222 ΔP ΔΔ ΔF, Δxpr2::pTEF-(SEQ ID NO:133)-tXPR2, Δfaol::pTEF-(SEQ ID NO:133)-tXPR2, Δtg13::pTEF-(SEQ ID NO:133)-tXPR2, Δfat1::pTEF-(SEQ ID NO: 133)-tXPR2-URA3]).

FIG. 15 includes a bar graph showing the production of five select individual analytes (E7Z9-12FAME, maroon; E7E9-12FAME, olive; E9Z11-14FAME, blue; E9E11-14FAME, pink; and E11Z13-16FAME, gold), according to particular embodiments of the disclosure. This chart shows that the Test Strain accumulates E7Z9-12FAME at titers about 5-fold higher than the control. An as-yet unidentified enzyme may be capable of chain shortening E9Z11-14 (blue) to E7Z9-12 (maroon) at low levels in the negative (−) control strain, which lacks RnACOX1 and RnACOX2. The Test Strain also accumulates the isomer E7E9-12 (olive) at higher titers than background, which suggests that the POX can also chain shorten E9E11-14 (pink; side product of DST299 when E9-14 is fed). E9Z11-14 (blue; the product of DST299 when E9-14 is fed) may also be elongated to E11Z13-16 (gold), though notably E11Z13-16 titers are higher when either of RnACOX1 and RnACOX2 is not present.

FIG. 16 includes the mass fragmentation of E7Z9-12ME (from biological sample shown in FIG. 14). The molecular ion [M]⁺ and the [M-31]⁺ (corresponding to loss of a methoxy group) peaks are labeled.

FIG. 17 includes the mass fragmentation of MTAD-derivatized E7Z9-12ME (from biological sample shown in FIG. 14). The mass fragmentation pattern of the MTAD adducts confirms the conjugated diene located at carbons 7 and 9.

FIG. 18 includes GC-MS fragmentation data DMDS evidence of E9-14FAME production. Dimethyl disulfide adducts of D9-14-producing desaturases are observed in data from recombinant yeast expressing DST192 G100L.

FIG. 19 includes a bar graph showing E9-14 titers in recombinant yeast expressing DST024, DST177, or DST178.

FIG. 20 includes bar graphs showing the titers of Z9-14, Z9-16, Z9-18, Z11-16, and Z11-18 produced in recombinant yeast expressing a variety of desaturases from saturated fatty acids.

FIG. 20A shows titers of Z9-14, Z9-16, and Z9-18 produced from methyl laurate (12ME), methyl myristate (14ME), and methyl palmitate (16ME) feeds in recombinant yeast expressing DST043, DST162, DST163, DST165, and DST166. FIG. 20B shows titers of Z9-14, Z9-16, Z9-18, and Z11-18, also produced from methyl laurate (12ME), methyl myristate (14ME), and methyl palmitate (16ME) feeds, by recombinant yeast expressing DST043, DST076, DST077, DST167, DST168, DST169, DST170, DST171, DST172, DST175, DST179, DST180, DST181, DST183, DST184, DST185, DST186, DST191, DST192 100G, and DST219.

FIG. 21 includes a representation of DST109 homology model with 14CoA bound in the substrate binding site. Mutating position E283 results in E9-14 DST activity, converting 14C substrates to E9-14C, such as 14CoA to E9-14CoA.

FIG. 22 includes a bar graph showing E11Z13-16 production from Z13-16 in Y. lipolytica expressing DST500. Data represent titers of E11Z13-16CoA produced from a Z13-16Acid feed.

FIG. 23 includes bar graphs illustrating a complete oxidation pathway from unsaturated C16 substrates catalyzed by the acyl-CoA oxidases herein. FIG. 23A shows E7Z9-12 production from E11Z13-16 through E9Z11-14 in Y. lipolytica expressing LbPOX5, RnACOX2, BaACX3, PxACX3, and PxACX3. Not shown are the data showing E7Z9-12 obtained by expression of Arabadopsis thaliana ACX1 (AtACX1) and A. thaliana ACX2 (AtACX2). Data represent titers of E5Z7-10, E9Z11-14, and E7Z9-12 produced from a E11Z13-16 feed. FIG. 23B shows E9-14 production from E11-16 in Y. lipolytica expressing LbPOX5, BaACX3, and PxACX3. Data represent titers of E9Z-14 produced from an E11-16 feed.

FIG. 24 includes a bar graph showing Z11-14 production from C14 in Y. lipolytica expressing DST299.

FIG. 25 includes a bar graph showing E9Z11-14 production from a Z11-14 feed in Y. lipolytica expressing DST500 from one or two gene copies.

FIG. 26 includes a bar graph showing E8E10-14 production from a C14 feed in Y. lipolytica expressing DST500.

FIG. 27 includes a bar graph showing Z11-14CoA production from 14ME in Y. lipolytica expressing DST299.

FIG. 28 includes a bar graph showing elongase catalyzed production of Z13-16 from Z11-14. Data are Z13-16CoA titers (mg/L) in Y. lipolytica expressing ELO1, ELO2, and DST299 fed 14ME substrate.

FIG. 29 includes a bar graph showing Z11-16 production from C16 in Y. lipolytica expressing DST499. Z11-16Acid was converted to cognate methyl ester (ME) and quantified.

### SEQUENCE LISTING

The nucleotide sequences listed in the accompanying Sequence Listing are shown using standard letter abbreviations for amino acids and nucleotide bases, as defined in WIPO Standard ST.25. Only one strand of each nucleotide sequence is shown, but the complementary strand and reverse complementary strands are understood to be included by any reference to the displayed strand. When a sequence allows for alternatives at a specific position (e.g., an amino acid is Xaa, Asx, or Glx, and a nucleotide is r, y, m, k, s, w, b, d, h, v, or n), all alternatives are specifically disclosed by the generic sequence (e.g., “agmc” specifically means that both “agac” and “agcc” are included by reference to the sequence, both together and separately individually). In the accompanying Sequence Listing:

SEQ ID NO:1 shows an exemplary amino acid sequence of the Z11-14 fatty acid desaturase referred to herein as a DST299.

SEQ ID NO:2 shows an exemplary amino acid sequence of the Z11-16 fatty acid desaturase referred to herein as a DST499.

SEQ ID NO:3 shows an exemplary amino acid sequence of a desaturase with Z9-14 activity, referred to herein as a DST192.

SEQ ID NO:4 shows an exemplary amino acid sequence of the E9-14 fatty acid desaturase referred to herein as DST192 G100L.

SEQ ID NO:5 shows an exemplary amino acid sequence of the Z11-16 fatty acid desaturase referred to herein as a DST109.

SEQ ID NO:6 shows an exemplary amino acid sequence of the E11-16 fatty acid desaturase referred to herein as DST109 V230A.

SEQ ID NOs:7-12 shows exemplary amino acid sequences of the E9-14 fatty acid desaturases referred to herein as a DST014, a DST024, a DST176, a DST177, a DST178, and a DST043, respectively.

SEQ ID NO:13 shows an exemplary amino acid sequence of the Z11-16 and Z11-14 fatty acid desaturase referred to herein as DST101.

SEQ ID NOs:14-27 show exemplary desaturase variants that are engineered to provide different regioselective and stereoselective activities, such as the E11-16 DST or E9-14 DST activity demonstrated herein for desaturases described as SEQ ID NOs:18-20.

SEQ ID NOs:28-49 show exemplary amino acid sequence of fatty acid desaturases with stereoselectivity that is engineered herein, which desaturases are referred to herein as a DST162, a DST163, a DST165, a DST166, a DST076, a DST077, a DST167, a DST168, a DST169, a DST170, a DST171, a DST172, a DST175, a DST179, a DST180, a DST181, a DST183, a DST184, a DST185, a DST186, a DST191, and a DST219.

SEQ ID NOs:50-53 show amino acid sequences of polypeptides containing a fatty acid desaturase domain identified from a L. botrana cDNA library.

SEQ ID NOs:54-76 show further desaturases that may be utilized in specific examples.

SEQ ID NO:77 shows a novel characteristic desaturase motif, PPTQ.

SEQ ID NO:78 shows the characteristic desaturase/conjugase motif, SPTQ.

SEQ ID NOs:79-88 show desaturase regioselectivity determinants.

SEQ ID NOs:89-100 show amino acid sequences of exemplary desaturase domains lining the substrate binding pocket.

SEQ ID NOs:101-110 show candidate desaturase sequences containing a fatty acid desaturase domain identified from a L. botrana cDNA library for which negative results were obtained.

SEQ ID NO:111 shows an exemplary amino acid sequence of the conjugase referred to herein as a DST500.

SEQ ID NO:112 shows an exemplary amino acid sequence of the fatty acyl-CoA oxidase referred to herein as a LbPOX5.

SEQ ID NO:113 shows an exemplary amino acid sequence of the fatty acyl-CoA oxidase referred to herein as a BaACX3.

SEQ ID NO:114 shows an exemplary amino acid sequence of the fatty acyl-CoA oxidase referred to herein as a PxACX1.

SEQ ID NO:115 shows an exemplary amino acid sequence of the fatty acyl-CoA oxidase referred to herein as a PxACX3.

SEQ ID NOs:116-119 show exemplary amino acid sequences of fatty acyl-CoA oxidase enzymes referred to herein as an RnACOX1, an RnACOX2, an AtACX1, and an AtACX2, respectively.

SEQ ID NOs:120-124 show exemplary amino acid sequences of L. botrana fatty acyl-CoA oxidase (POX) enzymes referred to herein as an LbPOX1, an LbPOX2, an LbPOX3, an LbPOX4, and an LbPOX6, respectively.

SEQ ID NO:125 shows an amino acid sequence of Y. lipolytica fatty acyl-CoA oxidase POX2.

SEQ ID NO:126 shows an amino acid sequence of Y. lipolytica fatty acid elongase ELO1.

SEQ ID NO:127 shows an amino acid sequence of Y. lipolytica fatty acid elongase ELO2.

SEQ ID NOs:128-132 show exemplary amino acid sequences of the L. botrana fatty acyl-CoA reductase enzymes referred to herein as an LbFAR1, an LbFAR2, an LbFAR3, an LbFAR4, and an LbFAR5, respectively.

SEQ ID NOs:133-228 show exemplary polynucleotides corresponding to polypeptides of particular embodiments.

### DETAILED DESCRIPTION

I. Overview of Several Embodiments

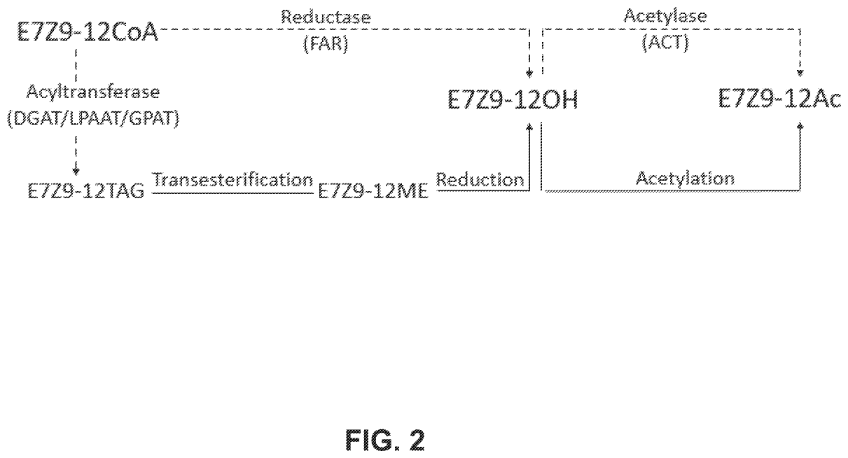

Microbial engineering was used to enable the production of insect pheromones using inexpensive feedstocks and scalable syntheses that sidestep the hazards and waste products encumbering traditional chemical synthesis (e.g., organic solvent waste). Described herein is a non-synthetic production method of the effective insect protection agent, E7Z9-12CoA. This method enables production of this economically important L. botrana active in a scalable and eco-friendly fermentation. FIG. 2 describes the downstream processing required to convert E7Z9-12CoA to E7Z9-12Ac.

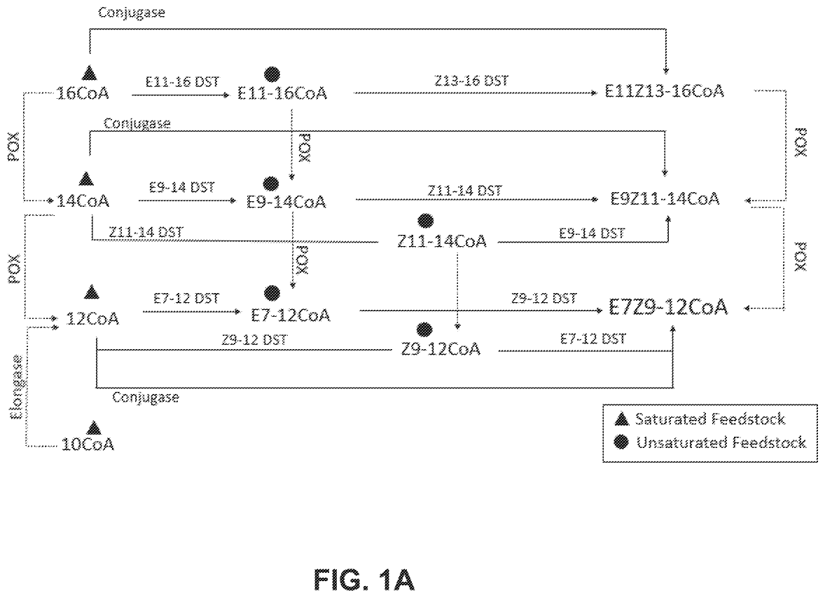

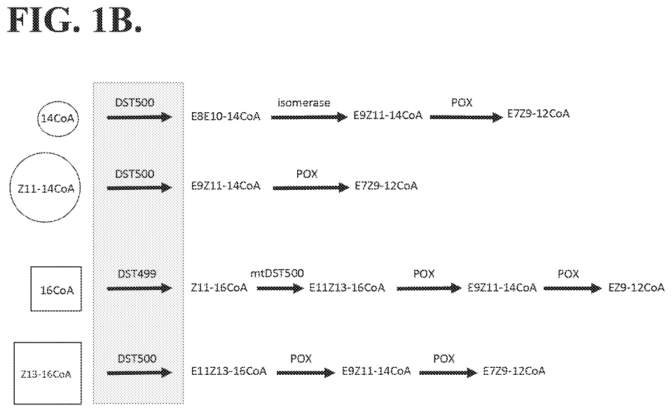

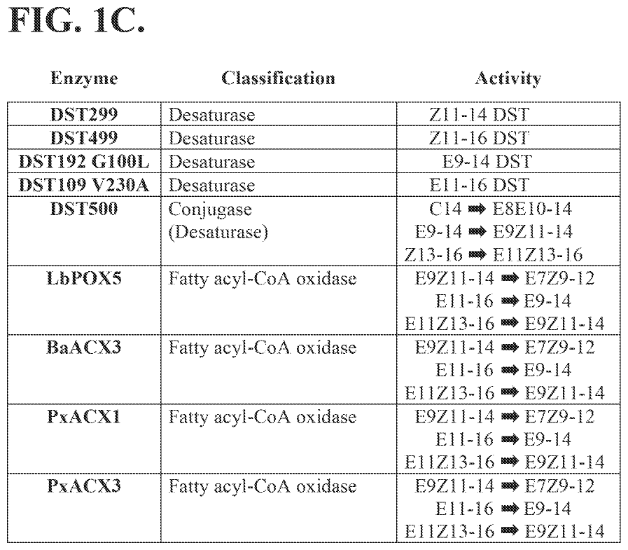

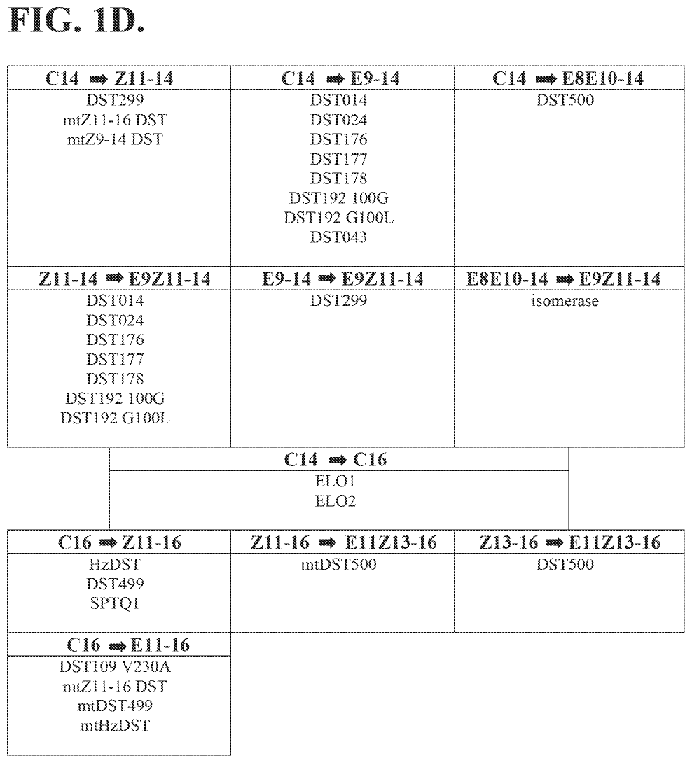

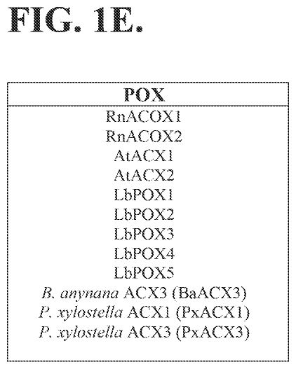

Metabolic engineering efforts involve co-opting native pathways and implanting heterologous pathways. For example, specific embodiments herein involve the introduction of E7Z9-12CoA biochemical pathways (FIG. 1) by expressing exogenous desaturases, conjugases, beta-oxidation enzymes, and acyl-CoA elongation enzymes, native and of heterologous origin. Desirable heterologous activities in microorganisms were obtained through enzyme selection and structural scaffold engineering. Pathway entry points are indicated with either a triangle (saturated feedstock) or circle (unsaturated feedstock) (FIG. 1). Substrates can be fed as either acids or alkyl esters to engineered microbes at these points along the pathway to E7Z9-12CoA.

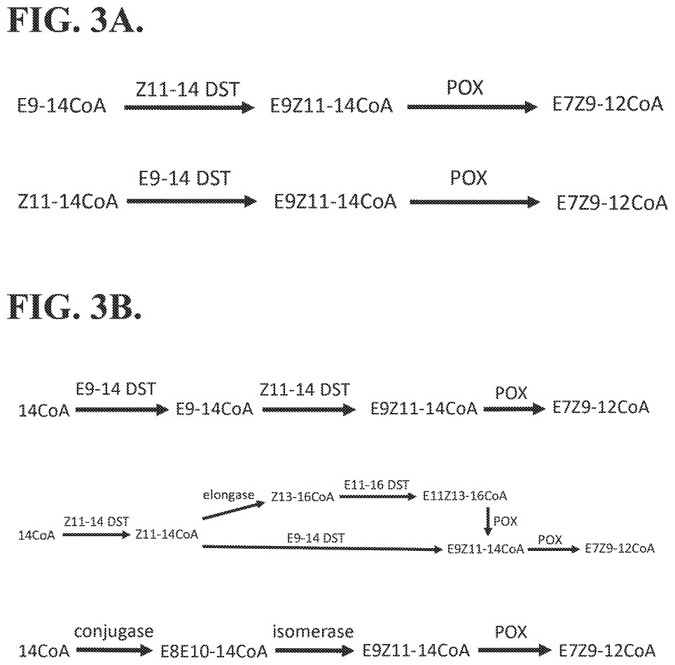

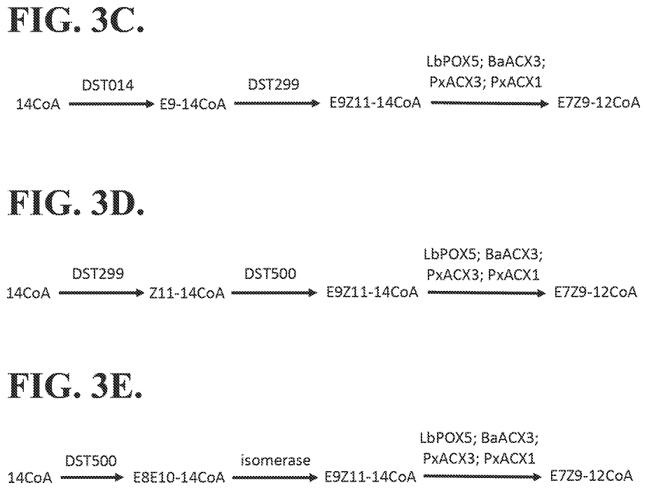

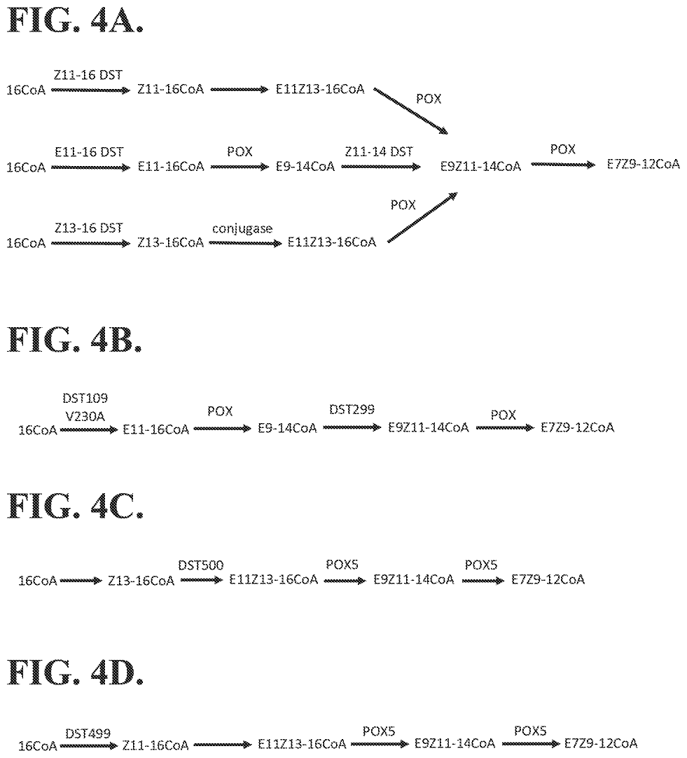

Key enzyme activities enable synthesis of E7Z9-12CoA, the direct precursor to E7Z9-12Ac. In particular embodiments herein, a novel Z11-14 desaturase (DST299), a novel Z11-16 desaturase (DST499), a novel conjugase (DST500), and an engineered E11-16 desaturase (DST109 V230A) are used alone or in combination to catalyze the formation of a E9Z11-14CoA intermediate from E9-14CoA, Z11-14CoA, Z13-16CoA, E8E10-14CoA, Z13-16CoA, 14CoA, and/or 16CoA. FIGS. 3-4. Several POX enzymes can chain shorten E9Z11-14CoA to E7Z9-12CoA. Embodiments herein utilize a microbe's endogenous lipase and acetyl-CoA synthetase activities, or acetyl-CoA synthetase activities imparted by heterologous acetyl-CoA synthetases, to convert feedstock substrates (i.e., fatty acids and fatty alkyl esters) to their co-enzyme A analogs. These CoA analogs can be acted upon by desaturases, conjugases, enzymes of the beta-oxidation system, and CoA elongation pathway enzymes to assemble E7Z9-12CoA (FIG. 1), the direct precursor to E7Z9-12Ac.

In some embodiments, a heterologous desaturase (“DST”) is utilized to introduce at least one double bond in the correct configuration at the specified carbon on the listed chain length (e.g., an E11-16 DST installs an E double bond between carbons 11 and 12 on 16CoA). Heterologous conjugases may be utilized to introduce double bonds directly in the correct orientation at the specified positions, or indirectly through an intermediate at the central carbon (e.g., a conjugase operating on 12CoA could introduce conjugated double bonds at carbons 7 and 9 directly, or through an intermediate at the central carbon 8 position, to form E7Z9-12CoA).

In other embodiments, an enzyme of the beta-oxidation system shortens the hydrocarbon chain of an acyl-CoA analog. Ledesma-Amaro & Nicaud (2016) Progress Lipid Res. 61:40-50. The beta-oxidation system/pathway comprises four enzymes: an acyl-CoA oxidase, an enoyl-CoA hydratase, a 3-hydroxyacyl-CoA dehydrogenase, and a thiolase. In particular embodiments, at least one exogenous acyl-CoA oxidase (“POX”) is utilized (for example, RnACOX1, RnACOX2, AtACX1, AtACX2, LbPOX1-5, BaACX3, PxACX1, and/or PxACX3), and a microorganism's endogenous machinery is co-opted to exert the final three activities in the beta-oxidation pathway, though all four activities are subject to modulation through engineering. In particular embodiments, LbPOX5 is utilized. Also or alternatively in particular embodiments, at least one of BaACX3, PxACX1, and PxACX3 is utilized.

In certain embodiments, the microbe's endogenous lipase cleaves alkyl esters to the corresponding carboxylic acid. Thevenieau et al. (2010) “Uptake and assimilation of hydrophobic substrates by the oleaginous yeast Yarrowia lipolytica,” in Handbook of Hydrocarbon and Lipid Microbiology, ed. Timmis K. N., editor. (Berlin, Heidelberg: Springer-Verlag), pp. 1513-27. Similarly, endogenous acetyl-CoA synthetase converts fatty acids to their CoA analogs in some embodiments. Tenagy et al. (2015) FEMS Yeast Res. 15:fov031.

The CoA elongation pathway lengthens the hydrocarbon chain of acyl-CoA analogs by two carbons. Additionally, the CoA elongation pathway is comprised of an elongase, a beta-ketoacyl-CoA reductase, a dehydratase, and an enoyl-CoA reductase. We have determined that a native CoA elongation pathway is operative in yeast; for example, Y. lipolytica. When utilized in this organism, its activity can be modulated through over-expression (e.g., of elo1 or elo2) and deletion. In some embodiments herein, ELO1 (or a homolog or ortholog thereof) ELO2 (or a homolog or ortholog thereof) is utilized in an E7Z9-12CoA production pathway to convert Z11-14CoA to Z13-16CoA, for example, such that the Z13-16CoA is then converted to E11Z13-16CoA by a conjugase (e.g., DST500). The E11Z13-16CoA may then be oxidized by an acyl-CoA oxidase (e.g., LbPOX5, BaACX3, PxACX1, and PxACX) to produce the E9Z11-14CoA intermediate and then the E7Z9-12CoA product.

In embodiments herein, either biological or chemical methods may be used to convert microbial E7Z9-12CoA into E7Z9-12Ac. FIG. 2. In the biological route, E7Z9-12CoA can be reduced with a fatty acyl-CoA reductase (“FAR”) to its alcohol cognate (E7Z9-12OH). An acetylase (“ACT”) appends an acetate group to the alcohol to form E7Z9-12Ac. In some embodiments, the FAR is an exogenous L. botrana FAR (e.g., L. botrana FAR1, L. botrana FAR2, L. botrana FAR3, L. botrana FAR4, and L. botrana FAR5). The upper biological pathway could also be acetylated chemically to generate E7Z9-12Ac. Separately, acyltrasferases can convert the CoA analogs into the triacylglyceride (“TAG”) form for storage in the cell. In the chemical route, E7Z9-12TAG is first transesterified to the methyl ester (E7Z9-12ME), which is reduced to the alcohol (E7Z9-12OH) and finally acetylated to E7Z9-12Ac. Notably, ACT can also acetylate E7Z9-12OH through the lower chemical pathway.

II. Abbreviations

ACT acetylase

CoA co-enzyme A

DST desaturase

FAR fatty acyl-CoA reductase

GC gas chromatography

GC-FID gas chromatography with flame ionization detector

ME methyl ester

MTAD 4-methyl-1,2,4-triazoline-3,5-dione

POX acyl-CoA oxidase

RMSD root mean square deviation

THMP tetrakis(hydroxymethyl)phosphine

III. Terms

Isolated: An “isolated” biological component (such as a polynucleotide, polypeptide, or small molecule) has been substantially separated, produced apart from, or purified away from other biological components in the cell of the organism in which the component naturally occurs (e.g., other chromosomal and extra-chromosomal DNA and RNA, and proteins), while effecting a chemical or functional change in the component (e.g., a small molecule may be isolated from a cell by incorporating the molecule in an agricultural formulation).

Polypeptide: As used herein, the term “polypeptide” refers to a polymeric form of amino acids, linked by peptide (amide) covalent bonds. An amino acid as found in a polypeptide may be a natural amino acid, or in certain examples, a non-natural amino acid. A “protein” as used herein refers to a discrete molecule consisting of one or more polypeptides.

Substrates: Some embodiments herein include substrates of one or more E7Z9-12 biosynthetic pathways and methods comprising feeding such substrates to genetically modified organisms (for example, in a cell culture such as a fermentation culture), or introducing them into a reaction volume adapted for in vitro protein synthesis, wherein either the genetically modified organism or reaction volume comprises components of a biosynthetic pathway herein. In particular embodiments, a substrate may be referred to as a “C14” or “C16” substrate. As these terms are used herein, they are defined such as to specifically encompass both saturated and unsaturated fatty acid molecules with the designated chain length, and encompasses racemic and enantiomerically pure unsaturated substrate compositions and unsaturated substrate compositions that are not enantiomerically pure, but are enriched for a particular stereoisomer of the substrate molecule. Furthermore, the term encompasses all fatty acid molecules able to be metabolized by the host organism or components of the reaction volume, such that they are directly or indirectly introduced into the biosynthetic pathway(s) engineered in the particular application. By way of non-limiting example, a “C14” and “C16” substrate may be a free fatty acid, methyl ester, fatty acid-CoA.

Polynucleotide: As used herein, the term “polynucleotide” refers to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein refers to a discrete molecule consisting of one or more polynucleotides. A polynucleotide is usually at least 10 bases in length, unless otherwise specified. The term includes single- and double-stranded forms of DNA.

Exogenous: The term “exogenous,” as used herein, refers to one or more molecule(s) (e.g., polynucleotides, polypeptides, and small molecules) that are not normally present within their specific environment or context. For example, if a genetically modified host cell contains a polypeptide that does not occur in the unmodified host cell in nature, then that polypeptide is exogenous to the host cell. The term exogenous, as used herein specifically with regard to polynucleotides, also refers to one or more polynucleotide(s) that are identical in sequence to a polynucleotide already present in a host cell, but which are located in a different cellular or genomic context than the polynucleotide with the same sequence already present in the host cell. For example, a polynucleotide that is integrated in the genome of the host cell in a different location than a polynucleotide with the same sequence is normally integrated in the genome of the host cell is exogenous to the host cell.

Heterologous: The term “heterologous,” as used herein, means of different origin. For example, if a genetically modified host cell contains a polypeptide that does not occur in the unmodified host cell in nature, then that polypeptide is heterologous (and exogenous) to the host cell. Furthermore, different polynucleotide elements (e.g., promoters, enhancers, coding sequences, and terminators) or polypeptide elements (e.g., targeting signals, functional and non-functional domains, transmembrane domains, amino-terminal domains, and carboxy-terminal domains) of an exogenous molecule may be heterologous to one another and/or to a host cell. The term heterologous, as used herein, therefore also includes polynucleotides that are identical in sequence to a polynucleotide already present in a host cell, but which are now linked to different additional sequences and/or are present at a different copy number, etc.

Sequence identity: The term “sequence identity” or “identity,” as used herein in the context of two nucleotide or amino acid sequences, may refer to the residues in the two sequences that are the same when aligned for maximum correspondence over a specified comparison window. As these terms are used herein, the percentage of “sequence identity” refers to the value determined by comparing two optimally aligned sequences (e.g., nucleotide sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleotide or amino acid sequences, the “Blast 2 sequences” function of the BlastN™ or BlastP™ program, respectively, may be employed using default parameters. Sequences with even greater similarity to the reference sequences will show increasing percentage identity when assessed by this method.

As used herein with regard to polypeptides, the term “substantially identical” refers to amino acid sequences that are more than about 80% identical. For example, a substantially identical amino acid sequence may be at least 79.5%; at least 80%; at least 81%; at least 82%; at least 83%; at least 84%; at least 85%; at least 86%; at least 87%; at least 88%; at least 89%; at least 90%; at least 91%; at least 92%; at least 93%; at least 94%; at least 95%; at least 96%; at least 97%; at least 98%; at least 99%; or at least 99.5% identical to the reference sequence. In specific embodiments, the amino acid sequence of a desaturase (e.g., DST299 and DST499), conjugase (e.g., DST500), acyl-CoA oxidase (e.g., LbPOX5, BaACX3, PxACX1, and PxACX3), or acyl-CoA elongase (e.g., ELO1 and ELO2) is substantially identical to a reference amino acid sequence to an extent defined by any of the foregoing integers.

Those in the art understand that conservative substitutions may be made to the primary amino acid sequence of a polypeptide without disrupting its activity to an undesirable extent, depending on the application, particularly in the case of polypeptides containing known and characterized sequence motifs, or those for which a structural model exists. As used herein, the term “conservative substitution” refers to a substitution where an amino acid residue is substituted for another amino acid in the same class. A non-conservative amino acid substitution is one where the residues do not fall into the same class, for example, substitution of a basic amino acid for a neutral or non-polar amino acid.

Classes of amino acids that may be defined for the purpose of performing a conservative substitution are known in the art. For example, aliphatic amino acids include Gly, Ala, Pro, Ile, Leu, Val, and Met; aromatic amino acids include His, Phe, Trp, and Tyr; hydrophobic amino acids include Ala, Val, Ile, Leu, Met, Phe, Tyr, and Trp; polar amino acids include Ser, Thr, Asn, Gln, Cys, Gly, Pro, Arg, His, Lys, Asp, and Glu; non-polar amino acids include Ala, Val, Leu, Ile, Phe, Trp, Pro, and Met; and electrically neutral amino acids include Gly, Ser, Thr, Cys, Asn, Gln, and Tyr.

In many examples, the selection of a particular second amino acid to be used in a conservative substitution to replace a first amino acid may be made in order to maximize the number of the foregoing classes to which the first and second amino acids both belong. Thus, if the first amino acid is Ser (a polar, non-aromatic, and electrically neutral amino acid), the second amino acid may be another polar amino acid (i.e., Thr, Asn, Gln, Cys, Gly, Pro, Arg, His, Lys, Asp, or Glu); another non-aromatic amino acid (i.e., Thr, Asn, Gln, Cys, Gly, Pro, Arg, His, Lys, Asp, Glu, Ala, Ile, Leu, Val, or Met); or another electrically neutral amino acid (i.e., Gly, Thr, Cys, Asn, Gln, or Tyr). However, it may be preferred that the second amino acid in this case be one of Thr, Asn, Gln, Cys, and Gly, because these amino acids share all the classifications according to polarity, non-aromaticity, and electrical neutrality. Additional criteria that may optionally be used to select a particular second amino acid to be used in a conservative substitution are known in the art. For example, when Thr, Asn, Gln, Cys, and Gly are available to be used in a conservative substitution for Ser, Cys may be eliminated from selection in order to avoid the formation of undesirable cross-linkages and/or disulfide bonds. Likewise, Gly may be eliminate from selection, because it lacks an alkyl side chain. In this case, Thr may be selected, e.g., in order to retain the functionality of a side chain hydroxyl group. The selection of the particular second amino acid to be used in a conservative substitution is ultimately, however, within the discretion of the skilled practitioner. With the foregoing guidance, she is able to identify substantially identical and functionally equivalent polypeptides without the exercise of inventive skill.

Specifically complementary: Polynucleotides may alternatively be described structurally herein as being “specifically complementary” to a reference nucleotide sequence. As used herein, the term “specifically complementary” indicates a sufficient degree of complementarity such that stable and specific hybridization occurs between the polynucleotide and an oligonucleotide consisting of the reference sequence. Hybridization between the polynucleotide and the oligonucleotide involves the formation of an anti-parallel alignment between their respective nucleobases. The polynucleotide and the oligonucleotide are then able to form hydrogen bonds with corresponding bases on the opposite strand to form a duplex molecule that, if it is sufficiently stable, is detectable using methods well known in the art. A polynucleotide need not be 100% complementary to its target nucleic acid to hybridize stably and specifically to the target. However, the amount of complementarity that must exist for hybridization to be specific is a function of the hybridization conditions used.

Hybridization conditions resulting in particular degrees of stringency will vary depending upon the nature of the hybridization method of choice and the composition and length of the hybridizing nucleic acids. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are known to those of ordinary skill in the art, and are discussed, for example, in Sambrook et al. (ed.) Molecular Cloning: A Laboratory Manual, 2^{nd} ed., vol. 1-3, Cold Spring Harbor Laboratory Press, Cold Spring Harbor, N.Y., 1989, chapters 9 and 11; and Hames and Higgins (eds.) Nucleic Acid Hybridization, IRL Press, Oxford, 1985. Further detailed instruction and guidance with regard to the hybridization of nucleic acids may be found, for example, in Tijssen, “Overview of principles of hybridization and the strategy of nucleic acid probe assays,” in Laboratory Techniques in Biochemistry and Molecular Biology- Hybridization with Nucleic Acid Probes, Part I, Chapter 2, Elsevier, N Y, 1993; and Ausubel et al., Eds., Current Protocols in Molecular Biology, Chapter 2, Greene Publishing and Wiley-Interscience, N Y, 1995.

As used herein, “stringent conditions” encompass conditions under which hybridization will only occur if there is no more than 20% mismatch between the sequence of the hybridization molecule and a homologous polynucleotide within the target nucleic acid molecule. “Stringent conditions” include further particular levels of stringency. Thus, as used herein, “moderate stringency” conditions are those under which molecules with at least 20% sequence mismatch will not hybridize; conditions of “high stringency” are those under which sequences with more than 10% mismatch will not hybridize; and conditions of “very high stringency” are those under which sequences with more than 5% mismatch will not hybridize.

The following are representative, non-limiting hybridization conditions.

High Stringency condition (detects polynucleotides that share at least 90% sequence identity): Hybridization in 5×SSC buffer at 65° C. for 16 hours; wash twice in 2×SSC buffer at room temperature for 15 minutes each; and wash twice in 0.5×SSC buffer at 65° C. for 20 minutes each.

Moderate Stringency condition (detects polynucleotides that share at least 80% sequence identity): Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.

Non-stringent control condition (polynucleotides that share at least 50% sequence identity will hybridize): Hybridization in 6×SSC buffer at room temperature to 55° C. for 16-20 hours; wash at least twice in 2×-3×SSC buffer at room temperature to 55° C. for 20-30 minutes each.

As used herein with regard to polynucleotides, the term “substantially identical” refers to nucleotide sequences that are more than about 60% identical. For example, a substantially identical nucleotide sequence may be at least 59.5%; at least 60%; at least 61%; at least 62%; at least 63%; at least 64%; at least 65%; at least 66%; at least 67%; at least 68%; at least 69%; at least 70%; at least 71%; at least 72%; at least 73%; at least 74%; at least 75%; at least 76%; at least 77%; at least 78%; at least 79%; at least 80%; at least 81%; at least 82%; at least 83%; at least 84%; at least 85%; at least 86%; at least 87%; at least 88%; at least 89%; at least 90%; at least 91%; at least 92%; at least 93%; at least 94%; at least 95%; at least 96%; at least 97%; at least 98%; at least 99%; or at least 99.5% identical to the reference sequence. In specific embodiments, the nucleotide sequence of a polynucleotide encoding a desaturase, conjugase, beta-oxidation enzyme, or acyl-CoA elongation enzyme is substantially identical to a reference nucleotide sequence to an extent defined by any of the foregoing integers. The property of substantial homology is closely related to specific hybridization. For example, a nucleic acid molecule is specifically hybridizable when there is a sufficient degree of complementarity to avoid non-specific binding of the nucleic acid to non-target polynucleotides under conditions where specific binding is desired, for example, under stringent hybridization conditions. Those in the art understand that, due to the redundancy of the genetic code, the first two nucleotides of a codon are often determinative of the amino acid encoded thereby. Thus, polynucleotides having nucleotide sequences with as little as 60% identity may be designed to encode essentially identical polypeptides.

Operably linked: A first polynucleotide is operably linked with a second polynucleotide when the first polynucleotide is in a functional relationship with the second polynucleotide. When recombinantly produced, operably linked polynucleotides are generally contiguous, and, where necessary to join two protein-coding regions, in the same reading frame (e.g., in a translationally fused ORF). However, polynucleotides need not be contiguous to be operably linked.

The term, “operably linked,” when used in reference to a regulatory genetic element and a coding polynucleotide, means that the regulatory element affects the expression of the linked coding polynucleotide. “Regulatory elements,” or “control elements,” refer to polynucleotides that influence the timing and level/amount of transcription, RNA processing or stability, or translation of the associated coding polynucleotide. Regulatory elements may include promoters, translation leaders, introns, enhancers, stem-loop structures, repressor binding polynucleotides, polynucleotides with a termination sequence, polynucleotides with a polyadenylation recognition sequence, etc. Particular regulatory elements may be located upstream and/or downstream of a coding polynucleotide operably linked thereto. Also, particular regulatory elements operably linked to a coding polynucleotide may be located on the associated complementary strand of a double-stranded nucleic acid molecule.

Promoter: As used herein, the term “promoter” refers to a region of DNA that may be upstream from the start of transcription, and that may be involved in recognition and binding of RNA polymerase and other proteins to initiate transcription. A promoter may be operably linked to a coding polynucleotide for expression in a cell, or a promoter may be operably linked to a polynucleotide encoding a signal peptide which may be operably linked to a coding polynucleotide for expression in a cell. “Inducible” promoters include those that are under environmental control. Examples of environmental conditions that may initiate transcription by inducible promoters include anaerobic conditions and the presence of light. In particular embodiments herein, a polynucleotide encoding a desaturase, conjugase, acyl-CoA oxidase, or elongase is operably linked to a promoter that is functional in yeast.

Transformation: As used herein, the term “transformation” refers to the transfer of one or more polynucleotide(s) into a cell. A cell is “transformed” when a nucleic acid molecule is transduced into the cell, such that a polynucleotide of the nucleic acid molecule becomes stably replicated by the cell, either by incorporation of the polynucleotide into the cellular genome, or by episomal replication. As used herein, the term “transformation” encompasses all techniques by which a nucleic acid molecule can be introduced into such a cell. Examples include, but are not limited to: transformation with plasmid vectors, electroporation (Fromm et al. (1986) Nature 319:791-3), lipofection (Feigner et al. (1987) Proc. Natl. Acad. Sci. USA 84:7413-7), microinjection (Mueller et al. (1978) Cell 15:579-85), and direct DNA uptake.

Transgene: An exogenous coding polynucleotide. In some examples, a transgene may be a polynucleotide that encodes a functional polypeptide (e.g., desaturases, conjugases, fatty acyl-CoA oxidases, fatty acyl-CoA reductases, and elongases) in a host cell. In these and other examples, a transgene may be comprised in an expression cassette containing regulatory elements (e.g., a promoter) operably linked to the transgene.

Vector: A nucleic acid molecule as introduced into a cell, for example, to produce a transformed cell. A vector may include genetic elements that permit it to replicate in the host cell, such as an origin of replication. Examples of vectors include, for example and without limitation, plasmids, cosmids, bacteriophages, and viruses that carry exogenous DNA into a cell. A vector may also include one or more genes, including transgenes and/or selectable marker genes, and other genetic elements known in the art. A vector may transform a cell, thereby causing the cell to express the polynucleotides and/or polypeptides encoded by the vector. A vector optionally includes materials to aid in achieving entry of the nucleic acid molecule into the cell (e.g., a liposome, protein coating, etc.).

Unless specifically indicated or implied, the terms “a,” “an,” and “the” signify “at least one,” as used herein.

Unless otherwise specifically explained, all technical and scientific terms used herein have the same meaning as commonly understood by those of ordinary skill in the art to which this disclosure belongs. Definitions of common terms in molecular biology can be found in, for example, Lewin's Genes X, Jones & Bartlett Publishers, 2009 (ISBN 10 0763766321); Krebs et al. (eds.), The Encyclopedia of Molecular Biology, Blackwell Science Ltd., 1994 (ISBN 0-632-02182-9); and Meyers R. A. (ed.), Molecular Biology and Biotechnology: A Comprehensive Desk Reference, VCH Publishers, Inc., 1995 (ISBN 1-56081-569-8). All percentages are by weight and all solvent mixture proportions are by volume unless otherwise noted. All temperatures are in degrees Celsius.

IV. Components of the E7Z9-12CoA/E7Z9-12Ac Biosynthetic Pathway

Disclosed herein are components of a E7Z9-12CoA biosynthetic pathway that may be utilized to produce pheromones in a microorganism, or for engineering to provide further components with specific desired activities. Embodiments may include one or more of the desaturase, conjugase, fatty acyl-CoA oxidase, reductase, and elongase polypeptides herein; for example, the polypeptides referred to herein as DST299, DST499, DST109, DST014, DST024, DST176, DST177, DST178, DST192 100G, DST192 100L (i.e., DST192 G100L), DST043, DST500, ELO1, ELO2, RnACOX1, AtACX1, PxACX1, RnACOX2, Y. lipolytica POX2, AtACX2, PxACX3, BaACX3, LbPOX1, LbPOX2, LbPOX3, LbPOX4, LbPOX5, and LbPOX6. Particular embodiments include at least one desaturase selected from the group consisting of DST299, DST499, and DST109 V230A, a DST500 conjugase, and/or at least one fatty acyl-CoA oxidase selected from the group consisting of LbPOX5, BaACX3, PxACX1, and PxACX3. The foregoing components may be selected to form E7Z9-12CoA through one of the metabolic pathways described herein, and may be engineered into a host organism (e.g., yeast) by recombinant molecular biological techniques to introduce one or more of the pathways into the host, or may be utilized in an in vitro synthesis platform to yield E7Z9-12CoA or E7Z9-12Ac.

DST299 is a novel desaturase that catalyzes, for example, the formation of Z11-14CoA and E9Z11-14CoA intermediates from 14CoA and E9-14CoA, respectively. FIG. 3. As the term is used herein, DST299 refers to a functional desaturase comprising an amino acid sequence that is at least about 90% identical to SEQ ID NO:1. For example, a DST299 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:1, preferably wherein the amino acid substitution(s) are conservative substitutions outside the substrate binding pocket. In specific examples, a DST299 desaturase comprises an amino acid sequence that is at least 95% or at least 98% identical to SEQ ID NO:1.

DST499 is a novel desaturase that catalyzes, for example, the formation of a Z11-16CoA intermediate from 16CoA. FIG. 4. As the term is used herein, DST499 refers to a functional desaturase comprising an amino acid sequence that is at least about 90% identical to SEQ ID NO:2. For example, a DST499 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:2, preferably wherein the amino acid substitution(s) are conservative substitutions outside the substrate binding pocket. In specific examples, a DST499 desaturase comprises an amino acid sequence that is at least 95% or at least 98% identical to SEQ ID NO:2.

DST024 is a desaturase that catalyzes, for example, the formation of E9-14CoA and E9Z11-CoA intermediates from 14CoA and Z11-14CoA, respectively. FIG. 3. As the term is used herein, DST024 refers to a functional desaturase comprising an amino acid sequence that is at least about 90% identical to SEQ ID NO:8. For example, a DST024 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:8, preferably wherein the amino acid substitution(s) are conservative substitutions outside the substrate binding pocket. In specific examples, a DST024 desaturase comprises an amino acid sequence that is at least 95% or at least 98% identical to SEQ ID NO:8.

DST176 is a desaturase that catalyzes, for example, the formation of E9-14CoA and E9Z11-CoA intermediates from 14CoA and Z11-14CoA, respectively. FIG. 3. As the term is used herein, DST176 refers to a functional desaturase comprising an amino acid sequence that is at least about 90% identical to SEQ ID NO:9. For example, a DST176 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:9, preferably wherein the amino acid substitution(s) are conservative substitutions outside the substrate binding pocket. In specific examples, a DST176 desaturase comprises an amino acid sequence that is at least 95% or at least 98% identical to SEQ ID NO:9.

DST177 is a desaturase that catalyzes, for example, the formation of E9-14CoA and E9Z11-CoA intermediates from 14CoA and Z11-14CoA, respectively. FIG. 3. As the term is used herein, DST177 refers to a functional desaturase comprising an amino acid sequence that is at least about 90% identical to SEQ ID NO:10. For example, a DST177 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:10, preferably wherein the amino acid substitution(s) are conservative substitutions outside the substrate binding pocket. In specific examples, a DST177 desaturase comprises an amino acid sequence that is at least 95% or at least 98% identical to SEQ ID NO:10.

DST178 is a desaturase that catalyzes, for example, the formation of E9-14CoA and E9Z11-CoA intermediates from 14CoA and Z11-14CoA, respectively. FIG. 3. As the term is used herein, DST178 refers to a functional desaturase comprising an amino acid sequence that is at least about 90% identical to SEQ ID NO:11. For example, a DST178 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:11, preferably wherein the amino acid substitution(s) are conservative substitutions outside the substrate binding pocket. In specific examples, a DST178 desaturase comprises an amino acid sequence that is at least 95% or at least 98% identical to SEQ ID NO:11.

DST192s are desaturases that catalyze, for example, the formation of E9-14CoA and E9Z11-CoA intermediates from 14CoA and Z11-14CoA, respectively (FIG. 3), or Z9-14CoA intermediates from 14CoA. As the term is used herein, DST192 refers to a functional desaturase comprising an amino acid sequence that is at least about 90% identical to SEQ ID NO:3. For example, a DST192 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:3, preferably wherein the amino acid substitution(s) are conservative substitutions outside the substrate binding pocket. In particular examples, a DST192 desaturase comprises a Gly or Lys residue at the amino acid position corresponding to position 100 of SEQ ID NO:3, for example, SEQ ID NO:4. In particular embodiments, a DST192 desaturase may exhibit increased E9-14 DST activity, wherein the DST192 desaturase comprises an amino acid sequence that is at least about 90% identical to SEQ ID NO:3 and comprises a Lys residue at the position corresponding to G100. In particular embodiments, the DST192 desaturase exhibiting increased E9-14 DST activity is SEQ ID NO:4. In specific examples, a DST192 desaturase comprises an amino acid sequence that is at least 95% or at least 98% identical to SEQ ID NO:3 and comprises a Lys residue at the position corresponding to G100.

DST043 is a desaturase that catalyzes, for example, the formation of E9-14CoA and E9Z11-CoA intermediates from 14CoA and Z11-14CoA, respectively. FIG. 3. As the term is used herein, DST043 refers to a functional desaturase comprising an amino acid sequence that is at least about 90% identical to SEQ ID NO:12. For example, a DST043 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:12, preferably wherein the amino acid substitution(s) are conservative substitutions outside the substrate binding pocket. In specific examples, a DST043 desaturase comprises an amino acid sequence that is at least 95% or at least 98% identical to SEQ ID NO:12.

DST109 is a desaturase that may be engineered to catalyze, for example, the formation of an E11-16CoA intermediate from 16CoA. FIG. 3. As the term is used herein, DST109 refers to a functional desaturase comprising an amino acid sequence that is at least about 90% identical to SEQ ID NO:5 or SEQ ID NO:17. For example, a DST109 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:5. In specific examples, a DST109 desaturase comprises an amino acid sequence that is at least 95% or at least 98% identical to SEQ ID NO:5. In particular examples, a DST109 desaturase may further comprise a Val or Ala residue at the amino acid position corresponding to position 230 of SEQ ID NO:17, for example, SEQ ID NO:6); an Arg or Ala residue at the amino acid position corresponding to position 243 of SEQ ID NO:17; and/or an Phe or Ala residue at the amino acid position corresponding to position 252 of SEQ ID NO:17. In some examples, the DST109 desaturase is not SEQ ID NO:5. In particular embodiments, a DST109 desaturase may exhibit increased E9-14 or E11-16 DST activity, wherein the DST109 desaturase comprises an amino acid sequence selected from the group consisting of SEQ ID NOs:18-21. In particular embodiments, the DST109 desaturase exhibiting increased E11-16 DST activity is SEQ ID NO:6.

DST014 is a further desaturase that catalyzes, for example, the formation of E9-14CoA and E9Z11-14CoA intermediates from 14CoA and Z11-14CoA, respectively. FIG. 3. As the term is used herein, DST014 refers to a functional desaturase comprising an amino acid sequence that is at least about 90% identical to SEQ ID NO:7 or SEQ ID NO:16. For example, a DST014 desaturase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:7. In specific examples, a DST014 desaturase comprises an amino acid sequence that is at least 95% or at least 98% identical to SEQ ID NO:7. In particular examples, a DST014 desaturase may further comprise a Tyr, Asn, His, Phe, Cys, Ser, or Leu residue at the amino acid position corresponding to position 64 of SEQ ID NO:16; an Ile, Leu, Glu, Gly, Thr, Ala, or Val residue at the amino acid position corresponding to position 68 of SEQ ID NO:16; an Ile, Val, or Ala residue at the amino acid position corresponding to position 71 of SEQ ID NO:16; an Asn, His, Lys, Asp, or Ser residue at the amino acid position corresponding to position 104 of SEQ ID NO:16; a His, Lys, Arg, or Ser residue at the amino acid position corresponding to position 144 of SEQ ID NO:16; a Lys, Ile, Val, or Leu residue at the amino acid position corresponding to position 150 of SEQ ID NO:16; a Leu, Phe, Tyr, or Trp residue at the amino acid position corresponding to position 217 of SEQ ID NO:16; a Cys, Leu, Met, Phe, or Ser residue at the amino acid position corresponding to position 218 of SEQ ID NO:16; an Ile, Leu, Glu, Gly, Thr, Ala, or Val residue at the amino acid position corresponding to position 219 of SEQ ID NO:16; and/or a Met, Cys, Thr, Ala, Val, Ser, or Cys residue at the amino acid position corresponding to position 247 of SEQ ID NO:16. In some examples, the DST014 desaturase is not SEQ ID NO:7. In particular embodiments, a DST014 desaturase may exhibit E11-16, Z11, Z13, Z14, Z9, or Z6 DST activity, wherein the DST014 desaturase comprises an amino acid sequence selected from the group consisting of SEQ ID NOs:22-26. In particular embodiments, the DST014 desaturase comprises SEQ ID NO:22.

Some embodiments herein may utilize at least one further desaturase, for example, to catalyze the formation of E9-14CoA and E9Z11-CoA intermediates from 14CoA and Z11-14CoA, respectively via E9-14 activity, or E7Z9-12CoA from E7-12CoA via Z9-14 activity. Examples of such desaturases comprise an amino acid sequence that is at least at least 89.5%, at least 90%, at least 91%, at least 92%, at least 93%, at least 94%, at least 95%, at least 96%, at least 97%, at least 98%, at least 99%, or 100% identical to SEQ ID NO:28 (DST162), SEQ ID NO:29 (DST163), SEQ ID NO:30 (DST165), SEQ ID NO:31 (DST166), SEQ ID NO:32 (DST076), SEQ ID NO:33 (DST077), SEQ ID NO:34 (DST167), SEQ ID NO:35 (DST168), SEQ ID NO:36 (DST169), SEQ ID NO:37 (DST170), SEQ ID NO:38 (DST171), SEQ ID NO:39 (DST172), SEQ ID NO:40 (DST175), SEQ ID NO:41 (DST179), SEQ ID NO:42 (DST180), SEQ ID NO:43 (DST181), SEQ ID NO:44 (DST183), SEQ ID NO:45 (DST184), SEQ ID NO:46 (DST185), SEQ ID NO:47 (DST186), SEQ ID NO:48 (DST191), or SEQ ID NO:49 (DST219), preferably wherein the amino acid substitution(s) are conservative substitutions outside the substrate binding pocket. In specific examples, an E9-14 or Z9-14 desaturase comprises an amino acid sequence that is at least 95% or at least 98% identical to an amino acid sequence selected from SEQ ID NOs:28-49.

DST500 is a novel polypeptide comprising an SPTQ (SEQ ID NO:78) motif that has been found to define a family of enzymes utilized herein to provide a broad scope of activities that allow access to E7Z9-12CoA (and thereby E7Z9-12Ac) through a variety of independently useful pathways from different fatty acid substrates. The 4-amino acid XXXQ motif is characteristic of Δ10,11 desaturases (Knipple et al. (2002) Genetics 162:1737-52; Matoušková et al. (2007) Insect Biochem. Mol. Biol. 37:601-10; Serra et al. (2007) Proc. Natl. Acad. Sci. U.S.A 104:16444-9). However, DST500 has been surprisingly found to exhibit 8,10-conjugase activity on 14CoA and also E9 desaturase activity. FIG. 1. For example, DST500 desaturates Z11-14CoA to form an E9Z11-14CoA intermediate, desaturates E9-14CoA to form an E9Z11-14CoA intermediate, conjugates 14CoA to form an E8E10-14CoA intermediate, and conjugates Z13-16CoA to form an E11Z13-16CoA intermediate. FIGS. 3-4. As the term is used herein, DST500 refers to a functional conjugase comprising an amino acid sequence that is at least about 90% identical to SEQ ID NO:111. For example, a DST500 conjugase may comprise an amino acid sequence that is at least 89.5%, at least 90%, at least 91%, at least 92%, at least 93%, at least 94%, at least 95%, at least 96%, at least 97%, at least 98%, at least 99%, or 100% identical to SEQ ID NO:111, preferably wherein the amino acid substitution(s) are conservative substitutions outside the substrate binding pocket. In specific examples, a DST500 conjugase comprises an amino acid sequence that is at least 95% or at least 98% identical to SEQ ID NO:111.

Fatty acyl-CoA oxidases utilized in some embodiments herein include, for example and without limitation, ACOX1, such as ACOX1 (e.g., Rattus norvegicus ACOX1 (RnACOX1), Arabidopsis thaliana ACOX1 (AtACX1), Plutella xylostella ACOX1 (PxACX1)), ACOX2 (e.g., R. norvegicus ACOX2 (RnACOX2), Y. lipolytica POX2 (POX2), and A. thaliana ACOX2 (AtACX2)), ACOX3 (e.g., P. xylostella ACOX3 (PxACX3) and Bicyclus anynana ACOX3 (BaACX3)), and any of L. botrana POX1-6 (LbPOX1, LbPOX2, LbPOX3, LbPOX4, LbPOX5, LbPOX6).

Accordingly, particular embodiments herein include a fatty acyl-CoA oxidase comprising an amino acid sequence that is at least at least 89.5%, at least 90%, at least 91%, at least 92%, at least 93%, at least 94%, at least 95%, at least 96%, at least 97%, at least 98%, at least 99%, or 100% identical to SEQ ID NO:116 (RnACOX1), SEQ ID NO:118 (AtACX1), SEQ ID NO:115 (PxACX1), SEQ ID NO:117 (RnACOX2), SEQ ID NO:125 (POX2), SEQ ID NO:119 (AtACX2), SEQ ID NO:115 (PxACX3), SEQ ID NO:113 (BaACX3), SEQ ID NO:120 (LbPOX1), SEQ ID NO:121 (LbPOX2), SEQ ID NO:122 (LbPOX3), SEQ ID NO:123 (LbPOX4), SEQ ID NO:112 (LbPOX5), or SEQ ID NO:124 (LbPOX6), preferably wherein the amino acid substitution(s) are conservative substitutions outside the substrate binding pocket. In specific embodiments, a fatty acyl-CoA oxidase comprises an amino acid sequence that is at least 95% or at least 98% identical to an amino acid sequence selected from SEQ ID NOs:112-125.

Particular embodiments utilize at least one of LbPOX5, BaACX3, PxACX1, and PxACX3, for example, to catalyze the formation of an E7Z9-12CoA and/or E9Z11-14CoA intermediate from E9Z11-14CoA or E11Z13-16CoA, respectively. Therefore, specific examples include a fatty acyl-CoA oxidase comprising an amino acid sequence that is at least at least 89.5%, at least 90%, at least 91%, at least 92%, at least 93%, at least 94%, at least 95%, at least 96%, at least 97%, at least 98%, at least 99%, or 100% identical to SEQ ID NO:112, SEQ ID NO:113, SEQ ID NO:114, or SEQ ID NO:115.

Fatty acid elongases utilized in some embodiments herein include, for example and without limitation, ELO1 and ELO2. Particular embodiments herein include a fatty acid elongase comprising an amino acid sequence that is at least at least 89.5%, at least 90%, at least 91%, at least 92%, at least 93%, at least 94%, at least 95%, at least 96%, at least 97%, at least 98%, at least 99%, or 100% identical to SEQ ID NO:126 (ELO1) or SEQ ID NO:127 (ELO2), preferably wherein the amino acid substitution(s) are conservative substitutions outside the substrate binding pocket. In specific embodiments, a fatty acid elongase comprises an amino acid sequence that is at least 95% or at least 98% identical to an amino acid sequence selected from SEQ ID NO:126 or SEQ ID NO:127.

Some embodiments herein utilize fatty acyl-CoA reductases, for example and without limitation, a L. botrana fatty acyl-CoA reductase (e.g., L. botrana FAR1, L. botrana FAR2, L. botrana FAR3, L. botrana FAR4, and L. botrana FAR5). As used herein, the term “L. botrana FAR1” refers to a functional fatty acyl-CoA reductase having at least 90% identity to SEQ ID NO:128. For example, a L. botrana FAR1 may comprise an amino acid sequence that is at least 89.5%, at least 90%, at least 91%, at least 92%, at least 93%, at least 94%, at least 95%, at least 96%, at least 97%, or at least 98% identical to SEQ ID NO:128. As used herein, the term “L. botrana FAR2” refers to a functional fatty acyl-CoA reductase having at least 90% identity to SEQ ID NO:129. For example, a L. botrana FAR2 may comprise an amino acid sequence that is at least 89.5%, at least 90%, at least 91%, at least 92%, at least 93%, at least 94%, at least 95%, at least 96%, at least 97%, or at least 98% identical to SEQ ID NO:129. As used herein, the term “L. botrana FAR3” refers to a functional fatty acyl-CoA reductase having at least 90% identity to SEQ ID NO:130. For example, a L. botrana FAR3 may comprise an amino acid sequence that is at least 89.5%, at least 90%, at least 91%, at least 92%, at least 93%, at least 94%, at least 95%, at least 96%, at least 97%, or at least 98% identical to SEQ ID NO:130. As used herein, the term “L. botrana FAR4” refers to a functional fatty acyl-CoA reductase having at least 90% identity to SEQ ID NO:131. For example, a L. botrana FAR4 may comprise an amino acid sequence that is at least 89.5%, at least 90%, at least 91%, at least 92%, at least 93%, at least 94%, at least 95%, at least 96%, at least 97%, or at least 98% identical to SEQ ID NO:131. As used herein, the term “L. botrana FAR5” refers to a functional fatty acyl-CoA reductase having at least 90% identity to SEQ ID NO:132. For example, a L. botrana FAR5 may comprise an amino acid sequence that is at least 89.5%, at least 90%, at least 91%, at least 92%, at least 93%, at least 94%, at least 95%, at least 96%, at least 97%, or at least 98% identical to SEQ ID NO:132.

Example nucleotide sequences corresponding to the desaturases, conjugases, fatty acyl-CoA oxidases, elongases, and fatty acyl-CoA reductases herein are provided as SEQ ID NOs:133-228, respectively. However, those in the art understand immediately that the redundancy of the genetic code defines a set of equivalent nucleotide sequences that may be used within the discretion of the practitioner to encode the useful biosynthetic components herein.

V. Microorganisms Comprising E7Z9-12CoA/E7Z9-12Ac Biosynthetic Pathway Components

Also disclosed herein are genetically modified microorganisms, comprising at least one exogenous component of E7Z9-12CoA/E7Z9-12Ac biosynthetic pathways. In particular embodiments, such a genetically modified microorganism may comprise at least one exogenous component of a E7Z9-12CoA biosynthetic pathway selected from the group consisting of a DST299, a DST109 (e.g., DST109 V230 and DST109 V230A), a DST014, a DST499, a DST024, a DST176, a DST177, a DST178, a DST192 (e.g., DST192 G100 and DST192 G100L), a DST043, a DST500, a RnACOX1, an AtACX1, a PxACX1, a RnACOX2, a Y. lipolytica POX2, an AtACX2, a PxACX3, a BaACX3, an LbPOX1, an LbPOX2, an LbPOX3, an LbPOX4, an LbPOX5, an LbPOX6, an LbFAR1, an LbFAR2, an LbFAR3, an LbFAR4, and an LbFAR5. In particular embodiments, a genetically modified microorganism comprises at least one desaturase selected from the group consisting of a DST299, a DST499, and a DST109 V230A, a DST500 conjugase, and/or at least one fatty acyl-CoA oxidase selected from the group consisting of an LbPOX5, a BaACX3, a PxACX1, and a PxACX3.

In specific embodiments, the genetically modified microorganism comprises at least one exogenous desaturase or conjugase and at least one POX. Specific embodiments include genetically modified microorganisms comprising a DST299 and at least one POX; for example, a genetically modified microorganism comprising a DST299, a DST500, and at least one of a LbPOX5, a BaACX3, a PxACX1, and a PxACX3, a genetically modified microorganism comprising a DST014, a DST299, and at least one of a LbPOX5, a BaACX3, a PxACX1, and a PxACX3, a genetically modified microorganism comprising a DST299, a DST109 V230A, and one of a LbPOX5, a BaACX3, a PxACX1, and a PxACX3, a genetically modified microorganism comprising a DST500 and at least one of a LbPOX5, a BaACX3, a PxACX1, and a PxACX3, and a genetically modified microorganism comprising a DST499 and at least one of a LbPOX5, a BaACX3, a PxACX1, and a PxACX3.

Particular embodiments herein utilize the foregoing genetically modified microorganisms either alone or in combination with other microorganisms (e.g., other genetically modified micoorganisms); for example, to provide different desired catalytic activities in a E7Z9-12CoA/E7Z9-12Ac biosynthetic pathway. Intermediate products may accordingly be isolated (e.g., purified) from particular genetically modified microorganisms and provided as substrate to a further genetically modified microorganism to produce a next intermediate, or E7Z9-12CoA or E7Z9-12Ac. In some examples, a genetically modified microorganism comprising Z11-14 DST activity (e.g., a genetically modified microorganism comprising DST299) produces Z11-14CoA from a C14 substrate, which Z11-14CoA is then converted to E9Z11-14CoA by a genetically modified microorganism comprising E9-14 DST activity (e.g., a genetically modified microorganism comprising DST014, DST024, DST176, DST177, DST178, DST192 100G, DST192 G100L, and/or DST043) or conjugase activity (e.g., a genetically modified microorganism comprising DST500). In some examples, a genetically modified microorganism comprising E9-14 DST activity (e.g., a genetically modified microorganism comprising DST014, DST024, DST176, DST177, DST178, DST192 100G, DST192 G100L, and/or DST043) produces E9-14CoA from a C14 substrate, which E9-14CoA is then converted to E9Z11-14CoA by a genetically modified microorganism comprising Z11-14 DST activity (e.g., a genetically modified microorganism comprising DST299). In further examples, a genetically modified microorganism comprising E11-16 DST activity (e.g., a genetically modified microorganism comprising DST109 V230A) produces E11-16CoA from a C16 substrate, wherein E9-14CoA produced from the E11-16CoA is then converted to E9Z11-14CoA by a genetically modified microorganism comprising Z11-14 DST activity (e.g., a genetically modified microorganism comprising DST299). In each of the foregoing and in further examples, an intermediate product isolated from a genetically modified microorganism comprising exogenous DST and/or conjugase activity may be provided to a microorganism comprising acyl-CoA oxidase activity; for example, a genetically modified microorganism comprising LbPOX5, BaACX3, PxACX3, and/or PxACX1), or a microorganism comprising endogenous acyl-CoA oxidase activity, such as Y. lipolytica comprising endogenous POX2.

A genetically modified microorganism according to embodiments herein may be a yeast, bacterium, or insect cell. For example, the microorganism may be selected from the group consisting of Sacharomyces, Scizosacchromyces pombe, Pichia pastoris, Hansanuela polymorpha, Yarrowia lipolytica, Candida albicans, Candida tropicalis, Candida viswanathii, and Amyelois transilella. A genetically modified microorganism may be a microorganism that is suitable for large-scale culture in a bioreactor. In some embodiments herein, the genetically modified microorganism is Y. lipolytica (for example, Y. lipolytica strain H222 (Clib80)). In particular embodiments, a genetically modified microorganisms is provided in a culture.

In particular embodiments, a genetically modified microorganism expresses at least one component of a E7Z9-12CoA biosynthetic pathway from multiple copies of a coding polynucleotide. For example, a genetically modified microorganism may express a DST299 from a plurality of (e.g., four) copies of a DST299 sequence. It is known in the art that a coding polynucleotide may be codon-optimized for a particular host organism to improve expression of the encoded mRNA and translated polypeptide therefrom, for example, by substituting infrequently used codons in the genome of the host organism with more frequently used codons in the genome. It is further known that multiple copies of identical or nearly identical coding polynucleotides in a host organism frequently leads to inhibition or silencing of expression. Therefore, in specific embodiments herein wherein a genetically modified microorganism expresses at least one component of a E7Z9-12CoA biosynthetic pathway from multiple copies of a coding polynucleotide, the nucleotide sequences of the multiple copies may be varied within to the tolerance of the redundant genetic code to alleviate such inhibition according to the discretion of the ordinarily skilled artisan.

In some embodiments, a genetically modified microorganism further comprises at least one endogenous or exogenous nucleic acid molecule encoding an acyltransferase that preferably stores <C18 fatty acyl-CoA. In some embodiments, the acyltransferase is selected from the group consisting of glycerol-3-phosphate acyl transferase (GPAT), lysophosphatidic acid acyltransferase (LPAAT), glycerolphospholipid acyltransferase (GPLAT), and diacylglycerol acyltransferases (DGAT). In some embodiments, a genetically modified microorganism further comprises at least one endogenous or exogenous nucleic acid molecule encoding an acylglycerol lipase that preferably hydrolyzes ester bonds of >C16, of >C14, of >C12, or of >C10 acylglycerol substrates.

In some embodiments, a genetically modified microorganism comprises a deletion, disruption, insertion, mutation, and/or reduction in the activity of one or more endogenous enzymes that catalyzes a reaction in a pathway that competes with the biosynthesis pathway for the production of a mono- or poly-unsaturated <Cis fatty alcohol. In further embodiments, the genetically modified microorganism comprises a deletion, disruption, mutation, and/or reduction in the activity of one or more endogenous enzyme selected from: (i) one or more acyl-CoA oxidase; (ii) one or more acyltransferase; (iii) one or more acylglycerol lipase and/or sterol ester esterase; (iv) one or more (fatty) alcohol dehydrogenase; (v) one or more (fatty) alcohol oxidase; and (vi) one or more cytochrome P450 monooxygenase.

In particular embodiments, one or more genes of the microbial host encoding acyl-CoA oxidases are deleted or down-regulated to eliminate or reduce the truncation of desired fatty acyl-CoAs beyond a desired chain-length. In some embodiments, the recombinant microorganism comprises a deletion, disruption, mutation, and/or reduction in the activity of one or more endogenous acyl-CoA oxidase enzyme selected from the group consisting of Y. lipolytica POX1 (YALI0E32835g); Y. lipolytica POX2 (YALI0F10857g); Y. lipolytica POX3 (YALI0D24750g); Y. lipolytica POX4 (YALI0E27654g); Y. lipolytica POX5 (YALI0C23859g); Y. lipolytica POX6 (YALI0E06567g); S. cerevisiae POX1 (YGL205W); Candida POX2 (Ca019.1655, Ca019.9224, CTRG_02374, and M18259); Candida POX4 (Ca019.1652, Ca019.9221, CTRG Q2377, and M12160); and Candida POX5 (Ca019.5723, Ca019.13146, CTRG_02721, and M12161).

In some embodiments, a genetically modified microorganism capable of producing a mono- or poly-unsaturated <Cis fatty alcohol, fatty aldehyde, and/or fatty acetate from an endogenous or exogenous source of saturated C6-C24 fatty acid is provided, wherein the recombinant microorganism expresses one or more acyl-CoA oxidase enzymes, and wherein the recombinant microorganism is manipulated to delete, disrupt, mutate, and/or reduce the activity of one or more endogenous acyl-CoA oxidase enzymes. In some embodiments, the one or more acyl-CoA oxidase enzymes being expressed are different from the one or more endogenous acyl-CoA oxidase enzymes being deleted or downregulated. In other embodiments, the one or more acyl-CoA oxidase enzymes that are expressed regulate chain length of the mono- or poly-unsaturated <Cis fatty alcohol, fatty aldehyde and/or fatty acetate.

In some embodiments, a genetically modified microorganism comprises a deletion, disruption, mutation, and/or reduction in the activity of one or more endogenous acyltransferase enzyme selected from the group consisting of Y. lipolytica YALI0000209g, Y. lipolytica YAL10E18964g, Y. lipolytica YALI0F19514g, Y. lipolytica YAL10C 14014g, Y. lipolytica YALI0E16797g, Y. lipolytica YALI0E32769g, Y. lipolytica YALI0D07986g, S. cerevisiae YBLO11w, S. cerevisiae YDL052c, S. cerevisiae YOR175C, S. cerevisiae YPR139C, S. cerevisiae YNR008w, S. cerevisiae YGR245c, Candida 1503_02577, Candida CTRG_02630, Candida Ca019.250, Candida Ca019.7881, Candida CTRG_02437, Candida Ca019.1881, Candida Ca019.9437, Candida CTRG_01687, Candida Ca019.1043, Candida Ca019.8645, Candida CTRG0475Q, Candida Ca019.13439, Candida CTRG_04390, Candida Ca019.6941, Candida CaO19.14203, and Candida CTRG_06209.

In some embodiments, a genetically modified microorganism capable of producing a mono- or poly-unsaturated ≤Cis fatty alcohol, fatty aldehyde and/or fatty acetate from an endogenous or exogenous source of saturated C6-C24 fatty acid is provided, wherein the genetically modified microorganism expresses one or more acyltransferase enzymes, and wherein the genetically modified microorganism is manipulated to delete, disrupt, mutate, and/or reduce the activity of one or more endogenous acyltransferase enzymes. In particular embodiments, one or more genes of the microbial host encoding GPATs, LPAATs, GPLATs, and/or DGATs are deleted or downregulated, and replaced with one or more GPATs, LPAATs, GPLATs, or DGATs that prefer to store short-chain fatty acyl-CoAs. In some embodiments, the one or more acyltransferase enzymes being expressed are different from the one or more endogenous acyltransferase enzymes being deleted or downregulated.

In some embodiments, one or more genes of the microbial host encoding acylglycerol lipases (mono-, di-, or triacylglycerol lipases) and/or sterol ester esterases are deleted or downregulated and replaced with one or more acylglycerol lipases that prefer long chain acylglycerol substrates. In some embodiments, the genetically modified microorganism comprises a deletion, disruption, mutation, and/or reduction in the activity of one or more endogenous acylglycerol lipase and/or sterol ester esterase enzyme selected from the group consisting of Y. lipolytica YAL10E32035g, Y. lipolytica YAL10D17534g, Y. lipolytica YAL10F10010g, Y. lipolytica YALIOC14520g, Y. lipolytica YALIOE00528g, S. cerevisiae YKL140w, S. cerevisiae YMR313c, S. cerevisiae YKR089c, S. cerevisiae YOR081C, S. cerevisiae YKL094W, S. cerevisiae YLL012W, S. cerevisiae YLR020C, Candida Ca019.2050, Candida Ca019.9598, Candida CTRG_01138, Candida W5Q_03398, Candida CTRG_00057, Candida Ca019.5426, Candida Ca019.12881, Candida CTRG_06185, Candida Ca019.4864, Candida Ca019.12328, Candida CTRG_03360, Candida Ca019.6501, Candida Ca019.13854, Candida CTRG_05049, Candida Ca019.1887, Candida Ca019.9443, Candida CTRG_01683, and Candida CTRG04630.

In some embodiments, the genetically modified microorganism comprises a deletion, disruption, mutation, and/or reduction in the activity of one or more endogenous cytochrome P450 monooxygenases selected from the group consisting of Y. lipolytica YALI0E25982g (ALK1), Y. lipolytica YALI0F01320g (ALK2), Y. lipolytica YALI0E23474g (ALK3), Y. lipolytica YALI0B.13816g (ALK4), Y. lipolytica YALI0B13838g (ALK5), Y. lipolytica YALI0B01848g (ALK6), Y. lipolytica YALI0AI 5488g (ALK7), Y. lipolytica YALI0CI2122g (ALK8), Y. lipolytica YALI0B06248g (ALK9), Y. lipolytica YAU0B207G2g (ALK10), Y. lipolytica YALI0C10054g (ALK11), and Y. lipolytica YALI0A20130g (ALK12).

In some embodiments, a genetically modified microorganism capable of producing a mono- or poly-unsaturated ≤Cis fatty alcohol, fatty aldehyde and/or fatty acetate from an endogenous or exogenous source of saturated C6-C24 fatty acid is provided, wherein the genetically modified microorganism expresses one or more acylglycerol lipase and/or sterol ester esterase enzymes, and wherein the genetically modified microorganism is manipulated to delete, disrupt, mutate, and/or reduce the activity of one or more endogenous acylglycerol lipase and/or sterol ester esterase enzymes. In some embodiments, the one or more acylglycerol lipase and/or sterol ester esterase enzymes being expressed are different from the one or more endogenous acylglycerol lipase and/or sterol ester esterase enzymes being deleted or downregulated. In some embodiments, the one or more endogenous or exogenous acylglycerol lipase and/or sterol ester esterase enzymes being expressed prefer to hydrolyze ester bonds of long-chain acylglycerols.

In some embodiments, the fatty acyl desaturase catalyzes the conversion of a fatty acyl-CoA into a mono- or poly-unsaturated intermediate selected from E5-10:Acyl-CoA, E7-12:Acyl-CoA, E9-14:Acyl-CoA, E11-16:Acyl-CoA, E13-18:Acyl-CoA, Z7-12:Acyl-CoA, Z9-14:Acyl-CoA, Z11-16:Acyl-CoA, Z13-18:Acyl-CoA, Z8-12:Acyl-CoA, Z10-14:Acyl-CoA, Z12-16:Acyl-CoA, Z14-18:Acyl-CoA, Z7-10:Acyl-coA, Z9-12:Acyl-CoA, Z11-14:Acyl-CoA, Z13-16:Acyl-CoA, Z15-18:Acyl-CoA, E7-10:Acyl-CoA, E9-12:Acyl-CoA, E11-14:Acyl-CoA, E13-16:Acyl-CoA, E15-18:Acyl-CoA, E5Z7-12:Acyl-CoA, E7Z9-12:Acyl-CoA, E9Z11-14:Acyl-CoA, E11Z13-16:Acyl-CoA, E13Z15-18:Acyl-CoA, E6E8-10:Acyl-CoA, E8E10-12:Acyl-CoA, E10E12-14:Acyl-CoA, E12E14-16:Acyl-CoA, Z5E8-10:Acyl-CoA, Z7E10-12:Acyl-CoA, Z9E12-14:Acyl-CoA, Z11E14-16:Acyl-CoA, Z13E16-18:Acyl-CoA, Z3-10:Acyl-CoA, Z5-12:Acyl-CoA, Z7-14:Acyl-CoA, Z9-16:Acyl-CoA, Z11-18:Acyl-CoA, Z3Z5-10:Acyl-CoA, Z5Z7-12:Acyl-CoA, Z7Z9-14:Acyl-CoA, Z9Z11-16:Acyl-CoA, Z11Z13-16:Acyl-CoA, and Z13Z15-18:Acyl-CoA. In further embodiments, the mono- or poly-unsaturated ≤Cis fatty alcohol is selected from the group consisting of E5-10:OH, Z8-12:OH, Z9-12:OH, Z11-14:OH, Z11-16OH, E11-14:OH, E8E10-12:OH, E7Z9-12OH, Z11Z13-16OH, Z9-14:OH, Z9-16:OH, and Z13-18:0H.

In some embodiments, the genetically modified microorganism further comprises at least one endogenous or exogenous nucleic acid molecule encoding an aldehyde forming fatty acyl-CoA reductase capable of catalyzing the conversion of the mono- or poly-unsaturated ≤Cis fatty acid into a corresponding ≤Cis fatty aldehyde. In particular embodiments, the aldehyde forming fatty acyl-CoA reductase is selected from the group consisting of Acinetobacter calcoaceticus A0A1C4HN78, A. calcoaceticus N9DA85, A. calcoaceticus R8XW24, A. calcoaceticus A0A1A0GGM5, A. calcoaceticus A0A117N158, and Nostoc punctiforme YP_001865324. In some embodiments, the genetically modified microorganism further comprises at least one endogenous or exogenous nucleic acid molecule encoding an alcohol oxidase or an alcohol dehydrogenase capable of catalyzing the conversion of the mono- or poly-unsaturated ≤Cis fatty alcohol into a corresponding ≤Cis fatty aldehyde. In certain embodiments, the ≤Cis fatty aldehyde is selected from the group consisting of Z9-16:Ald, Z11-16:Ald, Z11Z13-16:Ald, and Z13-18:Ald.

In some embodiments, the genetically modified microorganism further comprises at least one endogenous or exogenous nucleic acid molecule encoding an enzyme selected from an alcohol oxidase, an alcohol dehydrogenase capable of catalyzing the conversion of the mono- or poly-unsaturated ≤Cis fatty alcohol into a corresponding ≤Cis fatty aldehyde, and at least one nucleic acid molecule encoding an endogenous or exogenous acetyl transferase capable of catalyzing the conversion of the mono- or poly-unsaturated ≤Cis fatty alcohol into a corresponding ≤Cis fatty acetate. In particular embodiments, the mono- or polyunsaturated ≤Cis fatty aldehyde or ≤Cis fatty acetate is selected from the group consisting of E5-10:Ac, Z7-12:Ac, Z8-12:Ac, Z9-12:Ac, E7Z9-12:Ac, Z9-14:Ac, Z9E1244:Ac, E11-14:Ac, Z11-14:Ac, Z11-16Ac, Z9-16:Ac, Z9-16:Ald, Z11-16:Ald, Z11Z13-16:Ald, and Z13-18:Ald.

VI. Methods for Biosynthesis of E7Z9-12CoA/E7Z9-12Ac

Also disclosed herein are biosynthetic methods for producing an insect pheromone or precursor thereof. Such methods may comprise, for example, culturing a genetically modified microorganism as herein described, and feeding the culture with a saturated or unsaturated substrate. In some embodiments, the culture is fed with glucose, wherein the microorganism as herein described synthesizes fatty acids de novo from the glucose. In some embodiments, the culture is fed with 12CoA, 14CoA, and/or 16CoA, wherein the microorganism as herein described synthesizes the insect pheromone and/precursors from the fatty acyl-CoA substrate. In specific examples, the culture is fed with 14CoA and/or 16CoA. In particular embodiments, the method further comprises isolating an insect pheromone or precursor thereof produced from the substrate. For example, the method may further comprise isolating E7Z9-12CoA from the culture. In specific examples, the method comprises isolating the pheromone or precursor via distillation. Alternatively, isolating the pheromone or precursor may comprise membrane-based separation. In particular embodiments, a pheromone precursor (e.g., E7Z9-12CoA) is isolated, and is then converted into an active pheromone (e.g., E7Z9-12Ac) via chemical methods.

For example, in some embodiments, a fatty alcohol produced in the microorganism is further chemically converted (e.g., in the microorganism or via chemical synthesis) to one or more corresponding fatty acetate esters. In particular embodiments, chemically converting the fatty alcohol to the corresponding fatty acetate esters comprises contacting the fatty alcohol with acetic anhydride. Accordingly, some embodiments herein include a fatty alcohol, fatty aldehyde, and/or fatty acetate produced from one or more unsaturated lipids, which lipids were synthesized in a genetically modified microorganism as herein described.

In particular embodiments herein, a method for producing an insect pheromone or precursor thereof does not utilize significant amounts of organic solvents, proceeds in one step, and results in high yield of a particular product isomer, providing a significant improvement upon conventional production methods.

Further details regarding microorganisms suitable for use in embodiments herein, methods for utilizing such microorganisms to produce chemicals in a biosynthetic reaction, and semi-biosynthetic methods including the use of such microorganisms may be found in PCT International Patent Publication No. WO 2018/213554 A1, U.S. Patent Publication No. 2019/0136272 A1, the contents of each of which are incorporated herein by this reference in their entirety.

The following EXAMPLES are provided to illustrate certain particular features and/or embodiments. The EXAMPLES should not be construed to limit the disclosure to the particular features or embodiments exemplified.

### EXAMPLES

### Example 1: Identification of Desaturases Involved in L. botrana Pheromone Biosynthesis

Lobesia botrana moths were dissected using instruments and dissection workspaces wiped with RNase AWAY solution (Fisher 10328011) and rinsed with deionized water to prevent sample degradation due to RNases. Dissections were performed on two populations: 1) approximately 1-day old control females reared in a 24-hour photophase chamber that are not expected to actively produce pheromone, and 2) approximately 1-day old sample females reared in a greenhouse under conditions conducive to pheromone production (exposed to 2 natural photoperiods and dissected after the first hour of scotophase). Samples were chosen based on the premise that pheromone production initiates/increases with successive scotophases, and that day-old moths that have not experienced a scotophase will produce lower quantities of pheromone. After analyzing sequencing data, however, we determined that rearing conditions did not significantly change the type or read number of sequences recovered.

To dissect gland tissue, individual females were removed from their group enclosure and “knocked out” with CO₂. The pheromone gland was forced out by squeezing the abdomen with forceps until the intersegmental membrane and associated tissues protruded out of the tip of the abdomen. Fine-tipped forceps and dissection scissors were then used to detach the sclerotized cuticle at the base of the pheromone gland from the 8th abdominal segment. Tissues were harvested and immediately stored in approximately 100 μL RNAlater (Fisher AM7020) solution cooled on ice. Approximately 2 mg each sample was collected from ˜140 moths over a period of 4 hours per sample (control legs and sample glands were collected simultaneously). Samples were briefly spun in a microcentrifuge at 5000×g to ensure submersion of tissue in RNAlater, and stored at −15° C. Samples were then subjected to RNA extraction, cDNA generation, and high-throughput sequencing.

The sequencing results consisted of a pair of FASTQ files for each submitted sample. The pair of files consists of sequencing data read either from the left/5′ (R1) or right/3′ (R2) end of a DNA fragment. A common practice used to facilitate functional description of transcriptomes in organisms lacking a fully sequenced and annotated genome is to map the reads onto the genome of a related organism. However, the reads were not able to be mapped onto the genome of the related organism, Plutella xylostella. Only a few mitochondrial or central metabolism genes (e.g., ATP synthase subunits, NADH dehydrogenase, cyclooxygenase) were captured with this method, indicating that de novo assembly and annotation was necessary.

Successful annotation was achieved through first assembling each pair of FASTQ files using TRINITY. The resulting FASTA file was translated into potential open reading frames using TransDecoder™ to generate a .pep file. Finally, we identified non-redundant nucleotide and amino acid sequences from our results as containing a fatty acid desaturase domain. Table 1.

TABLE 1
Desaturase sequences retrieved via L. botrana RNA-seq

| Polypeptide | Polynucleotide |
|---|---|
| SEQ ID NO:1 | SEQ ID NO:133 |
| SEQ ID NO:50 | SEQ ID NO:169 |
| SEQ ID NO:51 | SEQ ID NO:170 |
| SEQ ID NO:52 | SEQIDN0:171 |
| SEQ ID NO:53 | SEQ ID NO:172 |
| | SEQ ID NO:228 |
| SEQ ID NO:101 | SEQ ID NO:196 |
| SEQ ID NO:102 | SEQ ID NO:197 |
| SEQ ID NO:103 | SEQ ID NO:198 |
| SEQ ID NO:104 | SEQ ID NO:199 |
| SEQ ID NO:105 | SEQ ID NO:200 |
| SEQ ID NO:106 | SEQ ID NO:201 |
| SEQ ID NO:107 | SEQ ID NO:202 |
| SEQ ID NO:108 | SEQ ID NO:203 |
| SEQ ID NO:109 | SEQ ID NO:204 |
| SEQ ID NO:110 | SEQ ID NO:205 |

From the list of fatty acid desaturases, a single polypeptide determined to have a role in L. botrana sex pheromone biosynthesis, DST299 (SEQ ID NO:1), contained a novel 4-amino acid PPTQ (SEQ ID NO:77) motif. Two additional unique L. botrana sex pheromone biosynthetic polypeptides were identified with a 4-amino acid SPTQ (SEQ ID NO:78) motif, DST499 (SEQ ID NO:2) and DST500 (SEQ ID NO:111).

In order to identify other components of the L. botrana pheromone biosynthetic pathway, the whole L. botrana genome was sequenced. Predictive intron splicing and fragment assembly identified a set of further full-length unique L. botrana desaturase sequences (SEQ ID NOs:54-76) in addition to DST299, DST499, and DST500. Representative L. botrana desaturases that were discovered are set forth in Table 2.

TABLE 2
L. botrana desaturases:

| Desaturase | Amino Acid | Nucleotide |
|---|---|---|
| Name | Sequence | Sequence |
| DST299 | SEQ ID NO: 1 | SEQ ID NO: 133 |
| DST499 | SEQ ID NO:2 | SEQ ID NO: 134 |
| DST500 | SEQ ID NO:111 | SEQ ID NO:206 |
| NPVE | SEQ ID NO:54 | SEQ ID NO: 173 |
| KPSEl | SEQ ID NO:55 | SEQ ID NO: 174 |
| LPGQ | SEQ ID NO:56 | SEQ ID NO: 175 |
| RAVE2 | SEQ ID NO:57 | SEQ ID NO: 176 |
| KPAE | SEQ ID NO:58 | SEQ ID NO: 177 |
| RPTQ2 | SEQ ID NO:59 | SEQ ID NO: 178 |
| GATD1 | SEQ ID NO:60 | SEQ ID NO: 179 |
| GATD2 | SEQ ID NO:61 | SEQ ID NO: 180 |
| KPSE2 | SEQ ID NO:62 | SEQ ID NO: 181 |
| NPRE | SEQ ID NO:63 | SEQ ID NO: 182 |
| QPVE | SEQ ID NO:64 | SEQ ID NO: 183 |
| QSRQ | SEQ ID NO:65 | SEQ ID NO: 184 |
| PPTQI | SEQ ID NO:66 | SEQ ID NO: 185 |
| RATE | SEQ ID NO:67 | SEQ ID NO: 186 |
| RAVE | SEQ ID NO:68 | SEQ ID NO: 187 |
| RPTQ1 | SEQ ID NO:69 | SEQ ID NO: 188 |
| SATQ2 | SEQ ID NO:70 | SEQ ID NO: 189 |
| SATQ3 | SEQ ID NO:71 | SEQ ID NO: 190 |
| SPTQ3 | SEQ ID NO:72 | SEQ ID NO: 191 |
| TPSQI | SEQ ID NO:73 | SEQ ID NO: 192 |
| TPSQ2 | SEQ ID NO:74 | SEQ ID NO: 193 |
| TPVE | SEQ ID NO:75 | SEQ ID NO: 194 |
| TPVE2 | SEQ ID NO:76 | SEQ ID NO: 195 |

Some of the desaturases were found to contain a 4-amino acid XXXQ motif that is characteristic of Δ10,11 desaturases. Knipple et al. (2002); Matoušková et al. (2007); Serra et al. (2007). For example, in sequences DST299, this motif is PPTQ (SEQ ID NO:77). In SEQ ID NOs:2, 72, and 111, the motif is SPTQ. NPVE (SEQ ID NO:54) was identified as a likely Z9 desaturase based on BLAST database searches. The desaturase of SEQ ID NO:53 did not capture the region containing the signature motif

Of the desaturases in Table 2, for example, DST299 was determined to have Z11-14 desaturase activity, DST499 was determined to have Z11-16 desaturase activity, DST500 was determined to have broad desaturase and conjugase activity, KPAE was determined to have Z5-14 desaturase activity, RPTQ2 was determined to have Z5-14 desaturase activity, KPSE1 was determined to have Z9-16/14 desaturase activity, NPVE was determined to have Z9-18 desaturase activity, DST499 was determined to have Z11-16 desaturase activity, and LPGQ was determined to have Z11-18/16 desaturase activity.

Also identified from the whole genome sequencing of L. botrana were fatty acyl-CoA oxidases (SEQ ID NOs:112 and 120-124) and fatty acyl-CoA reductases (SEQ ID NOs:128-132). Of the L. botrana fatty acyl-CoA oxidases, LbPOX5 was found to give the highest product yields when oxidizing 16CoA and 14CoA substrates to 14CoA and 12CoA product, respectively.

Fermentation Bioconversion

E9-14Acid was fed to a Y. lipolytica strain expressing DST299 from five chromosomal copies (H222 ΔP ΔΔ ΔF, Δxpr2::pTEF-(SEQ ID NO:133)-tXPR2, Mao1::pTEF (SEQ ID NO:133)-tXPR2, Δtg13::pTEF-(SEQ ID NO:133)-tXPR2, Δpox5::pTEF-(SEQ ID NO:133)-tXPR2, Δfat1::pIEF-(SEQ ID NO:133)-tXPR2-URA3). Positive transformants (N=4 clones per strain) were inoculated into 1 mL YPD in a 24-well culture plate with 3-mL glass vial inserts (Fisher Vial 06446C Serum Tubing 3-mL) and incubated for 24 hours in the Infors HT Mulitron Pro at 28° C. with 1000 rpm shaking. Cells were pelleted at 800×g, and YPD was decanted by pipetting. Cells were resuspended in warm bioconversion media (FERMI: 1 g/L Yeast Extract; 9.83 g/L KH₂PO₄; 6.34 g/L K₂HPO₄-3H₂O; 1.7 g/L YNB w/o aa, NH₄; 120 g/L glucose; 5 g/L glycerol (3.97 mL), 3.3 g/L ammonium sulfate, 8.54 mg/L iron sulfate (added from frozen 1000× stock solution), and incubated for an additional 6 hours in the Infors HT Multitron under the same conditions. After 6 hours, 25.5 μL methyl myristate (˜22 g/L) was added to the cultures, and the plate was returned to the incubator. Then, 250 μL culture was sampled into glass crimp top vials after 72 hours of bioconversion, and the samples were subjected to intracellular lipid analysis using base methanolysis.

Sample Processing and GC Analyses

Intracellular lipid was extracted using base methanolysis according to the following protocol: Cell culture was aliquoted into 2-mL GC vials, frozen at −80° C., and lyophilized overnight. Methanol (0.5 mL) containing the internal standard 15ME (1 mg/L) was added into the vials containing the dry cell pellet. Next, 10N KOH (29 μL) was added, mixed thoroughly (Mixmate, 2000 rpm, 10 minutes), and heated to 60° C. using a convection oven (40 minutes). After heating, the vials were cooled down to room temperature, and 2 equivalents of 24N sulfuric acid (35 μL) was added. The vials were shaken to ensure thorough mixing (Mixmate, 2000 rpm, 10 minutes), and then heated in a convection oven at 60° C. (40 minutes) to esterify the hydrolyzed metabolites. Extraction of the final metabolites in methyl ester form was carried out using hexane (1 mL).

An aliquot (60 μL) of the hexane layer was used for GC-FID analysis. The remaining hexane layer was placed in a vial and solvent was removed. The final oil sample was redissolved in 250 μL hexanes. A 30-μL aliquot of the concentrated sample was diluted with 30 hexane, and analyzed using GC-MS. The spiked sample was prepared by diluting 30 μL concentrated sample with 30 μL (E,Z)-9,11-methyl tetradecadienoate (E9Z11-14ME) standard solution in hexane. The E9Z11-14ME standard solution was prepared by esterifying 4.2 mg E9Z11-14Acid in methanol (0.5 mL) containing 15ME (1 mg/mL) in the presence of a catalytic amount of 24N sulfuric acid (29 μL) at 50° C. for 30 minutes. The resulting methyl ester was extracted with hexane (1 mL). Additional experiments to confirm the regiochemistry of the enzymatic product (E9Z11-14ME) were performed using 4-methyl-1,2,4-triazoline-3,5-dione (MTAD).

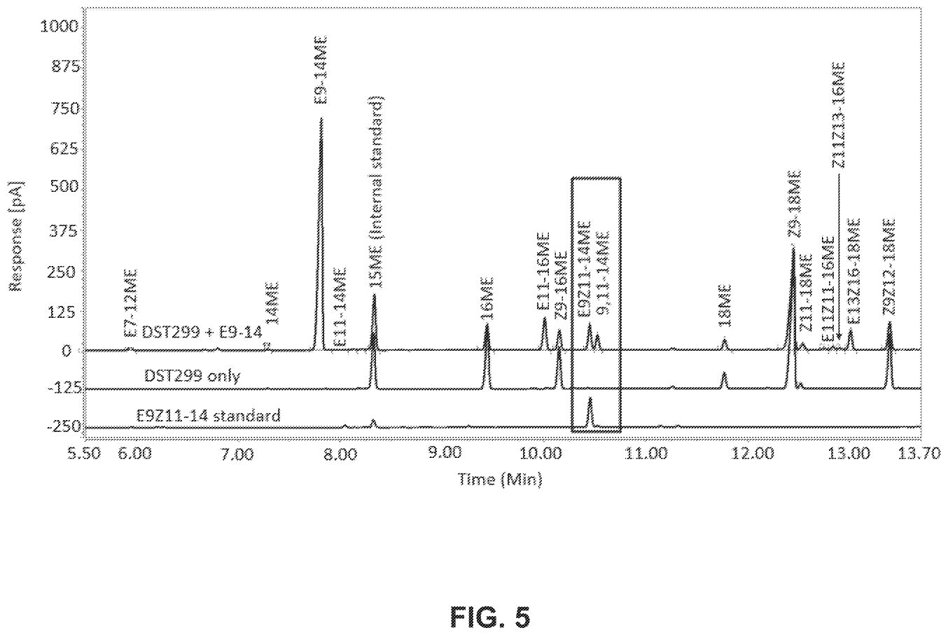

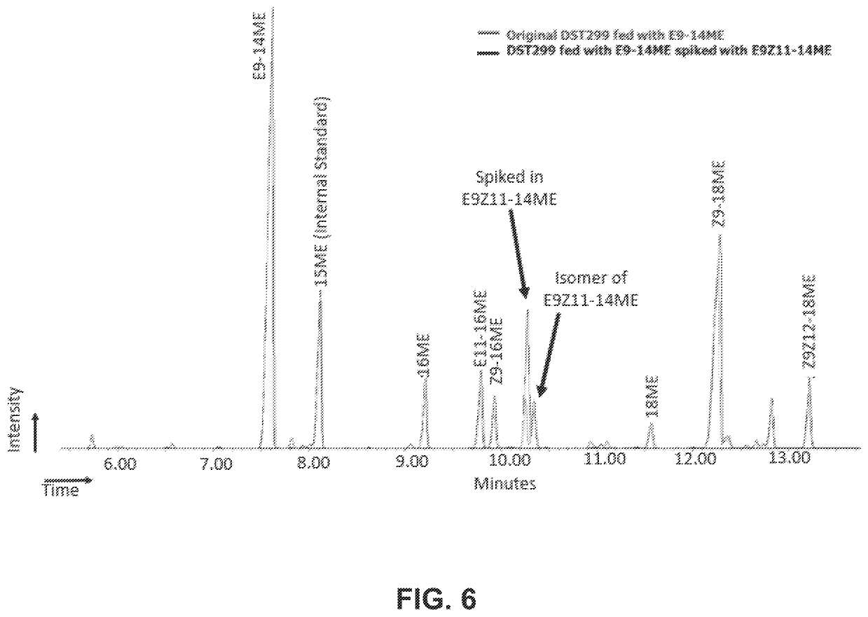

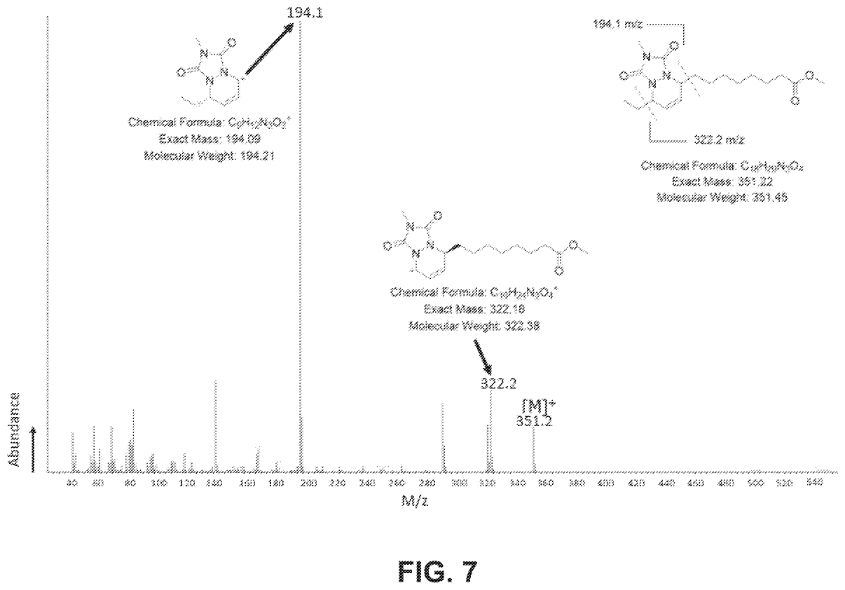

Comparing the DST299 GC chromatograms with and without the addition of substrate (E)-9-tetradecanoic acid (E9-14Acid) indicates that DST299 consumed E9-14Acid to produce E9Z11-14ME; the enzymatic product co-elutes with the authentic standard E9Z11-14ME. FIG. 5. DST299 produces approximately 330 mg/L E9Z11-14ME when fed E9-14. A spiked GC-MS study also confirmed the production of E9Z11-14ME. FIG. 6 (strain description provided below). To further verify the regiochemistry at the C9- and C11- position of the conjugated diene, the enzymatic product was derivatized with 4-methyl-1,2,4-triazoline-3,5-dione (MTAD). FIG. 7. The predicted mass fragmentation of the MTAD adduct matches with the experimental data, proving that the diene is conjugated at the C9- and C11- position of a C14 methyl ester.

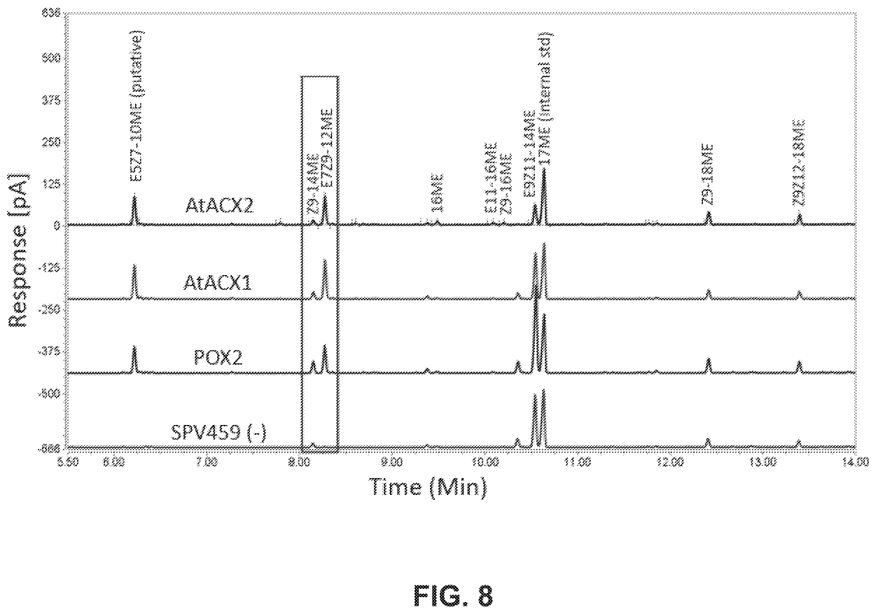

To access E7Z9-12CoA, we screened 5 acyl-CoA oxidases to select for variants capable of truncating E9Z11-14CoA to E7Z9-12CoA. FIGS. 8-10. Y. lipolytica harboring POX variants strains were: SPV0745 contained DST109 (SEQ ID NO:5) and POX2 (SEQ ID NO:125) [H222 ΔP ΔΔ ΔF, Δxpr2::pTEF-[Z11-16_DST]-tXPR2-URA3, SEQ ID NO:137]; SPV2452 and SPV2453 contained AtACX1 (SEQ ID NO:118) [H222 ΔP ΔΔ ΔF, pox5::pTEF-(SEQ ID NO:215)-tXPR2-URA3]; SPV2454 and SPV2455 contained AtACX2 (SEQ ID NO:120) [H222 ΔP ΔΔ ΔF, pox5::pTEF-(SEQ ID NO:213)-tXPR2-URA3]; SPV2424 and SPV2425 contained RnACOX1 (SEQ ID NO:116) [H222 ΔP ΔΔ ΔF, pox5::pTEF-(SEQ ID NO:211)-tXPR2-URA3]; and SPV2426 and SPV2427 contained RnACOX2 (SEQ ID NO:117) [H222 ΔP ΔΔ ΔF, pox5::pTEF-(SEQ ID NO:212)].

Strains were grown in YPD for 24 hours then switched to bioconversion media (BOX4: 2 g/L Yeast Extract; 1 g/L peptone; 6.34 g/L K₂HPO₄-3H₂O); 1.7 g/L YNP w/o aa, NH₄; 60 g/L glucose) for 48 hours, after which strains were collected for intracellular lipid analysis.

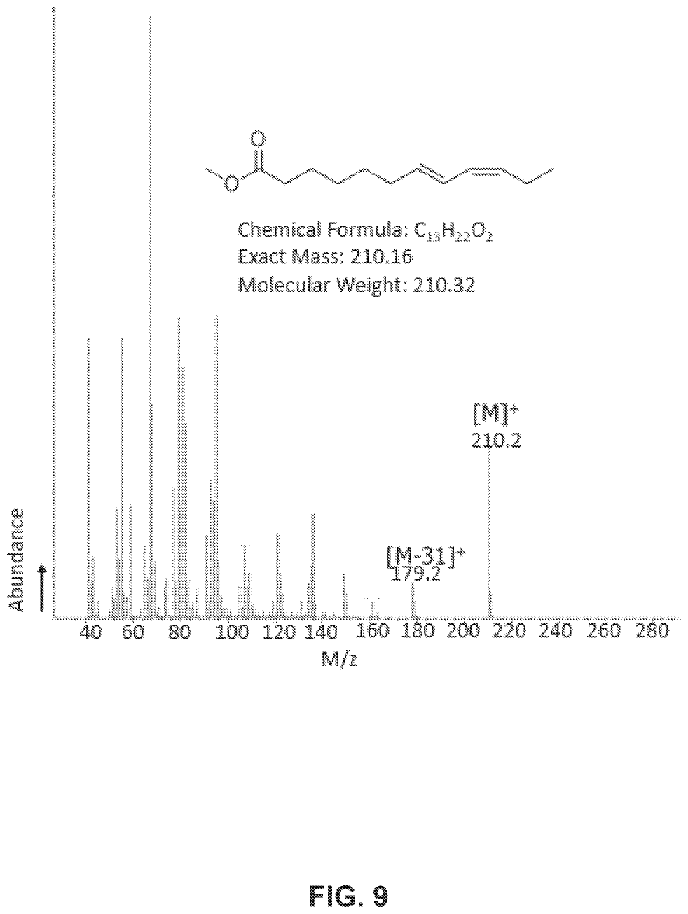

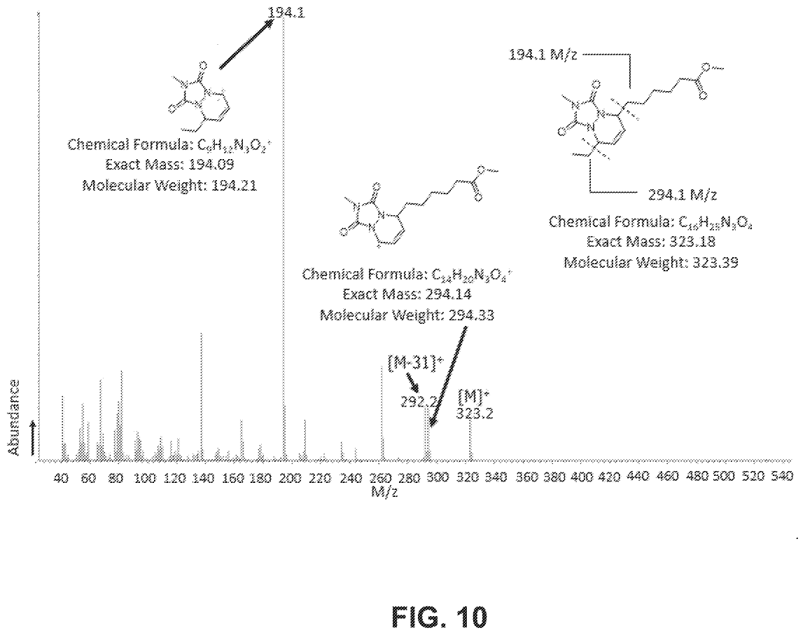

Intracellular lipid was extracted using base methanolysis with 17ME as the internal standard (see description above for detailed experimental procedure). Similar GC-FID (FIG. 8) and GC-MS (FIG. 9 and FIG. 10) studies were performed to detect and confirm the formation of E7Z12-12CoA via POX activity on E9Z11-14CoA. Using GC-FID analysis, we measured the E7Z9-12ME titer to be ˜570 mg/mL when E9Z11-14Acid was fed to a strain harboring POX activity. Mass fragmentation patterns (FIG. 9) were derived from product characterization of biological samples. Similar to the confirmation of E9Z11-14ME, derivatization of the sample with 4-methyl-1,2,4-triazoline-3,5-dione (MTAD) was performed to confirm the regiochemistry of the conjugated diene at positions C7 and C9. The predicted mass fragmentation of the MTAD adduct matches the experimental data, proving that the C12 diene is conjugated at positions C7 and C9. FIG. 10.

### Example 2: Engineering E9-14 and/or E11-16 Desaturase Activity

A comprehensive selection of publicly available desaturases to identify enzymes with distinct regio-, stereo-, and chain length specificities were screened. Upon characterizing the available enzymes, desired desaturase activities were not available to produce the pheromone E7Z9-12Ac from simple saturated precursors. Thus, an enzyme with E9-14 or E11-16 DST activity was engineered. Structural information was compared with the activity data obtained from a library screen to identify selectivity determinants. A desaturase protein backbone (DST014) was selected and a homology model was created based on the protein structure from Z9-18 selective desaturases (Protein Data Bank ID: 4ymk). DST014 wildtype (SEQ ID NO:7) is a selective E11-14 DST with E11-14 titers approximately 30 mg/L under standard assay format (a feed of ˜10 g/L 14FAME in S2 media). With a RMSD of 0.15 angstroms between homology model and Z9-18 desaturase, DST014 could serve as exemplary protein backbone for protein engineering.

Mutational hot spots were highlighted in the homology model to determine distinct regions that guide regio-, stereo-, and chain length specificity. FIG. 11. One such example is the conserved residue Y64, which caps the substrate channel, and limits the accepted substrate chain length. This residue may be moved along the α-helix to modify the accepted substrate chain length of the respective desaturase. These mutations convert DST014 into an E11-16 DST. Further, the ADP binding pocket was engineered to “push” the substrate down the tunnel and thereby alter the regioselectivity. Mutations to this region result in E9-14 DST activity. Subsequently, site saturation mutagenesis libraries and point mutants (Table 3) were ordered from Genscript, transformed into base strain H222 ΔP ΔΔ ΔF ΔU (SPV0300) and screened for the desired activity using a panel of substrates, including 14FAME and 16FAME.

TABLE 3
DST014 variants. Amino acid substitutions
within conserved sequence patterns are
underlined. Alternative residues found
in DST enzymes with different stereo-,
regio-, or chain-length selectivities
are shown in Column 3.

| | Conserved | Observed | |
|---|---|---|---|
| | sequence | residues | |
| | pattern | in DST | |
| | in DST014 | sequences | |
| | (residue | with | |
| | of interest | differing | |
| Resi- | is | regio- | |
| due | underlined) | selectivity | Function |
| Y64 | SLLMY | E11 (N, H, Y), | Substrate |
| | (SEQ ID | Z11 (Y, F, C), | tunnel |
| | NO: 79) | Z13 (F), | modifi- |
| | | Z14 (S, I), | cation |
| | | Z9 (Y, H), | |
| | | Z6 (I) | |
| I68 | YELSI | E11 (I, I, E), | Substrate |
| | (SEQ ID | Z11 (E, I), | tunnel |
| | NO: 80) | Z13 (G), | modifi- |
| | | Z14 (T), | cation |
| | | Z9 (G, A), Z6 | |
| | | (I, V) | |
| N104 | MSFQN | E11 (N), | Substrate |
| | (SEQ ID | Z11 (N), | tunnel |
| | NO: 81) | Z13 (H), Z14 | modifi- |
| | | (H, N), | cation |
| | | Z9 (K, D), | |
| | | Z6 (S) | |
| H144 | WLMTH | E11 (H, K), | ADP |
| | (SEQ ID | Z11 (R, K), | binding |
| | NO: 82) | Z13 (R), | pocket/ |
| | | Z14 (S, K), | substrate |
| | | Z9 (K, R), | binding/ |
| | | Z6 (R) | regio- |
| | | | selectivity |
| K150 | SEEVK | E11 (K, I), | ADP |
| | (SEQ ID | Z11 (K, I), | binding |
| | NO: 83) | Z13 (V), | pocket/ |
| | | Z14 (K), | substrate |
| | | Z9 (K, I), | binding/ |
| | | Z6 (I) | regio- |
| | | | selectivity |
| L217 | LHATL | E11 (I, F), | Regio- |
| | (SEQ ID | Z11 (I, F), | selectivity |
| | NO: 84) | Z13 (F), | |
| | | Z14 (Y, W), | |
| | | Z9 (W), | |
| | | Z6 (F) | |
| C218 | HATLC | E11 (I, C), | Regio- |
| | (SEQ ID | Z11 (I, C), | selectivity |
| | NO: 85) | Z13 (M), | |
| | | Z14 (F, I), | |
| | | Z9 (I, S), | |
| | | Z6 (S) | |
| M247 | LNTAM | E11 (M, C, | Substrate |
| | (SEQ ID | T, A, V), | tunnel |
| | NO: 86) | Z11 (T, V, A), | modifi- |
| | | Z13 (S), | cation |
| | | Z14 (S), | |
| | | Z9 (T, A, C), | |
| | | Z6 (S) | |
| 171A | SILGI | E11 (I), | Substrate |
| | (SEQ ID | Z11 (V, I), | tunnel |
| | NO: 87) | Z13 (I), | modifi- |
| | | Z14 (A, I), | cation |
| | | Z9 (I), | |
| | | Z6 (I) | |
| I219A | YELSI | E11 (I, I, E), | Substrate |
| | (SEQ ID | Z11 (E, I), | tunnel |
| | NO: 88) | Z13 (G), | modifi- |
| | | Z14 (T), | cation |
| | | Z9 (G, A), | |
| | | Z6 (V, I) | |

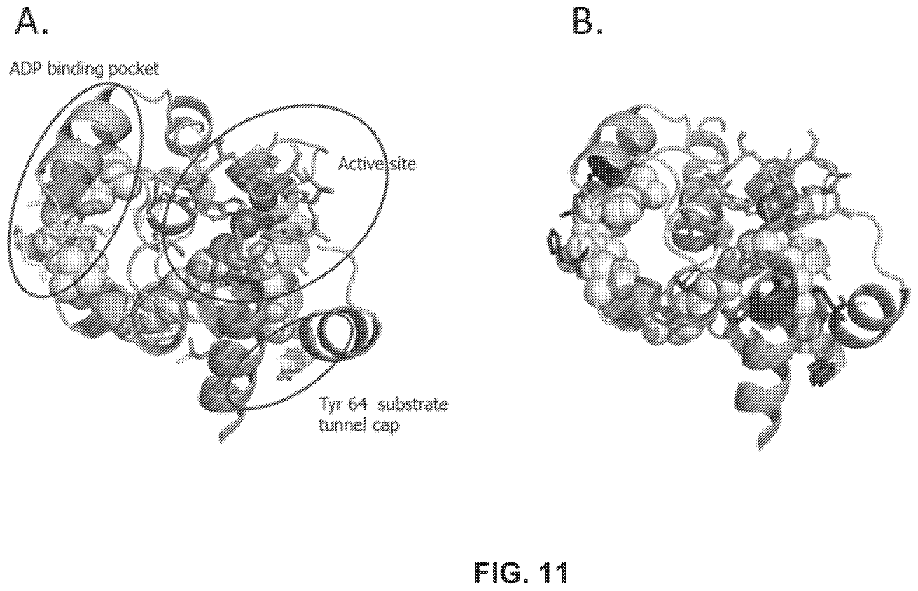

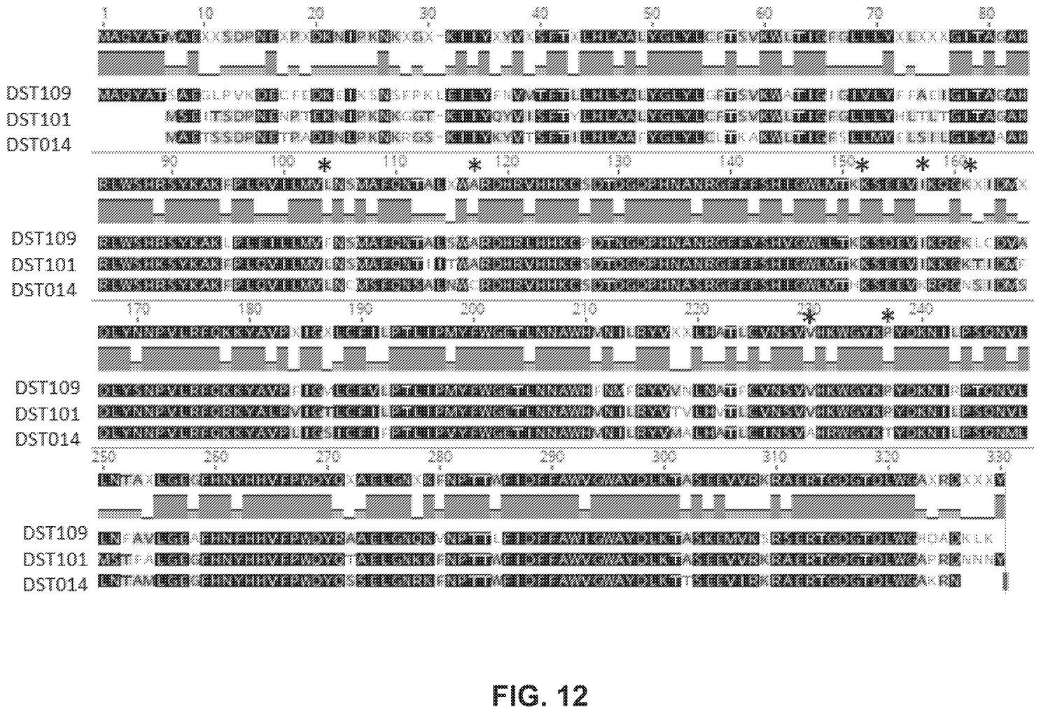

Several strategies were designed to engineer E11-16 and E9-14 DST activity on the DST109 scaffold. First, it was observed that the moth Ostrinia latipennis harbors at least three active DSTs with varied activities: DST109 (Z11-16 DST), DST101 (Z11-16 and Z11-14 DST) and DST014 (E11-14 DST). Strategies employed residue swapping and homology modeling (Swiss Model (Template—Protein Data Bank ID: 4YMK)) to alter the product profile of DST109. Residues were identified in DST109 (Z11-16 DST) and DST014 (E11-14 DST) that govern the product profile. FIG. 12. Point mutant DST109 V230A (SEQ ID NO:6) yielded slightly higher E11-16 titers versus wildtype (9 mg/L versus 6 mg/L). Four regions (1-4) (SEQ ID NOs:89-92) were also identified in DST109 that line the tail of the substrate binding pocket:

1.
(SEQ ID NO: 97; DST109 residues 74-82)
AEIGITAGA
2.
(SEQ ID NO: 98; DST109 residues 224-233)
FCVNSVVHKW
3.
(SEQ ID NO: 99; DST109 residues 250-259)
LNFAVLGEAF
4.
(SEQ ID NO: 100; DST109 residues 265-274)
VFPWDYRAAE

Alanine scanning mutagenesis was performed on residues predicted to line the enzyme's binding pocket. Two additional point mutants (DST109 R243A (SEQ ID NO:19), and DST109 F252A (SEQ ID NO:20)) were found with higher E11-16 titers than DST109 wildtype.

### Example 3: E9-14 FAME Synthesis by Metathesis

Preparation of Tris(hydroxymethyl)phosphine

A 250-mL three-necked round-bottomed flask was outfitted with a condenser containing a bubbler-sealed outlet, rubber septum with nitrogen inlet and magnetic stir bar. The vessel was charged with 43 mL (74.1-79.1 mmol) tetrakis(hydroxymethyl)phosphonium sulfate (1.72-1.84M). The flask was immersed in an oil bath. Methanol (25 mL) was added to the flask syringe through the rubber septum, which resulted in a cloudy, white solution. The contents were heated to a gentle reflux under a nitrogen atmosphere. Sodium hydroxide pellets (3.1 g, 76.5 mmol) were added to the flask over the course of 30 minutes, accompanied by the gradual addition of 40 mL methanol. The mixture was stirred for an additional 10 minutes, then cooled to room temperature with stirring. The precipitated sodium sulfate was removed by filtration and the THMP solution was stored under an inert atmosphere until needed.

Metathesis Catalyst Removal Procedure with THMP

50 molar equivalents of THMP per mole of ruthenium metathesis catalyst (M72; Umicore) was added to a metathesis reaction mixture. The mixture was stirred vigorously at 60-70° C. for 18-24 hours under nitrogen. The color of the reaction transitioned from dark brown to faint yellow or colorless after 18 hours. Nitrogen-degassed water (˜150 mL of water/L reaction mixture) was added, and the reaction was vigorously stirred for 10 minutes. Stirring was stopped and the phases separated. The bright orange aqueous phase was removed, 150 mL water again was added, and the solution was stirred vigorously for 10 minutes. Again, the phases separated, and the aqueous phase was removed. This procedure was repeated until the aqueous phase was colorless, which usually required 2 to 3 washings. The organic phase was washed with 50 mL 2.0 M HCl for 5 minutes (pH<1) and removed. The organic phase was then washed with 50 mL sodium bicarbonate-saturated water for 5 minutes (pH >7), removed, and further washed with brine. The reaction mixture was then ready for distillation.

Synthesis of E/Z-Dec-5-en-1-ol

Reagents 1-hexene (12.6 g, 150 mmol) and methyl 9-decenoate (9.2 g, 50 mmol) were charged to a 250-mL round bottom flask equipped with a stir bar and vacuum adapter. The reaction mixture was cooled to 5° C. while being degassing with argon. Umicore catalyst M72 (6.30 mg, 0.010 mmol, 200 ppm) was added to the reaction mixture, and a 4 Torr vacuum was applied to the reaction flask. The reaction proceeded at 5° C. until the reaction reached approximately 80% conversion, which took approximately 5 hours. The reaction mixture was treated with THMP using the procedure above prior to being purified by vacuum distillation. Potassium carbonate was added to the distillation pot. The olefinic bonds are stable to mildly basic environments but will isomerize in acidic environments during distillation.

### Example 4: DST/POX Microorganisms

E7Z9-12CoA was produced in fermentation reactions using a strain that harbors Z11-14 DST (DST299) and POX activities. FIG. 3. DST299 may desaturate E9-14CoA to form E9Z11-14CoA as shown above (FIGS. 5-7), and POX can chain-shorten E9Z11-14CoA to E7Z9-12CoA (FIGS. 8-10). However, side-products are expected to result in practice from off-target pathways, including but not limited to undesired desaturation reactions (e.g., E9E11-14CoA formation instead of E9Z11-14CoA formation) and chain-shortening of the substrate (i.e., E9-14CoA shortened to E7-12CoA).

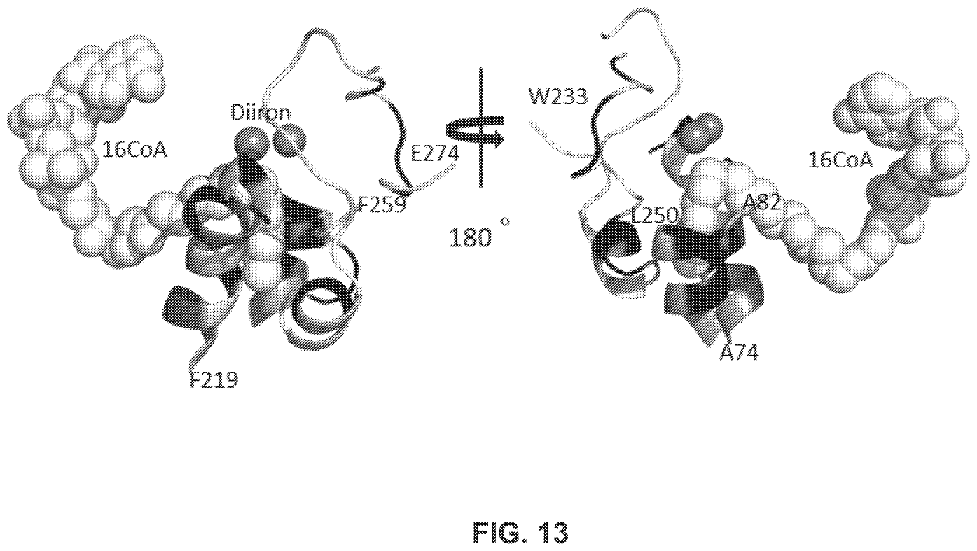

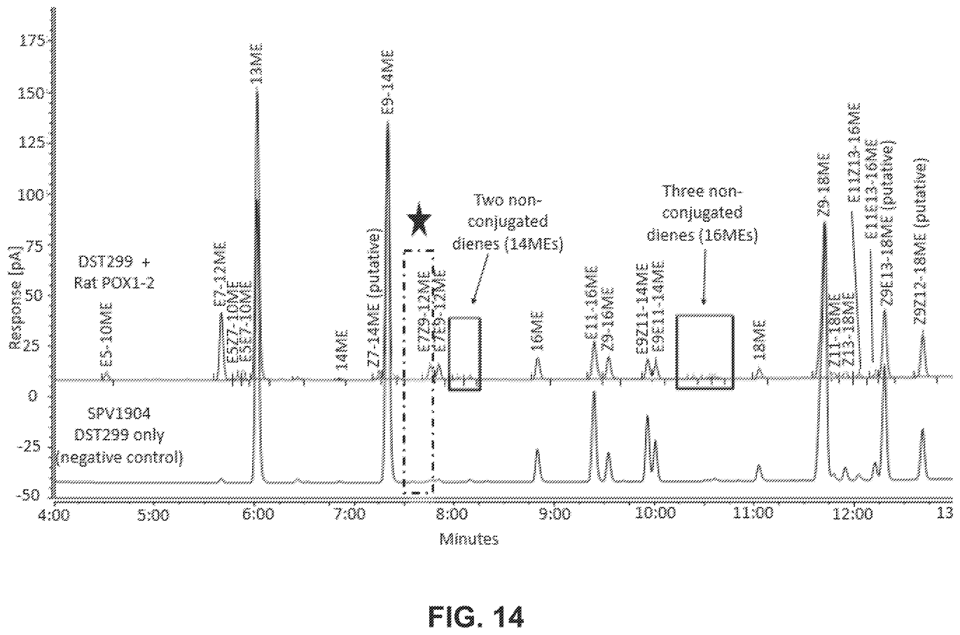

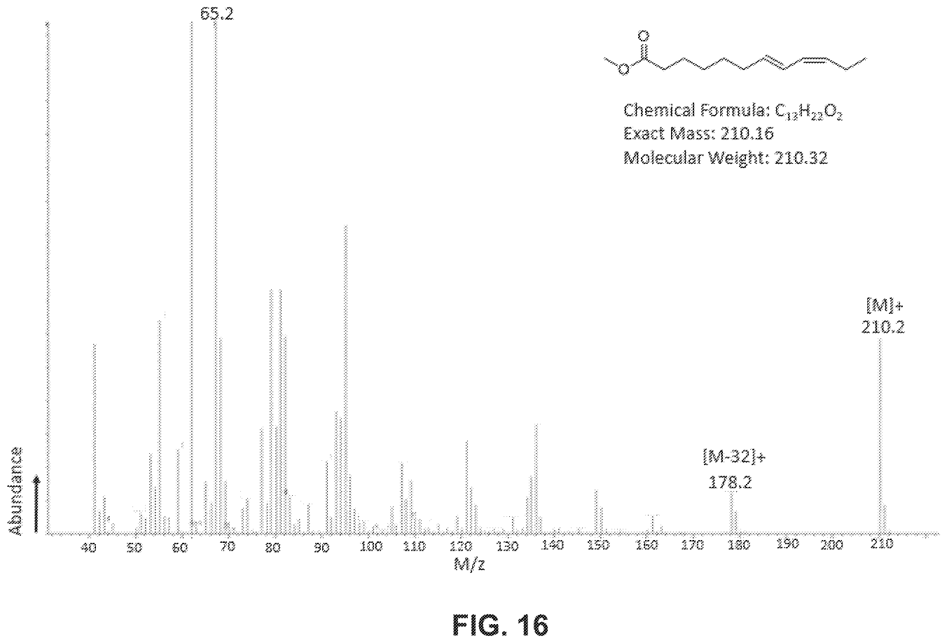

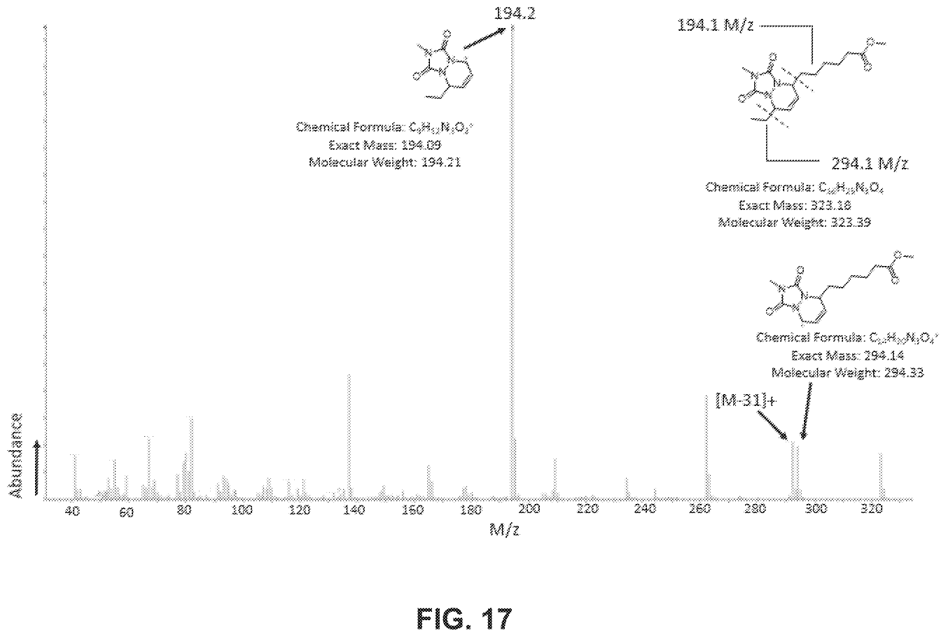

10.29±0.73 g/L E9-14ME was fed to Y. lipolytica containing DST299 (SEQ ID NO:1) and POX enzymes (SEQ ID NO:116 and SEQ ID NO:117), and a negative control Y. lipolytica strain containing DST299 without a POX enzyme. Three biological replicates of Test Strain (SPV2554, SPV2555 and SPV2557) and four technical replicates of negative control (SPV1904) were tested at 1-mL scale in 24-well plate format with 3-mL glass vial inserts (Fisher Vial 06446C Serum Tubing 3 mL). Strains were grown for 24 hours in YPD in an incubator shaker (1000 rpm, 28° C.), spun down at 1000 rpm for 5 minutes, resuspended in FERMI, and then allowed to incubate for six hours (1000 rpm, 28° C.) before the addition of substrate. Cultures were grown an additional 72 hours after the addition of substrate, after which 250 μL culture was sampled into glass crimp top vials and subjected to intracellular lipid analysis using base methanolysis as described previously. Under these conditions we surprisingly observed formation of E7Z9-12ME at 125±12 mg/L. FIGS. 14-17.

### Example 5: Biosynthesis of E7Z9-12CoA from C14 Substrate (Pathway 1)

Multiple pathways for the production of E7Z9-12 from 14C (FIG. 3) were engineered. In a first pathway, the E7Z9-12 product was obtained by functional co-expression of enzymes with E9-14 desaturase, Z11-14 desaturase, and acyl-CoA oxidase activities. FIG. 3.

E9-14 Intermediate Production

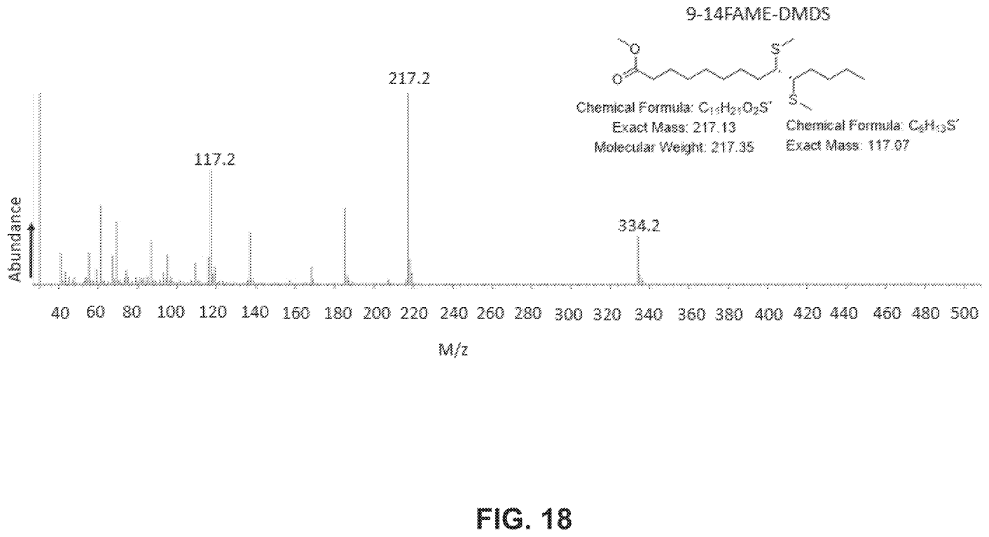

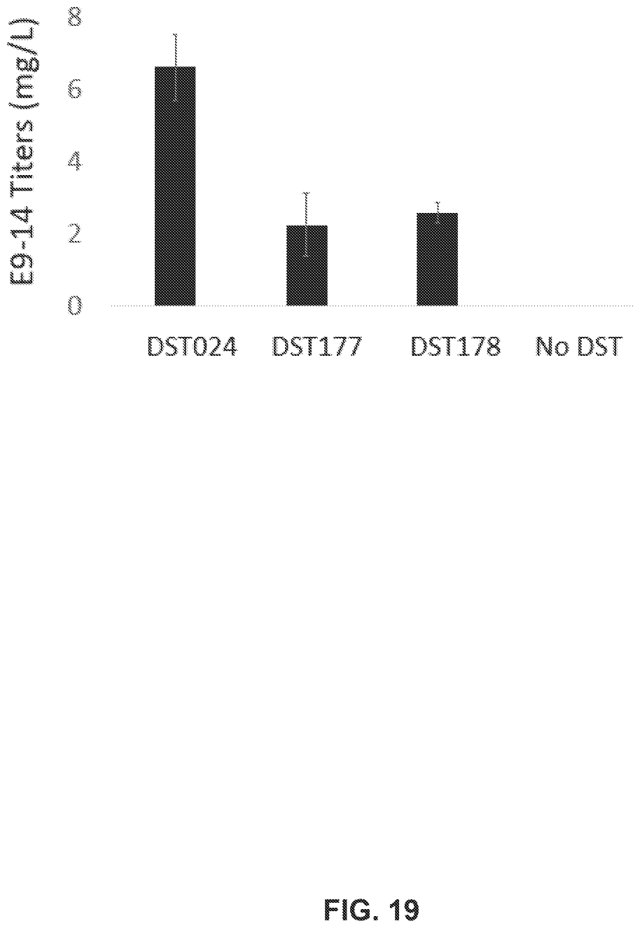

In one reaction step, an E9-14 DST produces E9-14CoA from a C14 substrate or produces E9Z11-14CoA from Z11-14CoA. E9-14 desaturase activity was provided by DST014, DST024, DST177, DST178, and DST192 G100L. Expression of DST014 (SEQ ID NO:7), DST024 (SEQ ID NO:8), DST177 (SEQ ID NO:10), DST178 (SEQ ID NO:11), and DST192 G100L (SEQ ID NO:4) in Y. lipolytica confirmed E9-14 DST activity for these enzymes through conversion of C14 to E9-14. GC-MS fragmentation data provided DMDS evidence of E9-14 production in Y. lipolytica strains expressing DST192 G100L. FIG. 18. High titers of E9-14 were produced by strains expressing each of DST014 (not shown), DST024, DST177, and DST178. FIG. 19.

Engineered E9-14 DST Activity

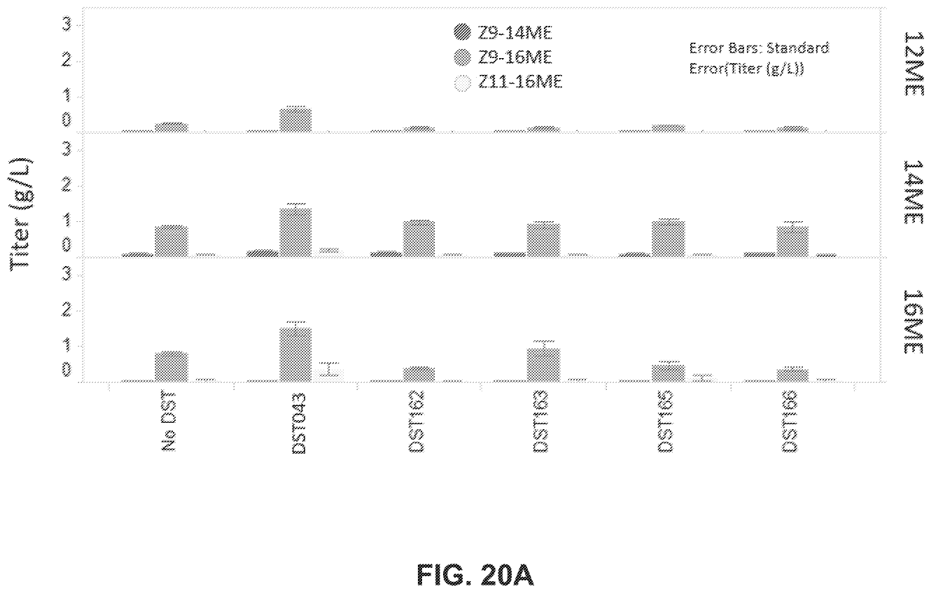

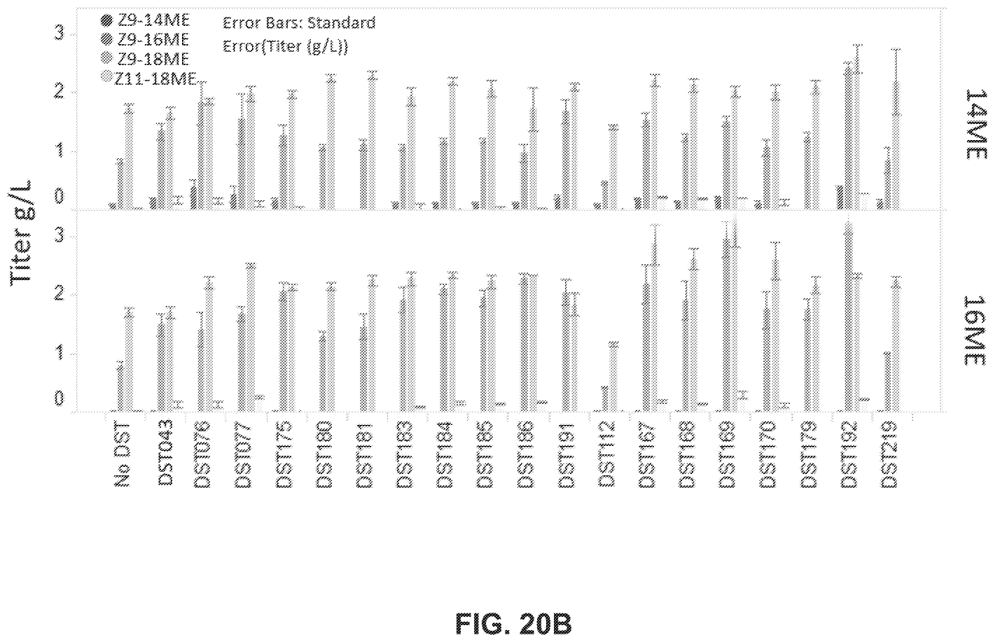

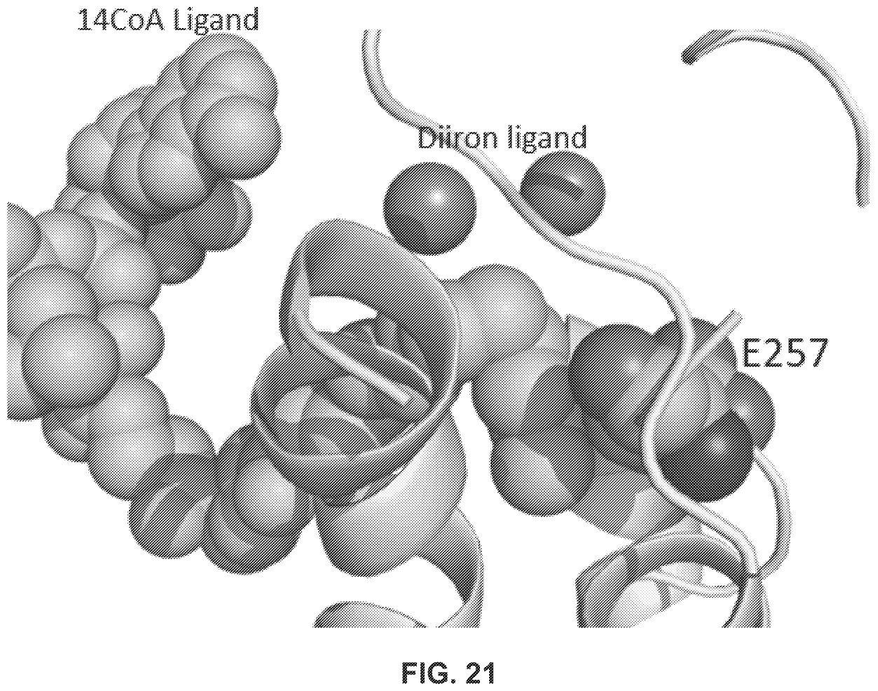

As described above, substitution of a Lys at position 192 for Gly in the representative Z9-14 desaturase DST192 G100L provided E9-14 desaturase activity. FIG. 18. A homology model built for DST192 identified residue E283 as a further determinant of stereoselectivity (FIG. 21). Substitution of amino acids corresponding to G100 and E283 in DST192 and others that are important for determining stereoselectivity confers E9-14 activity in Z9-14 desaturases; DST162 (SEQ ID NO:28), DST163 (SEQ ID NO:29), DST165 (SEQ ID NO:30), DST166 (SEQ ID NO:31), DST076 (SEQ ID NO:32), DST077 (SEQ ID NO:33), DST167 (SEQ ID NO:34), DST168 (SEQ ID NO:35), DST169 (SEQ ID NO:36), DST170 (SEQ ID NO:37), DST171 (SEQ ID NO:38), DST172 (SEQ ID NO:39), DST175 (SEQ ID NO:40), DST179 (SEQ ID NO:41), DST180 (SEQ ID NO:42), DST181 (SEQ ID NO:43), DST183 (SEQ ID NO:44), DST184 (SEQ ID NO:45), DST185 (SEQ ID NO:46), DST186 (SEQ ID NO:47), DST191 (SEQ ID NO:48), and DST219 (SEQ ID NO:49) (FIG. 20(A-B)).

E9Z11-14 Intermediate Production from E9-14

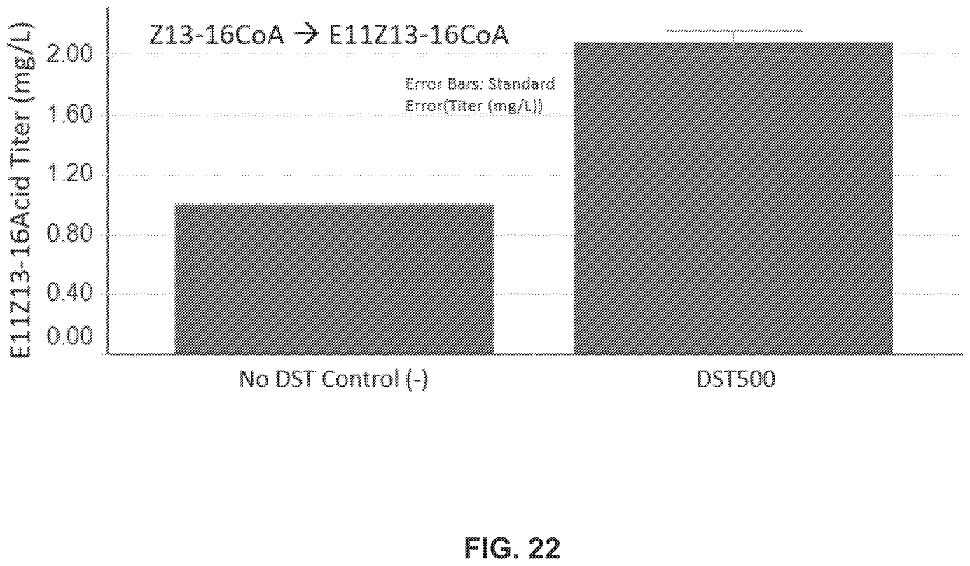

Synthesis of E9Z11-14 was achieved by expression of DST299 (FIG. 5) or conjugase DST500 (FIG. 22).

E7Z9-12 Production from E9Z11-14

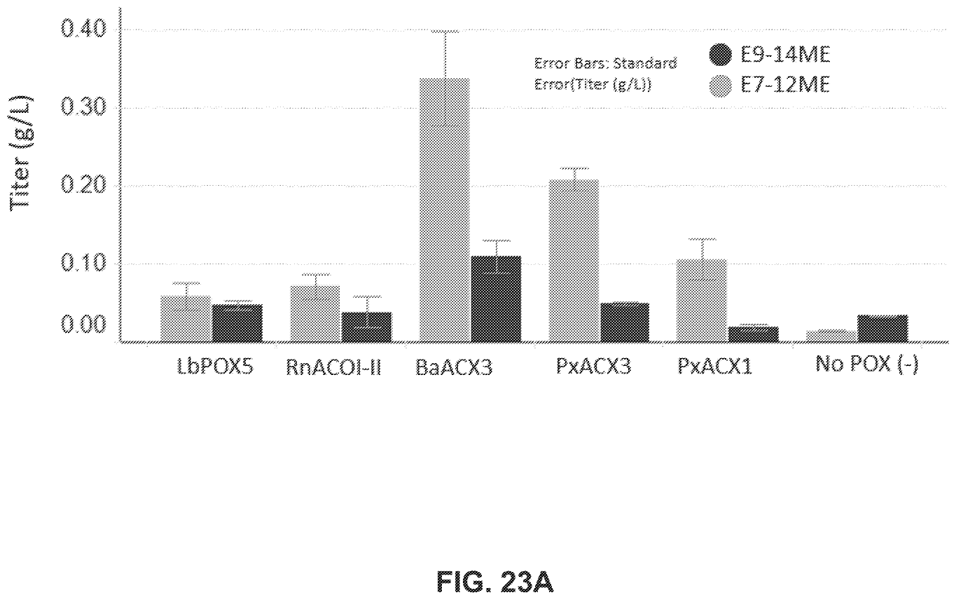

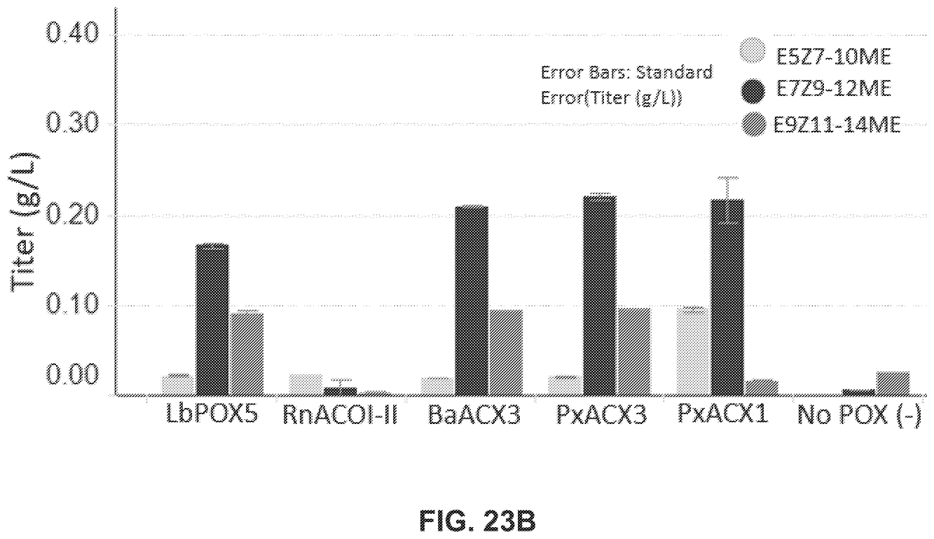

To confer the production of E7Z9-12CoA, which can be converted to bioactive E7Z9-12Ac by chemical or biosynthetic methods, acyl-CoA oxidases were expressed. Expression of RnACOX1, RnACOX2, AtACX1, AtACX2, LbPOX5, BaACX3, PxACX1, and PxACX3 resulted in the bioconversion of E9Z11-14CoA into E7Z9-12CoA. FIG. 8; FIG. 23. Other acyl-CoA oxidases homologous to Y. lipolytica POX2, RnACOX1, RnACOX2, AtACX1, AtACX2, LbPOX, BaACX3, PxACX1, PxACX3, and ACX3 are also suitable. FIG. 23.

### Example 6: Biosynthesis of E7Z9-12CoA from C14 Substrate (Pathway 2)

A second pathway for the production of E7Z9-12 from C14 was engineered by functional co-expression of enzymes with Z11-14 desaturase, conjugase, and acyl-CoA oxidase activities. FIG. 3.

Z11-14 Intermediate Production from C14

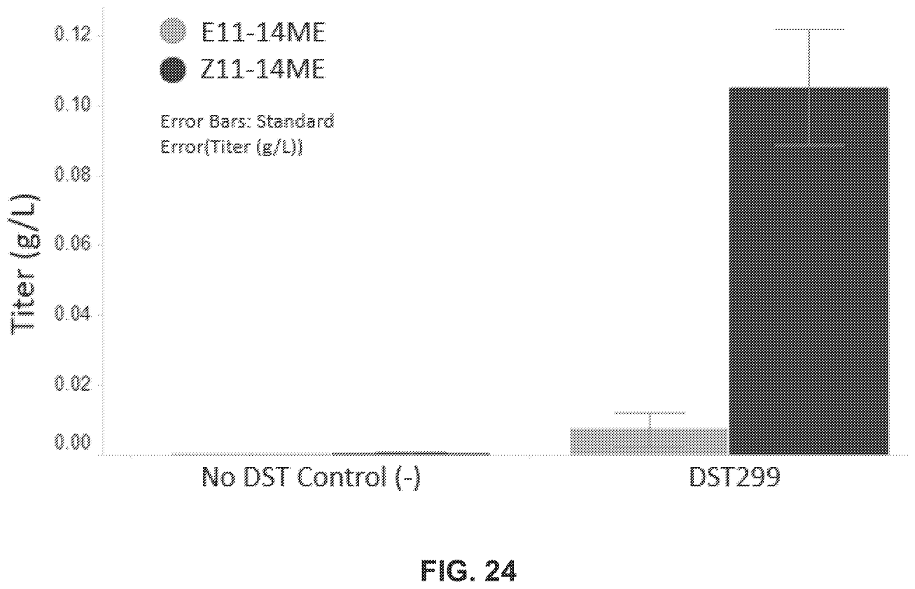

Functional expression of DST299 in Y. lipolytica yielded Z11-14 from 14C. FIG. 24.

E9Z11-14 Intermediate Production from Z11-14

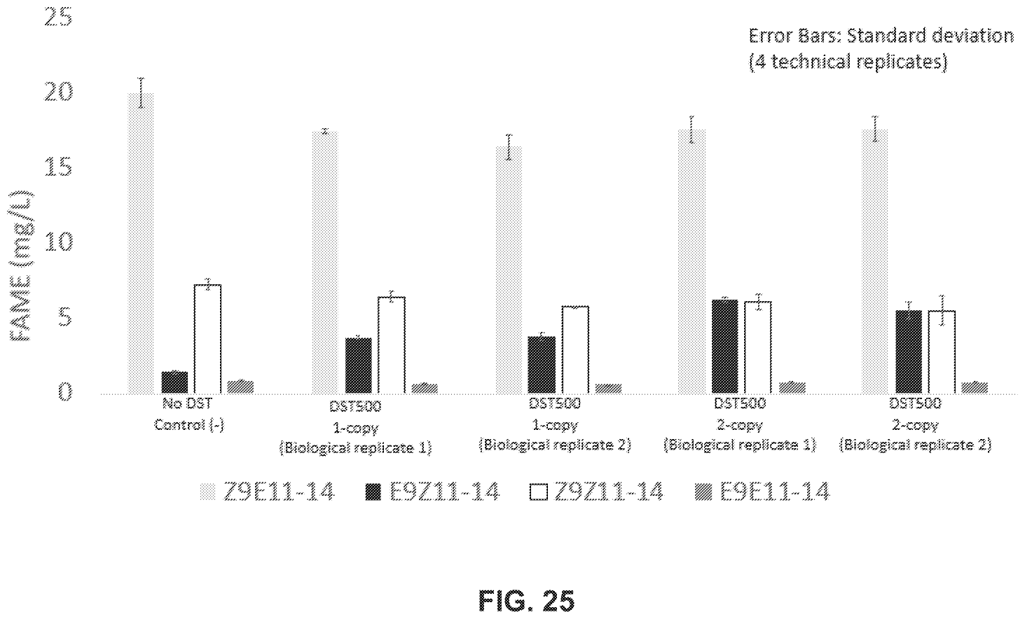

E9Z11-14 production from Z11-14 was achieved by expression of conjugase DST500 from either one or two gene copies. FIG. 25.

E7Z9-12 Production from E9Z11-14

To confer the production of E7Z9-12CoA, which can be converted to bioactive E7Z9-12Ac by chemical or biosynthetic methods, acyl-CoA oxidases were expressed. Expression of RnACOX1, RnACOX2, AtACX1, AtACX2, LbPOX5, BaACX3, PxACX1, and PxACX3 resulted in the bioconversion of E9Z11-14CoA into E7Z9-12CoA. FIG. 8; FIG. 23. Other acyl-CoA oxidases homologous to Y. lipolytica POX2, RnACOX1, RnACOX2, AtACX1, AtACX2, LbPOX, BaACX3, PxACX1, PxACX3, and ACX3 are also suitable. FIG. 23.

### Example 7: Biosynthesis of E7Z9-12CoA from C14 Substrate (Pathway 3)

A third pathway for the production of E7Z9-12 from C14 was engineered by functional co-expression of enzymes with conjugase, isomerase, and acyl-CoA oxidase activities. FIG. 3.

E8E10-14 Intermediate Production from C14

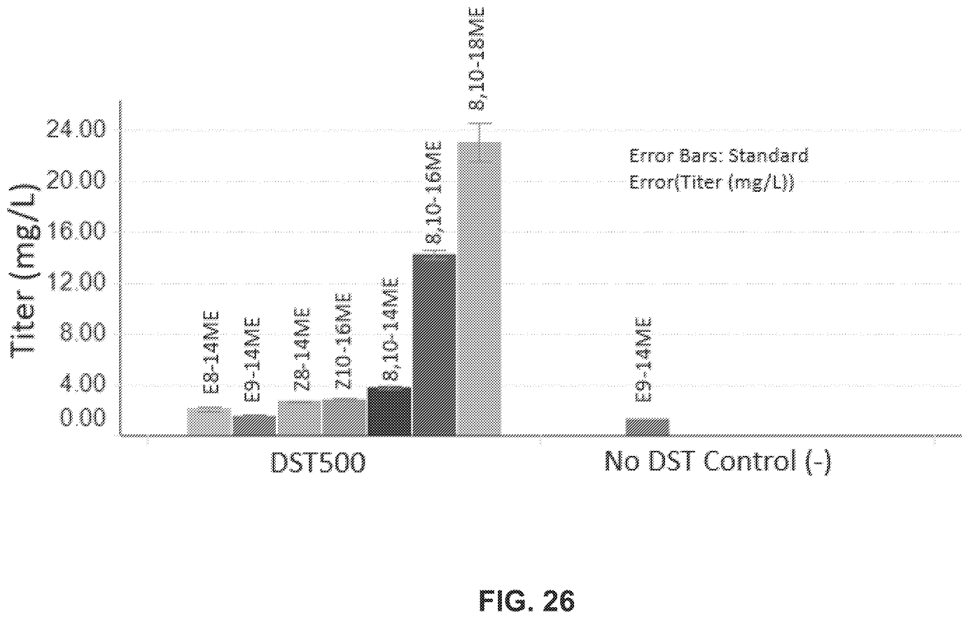

E8E10-14 production from C14 was achieved by expression of conjugase DST500. FIG. 26. E9Z11-14 production is achieved from E8E10-14 by expression of an isomerase with Z11-14 DST activity.

E7Z9-12 Production from E9Z11-14

To confer the production of E7Z9-12CoA, acyl-CoA oxidases were expressed. Expression of RnACOX1, RnACOX2, AtACX1, AtACX2, LbPOX5, BaACX3, PxACX1, and PxACX3 resulted in the bioconversion of E9Z11-14CoA into E7Z9-12CoA. FIG. 8; FIG. 23.

Other acyl-CoA oxidases homologous to Y. lipolytica POX2, RnACOX1, RnACOX2, AtACX1, AtACX2, LbPOX, BaACX3, PxACX1, PxACX3, and ACX3 are also suitable. FIG. 23.

### Example 8: Biosynthesis of E7Z9-12CoA from C14 Substrate (Pathway 3)

A fourth pathway for the production of E7Z9-12 from C14 was engineered by functional co-expression of enzymes with Z11-14 DST, conjugase, and acyl-CoA oxidase activities in a host expressing a Z11-14 elongase. FIG. 3.

Z11-14 Intermediate Production from C14

E8E10-14 production from C14 (14ME) was achieved by expression of DST299.

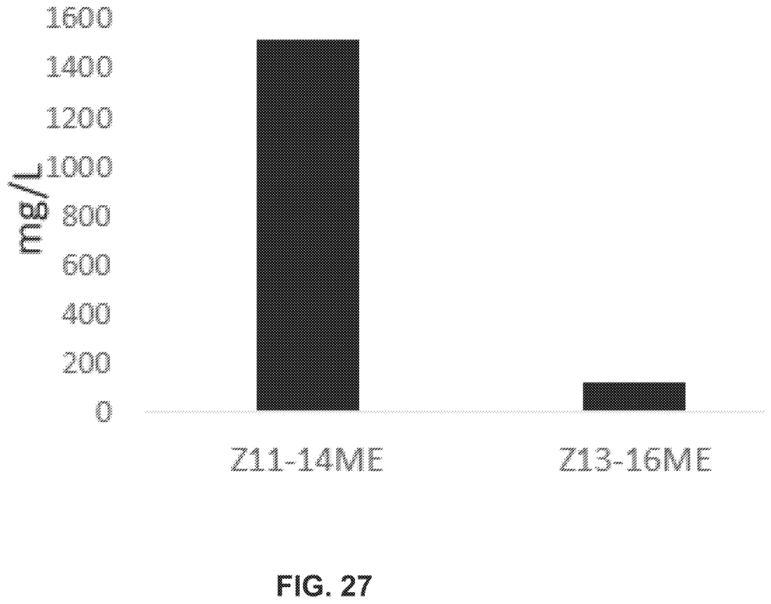

FIG. 27.

Z13-16 Intermediate Production from Z11-14

Z11-14CoA was converted to Z13-16CoA by elongases (i.e., ELO1 and ELO2).

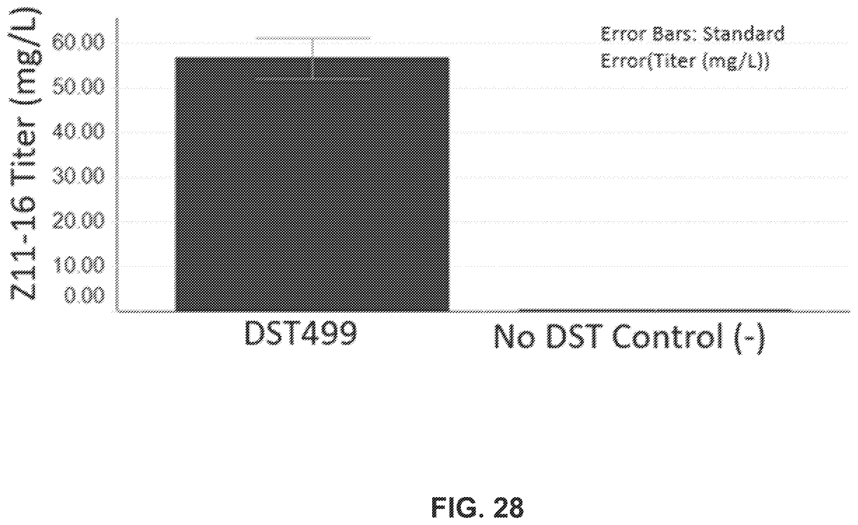

FIG. 28.

E11Z13-16 Intermediate Production from Z13-16

The diene E11Z13-16CoA was synthesized from Z13-16CoA through functional expression of DST500. FIG. 22.

E7Z9-12 Production from E11Z13-16

Expression of acyl-CoA oxidases RnACOX1, RnACOX2, AtACX1, AtACX2, LbPOX5, BaACX3, PxACX1, and PxACX3resulted in the bioconversion of E11Z13-16CoA into E7Z9-12CoA in two steps; oxidation of E11Z13-16CoA to E9Z11-14CoA, and oxidation of E9Z11-14CoA to E7Z9-12CoA (FIG. 8; FIG. 23). Other acyl-CoA oxidases homologous to Y. lipolytica POX2, RnACOX1, RnACOX2, AtACX1, AtACX2, LbPOX, BaACX3, PxACX1, PxACX3, and ACX3 are also suitable. FIG. 23.

### Example 9: Biosynthesis of E7Z9-12CoA from C16 Substrate (Pathway 1)

Multiple pathways for the production of E7Z9-12 from 16C (FIG. 4) were engineered. In a first pathway, the E7Z9-12 product was obtained by functional co-expression of enzymes with Z11-16 desaturase, conjugase, and acyl-CoA oxidase activities. FIG. 4.

Z11-16 Intermediate Production from C16

Conversion from 16ME to Z11-16CoA was achieved in Y. lipolytica through the overexpression of Z11-16 desaturase DST499. FIG. 29. Conversion from Z11-16CoA to E11Z13-16CoA is achieved through expression of a conjugase, such as a DST500 engineered for increased Z11-16 to E11Z13-16 activity.

E7Z9-12 Production from E11Z13-16

Expression of acyl-CoA oxidases RnACOX1, RnACOX2, AtACX1, AtACX2, LbPOX5, BaACX3, PxACX1, and PxACX3 resulted in the bioconversion of E11Z13-16CoA into E7Z9-12CoA in two steps; oxidation of E11Z13-16CoA to E9Z11-14CoA, and oxidation of E9Z11-14CoA to E7Z9-12CoA (FIG. 8; FIG. 23). Other acyl-CoA oxidases homologous to Y. lipolytica POX2, RnACOX1, RnACOX2, AtACX1, AtACX2, LbPOX, BaACX3, PxACX1, PxACX3, and ACX3 are also suitable. FIG. 23.

### Example 10: Biosynthesis of E7Z9-12CoA from C16 Substrate (Pathway 2)

A second pathway for the production of E7Z9-12 from C16 was engineered by functional co-expression of enzymes with E11-16 DST, Z11-16 DST, and acyl-CoA oxidase activities. FIG. 4.

E11-16 Intermediate Production from C16

16ME was converted to E11-16CoA by E11-16CoA desaturase DST109 V230A (See Example 2). E11-16 desaturase activity is also engineered from DST499 or another Z11-16 desaturase by mutating specific amino acid positions important for desaturase stereoselectivity, as determined by the alignments and homology models shown in FIGS. 12-13. Any of several known mutagenesis methods known in the art are used to introduce the mutation. Saturation mutagenesis approaches are utilized in DST499 to provide E11-16 activity..

E11-16CoA is converted to E9-14CoA by an acyl-CoA oxidase, such as RnACOX1, RnACOX2, AtACX1, AtACX2, LbPOX5, BaACX3, PxACX1, and PxACX3. FIG. 23B.

E9Z11-14 Intermediate Production from E9-14

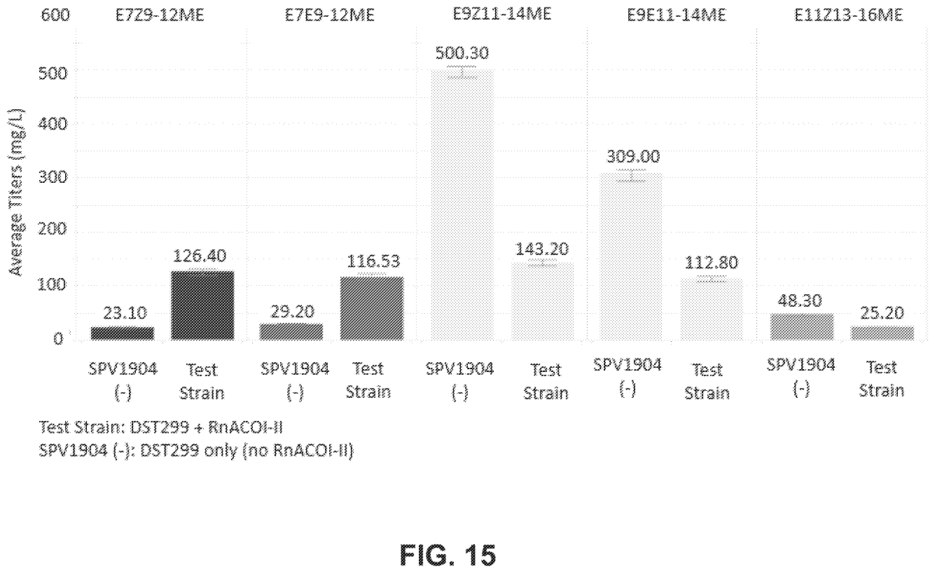

E9Z11-14CoA was produced from E9-14CoA by expression of DST299. FIG. 15.

E7Z9-12 Production from E9Z11-14

Expression of acyl-CoA oxidases conferred the production of E7Z9-12CoA from E9Z11-14CoA; expression of RnACOX1, RnACOX2, AtACX1, AtACX2, LbPOX5, BaACX3, PxACX1, and PxACX3 resulted in the bioconversion of E9Z11-14CoA into E7Z9-12CoA. FIG. 8; FIG. 23. Other acyl-CoA oxidases homologous to Y. lipolytica POX2, RnACOX1, RnACOX2, AtACX1, AtACX2, LbPOX, BaACX3, PxACX1, PxACX3, and ACX3 are also suitable. FIG. 23.

### Example 11: Biosynthesis of E7Z9-12CoA from C16 Substrate (Pathway 3)

A third pathway for the production of E7Z9-12 from C16 was engineered by functional co-expression of enzymes with Z13-16 DST, conjugase, and acyl-CoA oxidase activities. FIG. 4. Alternatively, Z13-16 is derived from jojoba oil.

E11Z13-16 Intermediate Production from C16

Z13-16CoA desaturated from 16ME by a Z13-16 desaturase, or derived from jojoba oil, is converted to E11Z13-16CoA by conjugase DST500, which converted a Z13-16Acid feed to E11Z13-16 (FIG. 22).

E7Z9-12 Production from E11Z13-16

Expression of acyl-CoA oxidases RnACOX1, RnACOX2, AtACX1, AtACX2, LbPOX5, BaACX3, PxACX1, and PxACX3 resulted in the bioconversion of E11Z13-16CoA into E7Z9-12CoA in two steps; oxidation of E11Z13-16CoA to E9Z11-14CoA, and oxidation of E9Z11-14CoA to E7Z9-12CoA (FIG. 8; FIG. 23). Other acyl-CoA oxidases homologous to Y. lipolytica POX2, RnACOX1, RnACOX2, AtACX1, AtACX2, LbPOX, BaACX3, PxACX1, PxACX3, and ACX3 are also suitable. FIG. 23.

1. A genetically modified microorganism that comprises at least one heterologous component of a E7Z9-12CoA biosynthetic pathway selected from the group consisting of:
A) a heterologous Z11-14 desaturase that converts a C14 substrate to Z11-14CoA or E9Z11-14CoA, wherein the Z11-14 desaturase comprises an amino acid sequence that is at least about 90% identical to SEQ ID NO:1, optionally wherein the genetically modified microorganism comprises C14 and/or C16 fatty-acyl CoA oxidase activity;
B) a heterologous Z11-16 desaturase that converts a C16 substrate to Z11-16CoA, wherein the Z11-16 desaturase comprises an amino acid sequence that is at least about 90% identical to SEQ ID NO:2, or comprises a heterologous E11-16 desaturase that converts a C16 substrate to E11-16CoA, wherein the E11-16 desaturase comprises an amino acid sequence that is at least about 90% identical to SEQ ID NO:6, optionally wherein the genetically modified microorganism comprises C14 and/or C16 fatty-acyl CoA oxidase activity;
C) a heterologous conjugase that converts a C14 substrate to E8E10-14CoA or E9Z11-14CoA and converts a C16 substrate to E11Z13-16CoA, wherein the conjugase comprises an amino acid sequence that is at least about 90% identical to SEQ ID NO:111, optionally wherein the genetically modified microorganism comprises C14 and/or C16 fatty-acyl CoA oxidase activity; and/or
D) a heterologous fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115,
optionally wherein the genetically modified microorganism further comprises a heterologous E9-14 desaturase that converts a C14 substrate to E9-14CoA or E9Z11-14CoA, preferably wherein the C14 substrate is 14CoA or Z11-14CoA, wherein the E9-14 desaturase comprises an amino acid sequence that is at least about 90% identical to SEQ ID NO:4, SEQ ID NO:7, SEQ ID NO:8, SEQ ID NO:9, SEQ ID NO:10, SEQ ID NO:11, or SEQ ID NO:12,
optionally wherein the genetically modified microorganism comprises at least one further heterologous polypeptide with Z11-14 desaturase activity, Z13-18 desaturase activity, Z11-16 desaturase activity, Z11-18 desaturase activity, Z9-18 desaturase activity, Z13-16 desaturase activity, Z9-16 desaturase activity, Z9-14 desaturase activity, E11-14 desaturase activity, or E11-16 desaturase activity.

2. The genetically modified microorganism
of claim 1(A), further comprising:
a heterologous E9-14 desaturase that converts a C14 substrate to E9-14CoA or E9Z11-14CoA, optionally wherein the C14 substrate is 14CoA or Z11-14CoA, wherein the E9-14 desaturase comprises an amino acid sequence that is at least about 90% identical to SEQ ID NO:4, SEQ ID NO:7, or SEQ ID NO:8,
a heterologous conjugase that converts a C14 substrate to E8E10-14CoA and E9Z11-14CoA and converts a C16 substrate to E11Z13-16CoA, wherein the conjugase comprises an amino acid sequence that is at least about 90% identical to SEQ ID NO:111, or
a heterologous E11-16 desaturase that converts a C16 substrate to E11-16CoA or E11Z13-16CoA, wherein the E11-16 desaturase comprises an amino acid sequence that is at least about 90% identical to SEQ ID NO:6,
optionally further comprising a heterologous fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115;
of claim 1(B), further comprising:
a heterologous fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115;
of claim 1(C), further comprising:
a heterologous fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115.

3. The genetically modified microorganism of claim or claim 2, wherein the genetically modified microorganism comprises a heterologous polypeptide with Z9-18 desaturase activity comprising an amino acid sequence that is at least about 90% identical to SEQ ID NO:54, a heterologous polypeptide with Z9-16 or Z9-14 desaturase activity comprising an amino acid sequence that is at least about 90% identical to SEQ ID NO:55, or a heterologous polypeptide with Z11-18 or Z11-16 desaturase activity comprising an amino acid sequence that is at least about 90% identical to SEQ ID NO:56.

4. A fermentation culture comprising the genetically modified microorganism of any of claims 1-3.

5. A cell-free reaction volume derived from a cell lysate of the genetically modified microorganism of any of claims 1-3.

6. The genetically modified microorganism of any of claims 1-3, further comprising at least one fatty acyl-CoA oxidase comprising an amino acid sequence that is at least about 90% identical to Y. lipolytica POX1, Y. lipolytica POX2, Y. lipolytica POX3, Y. lipolytica POX4, Y. lipolytica POX5, Y. lipolytica POX6, S. cerevisiae POX1, Candida POX2, Candida POX4, Candida POX5, SEQ ID NO:117, SEQ ID NO:118, SEQ ID NO:119, or SEQ ID NO:120, SEQ ID NO:121, SEQ ID NO:122, SEQ ID NO:123, SEQ ID NO:124, or SEQ ID NO:125.

7. A cell culture comprising the genetically modified microorganism of claim 6, preferably wherein the cell culture is a fermentation culture, more preferably wherein the cell culture is a yeast fermentation culture, most preferably wherein the wherein the cell culture is a fermentation culture comprising Y. lipolytica.

8. The genetically modified microorganism of any of claims 1-3, further comprising at least one enzyme selected from the group consisting of:
fatty acyl-CoA reductases (FAR) preferably wherein the FAR comprises an amino acid sequence that is at least about 90% identical to SEQ ID NO:128, SEQ ID NO:129, SEQ ID NO:130, SEQ ID NO:131, or SEQ ID NO:132;
conjugases, preferably wherein the conjugase is an endogenous enzyme, more preferably wherein the conjugase is a means for 12CoA directly introducing conjugated double bonds at carbons 7 and 9 of 12CoA, or through an intermediate at the central carbon 8 position, to form E7Z9-12CoA, even more preferably wherein the conjugase comprises an amino acid sequence that is at least about 90% identical to SEQ ID NO:112;
elongases (ELOs), preferably wherein the ELO comprises an amino acid sequence that is at least about 90% identical to SEQ ID NO:XXX or SEQ ID NO:XXX;
enoyl-CoA hydratases;
3-hydroxyacyl-CoA dehydrogenases;
a thiolase.

9. A reaction volume comprising at least one component of a E7Z9-12CoA biosynthetic pathway, preferably wherein the reaction volume is a bioreactor comprising cultured genetically modified microorganisms expressing the component(s) of a E7Z9-12CoA biosynthetic pathway as heterologous polypeptides comprises a cell extract, wherein the component(s) of a E7Z9-12CoA biosynthetic pathway are selected from the group consisting of:
I) a Z11-14 desaturase that converts a C14 substrate to Z11-14CoA or E9Z11-14CoA, wherein the Z11-14 desaturase comprises an amino acid sequence that is at least about 90% identical to SEQ ID NO:1, optionally wherein the reaction volume comprises a fatty-acyl CoA oxidase with C14 and/or C16 fatty-acyl CoA oxidase activity;
II) a Z11-16 desaturase that converts a C16 substrate to Z11-16CoA, wherein the Z11-16 desaturase comprises an amino acid sequence that is at least about 90% identical to SEQ ID NO:2, or comprises an E11-16 desaturase that converts a C16 substrate to E11-16CoA or E11Z13-16CoA, wherein the E11-16 desaturase comprises an amino acid sequence that is at least about 90% identical to SEQ ID NO:6, optionally wherein the reaction volume comprises a fatty-acyl CoA oxidase with C14 and/or C16 fatty-acyl CoA oxidase activity;
III) a conjugase that converts a C14 substrate to E8E10-14CoA and E9Z11-14CoA and converts a C16 substrate to E11Z13-16CoA, wherein the conjugase comprises an amino acid sequence that is at least about 90% identical to SEQ ID NO:111, optionally wherein the reaction volume comprises a fatty-acyl CoA oxidase with C14 and/or C16 fatty-acyl CoA oxidase activity; and/or
IV) a fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115,
optionally wherein the reaction volume further comprises an E9-14 desaturase that converts a C14 substrate to E9-14CoA or E9Z11-14CoA, preferably wherein the C14 substrate is 14CoA or Z11-14CoA, wherein the E9-14 desaturase comprises an amino acid sequence that is at least about 90% identical to SEQ ID NO:7, SEQ ID NO:8, SEQ ID NO:9, SEQ ID NO:10, SEQ ID NO:11, SEQ ID NO:4, or SEQ ID NO:12,
optionally wherein the reaction volume comprises at least one further polypeptide with Z11-14 desaturase activity, Z13-18 desaturase activity, Z11-16 desaturase activity, Z11-18 desaturase activity, Z9-18 desaturase activity, Z13-16 desaturase activity, Z9-16 desaturase activity, Z9-14 desaturase activity, E11-14 desaturase activity, or E11-16 desaturase activity.

11. The reaction volume of claim 10, further comprising a E9-14 desaturase that converts a C14 substrate to E9-14CoA or E9Z11-14CoA, wherein the E9-14 desaturase comprises an amino acid sequence that is at least about 90% identical to SEQ ID NO:4 or SEQ ID NO:7.

12. The reaction volume
of claim 10(I), further comprising:
a E9-14 desaturase that converts a C14 substrate to E9-14CoA or E9Z11-14CoA, preferably wherein the C14 substrate is 14CoA or Z11-14CoA, wherein the E9-14 desaturase comprises an amino acid sequence that is at least about 90% identical to SEQ ID NO:4 or SEQ ID NO:7,
a conjugase that converts a C14 substrate to E8E10-14CoA and E9Z11-14CoA and converts a C16 substrate to E11Z13-16CoA, wherein the conjugase comprises an amino acid sequence that is at least about 90% identical to SEQ ID NO:111, or
an E11-16 desaturase that converts a C16 substrate to E11-16CoA or E11Z13-16CoA, wherein the E11-16 desaturase comprises an amino acid sequence that is at least about 90% identical to SEQ ID NO:6,
preferably further comprising a fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115;
of claim 10(II), further comprising:
a fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115;
of claim 10(III), further comprising:
a fatty acyl-CoA oxidase that converts E9Z11-14CoA to E7Z9-12CoA and converts E11Z13-16CoA to E9Z11-14CoA, wherein the fatty acyl-CoA oxidase comprises an amino acid sequence that is at least about 90% identical to SEQ ID NO:112, SEQ ID NO:113, SEQ ID NO:114, or SEQ ID NO:115.

13. The reaction volume of any of claims 10-12, wherein the reaction volume comprises a polypeptide with Z9-18 desaturase activity comprising an amino acid sequence that is at least about 90% identical to SEQ ID NO:54, a polypeptide with Z9-16 or Z9-14 desaturase activity comprising an amino acid sequence that is at least about 90% identical to SEQ ID NO:55, or a polypeptide with Z11-18 or Z11-16 desaturase activity comprising an amino acid sequence that is at least about 90% identical to SEQ ID NO:56.

14. The reaction volume of any of claims 10-12, further comprising at least one fatty acyl-CoA oxidase comprising an amino acid sequence that is at least about 90% identical to Y. lipolytica POX1, Y. lipolytica POX2 (SEQ ID NO:125), Y. lipolytica POX3, Y. lipolytica POX4, Y. lipolytica POX5, Y. lipolytica POX6, S. cerevisiae POX1, Candida POX2, Candida POX4, Candida POX5, SEQ ID NO:116, SEQ ID NO:117, SEQ ID NO:118, or SEQ ID NO:119, SEQ ID NO:120, SEQ ID NO:121, SEQ ID NO:122, SEQ ID NO:123, or SEQ ID NO:124.

15. The reaction volume of any of claims 10-12, further comprising at least one enzyme selected from the group consisting of:
fatty acyl-CoA reductases (FARs), preferably wherein the FAR comprises an amino acid sequence that is at least about 90% identical to SEQ ID NO:128, SEQ ID NO:129, SEQ ID NO:130, SEQ ID NO:131, or SEQ ID NO:132;
conjugases, preferably wherein the conjugase is a means for 12CoA directly introducing conjugated double bonds at carbons 7 and 9 of 12CoA, or through an intermediate at the central carbon 8 position, to form E7Z9-12CoA, even more preferably wherein the conjugase comprises an amino acid sequence that is at least about 90% identical to SEQ ID NO:111;
elongases (ELOs), preferably wherein the ELO comprises an amino acid sequence that is at least about 90% identical to SEQ ID NO:126 or SEQ ID NO:127;
enoyl-CoA hydratases;
3-hydroxyacyl-CoA dehydrogenases; and
thiolases.

16. A biosynthetic method for producing an insect pheromone or insect pheromone precursor, the method comprising:
culturing at least one genetically modified microorganism of any of claims 1-3; and
feeding the culture with substrate fatty acids or fatty alkyl esters, preferably wherein the substrate fatty acids or fatty acid alkyl esters are C12, C14, or C16 fatty acids or fatty acid alkyl esters, and wherein
i) E11Z13-16ME, E9Z11-14ME, or E7Z9-12ME is produced in the culture from substrate fatty acids and fatty alkyl esters that are 16CoA, E11-16Acid, or E11-16ME,
ii) E9Z11-14ME or E7Z9-12ME is produced in the culture from substrate fatty acids and fatty alkyl esters that are 14CoA, E9-14Acid, or E9-14ME, or
iii) E7Z9-12ME is produced in the culture from substrate fatty acids and fatty alkyl esters that are 12CoA, E7-12Acid, or E7-12ME,

17. The biosynthetic method according to claim 16, wherein the substrate fatty acids or fatty alkyl esters comprise a plurality of different fatty acids or alkyl esters with different carbon chain lengths and or stereoisomers, wherein the different fatty acids or alkyl esters are fed sequentially to the culture, or fed simultaneously to the culture.

18. The biosynthetic method according to claim 16, wherein a methyl ester produced by the method is acetylated.

19. The biosynthetic method according to claim 18, wherein the methyl ester produced by the method is acetylated biologically in the genetically modified microorganism

20. A method for producing an insect pheromone or insect pheromone precursor, the method comprising:
introducing substrate fatty acids or fatty alkyl esters into the reaction volume of any of claims 10-12, thereby producing the insect pheromone or insect pheromone precursor in the reaction volume, preferably wherein the substrate fatty acids or fatty acid alkyl esters are C12, C14, or C16 fatty acids or fatty acid alkyl esters, and wherein
a) E11Z13-16ME, E9Z11-14ME, or E7Z9-12ME is produced in the reaction volume from substrate fatty acids and fatty alkyl esters that are 16CoA, E11-16Acid, or E11-16ME,
b) E9Z11-14ME or E7Z9-12ME is produced in the reaction volume from substrate fatty acids and fatty alkyl esters that are 14CoA, E9-14Acid, or E9-14ME, or
c) E7Z9-12ME is produced in the reaction volume from substrate fatty acids and fatty alkyl esters that are 12CoA, E7-12Acid, or E7-12ME,
preferably further comprising isolating the insect pheromone or insect pheromone precursor from the reaction volume.

21. The method according to claim 20, wherein the substrate fatty acids or fatty alkyl esters comprise a plurality of different fatty acids or alkyl esters with different carbon chain lengths and or stereoisomers, wherein the different fatty acids or alkyl esters are introduced sequentially to the culture, or introduced simultaneously to the culture.

22. Compound E11Z13-16ME, E11Z13-16CoA, E9Z11-14ME, E9Z11-14CoA, E7Z9-12ME, E7Z9-12CoA, or E7Z9-12Ac, obtained by the method according to claim 16.

23. The compound of claim 22, wherein the compound is E7Z9-12Ac.

24. An insect pest-protective formulation comprising the compound of claim 23.

25. The insect pest-protective formulation of claim 24, wherein the formulation further comprises one or more proteins, polypeptides, or small molecules which is toxic to the insect pest.
